(19) United States
(12) Patent Application Publication
(10) Pub. No.: US 2021/0170033 A1
(43) Pub. Date: Jun. 10, 2021

(54) NOVEL CONJUGATES OF A PHARMACEUTICAL AGENT AND A MOIETY CAPABLE OF BINDING TO A GLUCOSE SENSING PROTEIN

(71) Applicant: SANOFI, Paris (FR)

(72) Inventors: Stefan PETRY, Frankfurt am Main (DE); Elisabeth DEFOSSA, Frankfurt am Main (DE); Michael PODESCHWA, Frankfurt am Main (DE); Volker DERDAU, Frankfurt am Main (DE); Hans MATTER, Frankfurt am Main (DE); Nis HALLAND, Frankfurt am Main (DE); Kaihui HU HE, Frankfurt (DE); Matthias DREYER, Frankfurt am Main (DE); Maximilian BIELOHUBY, Frankfurt (DE); Melissa BESENIUS, Frankfurt (DE); Meltsje DE HOOP, Frankfurt am Main (DE)

(21) Appl. No.: 16/768,600

(22) PCT Filed: Nov. 30, 2018
(86) PCT No.: PCT/EP2018/083077

§ 371 Date: May 29, 2020

(30) Foreign Application Priority Data
Dec. 1, 2017 (EP) 17306673.9

(51) Int. Cl. A61K 47/54; A61P 3/10; C07H 15/18; C07H 13/08; A61K 38/28

(52) U.S. Cl. CPC A61K 47/549; A61P 3/10; A61K 38/28; C07H 13/08; A61K 47/545; C07H 15/18

(57) ABSTRACT

The invention describes novel conjugates of formula (I) of a pharmaceutical agent and a moiety capable of binding to a glucose sensing protein allowing a reversible release of the pharmaceutical agent depending on the glucose concentration.

The invention describes novel conjugates of a pharmaceutical agent and a moiety capable of binding to a glucose sensing protein allowing a reversible release of the pharmaceutical agent depending on the glucose concentration.

Over the last decades the number of patients suffering from diseases, particularly from type 1 or type 2 diabetes, has increased dramatically. Despite education and treatment the growth rate is exploding. The disease evolves slowly and in the beginning the pancreas can compensate decreasing insulin sensitivity by an increased release of insulin. At this stage oral antidiabetics like insulin sensitizers and—releasers can support this compensation mechanism, but cannot cure the disease. So after this period of time external insulin has to be injected.

Several insulins are on the market, which are classified by their duration of action. The intrinsic danger of hypoglycemia is counteracted by very flat insulin profiles (so called basal insulins), but is neither conceptionally addressed nor finally overcome by these basal insulins.

The development of a real glucose sensing insulin accomplishing a glucose dependent release from a depot simulating the natural release by the pancreas is still one of the holy grails in diabetes research. Such an insulin would generate a local (e.g. intraparenteral) or moving depot (blood stream) from where it is released in a glucose concentration dependent manner and finally recaptured by the system on decreasing glucose concentrations.

The blood glucose concentration is under hormonal regulation. While several hormones like glucagon, epinephrine, norepinephrine, cortisol, and hormones from the thyroid gland provoke elevated glucose levels, insulin is the only hormone which lowers glucose levels. In addition the glucose level is of course influenced by timing and composition of meals, physical stress, and infections.

In healthy persons the fasting blood glucose level is around 5 mM (900 mg/L) and can after a meal increase to 40 mM for several hours. In diabetic patients where blood glucose is out of control, the level can vary between 1-30 mM and can unpredictable fluctuate between the borders of hyperglycemia (>10 mM) and hypoglycemia (<3 mM). Despite the possibility of exact blood glucose measurement and titration of insulins, hypoglycemia is still a serious problem. This problem can be solved by glucose sensitive and—responsive delivery of pharmaceutical agents affecting the glucose level.

Non glucose-sensitive depots to protect drugs (small molecules and proteins like insulin) from degradation and elongate their half-life are used frequently in medicine. For insulin for example a static subcutaneous depot can be realized. Insulin is stored as insoluble hexamers. From this depot soluble monomers are released to the blood following law of mass equation.

An additional opportunity is the non-covalent binding of modified insulins to albumin. Since unmodified insulin is not binding to albumin, noncovalent hydrophobic binding is enabled by hydrophobic modification (e.g. by myristic acid). Coupling of fatty acids to insulin enable protection of insulin from degradation and dramatically increases half-life by hours to days.

The release of insulin from such a circulating depot can be described by the law of mass equation and is a function of the amount of insulin, the albumin depot, and the affinity of the insulin derivative to albumin. Since the depot is fixed, the amount and affinity of insulin have to be adjusted. The release of basal insulin can be controlled, but the release is glucose independent.

Within the last decade efforts have been started to establish glucose sensitive insulin depots. These efforts can be summarized and assigned to three classical principles:
Chemical recognition of glucose by boronic acids
Biochemical recognition of glucose by carbohydrate binding proteins such as lectins (Concanavalin A, wheat germ agglutinin)
Glucose converting enzymes like glucose oxidase or hexokinase. Here binding affinity can be used as a signal. More frequently associated pH shift or change of charge is measured.

These principles can be used for glucose measurement or to translate the signals into direct or indirect glucose release. Four possibilities for realization are described below.
Direct modification of insulins
“Glucose responsive” hydrogels, these are synthetic pores, which are modified with a glucose sensing molecule (boronic acid- or glucose oxidase based). These gels are filled with insulins. In the presence of glucose they expand, get leaky, and finally release insulin on increasing glucose levels.
“Device-approaches”: In this case insulin levels are only measured by a sensor.
Closed loop approaches: This describes a technical solution. A sensor measures glucose levels. The signal is transmitted to an independent insulin depot (e.g. a pump) which releases insulin triggered by the signal. An independent insulin reservoir is triggered and releases insulin, controlled by the sensor signal. An advantage may be a large insulin depot which is not necessarily in the body.

Several patent applications, e.g. WO2001/92334, WO2011/000823, or WO2003/048195 describe the use of boronic acid modified insulin derivatives in combination with albumin for a glucose sensitive insulin release. With this approach the floating insulin/albumin depot shall be further developed to a glucose sensing floating depot.

A different approach to glucose sensing insulin has been described in WO2010/088294, WO2010/88300, WO2010/107520, WO2012/015681, WO2012/015692, or WO2015/051052. These documents describe the concomitant administration of concanavalin A and a glucose binding protein preferably recognizing mannose.

Accordingly mannose modified insulins can be released by mannose from a depot. In addition an intrinsic mannose binding protein is described which may be responsible for the binding of mannose without the need of concanavalin.

Erythrocytes have been used as a vehicle for the transport of drugs, e.g. for tumor starvation, enzyme replacement and immunotherapy as described in WO2015/121348, WO2014/198788, and WO2013/139906.

Liu et al. (Bioconjugate Chem. 1997, 8, 664-672) discloses a glucose induced release of glucosylpoly (ethylene glycol) insulin bound to a soluble conjugate of concanavalin A wherein the insulin is linked at the B1 amino group with a poly (ethylene glycol) spacer to the 1-position of the sugar.

WO2012/177701 discloses conjugates of ⁶⁸Ga-DOTA labelled sugars for tissue specific disease imaging and radiotherapy.

WO2017/124102 discloses a glucose modified insulin for reversible binding to glucose transport proteins on erythrocytes.

The use of erythrocytes as a classical depot, by binding drugs to the surface of erythrocytes is described in WO 2013/121296. Here peptides are described, which bind to the surface with a very high affinity (K_{D}=6.2 nM). These peptides are used for immunomodulation e.g. in transplantation medicine.

WO2010/012153 discloses a phlorizin derivative, which are said to inhibit SGLT2 inhibitory activity and be used to treat metabolic diseases such as diabetes and its complications.

WO2010/031813 is entitled “Glycoside Derivatives and Uses thereof” and states that the compounds disclosed therein can be used in the treatment of metabolic disorders.

WO2009/121939 is entitled “C-Aryl Glycoside Compounds for the Treatment of Diabetes and Obesity”.

The present invention relates to a novel conjugate comprising a pharmaceutical agent and a sugar moiety.

Further the present invention relates to a novel conjugate comprising a pharmaceutical agent and a sugar moiety for use as a pharmaceutical.

Further the present invention relates to a novel conjugate comprising a pharmaceutical agent and a sugar moiety which binds to the insulin dependent glucose transporter GLUT1, which provides a release of the pharmaceutical agent dependent on the glucose concentration in blood. The insulin dependent glucose transporter GLUT1 is present on human erythrocytes. Binding of glucose to GLUT1 is reversible based on the blood glucose concentration.

In one embodiment the conjugate of the invention is bound to GLUT1 at low glucose concentrations of e.g. 1-10 mM, which are found under fasting conditions. Under these conditions, the stable floating depot of the active agent is formed. After an increase in the glucose concentration from e.g. 30 mM to 40 mM after a meal, the free glucose competes for the GLUT1 binding site and the conjugate is released in a glucose concentration dependent manner and the pharmaceutical agent is available to exert its effect. As the glucose concentration decreases again, the conjugate molecules are recaptured by GLUT1. Thus, the presence of undesired high amounts of free pharmaceutical agents is avoided.

The present invention relates to conjugates of formula (I):

P-[L₁]ₘ-[A₁]ₒ-[L₂]ₚ-[A₂]-[L₃]-[A₃]-[L₄]-S (I)

wherein P is an insulin or an insulinotropic peptide,
L₁ and L₂ are independently of each other a linker having a chain length of 1-25 atoms,
L₃ is a linker having a chain length of 2 or 3 atoms,
and L₄ is a linker having a chain length of 1, 2 or 3 atoms,
A₁ is a 5 to 6 membered monocyclic ring or a 9 to 12 membered bicyclic ring, wherein each ring is independently a saturated, unsaturated, or aromatic carbocyclic or heterocyclic ring and wherein each ring may carry at least one substituent,
A₂ and A₃ are independently of each other a 5 to 6 membered monocyclic ring or a 9 to 12 membered bicyclic ring, wherein each ring is independently an aromatic carbocyclic or aromatic heterocyclic ring and wherein each ring may carry at least one substituent,
S is a sugar moiety which binds to the insulin independent glucose transporter GLUT1, and
m, o, and p are independently of each other 0 or 1,
or a pharmaceutically acceptable salt or solvate thereof.

The present invention relates also to conjugates of formula (I):

P-[L₁]ₘ-[A₁]ₒ-[L₂]ₚ-[A₂]-[L₃]-[A₃]-[L₄]-S (I)

wherein P is an insulin or an insulinotropic peptide,
L₁ and L₂ are independently of each other a linker having a chain length of 1-25 atoms,
L₃ is a linker having a chain length of 2 or 3 atoms,
and L₄ is a linker having a chain length of 1, 2 or 3 atoms,
A₁, is a 5 to 6 membered monocyclic ring or a 9 to 12 membered bicyclic ring, wherein each ring is independently a saturated, unsaturated, or aromatic carbocyclic or heterocyclic ring and wherein each ring may carry at least one substituent,
A₂ and A₃ are independently of each other a 5 to 6 membered monocyclic ring or a 9 to 12 membered bicyclic ring, wherein each ring is independently an aromatic carbocyclic or aromatic heterocyclic ring and wherein each ring may carry at least one substituent,
S is a sugar moiety which binds to the insulin independent glucose transporter GLUT1, and comprises a terminal pyranose moiety which is attached via position 2, 3, 4, or 6 to L₄, and
m, o, and p are independently of each other 0 or 1,
or a pharmaceutically acceptable salt or solvate thereof.

Another aspect of the invention are compounds of formula (Ia) and (Ib):

R—(O═C)-[L₁]ₘ-[A₁]ₒ-[L₂]ₚ-[A₂]-[L₃]-[A₃]-[L₄]-S (Ia)

[L₁]ₘ-[A₁]ₒ-[L₂]ₚ-[A₂]-[L₃]-[A₃]-[L₄]-S (Ib)

wherein L₁, L₂, L₃, L₄, A₁, A₂, A₃, S, m, o, and p are defined as indicated above and R is H, halogen, OH, O-alkyl-, an anhydride forming group or another active ester forming group for coupling reactions, like 4-nitrophenylester, succinate or N-hydroxy benzotriazol.
or pharmaceutically acceptable salts or solvates thereof.

Compounds (Ia) and (Ib) are suitable as intermediates for the synthesis of the conjugates of formula (I).

Another aspect of the present invention is the conjugate of formula (I) as described above for the use in medicine, particularly in human medicine.

Another aspect of the present invention is a pharmaceutical composition comprising a conjugate of formula (I) as described above as an active agent and a pharmaceutically acceptable carrier.

Another aspect of the present invention is a method of preventing and/or treating a disorder associated with, caused by, and/or accompanied by a dysregulated glucose metabolism, comprising administering a conjugate of formula (I) or a composition as described above to a subject in need thereof, particularly a human patient.

Another aspect of the present invention is a method of preventing and/or treating diabetes type 1 or diabetes type 2.

The conjugates of formula (I) of the present invention comprise a pharmaceutical agent P, which is an insulin or an insulinotropic peptide directly or indirectly lowering the glucose concentration in blood.

The term “insulin” according to the present invention encompasses human insulin, porcine insulin, or analogs thereof, e.g. prandial insulins with fast action or basal insulins with long action. For example, the term “insulin” encompasses recombinant human insulin, insulin glargine, insulin detemir, insulin glulisine, insulin aspart, insulin lispro, etc. If P is an insulin, it may be attached via an amino group to form the conjugate of formula (I), e.g. via an amino side chain, particularly via the amino side chain of an insulin B29Lys residue or via the amino terminus of an insulin B1Phe residue.

Further, the pharmaceutical agent may be an insulinotropic peptide such as GLP-1, an exendin such as exendin-4, or a GLP-1 agonist such as lixisenatide, liraglutide.

The conjugate of formula (I) further comprises a sugar moiety which binds to the insulin independent glucose transporter GLUT1, also known as solute carrier family 2, facilitated glucose transporter member 1 (SLC2A1). The amino acid sequence of the human protein is NP_006507, which is encoded by a nucleic acid sequence NM_006516. GLUT1 is an integral membrane protein which facilitates diffusion of glucose into the erythrocyte. The highest expression of GLUT1 is found on the membrane of erythrocytes.

For interaction with GLUT1, the conjugate of formula (I) comprises a moiety binding to GLUT1 but preventing transport through the erythrocyte membrane. A sugar moiety binding to GLUT1 is preferably in an anomeric form, particularly in an anomeric 6-membered ring form such as a pyranose moiety. The sugar moiety typically comprises an anomeric O atom as well as a hydroxy group or a protected hydroxy group at position 3 and position 4 of a pyranose backbone. In one embodiment, the sugar moiety S of the conjugate of formula (I) comprises a terminal pyranose moiety which is attached via position 2, position 3, position 4, or position 6 of the pyranose backbone moiety.

Further, an aspect of the present invention is that introduction of two aromatic cyclic residues A₂ and A₃ connected by the short linker L₃ and wherein A₃ is adjacent to the sugar moiety by the short linker L₄ cause a substantial increase in the affinity to GLUT1 in comparison to glucose.

Thus, the present invention provides a pharmaceutical agent in form of a conjugate of formula (I) which forms an erythrocyte-based circulating depot that after administration releases/delivers the agent as a function of glucose concentration. Accordingly at low glucose concentrations (below 3 mM) no or only low concentration of free unbound levels of the conjugate should be detectable. On increasing blood glucose levels after a meal the conjugate is released from the circulating depot into the blood stream. The release is a consequence of a direct competition of glucose with the conjugate of formula (I). Thus, release is described by the law of mass equation and self-adjusted to tiniest changes in glucose levels. The same should be true for the re-capturing process of the conjugate of formula (I) on decreasing glucose levels.

These characteristics constitute an essential advantage in comparison to the glucose sensing depots from the prior art.

By means of the present invention, the drawbacks of prior art insulins with regard to glycemia are diminished or avoided. The control of glucose recognition and associated release/retrapping will be realized within a single molecule. This minimizes delays in release/retrapping. Glucose sensitive binding and—release is controlled by interaction with endogenous transport and recognition processes. The biological recognition system based on GLUT1 transport in erythrocytes is constantly regenerated by the organism.

The present conjugate of formula (I) binds to the ubiquitary glucose transporter GLUT1, which has a binding affinity to glucose in the same range as glucose oxidase, a protein frequently used in glucose recognition. GLUT1 is highly expressed in erythrocytes and is responsible for the basal supply of these cells. The size of the depot is large enough to accommodate the amount of pharmaceutical agent needed without affecting the erythrocyte glucose supply.

The affinity of the present conjugate of formula (I) is within an affinity window which guarantees binding at low (e.g. <3 mM) glucose levels. With increasing glucose levels (e.g. >10 mM) the conjugate of formula (I) is released accordingly. With decreasing glucose levels the unbound conjugate of formula (I) is recaptured by the transporter.

The release is following the law of mass equation and is dependent on the size of the depot, the loading, and the affinity of the conjugate of formula (I) to GLUT1. Since the depot is fixed, the free conjugate fraction is defined by the affinity to GLUT1.

In certain embodiments, the conjugate of formula (I) has an affinity of 10-500 nM to the insulin independent glucose transporter GLUT1 as determined by affinity measurements for example by a ligand displacement assay, by MST (microscale thermophoresis) technology.

In the conjugate of formula (I) of the present invention, the individual structural moieties P, A₁, A₂, A₃ and S may be connected by linkers L₁, L₂, L₃ and L₄.

If present, L₁ and L₂ are linkers having a chain length of 1-25 atoms, particularly 3 to 20 atoms, 3 to 10 atoms, or 3 to 6 atoms.

In some embodiments, L₁ and L₂ are independently of each other (C₁-C₂₅) alkylene, (C₂-C₂₅) alkenylene, or (C₂-C₂₅) alkynylene, wherein one or more C-atoms may be replaced by heteroatoms or heteroatom moieties, selected from O, NH, NH—BOC, N(C₁₋₄) alkyl, S, SO₂, O—SO₂, O—SO₃, O—PHO₂ or O—PO₃, and/or wherein one or more C-atoms may be substituted with (C₁₋₄) alkyl, (C₁₋₄) alkyloxy, oxo, carboxyl, halogen, e.g. F, Cl, Br, or I, or a phosphorus-containing group. The carboxyl group may be a free carboxylic acid group or a carboxylic acid ester, e.g. C₁-C₄ alkyl ester or a carboxamide or mono(C₁-C₄) alkyl or di(C₁-C₄) alkyl carboxamide group. An example of a phosphorus-containing group is a phosphoric acid or phosphoric acid (C₁₋₄) alkyl ester group.

In one embodiment the linker L₁ is —CO—(C₁-C₆)alkylene-, —CO—(C₁-C₄)ₓalkylene-(—CH₂—CH₂—O)_{y}—(C₂-C₆)alkylene or —CO—(C₁-C₄)ₓalkylene-(O—CH₂—CH₂)_{y}—NH—CO—(C₂-C₄) alkylene-(O—CH₂—CH₂)_{z}—NH—CO—, wherein x, y and z are independently of each other 0, 1, 2, 3 or 4 and wherein the chain length of L₁ is equal or less than 25 atoms.

In one embodiment, the linker L₁ is —CO—(CH₂)₃—, —CO—(CH₂)₅— or —CO—(CH₂—CH₂—O)₂—CH₂—CH₂—.

In one embodiment, the linker L₁ is —CO—CH₂—(O—CH₂—CH₂)₂—NH—CO—CH₂—(O—CH₂—CH₂)₂—NH—CO—.

In one embodiment, the linker L₂ is —(C₂-C₆) alkylene-CO—NH— or —(C₂-C₆) alkylene.

In one embodiment, the linker L₂ is —(CH₂)₂—CO—NH—, —(CH₂)₃—CO—NH—, —(CH₂)₃— or —CH₂—CH₂—.

In certain embodiments, the linker L₃ has a chain length of 2 to 3 atoms, For example L₃ may be a (C₂-C₃) alkylene, particularly a (C₂) alkylene group, wherein one C-atom may be replaced by a heteroatom or heteroatom moiety, particularly by O, NH, N(C₁₋₄) alkyl, S, SO₂, O—SO₂, O—SO₃, O—PHO₂ or O—PO₃, or one C-atom may be substituted by oxo.

In another embodiments, the linker L₃ is selected from —CH₂—CH₂—CH₂—, —CH₂—CH₂—, —CH₂—CH₂—O—, —O—CH₂—CH₂—, —CH₂—O—, —O—CH₂—, —CO—O—, —O—CO—, —CO—NH or —NH—CO—.

In another embodiments, the linker L₃ is selected from —CH₂—CH₂—O—, —CH₂—O—, —CO—O—or —CO—NH.

In another embodiments, the linker L₃ is selected from —CH₂—O—, —CO—O—, or —CO—NH.

In certain embodiments, the linker L₄ has a chain length of 1 to 3 or 1 to 2 atoms. For example, L₄ may be a (C₁-C₃) alkylene, particularly (C₁₋₂) alkylene group, wherein one or two C-atoms may be replaced by heteroatoms or heteroatom moieties, particularly by O, NH, N(C₁₋₄) alkyl, S, SO₂, O—SO₂, O—SO₃, O—PHO₂ or O—PO₃, and/or wherein one C-atoms may be substituted with (C₁₋₄) alkyl, (C₁₋₄) alkyloxy, oxo, carboxyl, or a phosphorus-containing group.

In one embodiment, the linker L₄ is —CO—O—. In another embodiment, the linker L₄ is —CO—NH—.

The conjugate of formula (I) of the present invention comprises at least two cyclic aromatic groups, particularly A₂ and A₃. An aspect of the present invention is that the presence of the two cyclic groups connected to each other with a short linker L₃ and wherein A₃ is adjacent to the sugar moiety S by the short linker L₄ significantly enhances the binding affinity of the sugar moiety S to the glucose transporter GLUT1. The cyclic groups A₂ and A₃ are independently of each other a 5 to 6 membered monocyclic ring, a 9 to 12 membered bicyclic ring, wherein each ring is an aromatic carbocyclic or aromatic heterocyclic ring and wherein each ring is independently of each other unsubstituted or substituted by 1 to 4 substituents selected from halogen, NO₂, CN, CF₃, —OCF₃, (C₁₋₄) alkyl, (C₁₋₄) alkoxy, (C₁₋₄)alkyl-(C₃₋₇)cycloalkyl, (C₃₋₇) cycloalkyl, OH, benzyl, —O-benzyl, carboxyl, (C₁₋₄) alkyl-carboxylester, carboxamide, —SO₂Me, NH₂, NH—BOC or mono (C₁₋₄) alkyl, or di (C₁₋₄) alkyl carboxamide.

In a further embodiment, A₂ and/or A₃ is an aromatic heterocyclic ring wherein 1 to 4 ring atoms, e.g. 1, 2, 3, or 4 ring atoms are selected from nitrogen, sulfur and/or oxygen and wherein the ring may be unsubstituted or may carry at least one substituent as described above.

In a further embodiment, A₂ and/or A₃, are independently of each other a 5 to 6 membered aromatic monocyclic ring, wherein the ring is a heteroalkyl ring, particularly selected from, pyrazolidinyl, imidazolidinyl, triazolidinyl, furanyl, wherein the ring may carry 1 to four substituents, or a 9 to 12 membered aromatic bicyclic ring wherein the ring is a naphthyl ring or a heteroalkyl ring with 1 to 4 ring atoms being selected from N, O, and/or S, and wherein the ring may carry one to four substituents.

In another embodiment, one of A₂ or A₃, is a 9 to 12 membered aromatic bicyclic ring wherein the ring is a heterocyclic ring with 1 to 4 ring atoms being selected from N, O, and/or S, and wherein the ring may carry one to four substituents selected from halogen, NO₂, CN, CF₃, —OCF₃, (C₁₋₄) alkyl, (C₁₋₄) alkoxy, (C₁₋₄)alkyl-(C₃₋₇)cycloalkyl, (C₃₋₇) cycloalkyl, OH, benzyl, —O-benzyl, carboxyl, (C₁₋₄) alkyl-carboxylester, carboxamide, —SO₂Me, NH₂, NH—BOC or mono (C₁₋₄) alkyl, or di (C₁₋₄) alkyl carboxamide.

In another embodiment, one of A₂ or A₃, is selected from benzimidazole, indazole, quinoline, imidazole, indole, pyridine, or isoquinoline, wherein the ring may carry one to four substituents selected from halogen, NO₂, CN, CF₃, —OCF₃, (C₁₋₄) alkyl, (C₁₋₄) alkoxy,

(C₁₋₄)alkyl-(C₃₋₇)cycloalkyl, (C₃₋₇) cycloalkyl, OH, benzyl, —O-benzyl, carboxyl, (C₁₋₄) alkyl-carboxylester, carboxamide, —SO₂Me, NH₂, NH—BOC or mono (C₁₋₄) alkyl, or di (C₁₋₄) alkyl carboxamide. In another embodiment, A₂ and/or A₃, is/are naphthalene.

In a further embodiment, A₁ is a 5 to 6 membered monocyclic ring, wherein the ring is a heteroalkyl ring, particularly selected from pyrrolidinyl, pyrazolidinyl, imidazolidinyl, triazolidinyl, furanyl, wherein the ring may carry 1 to four substituents, or a 9 to 12 membered aromatic bicyclic ring wherein the ring is a naphthyl ring or a heteroalkyl ring with 1 to 4 ring atoms being selected from N, O, and/or S, and wherein the ring may carry one to four substituents.

In a further embodiment A₁ is selected from phenyl, pyrrolidinyl, pyrazolidinyl, imidazolidinyl, triazolidinyl.

In a further embodiment A₁ is 1,2,3-triazolidinyl.

A further group of embodiments are conjugates of formula (I) wherein A₂ is an aromatic heterocycle and A₃ is phenyl, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein A₂ is phenyl and A₃ is an aromatic heterocycle, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein A₂ is phenyl and A₃ is phenyl, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein o is 1.

A further group of embodiments are conjugates of formula (I) wherein m is 1, o is 1 and p is 1.

A further group of embodiments are conjugates of formula (I) wherein o is 0 and p is 0.

A further group of embodiments are conjugates of formula (I) wherein m is 1, o is 0 and p is 0.

A further group of embodiments are conjugates of formula (I) wherein the group -A₂-L₃-A₃-L₄- is selected from

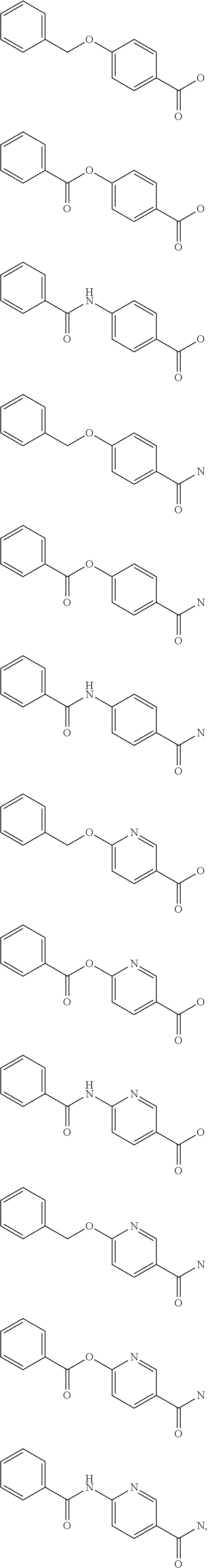

wherein each ring may be unsubstituted or carry one to four substituents selected from halogen, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

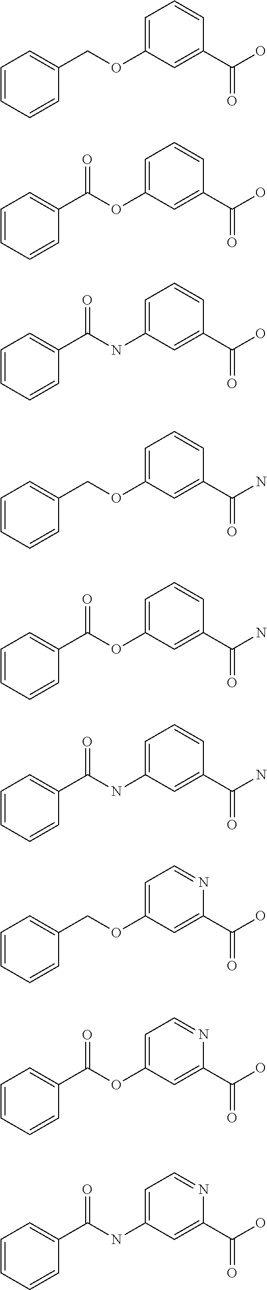

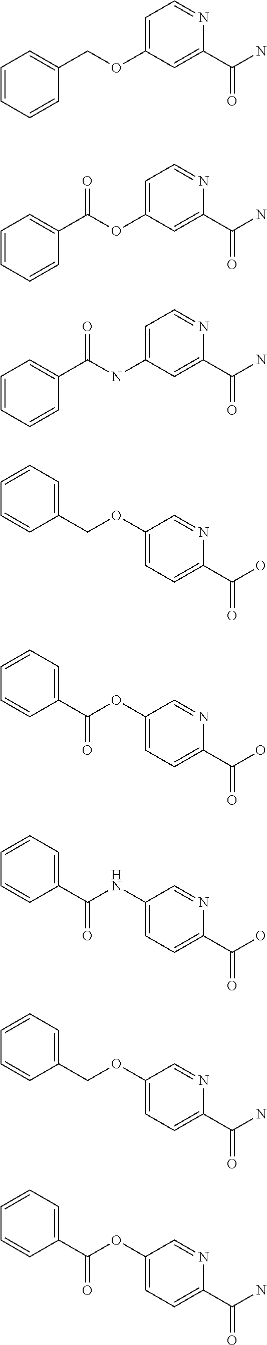

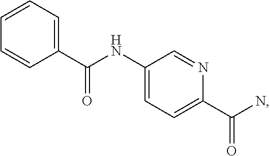

wherein each ring may be unsubstituted or carry one to four substituents selected from halogen, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein

the group -A₂-L₃-A₃-L₄- is selected from

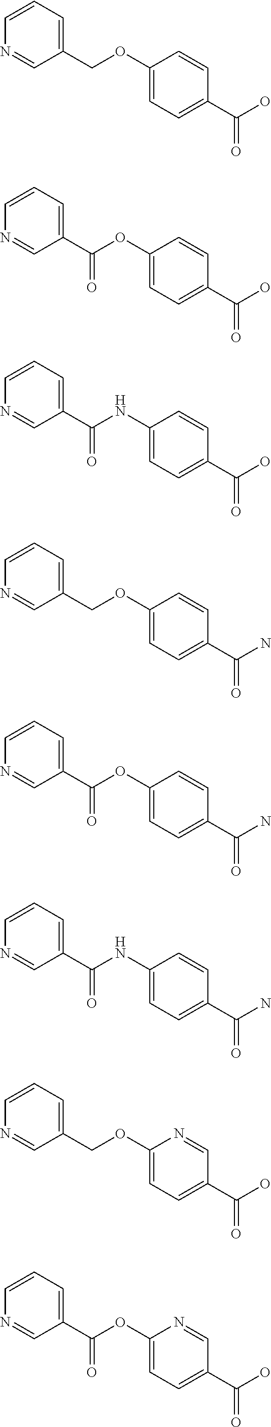

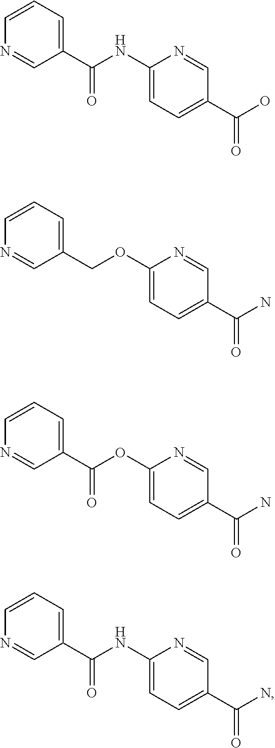

wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein the group -A₂-L₃-A₃-L₄- is selected from

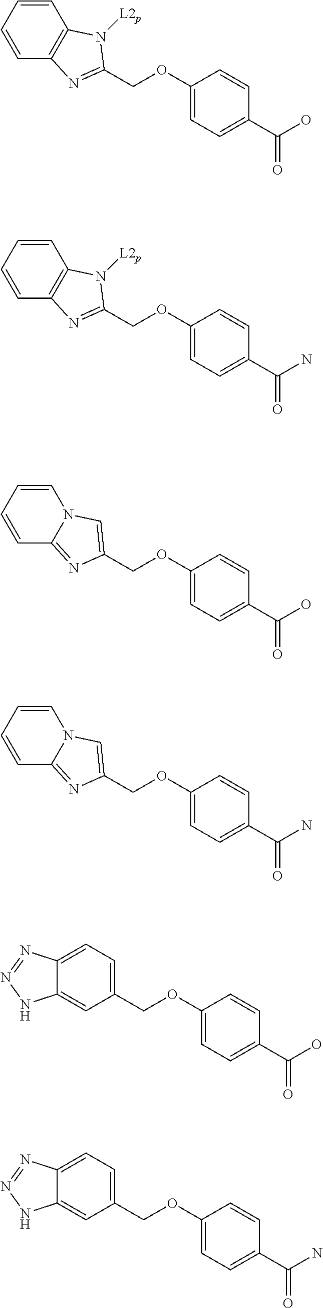

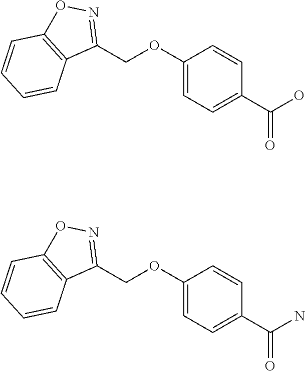 [SMILES: O=C(O)c1ccc(OCc2noc3ccccc23)cc1 ; NC(=O)c1ccc(OCc2noc3ccccc23)cc1]

wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

A further group of embodiments are conjugates of formula (I) wherein the group -A₂-L₃-A₃-L₄- is selected from

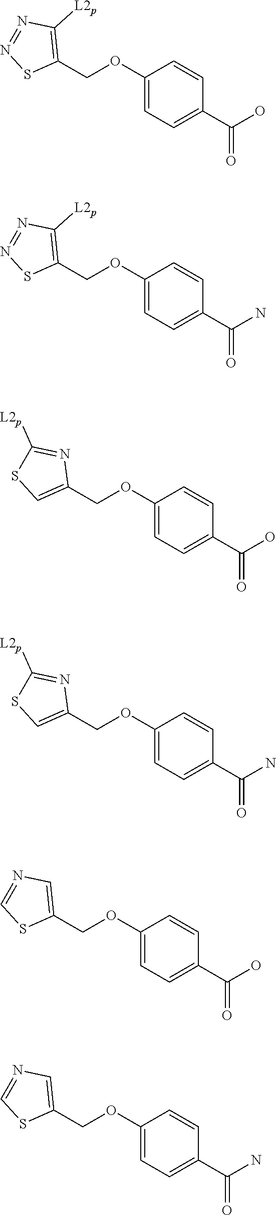

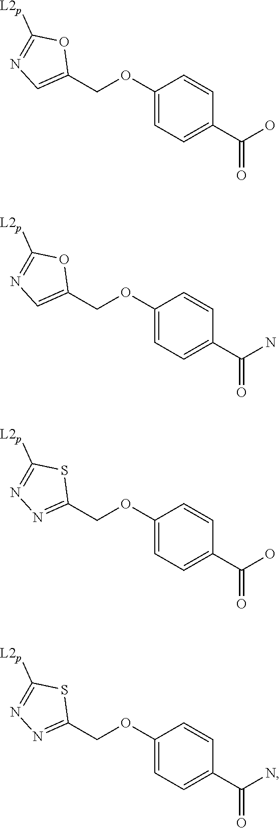

wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

The conjugate of formula (I) comprises a sugar moiety S which binds to the insulin independent glucose transporter GLUT1. This sugar moiety S may comprise a terminal pyranose moiety which is attached via position 2, 3, 4 or 6 to L₄.

In one embodiment the terminal pyranose moiety is attached via position 3 to L₄.

In one embodiment the terminal pyranose moiety is attached via position 4 to L₄.

In one embodiment the terminal pyranose moiety is attached via position 6 to L₄.

In one embodiment the terminal pyranose moiety is attached via position 2 to L₄.

In some embodiments, the sugar moiety S may comprise a terminal pyranose moiety S1 having a backbone structure of Formula (II)

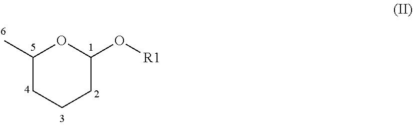

wherein 1, 2, 3, 4, 5, and 6 denote the positions of the C-atoms in the pyranose moiety,
R1 is H or a protecting group,
and wherein S1 is attached via position 2, 3, 4, or 6 to L₄.

The protecting group may be any suitable protecting group known in the art, e.g. an acyl group such as acetyl or benzoyl, an alkyl group such as methyl, an aralkyl group such as benzyl, or 4-methoxybenzyl (PMB).

OR1 can be present in alpha or beta position at C1 of the sugar moiety.

In some embodiments, R1 is selected from methyl, ethyl, CH₂—CH═CH₂, or CH₂CH₂—Si—(CH₃)₃.

In some embodiments, the terminal pyranose moiety may be selected from glucose, galactose, 6-deoxy-6-amino-glucose, or 2,6-dideoxy-2,6-diamino-glucose derivatives, wherein the terminal pyranose moiety is attached via position 2, 3, 4, or 6 to the conjugate of formula (I).

In another embodiment, the terminal pyranose moiety S1 is of the Formula (III):

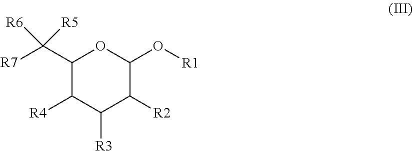

wherein R1 is H or a protecting group such as methyl or acetyl,
R2 and R7 are OR8, or NHR8 or an attachment site to L₄, wherein R8 is H or a protecting group such as acetyl or benzyl,
R3 and R4 are OR8 or an attachment site to the conjugate of formula (I), wherein R8 is H or a protecting group such as acetyl or benzyl,
or R1 and R2 and/or R3 and R4 form together with the pyranose ring atoms to which they are bound a cyclic group, e.g. an acetal,
R5 and R6 are H or form together with the carbon atom to which they are bound a carbonyl group, and
wherein one of R2, R3, R4, and R7 is the attachment site to L₄.

In another embodiment of the terminal pyranose moiety S1 of the formula (III), R1 is H.

In further embodiments of the terminal pyranose moiety S1 of the formula (III), R2, R3, R4, and R7 are OR8, or an attachment site to L₄.

In another embodiment of the terminal pyranose moiety S1 of the formula (III), position 6 of the pyranose moiety and particularly substituent R7 is the attachment site of the terminal pyranose moiety S1 to L₄.

In another embodiment of the terminal pyranose moiety S1 of the formula (III), position 2 of the pyranose moiety and particularly substituent R2 is the attachment site of the terminal pyranose moiety S1 to L₄.

In another embodiment of the terminal pyranose moiety S1 of the formula (III), position 3 of the pyranose moiety and particularly substituent R3 is the attachment site of the terminal pyranose moiety S1 to L₄.

In another embodiment of the terminal pyranose moiety S1 of the formula (III), position 4 of the pyranose moiety and particularly substituent R4 is the attachment site of the terminal pyranose moiety S1 to L₄.

In specific embodiments, the pyranose moiety S1 is of formula (IVa) or (IVb):

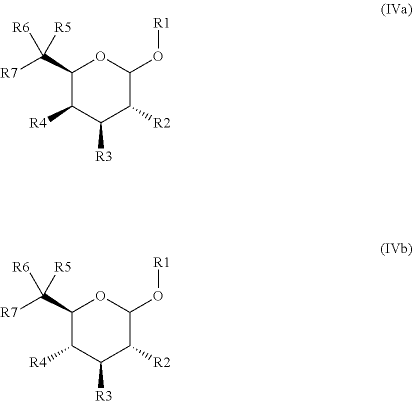

wherein R1, R2, R3, R4, R5, R6, and R7 are defined as indicated above.

The sugar moiety S of the conjugate of formula (I) may comprise one or more, e.g. 2, or 3 saccharide units. For example, the sugar moiety has a structure of formula (V):

—[S2]_{S}—S1 (V)

wherein
S2 is a mono- or disaccharide moiety, particularly comprising at least one hexose or pentose moiety,
S1 is a terminal pyranose moiety as defined above, and
s is 0 or 1.

The saccharide moiety S2 may be a pyranose moiety, particularly selected from glucose or galactose derivatives or a furanose moiety, particularly selected from fructose derivatives.

In specific embodiments, the saccharide moiety S2 is of formula (VIa) or (VIb):

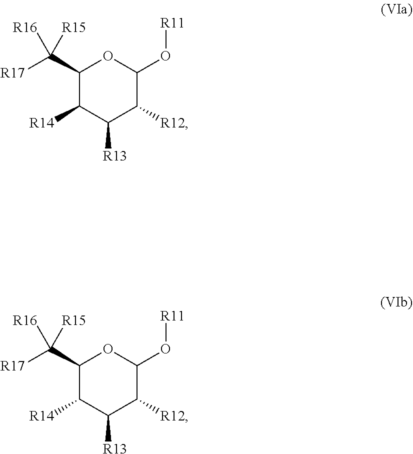

wherein R11 is a bond to S1,
R12 and R17 are OR8 or NHR8 or an attachment site to L₄, wherein R8 is H or a protecting group such as acetyl or benzyl,
R13 and R14 are OR8 or an attachment site to L₄, wherein R8 is H or a protecting group such as acetyl,
R15 and R16 are H or together form with the carbon atom to which they are bound a carbonyl group,
or R11 and R12 and/or R13 and R14 form together with the ring atoms to which they are bound a cyclic group such as an acetal,
and wherein one of R12, R13, R14, and R17 is an attachment site to L₄.

In further embodiments, the conjugate of formula (I) reversibly binds to the insulin independent glucose transporter GLUT1, dependent from the glucose concentration in the surrounding medium, which is blood after administration. In a further embodiment the conjugate of formula (I) of the present invention is not transported through the cell membrane upon binding to GLUT1. In a further embodiment the sugar moiety S comprises a single terminal saccharide moiety. In still further embodiments, the sugar moiety S does not comprise a mannose unit, particularly a terminal mannose unit.

Items

Item (i): A conjugate of formula (I)

P-[L₁]ₘ-[A₁]ₒ-[L₂]ₚ-[A₂]-[L₃]-[A₃]-[L₄]-S (I)

wherein P is an insulin or an insulinotropic peptide,
L₁ and L₂ are independently of each other a linker having a chain length of 1-25 atoms,
L₃ is a linker having a chain length of 2 or 3 atoms,
and L₄ is a linker having a chain length of 1, 2 or 3 atoms,
A₁, is a 5 to 6 membered monocyclic ring or a 9 to 12 membered bicyclic ring, wherein each ring is independently a saturated, unsaturated, or aromatic carbocyclic or heterocyclic ring and wherein each ring may carry at least one substituent,
A₂ and A₃ are independently of each other a 5 to 6 membered monocyclic ring or a 9 to 12 membered bicyclic ring, wherein each ring is independently an aromatic carbocyclic or aromatic heterocyclic ring and wherein each ring may carry at least one substituent,
S is a sugar moiety which binds to the insulin independent glucose transporter GLUT1, and comprises a terminal pyranose moiety which is attached via position 2, 3, 4, or 6 to L₄, and
m, o, and p are independently of each other 0 or 1,
or a pharmaceutically acceptable salt or solvate thereof.

Item (ii): A conjugate according to Item (i), wherein the sugar moiety S may comprise a terminal pyranose moiety S1 having a backbone structure of Formula (II)

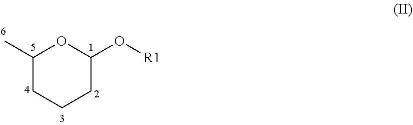

wherein 1, 2, 3, 4, 5, and 6 denote the positions of the C-atoms in the pyranose moiety,
R1 is H or a protecting group,
and wherein S1 is attached via position 2, 3, 4, or 6 to L₄.

Item (iii): The conjugate according to Item (i) or Item (ii), wherein the terminal pyranose moiety is selected from glucose, galactose, 6-deoxy-6-amino-glucose, or 2,6-dideoxy-2,6-diamino-glucose derivatives, wherein the terminal pyranose moiety is attached via position 2, 3, 4, or 6 to the conjugate of formula (I).

Item (iv): The conjugate according to Item (ii), wherein R1 is methyl.

Item (v): The conjugate according to any of Items (i) to (iv), wherein the linker L₄ is —CO—O—or the linker L₄ is —CO—NH—.

Item (vi): The conjugate according to any of Items (i) to (v), wherein the linker L₃ is selected from —CH₂—CH₂—O—, —CH₂—O—, —CO—O— or —CO—NH.

Item (vii): The conjugate according to any of Items (i) to (vi), wherein the linker L₃ is —CH₂—O—.

Item (viii): The conjugate according to any of Items (i) to (vii), wherein A₂ is a 9 to 12 membered bicyclic ring.

Item (ix): The conjugate according to any of Items (i) to (viii), wherein A₂ is a substituted or unsubstituted benzimidazole.

Item (x): The conjugate according to any of Items (i) to (vii), wherein A₂ is a substituted or unsubstituted phenyl.

Item (xi): The conjugate according to any of Items (i) to (viii), wherein A₂ is a substituted or unsubstituted imidazo[1,2-a]pyridine.

Item (xii): The conjugate according to any of Items (i) to (vii), wherein A₂ is a substituted or unsubstituted pyridine.

Item (xiii): The conjugate according to any of Items (i) to (vii), wherein A₂ is a substituted or unsubstituted thiadiazole.

Item (xiv): The conjugate according to any of Items (i) to (xiii), wherein A₃ is a substituted or unsubstituted phenyl.

Item (xv): The conjugate according to any of Items (i) to (xiv), wherein A₃ is a substituted phenyl.

Item (xvi): The conjugate according to any of Items (i) to (ix), (xi), (xiv) or (xv), wherein the group -A₂-L₃-A₃-L₄- is selected from

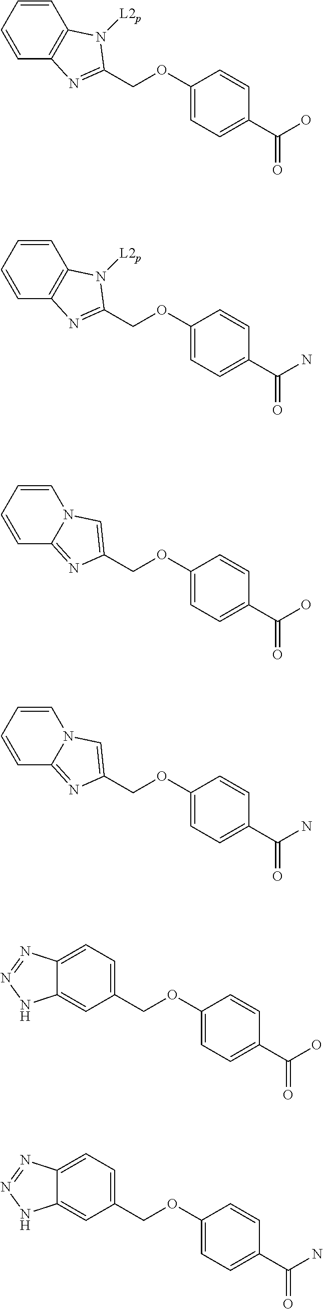

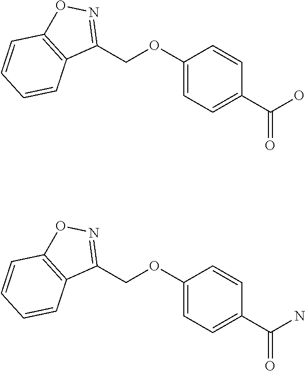 [SMILES: O=C(O)c1ccc(OCc2noc3ccccc23)cc1 ; NC(=O)c1ccc(OCc2noc3ccccc23)cc1]

wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

Item (xvii): The conjugate according to any of Items (i) to (vii), (xiii), (xiv), (xv), wherein the group -A₂-L₃-A₃-L₄- is selected from

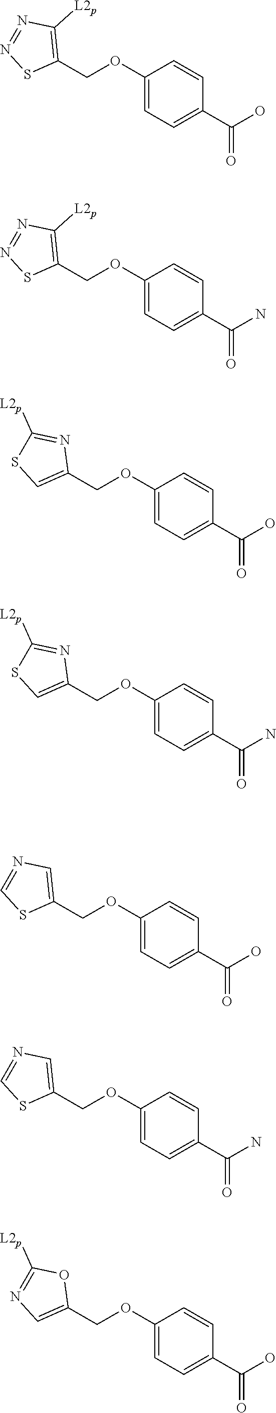

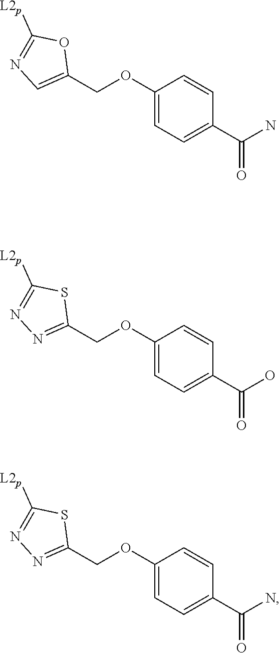

wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

Item (xviii): The conjugate according to Item (xvi) or Item (xvii), wherein the substituents are selected from halogen, (C₁₋₄) alkyl, (C₁₋₄) alkoxy or OH.

Item (xix): The conjugate according to any of the preceding Items, wherein P is an insulin peptide.

Item (xx): The conjugate according to any of the preceding Items, wherein p is 1.

Item (xxi): The conjugate according to any of the preceding Items, wherein m is 0, o is 0 and p is 1.

Item (xxii): The conjugate according to any of the preceding Items, wherein p is 1 and linker L₂ comprises an ester and/or an amide function.

Item (xxiii): The conjugate according to any of Items (i) to (xxi), wherein p is 1 and linker L₂ is a (C₂-C₂₄)alkynylene.

Item (xxiv): The conjugate according to any of the preceding Items, wherein linker L₂ has a chain length of 3 to 10 atoms, or 3 to 6 atoms.

Item (xxv): The conjugate according to any of the preceding Items, wherein linker L₂ comprises —CH₂—.

Item (xxvi): The conjugate according to any of the preceding Items, wherein linker L₂ comprises a saturated alkyl chain having from 2 to 16 carbon atoms.

Item (xxvii): The conjugate according to any of the preceding Items, wherein linker L₂ and/or linker L₁ comprises —C(═O)—.

Item (xxviii): The conjugate according to any of the preceding Items, wherein linker L₂ and/or linker L₁ comprises —NH—C(═O)—O—.

Item (xxix): The conjugate according to any of the preceding Items, wherein linker L₂ comprises —NH—C(═O)—(CH₂)₂—.

Item (xxx): The conjugate according to any of the preceding Items, wherein linker L₂ comprises —C(═O)—.

Item (xxxi): The conjugate according to any of Items (i) to (xxii) and (xxx), wherein L₂ is —(CH₂)₃—C(═O)—.

Item (xxxii): The conjugate according to any of Items (i) to (xx) and (xxii) to (xxxi), wherein o is 1 and A₁ is a substituted or unsubstituted phenyl.

Item (xxxiii): The conjugate according to any of the preceding Items, wherein the amino acid residue in P, to which the remainder of the conjugate is attached, is at the C-terminus of a peptide chain of P.

Item (xxxiv): The conjugate according to any of Items (i) to (xxxii) and (xxxiii), wherein the amino acid residue in P, to which the remainder of the conjugate is attached, is the penultimate residue to the C-terminus of a peptide chain of P.

Item (xxxv): The conjugate according to any of the preceding Items, wherein a lysine residue in P is the residue in P to which the remainder of the conjugate is attached.

Item (xxxvi): The conjugate according to Item (xxxv), wherein the lysine residue in P is the lysine residue in the motif -YTPKT-.

Item (xxxvii): The conjugate according to Item (xxxv) or Item (xxxvi), wherein the lysine residue in P, to which the remainder of the conjugate is attached, is the penultimate residue to the C-terminus of a peptide chain of P.

Item (xxxviii): The conjugate according to Item (xxxv), wherein the lysine residue in P, to which the remainder of the conjugate is attached, is at the C-terminus of a peptide chain of P.

Item (xxxix): The conjugate according to any of Items (i) to (xxxiv), wherein a phenylalanine residue in P is the residue in P to which the remainder of the conjugate is attached.

Item (xi): The conjugate according to Item (xxxix), wherein the phenylalanine residue in P is the phenylalanine residue in the motif FVNQ-.

Item (xli): The conjugate according to Item (xxxix) or Item (xl), wherein the phenylalanine residue in P, to which the remainder of the conjugate is attached, is at the N-terminus of a peptide chain of P.

Item (xlii): The conjugate according to any of the preceding Items, wherein P is attached to the remainder of the conjugate via the amino side chain of an insulin B29Lys residue or via the amino terminus of an insulin B1Phe residue.

Item (xliii): The conjugate according to any of Items (i) to (xx) and (xxii) to (xlii), wherein m is 1, o is 1 and p is 1.

Item (xliv): The conjugate according to any of Items (i) to (xx) and (xxii) to (xliii), wherein A₁ is a five-membered heterocycle.

Item (xlv): The conjugate according to any of Items (i) to (xx) and (xxii) to (xliv), wherein A₁ is a 1,2,3-triazole.

Item (xlvi): The conjugate according to any of Items (i) to (xx) and (xxii) to (xlv), wherein L₁ comprises —C(═O)—.

Item (xlvii): The conjugate according to any of Items (i) to (xx) and (xxii) to (xlvi), wherein L₁ is —(CH₂)₃—C(═O)—.

Item (xlviii): The conjugate according to any of Items (i) to (xx) and (xxii) to (xlii), wherein L₁ comprises —(CH₂)₃—C(═O)—NH—CH₂—.

Item (xlix): The conjugate according to any of the preceding Items, wherein L₁ and/or L₂ comprises —C(═O)—NH—(CH₂)₂—O—(CH₂)₂—O—CH₂—.

Item (I): The conjugate according to any of Items (i) to (xx) and (xxii) to (xlvi) and (xlvii) to (xlvix), wherein A₁ is a 1,2,3-triazole and L₁ comprises —(CH₂)₅—C(═O)—O—or comprises —(CH₂)₃—C(═O)—O—.

Item (Ii): The conjugate according to any of Items (i) to (xx) and (xxii) to (I), wherein A₁ is a 1,2,3-triazole and L₁ comprises —(CH₂)₅—C(═O)— or comprises —(CH₂)₃—C(═O)—.

Item (lii): The conjugate according to any of Items (i) to (xx) and (xxii) to (xliv) and (xlvi) to (xlvix), wherein A₁ is a pyrazole ring.

Another embodiment relates to a pharmaceutical composition comprising a conjugate according to any of Items (i) to (lii) as an active agent, and a pharmaceutical carrier.

Another embodiment relates to a method of preventing and/or treating a disorder associated with, caused by and/or accompanied by a dysregulated glucose metabolism, comprising administering to a subject in need thereof, a conjugate according to any of

Items (i) to (lii) or a pharmaceutical composition comprising a conjugate according to any of Items (i) to (lii) as an active agent, and a pharmaceutical carrier.

Another embodiment relates to a compound of formula (Ia)

R—(O═C)-[L₁]ₘ-[A₁]ₒ-[L₂]ₚ-[A₂]-[L₃]-[A₃]-[L₄]-S (Ia)

wherein L₁, L₂, L₃, L₄, A₁, A₂, A₃, S, m, o and p are defined as in any one of Items (i) to (lii),

R is H, halogen, OH, O-alkyl-, an anhydride forming group or another active ester forming group, like 4-nitrophenylester, succinate or N-hydroxy benzotriazol, or a pharmaceutically acceptable salt or solvate thereof.

Another embodiment relates to a compound of formula (Ib)

[L₁]ₘ-[A₁]ₒ-[L₂]ₚ-[A₂]-[L₃]-[A₃]-[L₄]-S (Ib)

wherein L₁, L₂, L₃, L₄, A₁, A₂, A₃, S, m, o and p are defined as in any one of Items (i) to (lii),
or a pharmaceutically acceptable salt or solvate thereof.

### EXAMPLE EMBODIMENTS

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ is a (C₂-C₂₄) saturated or unsaturated hydrocarbon chain,
L₃ is —CH₂—O—,

### L₄ is —CO—O— or —CO—NH—,

A₂ is a substituted or unsubstituted benzimidazole,
A₃ is a substituted phenyl and wherein the substituents are selected from halogen, (C₁₋₄) alkyl, (C₁₋₄) alkoxy or OH,
S is a sugar moiety which binds to the insulin independent glucose transporter GLUT1, and comprises a terminal pyranose moiety which is attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ is a (C₂-C₂₄) saturated or unsaturated hydrocarbon chain,
L₃ is —CH₂—O—,

### L₄ is —CO—O— or —CO—NH—,

A₂ is an unsubstituted phenyl,
A₃ is a substituted phenyl and wherein the substituents are selected from halogen, (C₁₋₄) alkyl, (C₁₋₄) alkoxy or OH,
S is a sugar moiety which binds to the insulin independent glucose transporter GLUT1, and comprises a terminal pyranose moiety which is attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ is a (C₂-C₂₄) saturated or unsaturated hydrocarbon chain,
L₃ is —CH₂—O—,

### L₄ is —CO—O— or —CO—NH—,

A₂ is a substituted or unsubstituted benzimidazole,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ is a (C₂-C₂₄) saturated or unsaturated hydrocarbon chain,
L₃ is —CH₂—O—,

### L₄ is —CO—O— or —CO—NH—,

A₂ is an unsubstituted phenyl,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ is a (C₂-C₂₄) saturated or unsaturated hydrocarbon chain,
L₃ is —CH₂—O—,

### L₄ is —CO—O— or —CO—NH—,

A₂ is a substituted or unsubstituted benzimidazole,
and wherein L₂ is attached to A₂ via the nitrogen atom at position 1 of the benzimidazole,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ comprises —(CH₂)_{f}—C(═O)—O—, wherein f is from 1 to 8,
L₃ is —CH₂—O—,

### L₄ is —CO—O— or —CO—NH—,

A₂ is a substituted or unsubstituted benzimidazole,
and wherein L₂ is attached to A₂ via the nitrogen atom at position 1 of the benzimidazole,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m is 1 and L1 comprises —(CH₂)_{f}—, wherein f is from 1 to 8; optionally L1 comprises —(CH₂)₅—C(═O)—O—or comprises —(CH₂)₃—C(═O)—O—; optionally L₁ comprises —(CH₂)₅—C(═O)— or comprises —(CH₂)₃—C(═O)—,
o is 1 and A1 is a triazole,
p is 1 and L₂ comprises —(CH₂)_{f}—, wherein f is from 1 to 8,
L₃ is —CH₂—O—,

### L₄ is —CO—O— or —CO—NH—,

A₂ is a substituted or unsubstituted benzimidazole,
and wherein L₂ is attached to A₂ via the nitrogen atom at position 1 of the benzimidazole,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.

An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,

### M, o and p are all 0,

L₃ is —CH₂—O—,

### L₄ is —CO—O— or —CO—NH—,

A₂ is a substituted or unsubstituted imidazo[1,2-a]pyridine,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.
An embodiment relates to a conjugate of formula (I)

P-[L₁]ₘ-[A₁]ₒ-[L₂]ₚ-[A₂]-[L₃]-[A₃]-[L₄]-S (I)

wherein P is an insulin peptide,
m and o are both 0,
p is 1 and L₂ comprises —C(═O)—O—,
L₃ is —CH₂—O—,

### L₄ is —CO—O— or —CO—NH—,

A₂ is a substituted or unsubstituted thiadiazole,
A₃ is a substituted phenyl and wherein the substituents are selected from halogen, (C₁₋₄) alkyl, (C₁₋₄) alkoxy or OH,
S is glucose attached via position 2, 3, 4, or 6 to L₄, and
or a pharmaceutically acceptable salt or solvate thereof.

### Definitions

“Alkyl” means a straight-chain or branched carbon chain. Alkyl groups may be unsubstituted or substituted, wherein one or more hydrogens of an alkyl carbon may be replaced by a substituent such as halogen. Examples of alkyl include methyl, trifluoromethyl, ethyl, n-propyl, isopropyl, n-butyl, isobutyl, sec-butyl, tert-butyl, n-pentyl, and n-hexyl.

“Alkylene” means a straight-chain or branched carbon chain bonded to each side. Alkylene groups may be unsubstituted or substituted.

“Aryl” refers to any substituent derived from a monocyclic or polycyclic or fused aromatic ring, including heterocyclic rings, e.g. phenyl, thiophene, indolyl, naphthyl, pyridyl, which may optionally be further substituted.

“Acyl” means a chemical functional group of the structure R—(C═O)—, wherein R is an alkyl, aryl, or aralkyl.

“Halogen” means fluoro, chloro, bromo, or iodo. Preferably, halogen is fluoro or chloro.

A “5 to 7 membered monocyclic ring” means a ring with 5, 6, or 7 ring atoms that may contain up to the maximum number of double bonds (aromatic or non-aromatic ring which is fully, partially or un-saturated) wherein at least one ring atom up to 4 ring atoms may be replaced by a heteroatom selected from the group consisting of sulfur (including —S(O)—, —S(O)₂—), oxygen and nitrogen (including ═N(O)—). Examples for 5 to 7 membered rings include carbocycles such as cyclopentane, cyclohexane, and benzene, or heterocycles such as furan, thiophene, pyrrole, pyrroline, imidazole, imidazoline, pyrazole, triazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepame, azepine, or homopiperazine.

“9 to 12 membered bicyclic ring” means a system of two rings with 9 to 12 ring atoms, where at least one ring atom is shared by both rings and that may contain up to the maximum number of double bonds (aromatic or non-aromatic ring which is fully, partially or un-saturated) wherein at least one ring atom up to 6 ring atoms may be replaced by a heteroatom selected from the group consisting of sulfur (including —S(O)—, —S(O)₂—), oxygen, and nitrogen (including ═N(O)—) and wherein the ring is linked to the rest of the molecule via a carbon or nitrogen atom. Examples for 9 to 12 membered rings include carbocycles such as naphthalene and heterocycles such as indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine, or pteridine. The term 9 to 12 membered bicyclic ring e also includes spiro structures of two rings like 1,4-dioxa-8-azaspiro[4.5]decane or bridged heterocycles like 8-aza-bicyclo[3.2.1]octane.

The term “protecting group” means a chemical protecting group for protecting OH— groups, known in the art of sugar chemistry as described in Theodora W. Greene, Peter G. M. Wuts, Protective Groups in Organic Synthesis, 3rd Edition, John Wiley &Sonc, Inc. 1999. Examples of a protecting group are: acetyl, benzyl, or p-methoxybenzyl; or isopropylidene groups for protecting two hydroxy groups.

The term “leaving group” is known to persons skilled in the art and means a chemical leaving group for substitution reactions of SN1 or SN2 type like halogen, O-SO2-Me, O-SO2-p-tolyl, or the like.

The term “anhydride forming group” means a chemical group which forms with the carbonyl group to which it is attached an anhydride. An example is acetic anhydride which acetylates said carbonyl group.

The term “active ester forming group” means a chemical group which forms with the carbonyl group to which it is attached an ester which activates said carbonyl group for a coupling reaction with an amino group containing compound forming an amide group.

Examples of active ester forming groups are 4-nitrophenylester, N-hydroxybenzotriazol (HOBt), 1-hydroxy-7-azabenzotriazol or N-hydroxysuccinimid (HOSu).

The term “pharmaceutically acceptable” means approved by a regulatory agency such as the EMEA (Europe) and/or the FDA (US) and/or any other national regulatory agency for use in animals, and/or in humans.

The conjugate of formula (I) of the present invention is suitable for use in medicine, e.g. in veterinary medicine or in human medicine. Particularly, the conjugate of formula (I) is suitable for human medicine. Due to the glucose dependent release/recapture mechanism, the conjugate of formula (I) is particularly suitable for use in the prevention and/or treatment of disorders associated with, caused by, and/or accompanied by a dysregulated glucose mechanism, for example for use in the prevention and/or treatment of diabetes mellitus, particularly of diabetes type 1 or type 2.

The invention also provides a pharmaceutical composition comprising a conjugate of formula (I) as described above as an active agent and a pharmaceutically acceptable carrier.

The term “pharmaceutical composition” indicates a mixture containing ingredients that are compatible when mixed and which may be administered. A pharmaceutical composition includes one or more medicinal drugs. Additionally, the pharmaceutical composition may include one or more pharmaceutically acceptable carriers such as solvents, adjuvants, emollients, expanders, stabilizers, and other components, whether these are considered active or inactive ingredients.

The conjugates of formula (I) of the present invention, or salts thereof, are administered in conjunction with an acceptable pharmaceutical carrier as part of a pharmaceutical composition. A “pharmaceutically acceptable carrier” is a compound or mixture of compounds which is physiologically acceptable while retaining the therapeutic properties of the substance with which it is administered. Standard acceptable pharmaceutical carriers and their formulations are known to one skilled in the art and described, for example, in Remington: The Science and Practice of Pharmacy, (20th ed.) ed. A. R. Gennaro A. R., 2000, Lippencott Williams & Wilkins. One exemplary pharmaceutically acceptable carrier is physiological saline solution.

Acceptable pharmaceutical carriers include those used in formulations suitable for oral, rectal, nasal, or parenteral (including subcutaneous, intramuscular, intravenous, intradermal, and transdermal) administration. The compounds of the present invention will typically be administered parenterally.

The term “pharmaceutically acceptable salt” means salts of the conjugates of formula (I) of the invention which are safe and effective for use in mammals. Pharmaceutically acceptable salts may include, but are not limited to, acid addition salts and basic salts. Examples of acid addition salts include chloride, sulfate, hydrogen sulfate, (hydrogen) phosphate, acetate, citrate, tosylate, or mesylate salts. Examples of basic salts include salts with inorganic cations, e.g. alkaline or alkaline earth metal salts such as sodium, potassium, magnesium, or calcium salts and salts with organic cations such as amine salts. Further examples of pharmaceutically acceptable salts are described in Remington: The Science and Practice of Pharmacy, (20th ed.) ed. A. R. Gennaro A. R., 2000, Lippencott Williams & Wilkins or in Handbook of Pharmaceutical Salts, Properties, Selection and Use, e.d. P. H. Stahl, C. G. Wermuth, 2002, jointly published by Verlag Helvetica Chimica Acta, Zurich, Switzerland, and Wiley-VCH, Weinheim, Germany.

The term “solvate” means complexes of the conjugates of formula (I) of the invention or salts thereof with solvent molecules, e.g. organic solvent molecules and/or water.

The compounds of the present invention will be administered in a “therapeutically effective amount”. This term refers to a nontoxic but sufficient amount of the conjugate of formula (I) to provide the desired effect. The amount of a conjugate of formula (I) of the formula (I) necessary to achieve the desired biological effect depends on a number of factors, for example the specific conjugate of formula (I) chosen, the intended use, the mode of administration, and the clinical condition of the patient. An appropriate “effective” amount in any individual case may be determined by one of ordinary skill in the art using routine experimentation.

Pharmaceutical compositions of the invention are those suitable for parenteral (for example subcutaneous, intramuscular, intradermal, or intravenous), oral, rectal, topical, and peroral (for example sublingual) administration, although the most suitable mode of administration depends in each individual case on the nature and severity of the condition to be treated and on the nature of the conjugate of formula (I)) used in each case.

Suitable pharmaceutical compositions may be in the form of separate units, for example capsules, tablets, and powders in vials or ampoules, each of which contains a defined amount of the conjugate of formula (I); as powders or granules; as solution or suspension in an aqueous or nonaqueous liquid; or as an oil-in-water or water-in-oil emulsion. It may be provided in single dose injectable form, for example in the form of a pen. The compositions may, as already mentioned, be prepared by any suitable pharmaceutical method which includes a step in which the active ingredient and the carrier (which may consist of one or more additional ingredients) are brought into contact.

The conjugates of formula (I) of the present invention can be widely combined with other pharmacologically active compounds, such as all drugs mentioned in the Rote Liste 2016 e.g. with all antidiabetics mentioned in the Rote Liste 2016, chapter 12.

The active ingredient combinations can be used especially for a synergistic improvement in action. They can be applied either by separate administration of the active ingredients to the patient or in the form of combination products in which a plurality of active ingredients are present in one pharmaceutical preparation. When the active ingredients are administered by separate administration of the active ingredients, this can be done simultaneously or successively.

General Methods for the Synthesis of Conjugates of Formula (I)

General methods for the synthesis of conjugates of formula (I) and intermediates thereof are described in the following schemes:

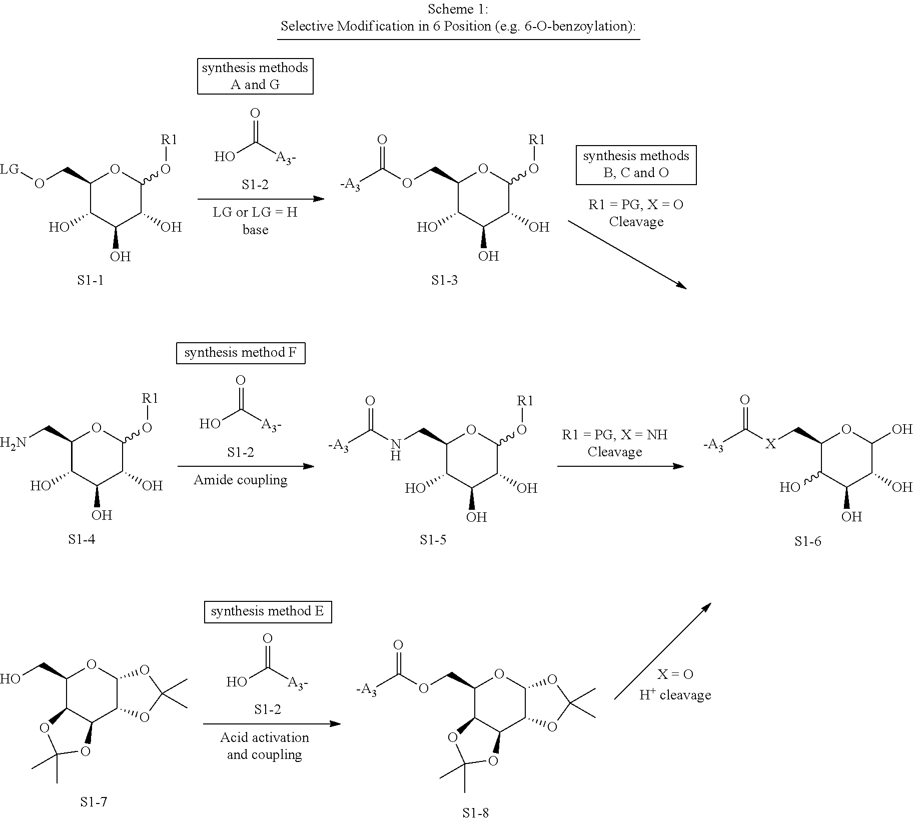

Introduction of substituents in position 6 of carbohydrates is straight forward. As a standard procedure applicable for most carbohydrates we start with partially protected pyranosides e.g. methyl-6-O-toluenesulfonyl-D-pyranosides (S1-1), which can be prepared directly from the corresponding sugar using standard procedures. The benzoic acid is deprotonated (e.g. NaH) and the corresponding carboxylate directly substitutes the leaving group of the sugar moiety to form the ester S1-3.

Activated carbohydrate precursors of formula S1-1 serve as building blocks to yield 6-amino-6-deoxy derivatives (S1-4) after introduction of an azido group in position 6 and subsequent reduction. Such building blocks can be selectively converted to the corresponding amides (S1-5).

In both cases the acetal can be cleaved under acidic conditions to yield the modified free sugar S1-6. In case R1 is an allyl group deprotection can be done with Pd(II)Cl₂ in methanol or other deprotection methods known to persons skilled in the art to yield compounds of formula S1-6. In case R1 is a trimethylsilylethyl group deprotection can be done under acidic conditions (e.g. trifluoroacetic acid) to yield compounds of formula S1-6.

For modification of galactose an alternative route can be applied. Direct introduction of isopropylidene leads to diprotected derivatives S1-7 leaving the 6 position unprotected. These can be directly converted to the corresponding esters using activated acid derivatives. In this case the release of the protecting groups (acidic conditions like hydrochloric acid) directly yields the free sugar derivatives S1-6.

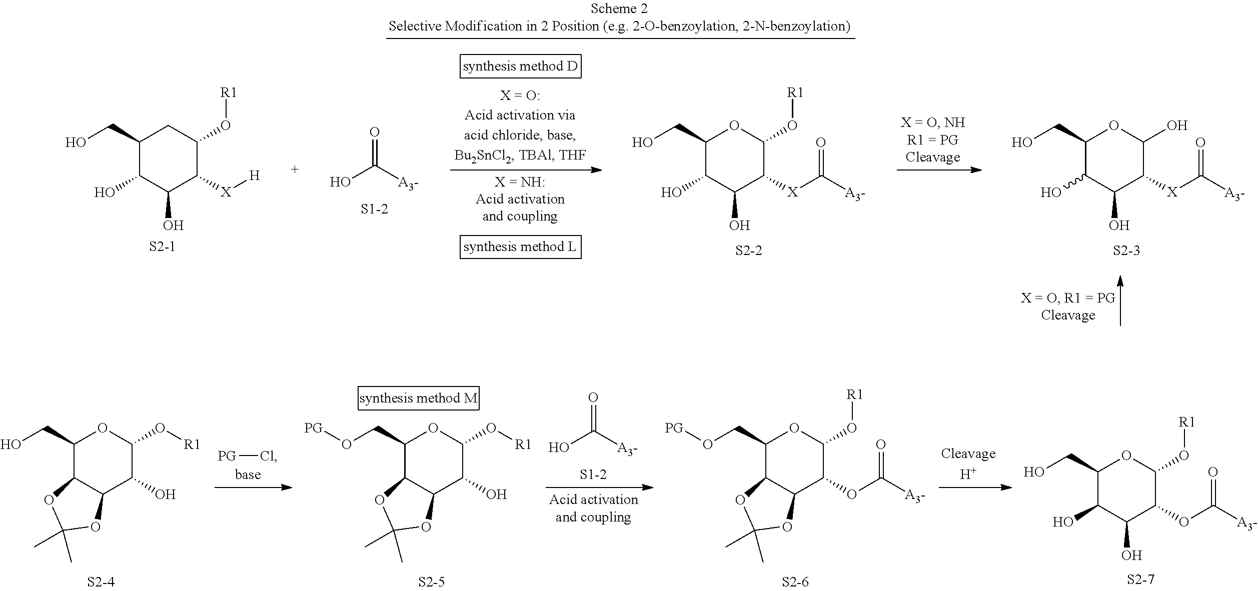

The compounds can be prepared by the general synthetic route depicted in scheme 2. The starting materials are commercially available, known in the literature or can be prepared by known methods. E. g. 1-methyl 2-acetamido-2-deoxy-α-D-glycopyranoside was synthesized by a protocol, previously reported by Zhu et al. (J. Org. Chem. 2006, 71, 466-479). Saponification with aqueous NaOH under reflux yielded the starting material 1-methyl 2-amino-2-deoxy-α-D-glycopyranoside.

The regioselective esterification/benzoylation (X═O) was carried out using a method (synthesis method D), which was reported by Muramatsu et al. (J. Org. Chem. 2013, 78, 2336-2345), using a Sn-reagent to yield predominantly 2-benzoylated derivatives S2-2. Amidation under standard conditions (synthesis method L) of 1-methyl 2-amino-2-deoxy-α-D-glycopyranoside (X═N) yielded pure 2-amidated products S2-2.

The isopropylidene-α-D-galactopyranoside derivative S2-4 was used as starting material for the synthesis of 2-benzoylated galactopyranoside derivatives. Protection of the 6 position, followed by esterification in 2 position gave the protected derivative S2-6. Cleavage under acidic conditions yielded S2-7.

In case R1 is a protecting group as described above, cleavage of compounds S2-2 and S2-7, respectively, can be carried out as described in scheme 1 to yield compounds of formulas S2-3 (see synthesis method N in the experimental part).

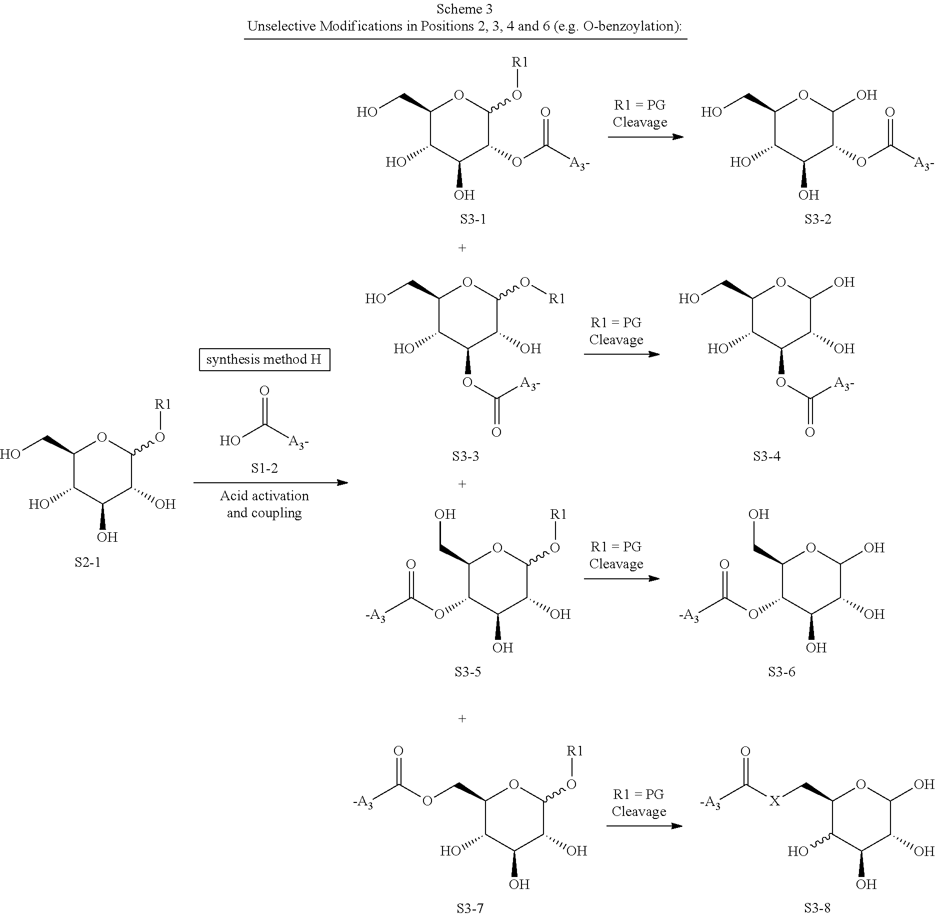

Unselective benzoylation was carried out under method H, using dicyclohexylcarbodiimide as coupling reagent in the presence of 4-DMAP. The crude reaction product contains a mixture of —O, 3-O, 4-O and 6-O-benzoylated compounds, which were separated by standard purification techniques to yield regioselective pure S3-1, S3-3, S3-5, and S3-7.

In case R1 is a protecting group as described above cleavage of compounds S3-1, S3-3, S3-5 and S3-7, respectively, can be carried out as described in scheme 1 to yield compounds of formulas S3-2, S3-4, S3-6, and S3-8, respectively, (see synthesis method N in the experimental part).

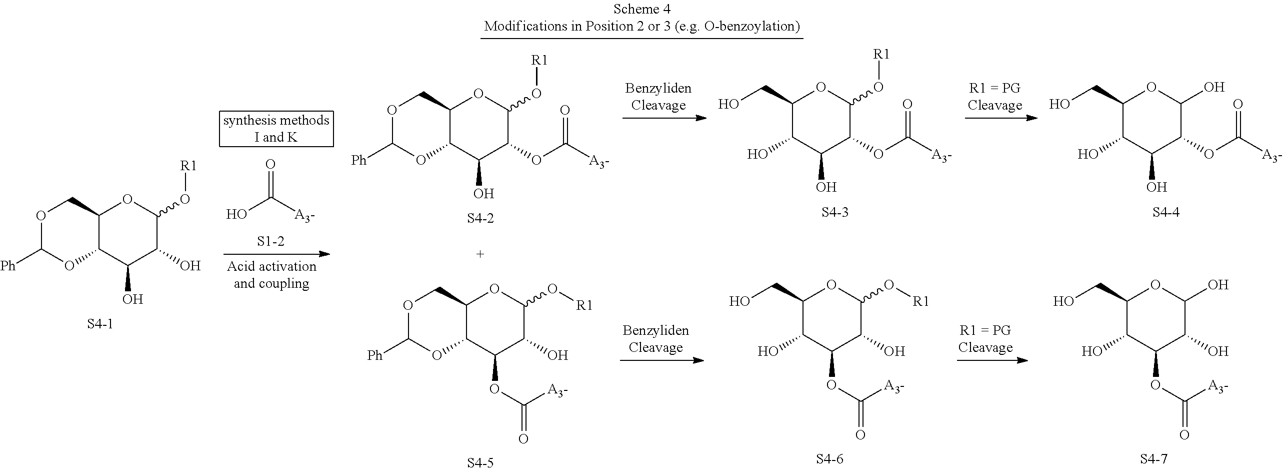

In the literature several methods are described for selective benzoylation of glucopyranosides like S4-1. Depending on the carbohydrate (gluco- or galactopyranoside) and the conditions used, both positions can be addressed directly. HOBt activated benzoic acids are coupled predominant at the 2-position of a glucopyranoside and at the 3 position of a galactopyranoside (S. Burugupalli et al. Org. Biomol. Chem. 2016, 14, 97, Investigation of benzoyloximes as benzoylating reagents: benzoyl-Oxyma as a selective benzoylating reagent; S. Kim et al. J. Org. Chem. 50(10), 1751-2, 1985, Selective benzoylation of diols with 1-(benzoyloxy)benzotriazole). The use of chiral (benzotetramisole, both enantiomers tested) and achiral reagents are investigated to address selectively the 2 and 3 position (G. Xiao et al. J. Am. Chem. Soc. 2017, 139, 4346-4349, Selective Acylation of carbohydrates directed by Cation-n interaction; G. Hu and A. Vasella, Helvetica Chimica Acta, 85(12), 4369-4391; 2002, Regioselective benzoylation of 6-O-protected and 4, 6-O-diprotected hexopyranosides as promoted by chiral and achiral ditertiary 1,2-diamines). In our hands aromatic acids of formula S1-2 were activated with HOBt and (3-dimethylamino-propyl)-N′-ethylcarbodiimide in inert solvents like dichloromethane, addition of the 4,6-protected glucopyranosides of formula S4-1 under basic conditions, for example triethylamine, to yield predominantly compounds of formula S4-2. The activation of aromatic acids of formula S1-2 as acid chloride, using acidic conditions like thionyl chloride or neutral conditions like Ghosez reagent, and reaction with glucopyranosides of the formula S4-1 yielded in mixtures of 2-O— and 3-O-benzoylated compounds of formulas S4-2 and S4-5.

The separated compounds S4-2 and S4-5 were selectively cleaved to compounds of formula S4-3 and S4-6 using mild acidic conditions like p-toluene-sulfonic acid in dichloromethane, hydrochloric acid (0.1 M) in acetonitrile, or catalytic amounts of tin dichloride in acetonitrile to yield compounds of formulas S4-3 and S4-6. In case R1 is a protecting group like described above cleavage of compounds S4-3 and S4-6, respectively, can be carried out as described in scheme 1 to yield compounds of formulas S4-4 and S4-7, respectively, (see synthesis method N in the experimental part).

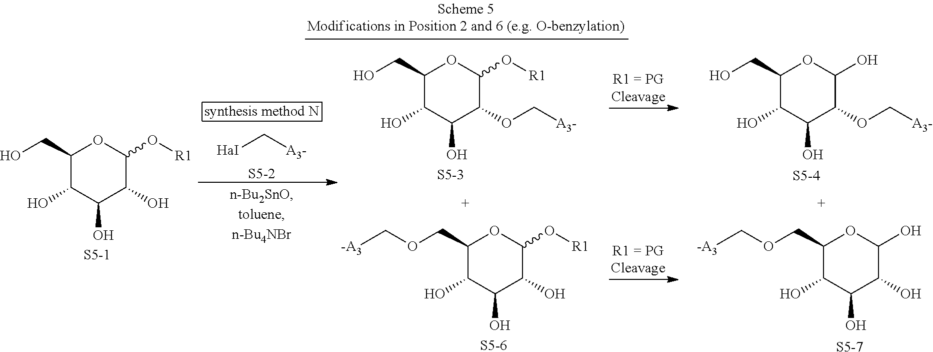

Starting from methyl-D-glucopyranoside (S5-1) benzylation with compounds of formula S5-2 wherein Hal is a halide like fluoro, chloro, bromo, or iodido can be done predominantly at position 2 of the carbohydrate molecule with minor side products at position 6 using organo tin compounds like di-n-butyltin oxide in solvents like toluene under reflux conditions to yield compounds of formula S5-3 (major product) and S5-6 (side product) (Y. Zhou et al. Tetrahedron 2013, 2693-2700, Halide promoted organotin-mediated carbohydrate benzylation: mechanism and application). The regioselectivity for this organo tin mediated benzylation reaction is the same for alpha and beta methyl glucopyranosides. In case R1 is a protecting group as described above cleavage of compounds S5-3 and S5-6, respectively, can be carried out as described in scheme 1 to yield compounds of formulas S5-4 and S5-7, respectively, (see synthesis method N in the experimental part).

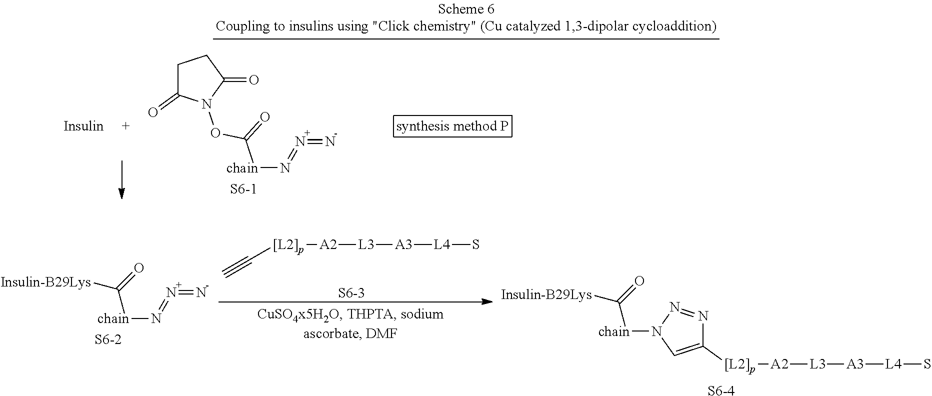

The synthesis of compounds S6-2 can be carried out by reaction of compounds S6-1 with insulin under basic conditions, e.g. pH 10. Therefore the insulin is dissolved in a dimethylformamide-water mixture and brought to pH 10 by an organic base like triethylamine. At low temperatures (e.g. 0° C.) the activated azido-dioxopyrrolidines S6-1 are added to yield compounds of formula S6-2.

Compounds of S6-4 can be synthesized using copper catalyzed [3+2]-cycloaddition conditions, also known as azide-alkyne or click cycloaddition. S6-2 and alkynes S6-3, are reacted with CuSO₄*5H₂O, tris(3-hydroxypropyltriazolylmethyl)amine (THPTA) and sodium ascorbate to yield compounds of formula S6-4.

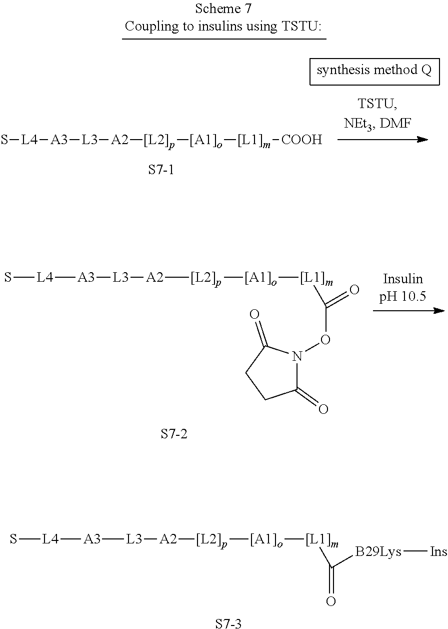

Another possibility to synthesize the compounds of formula (I) is to activate compounds of formula S7-1 with an acid activation reagent like TSTU to form the NHS ester S7-2. Coupling of the NHS ester S7-2 can be carried out as described in Scheme 6 to yield compounds of formula S7-3.

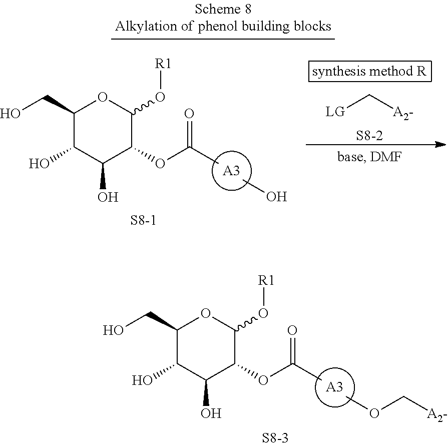

Another possibility to synthesize compounds of formula (I) is to alkylate phenol building blocks of formula S8-1, which can be synthesized using the methods described above, by deprotonation of S8-1 with a base like e.g. potassium carbonate in an aprotic solvent like DMF, and addition of S8-2, wherein LG is a leaving group like chloro, bromo, iodido, mesyl, tosyl or the like to yield compounds of the formula S8-3.

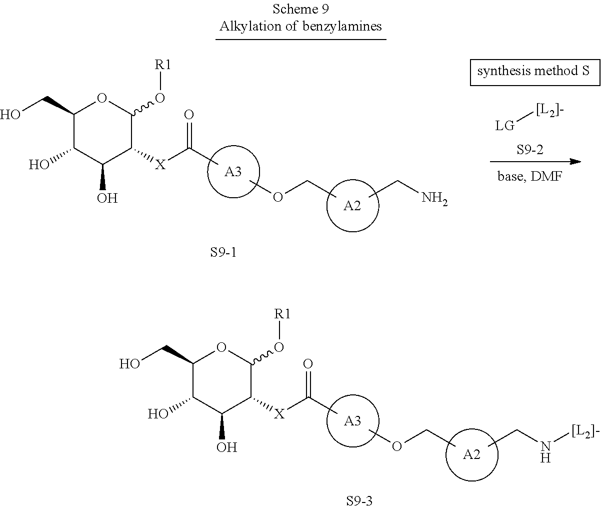

Another possibility to synthesize compounds of formula (I) is to alkylate benzylamines of formula S9-1, which can be synthesized using the methods described above, by deprotonation of S9-1 with a base like e.g. potassium carbonate in an aprotic solvent like DMF, and addition of S9-2, wherein LG is a leaving group like chloro, bromo, iodido, mesyl, tosyl or the like to yield compounds of the formula S9-3.

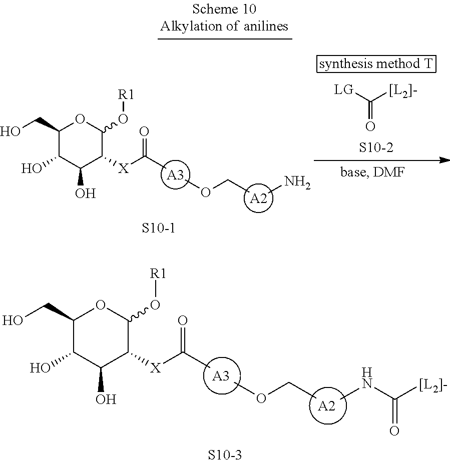

Another possibility to synthesize compounds of formula (I) is to alkylate anilines of formula S10-1, which can be synthesized using the methods described above, by activation of the corresponding acid of 510-2, e.g. by reaction with an acid chloride like isobutyl chloroformate or other methods known to persons skilled in the art, in presence of a base like e.g. potassium carbonate in an aprotic solvent like DMF, to yield compounds of the formula S10-3.

### Abbreviations

BEP 2-bromo-1-ethyl pyridinium tetrafluoroborate
br. Broad
d Doublet
dd Double doublet
ddd Double double doublet
DDQ 2,3-dichloro-5,6-dicyano-1,4-benzochinone
DMSO dimethyl sulfoxide
dt Double triplet
ELSD Evaporative Light Scattering Detector
Eq. Equivalent/s
ES-API Electro spray atmospheric pressure ionization
h Hour/hours
FCS Fetal calf serum
HATU 1-[bis(dimethylamino)methylene]-1H-1,2,3-triazolo[4,5-b]pyridinium 3-oxide hexafluorophosphate
HEK Human embryonic kidney cells.
HOBt 1-hydroxybenzotriazole
HPLC High pressure liquid chromatography
Hz Hertz
J coupling constant
KRB Krebs-Ringer bicarbonate buffer
LG leaving group
LC/MS Liquid chromatography/mass spectra
m Multiplet
M Molar (mol/liter)
m/e Mass per charge
MEM Minimum-Essential-Medium
MHz Megahertz
min Minutes
MPLC Medium pressure liquid chromatography
NEAA Non-essential amino acids
NMR Nuclear magnetic resonance
PBS Phosphate buffered saline
PG Protecting group
q Quadruplet
quint. Quintuplet
rpm rounds per minutes
s Singlet
t Triplet
td doublet of triplets
TBTU N,N,N′,N′-Tetramethyl-O-(benzotriazol-1-yl)uronium tetrafluoroborate
TFA Trifluoro acetic acid
TLC Thin layer chromatography
THPTA tris(3-hydroxypropyltriazolylmethyl)amine
TOTU O-[(ethoxycarbonyl)cyanomethylenamino]-N,N,N′,N′-tetramethyluronium tetrafluoroborate
TSTU O—(N-Succinimidyl)-N,N,N′,N′-tetramethyluronium tetrafluoroborate
t_{R} Retention time
R_{f} Relative to front value
UV Ultra violet
v/v Volume by volume

Experimental Part

Chromatographic and Spectroscopic Methods

TLC/UV-Lamp

Thin layer chromatography (TLC) was performed on glass or aluminum plates from Merck coated with silica gel 60 F254. Compounds where detected using an UV-Lamp (Lamag) at different wavelengths (254 nm and 366 nm).

Compounds which could not be detected by UV were stained by different methods: (a) 10% H₂SO₄ in ethanol, (b) 1% KMnO₄-solution, (c) molybdatophosphoric acid-cerium(IV)sulfate solution in sulfuric acid (6 mL concentrated sulfuric acid and 94 mL H₂O, 2.5 g molybdatophosphoric acid, 1 g cerium(IV)sulfate).

MPLC

Normal Phase Chromatography was performed using CombiFlash® Rf (Teledyne ISCO). The gradients used are given in the description of the examples.

HPLC

For preparative reversed phase HPLC an Agilent 1200 preparative HPLC machine and an AEKTA™ Avant machine was used. Separation was performed using the gradients given in the experimental descriptions.

NMR

400 MHz: NMR spectra were recorded on a Bruker AVANCE II 400 spectrometer operating at a proton frequency of 400.13 MHz and a ¹³C-carbon frequency of 100.61 MHz. The instrument was equipped with a 5 mm BBO room temperature probe head.

500 MHz: NMR spectra were recorded on a Bruker AVANCE III 500 spectrometer operating at a proton frequency of 500.30 MHz and a ¹³C-carbon frequency of 125.82 MHz. The instrument was equipped with a 5 mm TCI cryo probe head.

600 MHz: NMR spectra were recorded on a Bruker AVANCE III 600 spectrometer operating at a proton frequency of 600.10 MHz and a ¹³C-carbon frequency of 150.91 MHz. The instrument was equipped with a 5 mm TXI room temperature probe head.

LC/MS

For retention time and mass detection an LC/MS-system from Waters Acquity SDS was used. The injection volume was 0.5 μl. Molecular weights are given in gram per mol [g/mol], detected masses in mass per charge [m/e].

LC/MS-Method A

Gradient program: 95% H₂O (0.05% formic acid) to 95% acetonitrile (0.035% formic acid) in 2.0 min, 95% acetonitrile (0.035% formic acid) till 2.6 min, flow rate: 0.9 mL/min, column: 2.1×50 mm Waters ACQUITY UPLC BEH C₁₈ 1.7 μm, 55° C.

LC/MS-Method B

Gradient program: 96% H₂O (0.05% trifluoroacetic acid) to 95% acetonitrile in 2.0 min, 95% acetonitrile till 2.4 min, flow rate: 1.0 mL/min, column: 2.1×20 mm YMC J'sphere ODSH80 4 μm, 30° C.

LC/MS-Method C

Gradient program: 93% H₂O (0.05% trifluoroacetic acid) to 95% acetonitrile (0.05 trifluoroacetic acid) in 1 min, 95% acetonitrile till 1.45 min, flow rate: 1.1 mL/min, column: 10×2.0 mm LunaC₁₈ 3 μm.

LC/MS-Method D

Gradient program: 98% H₂O (0.05% formic acid) to 98% acetonitrile (0.035% formic acid) in 3.8 min, 98% acetonitrile (0.035% formic acid) till 4.5 min, flow rate: 1.0 mL/min, column: 2.1×50 mm Waters ACQUITY UPLC BEH C₁₈ 1.7 μm, 55° C.

LC/MS-Method E: (Example 217-220)

Gradient program: gradient program: 98% H₂O (0.1% formic acid) to 98% acetonitrile (0.1% formic acid) in 2.8 min, 98% acetonitrile (0.1% formic acid) till 4.8 min, flow rate: 1.0 mL/min, column: 4.6×50 mm X-Select CSH C₁₈ 2.5 μm, Inj volume: 5.0 μL. ELSD Conditions: (Example 217-220)

Gradient program: 98% H₂O (0.05% NH₃) to 100% acetonitrile (0.05% NH₃) in 3.5 min, 100% acetonitrile (0.05% NH₃) till 4.5 min, flow rate: 1.2 mL/min, column: 4.6×50 mm X-Bridge C₁₈ 3.5 μm; Inj volume: 0.2 μL.

LC/MS-Method F (Insulines)

Gradient program: 85% H₂O (0.05% formic acid) to 50% acetonitrile (0.035% formic acid) in 8.3 min, 50% acetonitrile (0.035% formic acid) to 90% acetonitrile (0.035% formic acid) till 8.5 min, flow rate: 0.5 mL/min, column: 2.1×100 mm Waters ACQUITY UPLC PEPTIDE BEH C₁₈ 300 A, 1.7 μm, 40° C.

Syntheses

### Method A: 6-O-Benzoylation of carbohydrate-6-O-tosylate

General Description:

Sodium hydride (17.22 mg, 430.58 μmol) is added at 0° C. under argon atmosphere to a solution of the benzoic acid (430.58 μmol) in N,N-dimethylformamide (5 mL). Subsequently 6-O-(tosyl)-methyl-α-D-glucopyranoside (100 mg, 287.05 μmol) is added and the solution is stirred at 80° C. for 16 h. The reaction is monitored by LC/MS. CH₂Cl₂ (25 mL) is added and the organic phase is washed twice with H₂O. The organic phase is dried with Na₂SO₄, filtered and evaporated.

### Example 1

### 6-O-(4-Benzyloxybenzoyl)-methyl-β-D-glucopyranoside

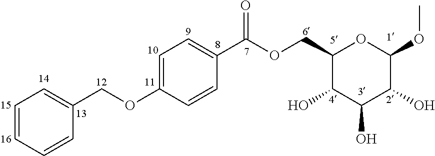 [SMILES: CO[C@@H]1O[C@H](COC(=O)c2ccc(OCc3ccccc3)cc2)[C@@H](O)[C@H](O)[C@H]1O]

Example 1 was synthesized from 4-benzyloxybenzoic acid (98 mg, 430.6 μmol) and 6-O-(tosyl)-methyl-α-D-glucopyranoside (100 mg, 287.1 μmol) following the procedure described in synthesis method A. The crude mixture was purified by HPLC (Waters SunFire Prep OBD C₁₈, 5 μm, 50×100 mm, eluents: A: H₂O+0.1% trifluoroacetic acid and B: acetonitrile, flow 120 mL/min, gradient: 0-2 min: 5% B, 2-2.5 min 5% to 15% B, 2.5-10.5 min: 15% to 65% B, 10.5-11 min 65% to 99% B, 11-13 min 99% B).

Yield: 66 mg (163.2 μmol, 57%).

LC/MS (ES-API): m/z=405.20 [M+H]⁺; calculated: 404.15; t_{R} (λ=220 nm): 1.61 min (LC/MS-method A).

¹H-NMR (500 MHz, DMSO-d₆): δ [ppm]=7.92 (d, 2H, AA′BB′ system, C9-H), 7.46 (d, 2H, C14-H), 7.40 (t, 2H, C15-H), 7.34 (t, 1H, C16-H), 7.15 (d, 2H, AA′BB′ system, C10-H), 5.19 (s, 2H, OCH₂), 4.51 (dd, 1H, C6′-Ha), 4.27 (dd, 1H, C6′-Hb), 4.11 (d, 1H, C1′-H), 3.47 (dd, 1H, C5′-H), 3.34 (s, 3H, OCH₃), 3.20 (dd, 1H, C4′-H), 3.18 (dd, 1H, C3′-H), 2.99 (dd, 1H, C2′-H).

¹³C NMR (500 MHz, DMSO-d₆): δ [ppm]=165.25 (C7), 162.23 (C11), 136.40 (C13), 132.21 (C9), 128.48 (C15), 128.03 (C16), 127.85 (C14), 122.16 (C8), 114.84 (C10), 103.89 (C1′), 76.36 (C3′), 73.65 (C5′), 73.30 (C2′), 70.08 (C4′), 69.51 (C12), 63.90 (C6′), 55.85 (OCH₃).

### Method B: Cleavage of 1-O-allyl

General Description (Bio. & Med. Chem., 2005 (13), 121-130):

To a solution of 6-O-(4-benzyloxy-benzoyl)-allyl-α-D-glucopyranoside (35 mg, 81.31 μmol) in MeOH (2 mL) palladium(II)-chloride (2.88 mg, 16.26 μmol) is added under argon atmosphere. The reaction mixture is stirred for 3 h at 25° C. and the reaction monitored by LC/MS. The solvent is evaporated.

### Example 47

### 6-O-(4-(Benzyloxy-3-methoxy-5-chloro)-benzoyl)-D-glucopyranose

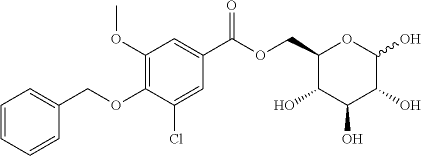 [SMILES: COc1cc(C(=O)OC[C@H]2OC(O)[C@H](O)[C@@H](O)[C@@H]2O)cc(Cl)c1OCc1ccccc1]

Example 47 was synthesized from 6-O-(4-benzyloxy-benzoyl-3-methoxy-5-chloro)-allyl-α-D-glucopyranoside (Example 9) (50 mg, 101 μmol) following the procedure described in synthesis method B. The crude residue was purified by HPLC (Waters SunFire Prep OBD C₁₈, 5 μm, 50×100 mm, eluents: A: H₂O+0.1% trifluoroacetic acid and B:

acetonitrile, flow 120 mL/min, gradient: 0-2 min: 5% B, 2-2.5 min 5% to 10% B, 2.5-10.5 min: 10% to 70% B, 10.5-11 min 70% to 99% B, 11-13 min 99% B)

Yield: 15.4 mg (33.9 μmol, 34%).

LC/MS (ES-API): m/z=453.17 [M−H]⁻; calculated: 453.10; t_{R} (λ=220 nm): 1.57 min (LC/MS-method A).

¹H-NMR (400 MHz, DMSO-d₆): δ [ppm]=7.52 (m, 6H), 7.37 (m, 4H), 6.67 (d br., J=6.48 Hz, C1′-OH), 6.35 (d br., J=4.52 Hz, C1′-OH), 5.21 (s br., 1H, OH), 5.13 (s, 2H, OCH₂), 4.93 (s br., 1H, OH), 4.77 (s br., 1H, OH), 4.52 (m br., 2H), 4.31 (m, 2H), 3.94 (s, 3H, OCH₃), 3.87 (m, 1H), 3.46 (m, 1H), 3.17 (d br., J=8.80 Hz, 1H).

### Method C: Cleavage of 1-O-trimethylsilylethyl

General Description:

To a solution of 6-O-benzoyl-(2-(trimethylsilyl)ethyl)-α-D-glucopyranoside (122.29 μmol) in CH₂Cl₂ (1.8 mL) trifluoroacetic acid (200 μl, 2.60 mmol) is added under argon atmosphere. The reaction mixture is stirred for 1 h at 25° C. The reaction is monitored by

LC/MS-method B. Finally the reaction-mixture is diluted with acetonitrile and H₂O and freeze dried.

### Example 54

### 6-O-(4-Benzoylamino-2-methl)-benzoyl)-D-glucopyranose

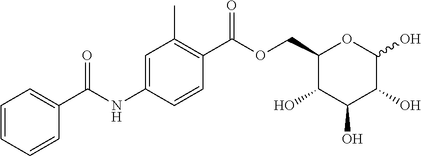 [SMILES: Cc1cc(NC(=O)c2ccccc2)ccc1C(=O)OC[C@H]1OC(O)[C@H](O)[C@@H](O)[C@@H]1O]

Example 54 was synthesized from 6-O-(4-(benzoylamino-2-methyl)-benzoyl)-(2-(trimethylsilyl)ethyl)-α-D-glucopyranoside (60 mg, 115.9 μmol) following the procedure described in synthesis method C.

Yield: 45 mg (107.8 μmol, 93%).

LC/MS (ES-API): m/z=453.17 [M−H]⁻; calculated: 453.10; t_{R} (λ=220 nm): 1.57 min (LC/MS-method A).

¹H-NMR (400 MHz, DMSO-d₆): δ [ppm]=10.44 (s, 1H, CO—NH), 7.94 (m, 2H), 7.88 (m, 1H), 7.77 (m, 2H), 7.57 (m, 3H), 6.63 (s br., C1-OH), 6.34 (s br., C1-OH), 4.94 (d, J=3.42 Hz, 1H, C1′-H), 4.48 (m, 2H), 4.35 (d, J=7.70 Hz, 1H), 4.26 (m, 2H), 3.90 (m, 1H), 3.50 (s, 1H), 3.45 (m, 1H), 3.19 (m, 1H), 2.94 (m, 1H), 2.54 (s, 3H, CH₃).

### Method D: Benzoylation with Tin Derivative

General Description (JOC 2013 78(6), 2336-45; DIPEA Instead of PEMP):

Methyl- or allyl-α-D-glycopyranoside (0.5 mmol) is stirred with di-n-butyl-tin dichloride (50 μmol) in tetrahydrofuran (4 mL) for 10 minutes. Tetrabutylammonium iodide (250 μmol), benzoyl chloride (650 μmol) and diisopropylethylamine (650 μmol) are added and the reaction mixture is stirred at 25° C. for 16 h. Saturated ammonium chloride solution is added. The reaction products are extracted with ethyl acetate (3×6 mL) and the combined organic layers washed with H₂O and finally evaporated.

### Example 65

### 2-O-(4-Benzyloxy-benzoyl)-methyl-α-D-glucopyranoside

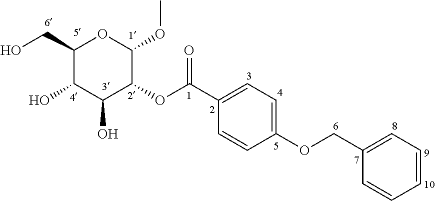 [SMILES: CO[C@H]1O[C@H](CO)[C@@H](O)[C@H](O)[C@H]1OC(=O)c1ccc(OCc2ccccc2)cc1]

Example 65 was synthesized from 4-benzyloxybenzoic acid (320.7 mg, 1.3 mmol) and methyl-α-D-glucopyranoside (194.2 mg, 1 mmol) following the procedure described in synthesis method D. The product was purified by MPLC (silica SiO₂60, eluents n-heptane, ethyl acetate, flow 35 mL/min, gradient: 0-100% ethyl acetate in 11.5 min).

Yield: 380 mg (0.940 mmol, 94%).

LC/MS (ES-API): m/z=405.26 [M+H]⁺; calculated: 405.15; t_{R} (λ=220 nm): 1.57 min (LC/MS-method A).

¹H-NMR (600 MHz, DMSO-d₆): δ [ppm]=7.95 (d, J=8.93 Hz, 2H, AA′BB′ system, C3-H), 7.47 (d, 2H, C8-H), 7.40 (t, 3H, C9-H), 7.35 (t, 1H, C10-H), 7.15 (d, J=8.93 Hz, 2H, AA′BB′ system, C4-H), 5.24 (d, 1H, C3′-OH), 5.21 (s, 2H, O-C6-H2), 5.16 (d, 1H, C4′-OH), 4.85 (d, J=3.67 Hz, 1H, C1′-H), 4.62 (dd, J=10.03, 3.67 Hz, 1H, C2′-H), 4.57 (t, J=5.93 Hz, 1H, C6′-OH), 3.75 (td, J=9.29, 5.62 Hz, 1H, C3′-H), 3.68 (ddd, J=11.65, 5.65, 1.77 Hz, 1H, C6′-Ha), 3.51 (m, 1H, C6′-Hb), 3.41 (dd, 1H, C5′-H), 3.26 (s, 3H, C1′-OCH₃), 3.25 (m, 1H, C4′-H)

¹³C NMR (600 MHz, DMSO-d₆) δ [ppm]=165.19 (C1), 121.96 (C2), 131.51 (C3), 114.74 (C4), 162.30 (C5), 69.48 (C6), 136.39 (C7), 127.76 (C8), 128.45 (C9), 127.98 (C10), 96.42 (C1′), 54.29 (OCH₃), 73.95 (C2′), 70.53 (C3′), 70.16 (C4′), 72.69 (C5′), 60.60 (C6′).

### Method E: 6-O-benzoylation of Protected Galactopyranoside

General Description:

To a solution of benzoic acid (1.92 mmol) in CH₂Cl₂ (7 mL) and N,N-dimethylformamide (5 mL) 1,2:3,4-di-O-isopropylidene-α-D-galactopyranose (500 mg, 1.92 mmol) is added at 0° C. under argon atmosphere and the reaction mixture stirred. After 5 minutes N,N-4-dimethylamino-pyridine (46.94 mg, 384.19 μmol) and dicyclohexylcarbodiimide (396.4 mg, 1.92 mmol) are added. The mixture is left at 0° C. for additional 10 minutes and then allowed to reach 25° C. and stirred for 16 h. The reaction is monitored by two independent methods, LC/MS (method B) and TLC (n-heptane/ethyl acetate=2/1). H₂O (10 mL) is added and the product extracted with CH₂Cl₂ (2×5 mL). The combined organic layers are dried (Na₂SO₄) and evaporated.

Deprotection of Galactoside:

General Description:

### Synthesis of 6-O-acylated-galactopyranose derivatives

2 M HCl (1.59 mL, 3.19 mmol) is added to 6-O-(benzoyl)-1,2:3,4-di-O-isopropylidene-α-D-galactopyranose (212.53 μmol). The reaction mixture is stirred over three days at 25° C. Reaction control is done by LC/MS-method B. The reaction mixture is diluted with H₂O and freeze dried.

### Example 89

### 6-O-(4-Benzyloxy-benzoyl)-D-galactopyranose

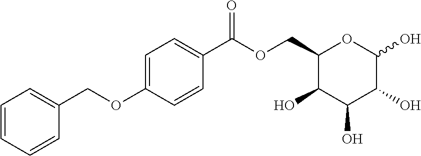 [SMILES: O=C(OC[C@H]1OC(O)[C@H](O)[C@@H](O)[C@H]1O)c1ccc(OCc2ccccc2)cc1]

Example 89 was synthesized from 4-benzyloxybenzoic acid (438.5 mg, 1.92 mmol) and 1,2:3,4-di-O-isopropylidene-α-D-galactopyranose (500 mg, 1.92 mmol) following the procedure described in the first step of synthesis method E. The crude mixture was purified using MPLC (SiO₂60, 80 g; A: n-heptane; B: ethyl acetate; flow: 60 mL/min; gradient: 100% A till 2 min, 0 to 50% B till 32 min, 50% B till 37 minutes). Deprotection was done with 6-O-(4-benzyloxy-benzoyl)-1,2:3,4-di-O-isopropylidene-α-D-galactopyranose (100 mg, 212.5 μmol) and HCl as described in synthesis method E. The crude mixture was purified using MPLC (SiO₂60, 24 g; A: CH₂Cl₂; B: MeOH; flow: 35 mL/min; gradient: 100% A till 2 min, 0 to 100% B till 22 min, 100% B till 29 min).

Yield: 35 mg (89.7 μmol, 21%).

LC/MS (ES-API): m/z=405.26 [M+H]⁺; calculated: 405.15; t_{R} (λ=220 nm): 1.13 min (LC/MS-method A).

¹H-NMR (400 MHz, DMSO-d₆): δ [ppm]=7.90 (d, 2H, AA′BB′ system), 7.40 (m, 5H), 7.12 (d, 2H, AA′BB′ system), 6.60 (d br., C1-OH), 6.21 (d br., C1-OH), 5.19 (s, 2H, OCH₂), 4.98 (d, C1′-H), 4.59 (d, 1H, OH), 4.50 (d, 1H, OH), 4.30 (m, 2H), 4.14 (m, 1H), 3.75 (m, 1H), 3.58 (m, 2H), 3.26 (m, 1H).

### Method F: 6-N-Benzoylation of methyl-6-amino-6-deoxy-α-D-glucopyranoside

General Description:

A solution of benzoic acid (647 μmol) and methyl-6-amino-6-deoxy-α-D-glucopyranoside (125 mg, 647 μmol) in CH₂Cl₂ (10 mL) is stirred. Dicyclohexylcarbodiimide (204 mg, 970.5 μmol) is added and the reaction mixture left for 15 h. The reaction is monitored by LC/MS-method. The solvent is evaporated.

### Example 104

### 6-Deoxy-6-[(4-benzyloxy-benzoyl)-amino]-methyl-α-D-glucopyranoside

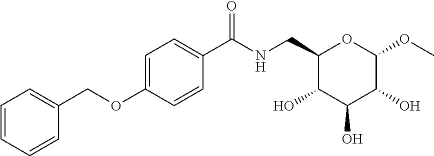 [SMILES: CO[C@H]1O[C@H](CNC(=O)c2ccc(OCc3ccccc3)cc2)[C@@H](O)[C@H](O)[C@H]1O]

Example 104 was synthesized from 4-benzyloxybenzoic acid (150 mg, 657 μmol) and methyl-6-amino-6-deoxy-α-D-glucopyranoside (127 mg, 657 μmol) as described in synthesis method F. The crude mixture was purified using MPLC (SiO₂60, 24 g; A: n-heptane; B: ethyl acetate; flow: 35 mL/min; gradient: 100% A till 1 min, 0 to 100% B till 12.5 min, 100% B till 13 minutes).

Yield: 83 mg (205.7 μmol, 31%).

LC/MS (ES-API): m/z=405.26 [M+H]⁺; calculated: 405.15; t_{R} (λ=220 nm): 1.13 min (LC/MS-method A).

¹H-NMR (400 MHz, DMSO-d₆): δ [ppm]=8.31 (t, J=5.69 Hz, 1H, CO—NH), 7.83 (d, J=8.80 Hz, 2H, AA′BB′ system), 7.40 (d, 2H), 7.40 (t, 2H), 7.33 (t, 1H), 7.06 (d, J=8.80 Hz, 2H, AA′BB′ system), 5.16 (s, 2H, OCH₂), 5.05 (d, J=5.26 Hz, 1H, OH), 4.80 (d, 1H, OH), 4.71 (d, 1H, OH), 4.51 (d, J=3.67 Hz, 1H, OH), 3.67 (ddd, J=13.69, 5.38, 2.57 Hz, 1H), 3.52 (m, 1H), 3.23 (m, 2H), 3.20 (m, 1H), 3.19 (s, 3H, C1′-OCH₃), 2.96 (M, 1H)

### Method G: Benzoylation of methyl-α-D-glycopyranoside with Benzoic Acid Chloride

General Description:

To a solution of the benzoic acid (260.9 μmol) in CH₂Cl₂ (6 mL) SO₂Cl₂ (1 mL) is added and the mixture refluxed for 1 h. The solvent is evaporated in vacuo and the residue is codistilled with toluene (3 x). The residue is taken up in tetrahydrofuran (3 mL) and added to a solution of methyl-α-D-glucopyranoside (75.98 mg, 391.28 μmol) in tetrahydrofuran (5 mL). After stirring for 5 minutes sodium hydride (20.87 mg, 521.71 μmol) is added and the reaction mixture stirred at 100° C. for 16 h. Water is added to the reaction mixture and the organic solvent is evaporated. The aqueous phase is extracted with CH₂Cl₂ (3×5 mL), the combined organic phases are dried (Na₂SO₄) and evaporated.

### Example 153a

### 6-O-(4-Benzyloxy-3,5-dichloro-2-methoxy-6-methyl-benzoyl)-methyl-α-D-glucopyranoside

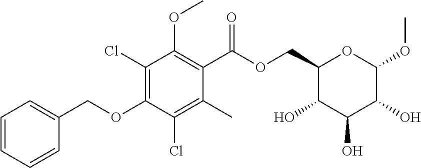 [SMILES: COc1c(Cl)c(OCc2ccccc2)c(Cl)c(C)c1C(=O)OC[C@H]1O[C@H](OC)[C@H](O)[C@@H](O)[C@@H]1O]

Example 153a was synthesized from 4-benzyloxy-3,5-dichloro-2-methoxy-6-methyl benzoic acid (89 mg, 261 μmol) and methyl-α-D-glucopyranoside (76 mg, 391 μmol) following the procedure described in synthesis method G. The product was purified by preparative chiral HPLC (Chiralcel OJ-H/88, 4.6×260 mm, flow 1 mL/min, eluents: n-heptane+ethanol+MeOH=2+1+1).

Yield: 61.4 mg (0.109 mmol, 42%).

LC/MS (ES-API): m/z=561.11 [M−H+formic acid]⁻; calculated: 561.09; t_{R} (λ=220 nm): 1.80 min (LC/MS-method A).

¹H-NMR (400 MHz, DMSO-d₆): δ [ppm]=7.55 (d, J=6.48 Hz, 2H), 7.42 (m, 3H), 5.21 (br d, J=5.75 Hz, 1H), 5.04 (s, 2H), 4.86 (d, J=5.14 Hz, 1H), 4.79 (d, J=6.36 Hz, 1H), 4.62 (dd, J=11.62, 1.83 Hz, 1H), 4.55 (d, J=3.55 Hz, 1H), 4.36 (dd, J=11.62, 5.87 Hz, 1H), 3.83 (s, 3H), 3.65 (m, 1H), 3.40 (m, 1H), 3.26 (s, 3H), 3.21 (m, 1H), 3.13 (m, 1H), 2.67 (quint, J=1.92 Hz, 1H), 2.33 (quint, J=1.81 Hz, 1H), 2.29 (s, 3H), 2.07 (s, 1H).

### Method H

General Description:

To a solution of the benzoic acid (2.27 mmol) in CH₂Cl₂ (10 mL) and N,N-dimethylformamide (8 mL) allyl-D-glycopyranoside (500 mg, 2.27 mmol), N,N-dimethylaminopyridine (55.45 mg, 454 μmol) and dicyclohexylcarbodiimide (468.5 mg, 2.27 mmol) are added at 0° C. under argon atmosphere and the reaction mixture is stirred for 5 h at 0° C. and then left for 24 h at 25° C. H₂O is added and the product extracted with CH₂Cl₂ (2×25 mL). The organic phases are combined and dried (Na₂SO₄). The solvent is evaporated.

### Example 157a

### 2-O-(4-Benzyloxy-benzoyloxy)-allyl-β-D-glucopyranoside

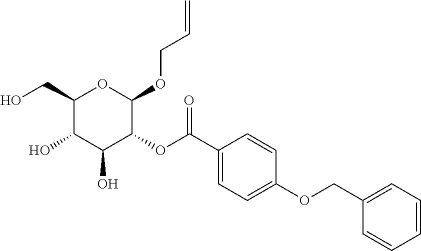 [SMILES: C=CCO[C@@H]1O[C@H](CO)[C@@H](O)[C@H](O)[C@H]1OC(=O)c1ccc(OCc2ccccc2)cc1]

Example 157a was synthesized from 4-benzyloxybenzoic acid (518.2 mg, 2.27 mmol) and allyl-β-D-glycopyranoside (500 mg, 2.27 mmol) following the procedure described in synthesis method H. The crude product was purified by flash column chromatography (Silica, n-heptane/ethyl acetate, 1. Purification: gradient: 0-2 min: 100% n-heptane, 2-25 min: 0-50% ethyl acetate, 25-35 min: n-heptane/ethyl acetate 50/50%; 2. Purification:

gradient: 0-15 min: n-heptane/ethyl acetate 50/50% to 100% ethyl acetate, 15-18 min 100% ethyl acetate). 6-O-(4-Benzyloxy-benzoy)-allyl-β-D-glucopyranoside (example 2) was also isolated in 6% yield (60 mg), for example 157a was isolated in 6% yield and for 157b and 157c see table below.

Yield: 57 mg (0.132 mmol, 6%).

LC/MS (ES-API): m/z=373.1 [M−OAll]⁺; calculated: 373.39; t_{R} (λ=220 nm): 0.80 min (LC/MS-method C).

1H NMR (400 MHz, DMSO-d₆): δ [ppm]=7.92 (d, 2H, AA′BB′ system), 7.48 (d, 2H, aromatic H), 7.40 (t, 2H, aromatic H), 7.35 (t, 1H, aromatic H), 7.25 (d, 2H, AA′BB′ system), 5.74 (m, 1H, CH═CH₂), 5.29 (d, 1H, OH), 5.20 (s, 2H, OCH₂), 5.08-5.19 (m, 2H, OH, CH═CH₂), 5.00 (m, 1H, CH═CH₂), 4.77 (dd, 1H, C2′-H), 4.60 (t, 1H, C6′-OH), 4.54 (d, 1H, C1′-H), 4.22 (m, 1H, CH₂—CH═CH₂), 4.02 (m, 1H, CH₂—CH═CH₂), 3.72 (dd, 1H, C6′-Ha), 3.50 (m, 2H, C6′-Hb, C5′-H), 3.22 (m, 2H, C3′-H, C4′-H).

### Method I: Coupling of 4,6-O-benzylidene-methyl-α-D-glucopyranoside and benzoic acid using 1-hydroxybenzotriazole (HOBt)

General Description:

To a solution of the benzoic acid (274.1 μmol) in CH₂Cl₂ (5 mL) HOBt (46.2 mg, 301.5 μmol) and (3-dimethylamino-propyl)-N′-ethylcarbodiimide (57.8 mg, 301.5 μmol) are added under argon atmosphere. After 2 h at 25° C., 4,6-O-benzylidene-methyl-α-D-glucopyranoside (85.1 mg, 301.5 μmol) and triethylamine (42 μl, 301.5 μmol) are added and the reaction mixture is stirred for 40 h. H₂O (25 mL) is added and the reaction mixture extracted with CH₂Cl₂ (2×25 mL) The combined organic phases are dried (Na₂SO₄) and evaporated.

### Cleavage of 4,6-O-benzylidene acetal

General Description:

Tin dichloride (3.7 mg, 19 μmol) is added to a solution of 2-O-benzoyl-4,6-O-benzylidene-methyl-α-D-glucopyranoside (0.191 mmol) in acetonitrile (10 mL) under argon atmosphere at 25° C. After 30 minutes at 25° C. H₂O (10 mL) is added and the reaction mixture is freeze dried.

Alternatively deprotection can be performed using para-toluene sulfonic acid:

4,6-O-Benzylidene-3-O-benzoyl-methyl-α-D-glucopyranoside or 4,6-O-benzylidene-2-O-benzoyl-methyl-α-D-glucopyranoside (17.4 μmol) is stirred with para-toluene sulfonic acid (2.9 mg, 17.0 μmol) in CH₂Cl₂ (1 mL) at 25° C. for 10 minutes. The reaction mixture is evaporated and the product is purified by HPLC (Merck PurosphereStar 18 e, 75×25 mm, 3 μm, eluents: A: H₂O+0.05% trifluoroacetic acid and B: acetonitrile+0.05% trifluoroacetic acid, gradient: 0-1.2 min: 20% B, 20 mL/min, 1.2-1.7 min 20% B, 30 mL/min, 1.7-7 min: 20-90% B, 32 mL/min, 7-9 min 90-100% B, 32 mL/min, 9-10 min: 100% B, 32 mL/min).

### Example 161

### 2-O-(4-(3-t-Butyloxy-benzyloxy)-3-chloro-5-methoxy-benzoyloxy)-methyl-α-D-glucopyranoside

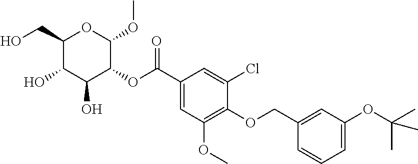 [SMILES: COc1cc(C(=O)O[C@H]2[C@@H](OC)O[C@H](CO)[C@@H](O)[C@@H]2O)cc(Cl)c1OCc1cccc(OC(C)(C)C)c1]

Example 161 was synthesized from 4-(3-t-butyloxy-benzyloxy)-3-chloro-5-methoxy-benzoic acid (100 mg, 274 μmol) and 4,6-O-benzylidene-methyl-α-D-glucopyranoside (85 mg, 301.5 μmol) following the procedure described in synthesis method I. The residue from HOBt coupling was purified by flash column chromatography (Silica, n-heptane/ethyl acetate). Gradient: 0-1 min: 100% n-heptane, 1-12 min: 0-30% ethyl acetate, 12-15 min: 30% ethyl acetate, flow 30 mL/min. The crude product from benzylidene cleavage was purified by HPLC (Agilent Prep-C₁₈ 10 μm 30×250 mm; A: H₂O+0.05% trifluoroacetic acid; B: acetonitrile+0.05% trifluoroacetic acid; flow: 70 mL/min; gradient: 0-2 min 5% B, 2-25 min 5% to 95% B; 25-30 min 95% B, 30-32 min 95% to 100% B, 23-33 min 100% B).

Yield: 28.9 mg (53.4 μmol, 28%).

LC/MS (ES-API): m/z=585.4/587.3 [M−H+formic acid]⁻; calculated: 585.19; t_{R} (λ=220 nm): 2.30 min (LC/MS-method D).

¹H-NMR (600 MHz, DMSO-d₆): δ [ppm]=7.63 (d, J=1.83 Hz, 1H), 7.54 (d, J=2.02 Hz, 1H), 7.28 (t, J=7.74 Hz, 1H), 7.15 (d, J=7.52 Hz, 1H), 7.05 (d, J=1.83 Hz, 1H), 6.93 (d, J=8.07 Hz, 1H), 5.38 (m, 1H, OH), 5.19 (d br., J=5.50 Hz, 1H, OH), 5.12 (s, 2H, OCH₂), 4.87 (d, J=3.67 Hz, 1H, C1′-H), 4.59 (m, 2H, C6′-OH, C2′-H), 3.93 (m, 3H, C1-OCH₃), 3.76 (t br., J=8.44, 8.44 Hz, 1H), 3.68 (m, 1H), 3.51 (dt, J=11.87, 5.89 Hz, 1H), 3.42 (m, 1H), 3.30 (m, 1H), 3.26 (s, 3H), 1.27 (s, 9H, O—C(CH₃)₃).

### Method K: Benzoylation of 4,6-O-Benzylidene-methyl-α-D-glucopyranoside with Benzoic Acid Chloride

General Description:

The benzoic acid (367.2 μmol) and thionyl chloride (268 μl, 3.67 mmol) are stirred at 60° C. for 30 minutes. The reaction mixture is evaporated and the residue dissolved in CH₂Cl₂ (2 mL). This solution is added to a solution of 4,6-benzylidene-methyl-α-D-glucopyranoside (103.7 mg, 367.2 μmol) and triethylamine (153.6 μl, 1.1 mmol) in CH₂Cl₂ (4 mL). The reaction mixture is stirred for 16 h at 25° C. The solvent is evaporated.

### Cleavage of 4,6-O-benzylidene Group Using Trifluoroacetic Acid

General Description:

4,6-O-Benzylidene-3-O-benzoyl-methyl-α-D-glucopyranoside or 4,6-O-benzylidene-2-O-benzoyl-methyl-α-D-glucopyranoside (39.1 μmol) is stirred with trifluoroacetic acid (10 eq., 33.5 μl, 391.4 μmol) in CH₂Cl₂ (1 mL) at 25° C. for 2 h. The reaction mixture is freeze dried.

Alternatively Hydrochloric Acid can be Used for Cleavage:

4,6-O-Benzylidene-3-O-benzoyl-methyl-α-D-glucopyranoside (117.3 μmol) or 4,6-O-benzylidene-2-benzoyl-methyl-α-D-glucopyranoside is stirred with hydrochloric acid (2 M, 2 mL) in acetonitrile (2 mL) at 25° C. for 16 h. The reaction mixture is freeze dried and the product is purified by HPLC (Merck Hibar Lichrospher 100 RP-18e, 10 μm, 25×250 mm, flow 60 mL/min; eluents H₂O+0.05% trifluoroacetic acid and acetonitrile, 0-1.5 min 10% acetonitrile; 1.5-17 min 10-90% acetonitrile, 17-18.5 min 90% acetonitrile).

### Example 162a and 162b

### 3-O-(4-Benzyloxy-2-methoxy-6-methyl-benzoyloxy)-methyl-α-D-glucopyranoside and 2-O-(4-benzyloxy-2-methoxy-6-methyl-benzoyloxy)-methyl-α-D-glucopyranoside

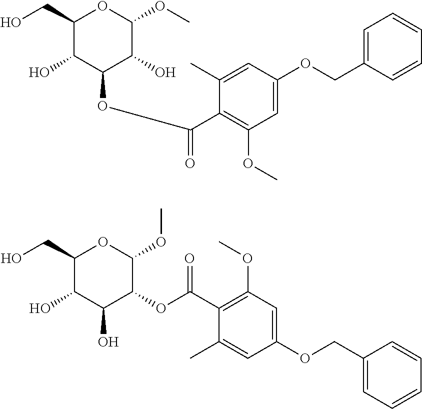 [SMILES: COc1cc(OCc2ccccc2)cc(C)c1C(=O)O[C@@H]1[C@@H](O)[C@@H](OC)O[C@H](CO)[C@H]1O ; COc1cc(OCc2ccccc2)cc(C)c1C(=O)O[C@H]1[C@@H](OC)O[C@H](CO)[C@@H](O)[C@@H]1O]

Example 162a and 162b were synthesized from 4-benzyloxy-2-methoxy-6-methyl benzoic acid (100 mg, 367 μmol) and 4,6-O-benzylidene-methyl-α-D-glucopyranoside (104 mg, 367 μmol) following the procedure described in synthesis method K and the 2-O and 3-0 benzoylated products from the benzoylation reaction were isolated by HPLC (Merck Hibar Lichrospher 100 RP-18e 10 μm, 250×25 mm, eluents: A: H₂O+0.037% trifluoroacetic acid and B: acetonitrile, flow 60 mL/min, gradient: 0-2 min: 5% B, 2-26.5 min 5% to 95% B, 26.5-28.5 min: 95% B). After deprotection using trifluoroacetic acid the crude product was purified by HPLC (Merck Hibar Lichrospher 100 RP-18e 10 μm 250-25, 60 mL/min; eluents H₂O+0.05% trifluoroacetic acid and acetonitrile, 0-1.5 min 10% acetonitrile; 1.5-17 min 10-90% acetonitrile, 17-18.5 min 90% acetonitrile).

### Example 162a

Yield: 23 mg (51.3 μmol, 23%).

LC/MS (ES-API): m/z=449.29 [M+H]⁺; calculated: 449.18; t_{R} (λ=220 nm): 1.89 min (LC/MS-method D).

¹H-NMR (600 MHz, DMSO-d₆): δ [ppm]=7.45 (d, J=7.15 Hz, 2H), 7.40 (t, J=7.70 Hz, 2H), 7.34 (t, J=7.34 Hz, 1H), 6.53 (d, J=2.02 Hz, 1H), 6.49 (d, J=2.02 Hz, 1H), 5.17 (t, J=9.72 Hz, 1H, C3′-H), 5.13 (s, 2H, OCH₂), 4.97 (s br., 1H, OH), 4.73 (s br., 1 H, OH), 4.60 (d, J=3.48 Hz, 1H, C1′-H), 3.71 (s, 3H, OCH₃), 3.64 (dd, J=11.74, 1.65 Hz, 1H), 3.50 (dd, J=11.55, 5.32 Hz, 1H), 3.45 (ddd, J=9.72, 5.32, 1.65 Hz, 1H), 3.42 (dd, J=10.09, 3.67 Hz, 1H), 3.3 (m, 4H), 2.26 (s, 3H, CH₃)

### Example 162b

Yield: 5 mg (5.2 μmol, 3%).

LC/MS (ES-API): m/z=493.33 [M−H+formic acid]⁻; calculated: 493.19; t_{R} (λ=220 nm): 1.89 min (LC/MS-method D).

¹H-NMR (600 MHz, DMSO-d₆): δ [ppm]=7.45 (d, J=7.15 Hz, 2H), 7.40 (t, J=7.70 Hz, 2H), 7.34 (t, J=7.70 Hz, 1H), 6.56 (d, J=1.83 Hz, 1H, aromatic H), 6.51 (d, J=2.02 Hz, 1H), 5.13 (s, 2H, OCH₂), 5.12 (s br., 2H, OH), 4.83 (d, J=3.67 Hz, 1H, C1′-H), 4.60 (dd, J=10.09, 3.67 Hz, 1H), 4.56 (s br., 1H, OH), 3.73 (s, 3H, OCH₃), 3.66 (dd, J=11.37, 1.47 Hz, 1H), 3.61 (dd, J=9.35, 9.54 Hz, 1H), 3.49 (dd, J=11.74, 5.69 Hz, 1H), 3.38 (ddd, J=9.72, 5.50, 1.65 Hz, 1H), 3.35 (m, 1H), 3.29 (s, 3H, C1-OCH₃), 3.22 (dd, J=9.35, 8.99 Hz, 1H), 2.22 (s, 3H, CH₃).

### Method L: Benzoylation of methyl-α-D-glucosamine

General Description:

A solution of the benzoic acid (304.8 μmol), N,N-diisopropylethylamine (106.5 μl, 610 μmol) and HATU (139.1 mg, 365.8 μmol) in N,N-dimethylformamide (1 mL) is stirred for 10 min and added to a suspension of methyl-α-D-glucosamine (70 mg, 304.8 μmol) and N,N-diisopropylethylamine (106.5 μl, 610 μmol) in N,N-dimethylformamide (2 mL). The reaction mixture is stirred at 25° C. for 1 h. The solvent is evaporated.

### Example 171

### 2-Deoxy-2-(4-benzyloxy-3,5-dichloro-benzoylamino)-methyl-α-D-glucopyranoside

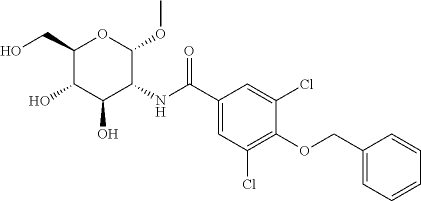 [SMILES: CO[C@H]1O[C@H](CO)[C@@H](O)[C@H](O)[C@H]1NC(=O)c1cc(Cl)c(OCc2ccccc2)c(Cl)c1]

Example 171 was synthesized from 4-benzyloxy-3,5-dichloro-benzoic acid and methyl-α-D-glucosamine following the procedure described in synthesis method L. The crude product is purified by HPLC (Agilent Prep C₁₈, 10 μm, 30×250 mm, flow 75 mL/min, eluents: H₂O and acetonitrile, gradient: 0-12.5 min 10 to 90% B, 12.5-15 min 90% B).

Yield: 48 mg (101.8 μmol, 33%).

LC/MS (ES-API): m/z=470.2/472.1 [M−H]⁻; calculated: 470.07; t_{R} (λ=220 nm): 1.96 min (LC/MS-method D).

¹H-NMR (600 MHz, DMSO-d₆): δ [ppm]=8.51 (d, J=7.9 Hz, 1H), 8.06 (s, 2H), 7.53 (m, 2H), 7.44-7.34 (m, 3H), 5.10 (s, 2H), 5.05 (d, J=5.5 Hz, 1H), 4.83 (d, J=5.7 Hz, 1H), 4.65 (d, J=3.5 Hz, 1H), 4.54 (t, J=5.7 Hz, 1H), 3.86 (m, 1H), 3.68 (m, 2H), 3.50 (m, 1H), 3.37 (m, 1H), 3.25 (s, 3H), 3.19 (m, 1H).

### Method M C2-Benzoylation of protected methyl-α-D-galactoside

General Description:

6-O-Silylation of methyl-3,4-O-isopropylidene-α-D-galactoside

A solution of methyl-3,4-O-isopropylidene-α-D-galactoside (500 mg, 2.13 mmol), triethylamine (446.3 μl, 3.2 mmol), dimethylaminopyridine (52.2 mg, 426.9 μmol), and t-butyl-dimethyl-silylchloride (321.7 mg, 2.13 mmol) in CH₂Cl₂ (10 mL) is stirred under argon atmosphere for 16 h. The solvent is evaporated.

### 2-O-Benzoylation

The benzoic acid (315.6 μmol) and thionyl chloride (682.7 μl, 5.74 mmol) are stirred at 60° C. for 1 h. The reaction mixture is evaporated and the residue dissolved in CH₂Cl₂ (3 mL). This solution is added to a solution of methyl-6-(t-butyl-dimethylsilyl)-3,4-O-isopropylidene-α-D-galactoside (100 mg, 286.9 μmol) and triethylamine (87.1 mg, 860.8 μmol) in CH₂Cl₂ (3 mL). After 2 days at 25° C. the solvent is evaporated.

Cleavage of Protecting Groups

6-t-Butyl-dimethylsilyl-3,4-O-isopropylidene-2-benzoyl-methy-α-D-galactopyranoside (27.4 μmol), acetonitrile (500 μl) and 2 M hydrochloric acid (500 μl, 1.0 mmol) are stirred at 25° C. for 16 h. The reaction mixture is finally freeze dried.

### Example 188

### 2-O-(4-Benzyloxy-3,5-dichloro-2-methoxy-benzoyl)-methyl-α-D-galactopyranoside

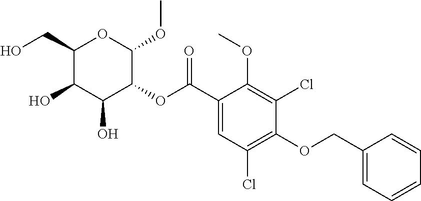 [SMILES: COc1c(C(=O)O[C@H]2[C@@H](OC)O[C@H](CO)[C@H](O)[C@@H]2O)cc(Cl)c(OCc2ccccc2)c1Cl]

Example 188 was synthesized from 4-benzyloxy-3,5-dichloro-2-methoxy-benzoic acid and methyl-3,4-O-isopropylidene-α-D-galactoside following the procedure described in synthesis method M. The crude product from silylation reaction was purified by flash column chromatography (MPLC, Silica, SiO₂60, CH₂Cl₂/MeOH, gradient: 0-5 min: 100% CH₂Cl₂, 5-30 min: 0-5% MeOH, 30-35.5 min: 5% MeOH). The crude product from 2-benzoylation reaction was purified by HPLC (Merck Hibar Lichrospher 100 RP-18e 10 μm 250-25, 60 mL/min; eluents: H₂O+0.05 trifluoroacetic acid and acetonitrile, 0-2 min: 5% acetonitrile, 2.0-26.5 min: 5-95% acetonitrile, 26.5-28.5 min: 100% acetonitrile). After cleavage of the protecting group the product was freeze dried.

Yield: 13 mg (27.4 μmol, quant.).

LC/MS (ES-API): m/z=547.3/549.2/551.3 [M−H+formic acid]⁻; calculated: 547.10; t_{R} (λ=220 nm): 2.17 min (LC/MS-method D).

¹H-NMR (600 MHz, DMSO-d₆): δ [ppm]=7.88 (s, 1H), 7.54 (d, J=6.79 Hz, 2H), 7.42 (m, 3H), 5.12 (s, 2H, OCH₂), 5.10 (m, 1H), 4.89 (d, J=3.67 Hz, 1H, OH), 4.80 (d, J=4.58 Hz, 1H, OH), 4.63 (t, J=5.69, 1 H, C6′-OH), 3.40 (m, 1H), 3.86 (s, 3H, OCH₃), 3.82 (m, 1H), 3.64 (m, 1H), 3.54 (m, 2H, C6′-HaHb), 3.29 (m, 1H), 3.28 (s, 3H, C1′-OCH₃).

### Method N 2-O-benzylation of methyl-α- or β-D-glucopyranoside

General Description:

A solution of methyl-D-glucopyranoside (300 mg, 1.54 mmol) and di-n-butyl-tin oxide (431.7 mg, 1.70 mmol) in toluene (5 mL) is refluxed for 1 h. Benzylchloride (2.32 mmol) and tetrabutylammonium bromide (254.1 mg, 772.5 μmol) are added and the mixture is stirred at 100° C. for 16 h. The reaction mixture is diluted with saturated NaHCO₃ solution and the product is extracted with ethyl acetate (3×5 mL). The combined organic phases are dried (Na₂SO₄), filtered and evaporated.

### Example 192

### 2-O-(4-Benzyloxy-benzyl)-methyl-α-D-glucopyranoside

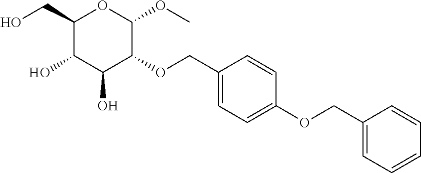 [SMILES: CO[C@H]1O[C@H](CO)[C@@H](O)[C@H](O)[C@H]1OCc1ccc(OCc2ccccc2)cc1]

Example 192 was synthesized from 4-benzyloxy-benzyl chloride and methyl-α-D-glucopyranoside following the procedure described in synthesis method N. The reaction mixture was purified by HPLC (Waters SunFire Prep OBD C₁₈, 5 μm, 50×100 mm, eluents: A: H₂O+0.1% trifluoroacetic acid and B: acetonitrile, flow 120 mL/min, gradient: 0-2.5 min: 10% B, 2.5-10.5 min 10% to 100% B, 10.5-13 min: 100% B).

Yield: 67.3 mg (186.9 μmol, 17%).

LC/MS (ES-API): m/z=435.15 [M−H+formic acid]⁻; calculated: 435.10; t_{R} (λ=220 nm): 1.50 min (LC/MS-method A).

¹H-NMR (400 MHz, DMSO-d₆): δ [ppm]=7.44 (d, J=7.62 Hz, 2H), 7.38 (t, J=6.85 Hz, 2H), 7.33 (d, J=6.85 Hz, 1H), 7.28 (d, J=8.38 Hz, 2H), 6.98 (d, J=8.68 Hz, 2H), 5.10 (s, 2H), 4.62-4.48 (m, 3H), 3.61 (d, J=11.12 Hz, 1H), 3.51 (t, J=9.18 Hz, 1H), 3.42 (dd, J=11.57, 5.58 Hz, 1H), 3.28 (m, 2H), 3.23 (s, 3H), 3.13 (dd, J=9.57, 3.46 Hz, 1H), 3.07 (dd, J=9.77, 8.57 Hz, 1H).

### Method O Deprotection of 1-O-allyl

General Description:

To a solution of 6-O-(4-benzyloxy-benzoyl)-allyl-α-D-glucopyranoside (50 mg, 116.2 μmol) in ethanol (5 mL) dihydridotetrakis(triphenylphosphine)ruthenium(II) (7.04 mg, 5.81 μmol) is added under argon atmosphere and the reaction mixture is stirred at 95° C. for 2 h. The reaction is monitored by LC/MS (method A). After additional addition of para-toluene sulfonic acid (2 mg, 11.6 μmol), stirring of the reaction mixture at 95° C. for 3 h, addition of para-toluene sulfonic acid (15 mg, 174.0 μmol) and heating to 95° C. for 3 h the reaction is stopped. The reaction mixture is evaporated.

### Example 195

### 6-O-(4-Benzyloxy-benzoyl)-ethyl-D-glucopyranosides

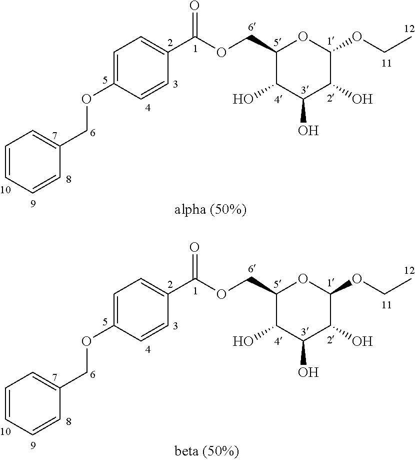 [SMILES: CCO[C@H]1O[C@H](COC(=O)c2ccc(OCc3ccccc3)cc2)[C@@H](O)[C@H](O)[C@H]1O ; CCO[C@@H]1O[C@H](COC(=O)c2ccc(OCc3ccccc3)cc2)[C@@H](O)[C@H](O)[C@H]1O]

Example 195 was synthesized from 4-O-benzyloxy-benzoyl-1-O-allyl-α-D-glucopyranoside (example 2) following the procedure described in synthesis method O. The reaction mixture was purified by flash column chromatography (MPLC, Silica

SiO₂60, 12 g, flow 30 mL/min, eluent ethyl acetate/MeOH=9:1).

Yield: 17 mg (40.6 μmol, 35%).

LC/MS (ES-API): m/z=463.21 [M−H+formic acid]⁻; calculated: 463.19 t_{R} (λ=220 nm): 1.64 min (LC/MS-method A).

α anomer:

¹H NMR (400 MHz, DMSO-d₆): δ [ppm]=7.91 (m, 2H, C3-H, AA′BB′ system), 7.46 (d, 2H, C8-H), 7.40 (t, 2H, C9-H), 7.35 (t, 1H, C10-H), 7.14 (d, 2H, C4-H, AA′BB′ system), 5.20 (s, 1H, C4′-OH), 5.19 (s, 2H, 006-H2), 4.87 (d, 1H, C3′-OH), 4.72 (d, 1H, C2′-OH), 4.67 (d, J=3.7 Hz, 1H, C1′-H), 4.49 (m, 1H, C6′-Ha), 4.25 (m, 1H, C6′-Hb), 3.72 (m, 1H, C5′-H), 3.63 (m, 2H, C11-H2), 3.43 (m, 1H, C3′-H), 3.23 (m, 1H, C2′-H), 3.18 (m, 1H, C4′-H), 1.14 (t, 3H, C12-H3).

β anomer:

¹H NMR (400 MHz, DMSO-d₆): δ [ppm]=7.90 (m, 2H, C3-H, AA′BB′ system), 7.46 (d, 2H, C8-H), 7.40 (t, 2H, C9-H), 7.35 (t, 1H, C10-H), 7.14 (d, 2H, C4-H, AA′BB′ system), 5.22 (s, 1H, C4′-OH), 5.19 (s, 2H, 006-H2), 5.06 (d, 1H, C2′-OH), 5.05 (d, 1H, C3′-OH), 4.50 (m, 1H, C6′-Ha), 4.25 (m, 1H, C6′-Hb), 4.19 (d, J=7.8 Hz, 1H, C1′-H), 3.49 (m, 2H, C11-H2), 3.46 (m, 1H, C5′-H), 3.19 (m, 1H, C3′-H), 3.18 (m, 1H, C4′-H), 2.98 (m, 1H, C2′-H), 1.10 (t, 3H, C12-H3).

### Example 217

### 6-O-(4-Benzyloxy-benzoyl)-methyl-β-D-galactopyranoside

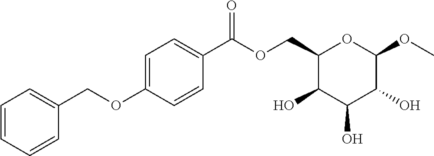 [SMILES: CO[C@@H]1O[C@H](COC(=O)c2ccc(OCc3ccccc3)cc2)[C@H](O)[C@H](O)[C@H]1O]

### Step-1: 6-(4-Benzyloxy-benzoyl)-3,4-O-isopropylidene-methyl-β-D-galactopyranoside

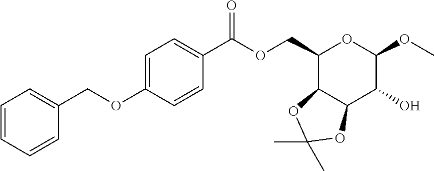 [SMILES: CO[C@@H]1O[C@H](COC(=O)c2ccc(OCc3ccccc3)cc2)[C@@H]2OC(C)(C)O[C@@H]2[C@H]1O]

To 3,4-O-isopropylidene-methyl-α-D-galactopyranoside (3 g, 12.82 mmol) in CH₂Cl₂/N,N-dimethylformamide (1:1; 60 mL), dicyclohexylcarbodiimide (2.64 g, 12.82 mmol), 4-benzyloxybenzoic acid (2.92 g, 12.82 mmol) and dimethylaminopyridine (312 mg, 2.56 mmol) were added at 25° C. and stirred for 1 h. After completion of the reaction, the reaction mixture was filtered and the residue obtained was washed with CH₂Cl₂. The filtrate collected was washed (sat. aqueous NaHCO₃ followed by H₂O). The separated organic layer was dried over anhydrous Na₂SO₄, filtered and concentrated under reduced pressure. The crude compound was purified by silica gel column chromatography (MPLC) eluting with 0-50% ethyl acetate in n-hexane.

Yield: 4.5 g (79%), white solid

LC/MS: m/z=467.00 [M+Na]⁺; calculated: 467.49 t_{R} (λ=220 nm): 1.96 min (LC/MS-method E).

¹H NMR (400 MHz, DMSO-d₆) δ [ppm]=7.92 (d, J=8.80 Hz, 2H), 7.44-7.47 (m, 2H), 7.33-7.42 (m, 3H), 7.14 (d, J=8.80 Hz, 2H), 5.35 (d, J=4.89 Hz, 1H), 5.18 (s, 2H), 4.35-4.44 (m, 2H), 4.19 (d, J=5.38 Hz, 1H), 4.15 (dd, J=4.40, 7.34 Hz, 1H), 4.10 (d, J=8.31 Hz, 1H), 3.98 (t, J=6.11 Hz, 1H), 3.34 (s, 3H), 3.18-3.24 (m, 1H), 1.40 (s, 3H), 1.26 (s, 3H).

### Step-2: 6-O-(4-Benzyloxy-benzoyl)-methyl-β-D-galactopyranoside

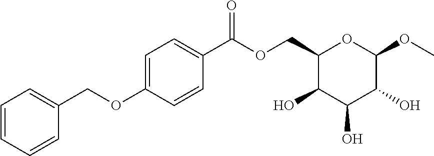 [SMILES: CO[C@@H]1O[C@H](COC(=O)c2ccc(OCc3ccccc3)cc2)[C@H](O)[C@H](O)[C@H]1O]

To 6-O-(4-benzyloxy-benzoyl)-3,4-O-isopropylidene-methyl-β-D-galactopyranoside (4.5 g, 10.12 mmol) in acetonitrile (50 mL), a 2 M HCl solution (20 mL) in H₂O (20 mL) was added and stirred at 25° C. for 1 h. After completion of the reaction, the reaction mixture was evaporated under reduced pressure resulting in the crude compound. The crude compound was purified by silica gel column chromatography (MPLC) eluting with 0-5% MeOH in CH₂Cl₂.

Yield: 810 mg (20%), off white solid

LC/MS: m/z=427.00 [M+Na]⁺; calculated: 427.42 t_{R} (λ=220 nm): 1.67 min (LC/MS-method E).

¹H NMR (400 MHz, DMSO-d₆) δ [ppm]=7.89 (d, J=9.03 Hz, 2H), 7.41-7.45 (m, 2H), 7.37 (t, J=7.22 Hz, 2H), 7.32 (d, J=7.22 Hz, 1H), 7.11 (d, J=9.03 Hz, 2H), 5.16 (s, 2H), 4.92 (d, J=4.06 Hz, 1H), 4.75 (d, J=4.97 Hz, 1H), 4.66 (d, J=4.51 Hz, 1H), 4.24-4.37 (m, 2H), 4.03 (d, J=6.77 Hz, 1H), 3.72 (t, J=6.32 Hz, 1H), 3.64-3.68 (m, 1H), 3.29-3.31 (m, 2H), 3.28 (s, 3H).

### Example 218

### 2-O-(4-Benzyloxy-benzoyl)-methyl-β-D-galactopyranoside

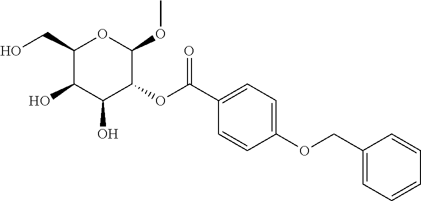 [SMILES: CO[C@@H]1O[C@H](CO)[C@H](O)[C@H](O)[C@H]1OC(=O)c1ccc(OCc2ccccc2)cc1]

### Step-1: 6-O-(t-Butyl-dimethyl-silyloxy)-3,4-O-isopropylidene-methyl-β-D-galactopyranoside

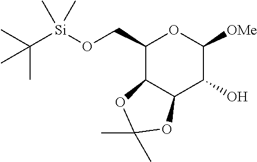 [SMILES: CO[C@@H]1O[C@H](CO[Si](C)(C)C(C)(C)C)[C@@H]2OC(C)(C)O[C@@H]2[C@H]1O]

To 3,4-O-isopropylidene-methyl-β-D-galactopyranoside (7 g, 29.91 mmol) in CH₂Cl₂ (100 mL), triethyl amine (6.2 mL, 44.87 mmol), t-butyl-dimethyl-silyl chloride (4.5 g, 29.91 mmol) and dimethylaminopyridine (729 mg, 5.980 mmol) were added at 25° C. and stirred for 18 h. After completion of the reaction, the reaction mixture was concentrated under reduced pressure. The crude compound was purified by MPLC (Silica, eluting with 0-5% MeOH in CH₂Cl₂).

Yield: 8.2 g (78%), white solid.

¹H NMR (400 MHz, DMSO-d₆) δ [ppm]=5.24 (d, J=4.89 Hz, 1H), 4.06 (dd, J=1.47, 5.38 Hz, 1H), 4.01 (d, J=7.83 Hz, 1H), 3.87-3.92 (m, 1H), 3.66-3.79 (m, 3H), 3.34 (s, 3H), 3.15 (dt, J=5.14, 7.46 Hz, 1H), 1.35 (s, 3H), 1.21 (s, 3H), 0.84 (s, 9H), 0.03 (s, 6H).

### Step-2: 2-O-(4-Benzyloxy-benzoyl)-6-(t-butyl-dimethyl-silyloxy)-3,4-O-isopropylidene-methyl-β-D-galactopyranoside

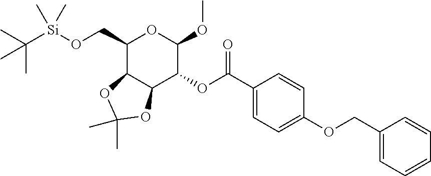 [SMILES: CO[C@@H]1O[C@H](CO[Si](C)(C)C(C)(C)C)[C@@H]2OC(C)(C)O[C@@H]2[C@H]1OC(=O)c1ccc(OCc2ccccc2)cc1]

To 4-benzyloxybenzoic acid (2.5 g. 10.95 mmol) in CH₂Cl₂ (30 mL) at 0° C., oxalyl chloride (1.9 mL, 21.90 mmol) was added followed by a catalytic amount of N,N-dimethylformamide (0.5 mL) and the mixture stirred at 25° C. for 2 h. The reaction mixture was concentrated under reduced pressure resulting in the formation of corresponding acid chloride.

To 4-benzyloxy-benzoyl chloride (2.69 g, 10.92 mmol) in CH₂Cl₂ (30 mL), a mixture of triethyl amine (6.1 mL, 43.85 mmol), dimethylaminopyridine (267 mg, 2.192 mmol) and 6-(t-butyl-dimethyl-silyloxy)-3,4-O-isopropylidene-methyl-β-D-galactopyranoside (4.2 g, 12.06 mmol) in CH₂Cl₂ (40 mL) was added at 25° C. The reaction mixture was stirred for 1 h. After completion of the reaction, the reaction mixture was diluted with H₂O and extracted with CH₂Cl₂ (3×). The combined organic layer was dried (Na₂SO₄), filtered and concentrated under reduced pressure. The crude compound was purified by silica gel column chromatography (MPLC) eluting with 0-50% ethyl acetate in n-hexane.

Yield: 2.5 g (40%), white solid.

¹H NMR (400 MHz, DMSO-d₆) δ [ppm]=8.31-8.35 (m, 1H), 8.05 (d, J=8.80 Hz, 1H), 7.89-7.93 (m, 1H), 7.45 (d, J=6.85 Hz, 2H), 7.32-7.42 (m, 3H), 7.13 (d, J=8.80 Hz, 1H), 5.24 (s, 1H), 5.19 (s, 1H), 5.16-5.20 (m, 1H), 4.94 (t, J=7.83 Hz, 1H), 4.63 (t, J=8.07 Hz, 1H), 4.36 (d, J=8.31 Hz, 1H), 4.16-4.27 (m, 2H), 3.90-3.98 (m, 1H), 3.73-3.82 (m, 2H), 3.34 (s, 3H), 0.87 (s, 4H), 0.85 (s, 5H), 0.06 (s, 3H), 0.05 (s, 3H).

### Step-3: 2-O-(4-Benzyloxy-benzoyl)-methyl-β-D-galactopyranoside

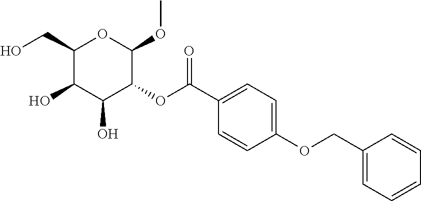 [SMILES: CO[C@@H]1O[C@H](CO)[C@H](O)[C@H](O)[C@H]1OC(=O)c1ccc(OCc2ccccc2)cc1]

To a solution of 2-O-(4-benzyloxy-benzoyl)-6-(t-butyl-dimethyl-silyloxy)-3,4-O-isopropylidene-methyl-β-D-galactopyranoside (2.5 g, 4.819 mmol) in acetonitrile (30 mL), 2 M HCl (10 mL) was added. The mixture was stirred at 25° C. for 1 h. After completion of the reaction, the reaction mixture was evaporated under reduced pressure resulting in the crude compound. The crude compound was purified by silica gel column chromatography (MPLC) eluting with 0-5% MeOH in CH₂Cl₂.

Yield: 1.3 g (66%), off white solid.

LC/MS: m/z=427.13 [M+Na]⁺; calculated: 427.42 t_{R} (λ=220 nm): 1.64 min (LC/MS-method E).

¹H NMR (400 MHz. DMSO-d₆) δ [ppm]=7.91 (d, J=8.80 Hz. 2H), 7.43-7.48 (m, 2H), 7.39 (t, J=7.34 Hz, 2H), 7.33-7.36 (m, 1H), 7.12 (d, J=9.29 Hz, 2H), 5.20 (s, 2H), 5.04 (dd, J=8.31, 9.78 Hz, 1H), 4.96 (d, J=6.36 Hz, 1H), 4.73 (d, J=4.40 Hz, 1H), 4.65 (t, J=5.62 Hz, 1H), 4.38 (d, J=8.31 Hz, 1H), 3.74 (t, J=3.67 Hz, 1H), 3.66 (ddd, J=3.42, 6.72, 9.90 Hz, 1H), 3.51-3.59 (m, 2H), 3.45-3.51 (m, 1H), 3.31 (s, 3H).

### Example 219

### 6-O-(4-Benzyloxy-benzoyl)-methyl-α-D-galactopyranoside

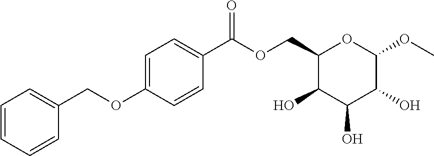 [SMILES: CO[C@H]1O[C@H](COC(=O)c2ccc(OCc3ccccc3)cc2)[C@H](O)[C@H](O)[C@H]1O]

### Step-1: 6-O-(4-Benzyloxy-benzoyl)-3,4-O-isopropylidene-methyl-α-D-galactopyranoside

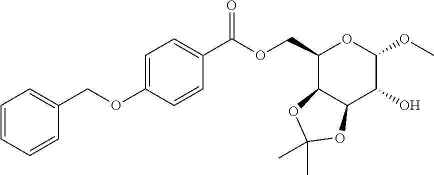 [SMILES: CO[C@H]1O[C@H](COC(=O)c2ccc(OCc3ccccc3)cc2)[C@@H]2OC(C)(C)O[C@@H]2[C@H]1O]

To 3,4-O-isopropylidene-methyl-f3-D-galactopyranoside (2 g, 8.537 mmol) in CH₂Cl₂/N,N-dimethylformamide (1:1; 40 mL), CH₂Cl₂ (1.75 g, 8.537 mmol), 4-benzyloxybenzoic acid (1.94 g, 8.537 mmol) and dimethylaminopyridine (208 mg, 1.70 mmol) were added at 25° C. and the mixture was stirred for 1 h. The reaction mixture was filtered and the residue obtained was washed with CH₂Cl₂. The filtrate collected was washed with saturated aqueous NaHCO₃ solution followed by H₂O. The separated organic layer was dried (Na₂SO₄), filtered and concentrated under reduced pressure. The crude compound was purified by silica gel column chromatography (MPLC) eluting with 0-50% ethyl acetate in n-hexane.

Yield: 3 g (79%), white solid.

### Step-2: 6-O-(4-Benzyloxy-benzoyl)-methyl-α-D-galactopyranoside

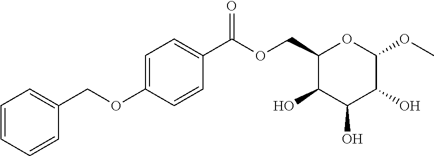 [SMILES: CO[C@H]1O[C@H](COC(=O)c2ccc(OCc3ccccc3)cc2)[C@H](O)[C@H](O)[C@H]1O]

To 6-O-(4-benzyloxy-benzoyl)-3,4-O-isopropylidene-methyl-α-D-galactopyranoside (3 g, 6.756 mmol) in acetonitrile (30 mL) a 2 M HCl solution (14 mL) in H₂O (20 mL) was added and stirred at 25° C. for 1 h. The reaction mixture was evaporated under reduced pressure resulting in the crude compound. The crude compound was purified by silica gel column chromatography (MPLC) eluting with 0-5% MeOH in CH₂Cl₂.

Yield: 1.15 g (42%), off white solid.

LC/MS: m/z=426.95 [M+Na]⁺; calculated: 427.42 t_{R} (λ=220 nm): 1.63 min (LC/MS-method E).

¹H NMR (400 MHz, DMSO-d₆) δ [ppm]=7.91 (d, J=8.80 Hz, 2H), 7.44-7.48 (m, 2H), 7.40 (t, J=7.27 Hz, 2H), 7.32-7.37 (m, 1H), 7.13 (d, J=8.80 Hz, 2H), 5.19 (s, 2H), 4.70 (d, J=4.16 Hz, 1H), 4.62 (d, J=4.52 Hz, 1H), 4.59 (d, J=3.30 Hz, 2H), 4.26-4.38 (m, 2H), 3.92 (dd, J=4.10, 7.76 Hz, 1H), 3.75-3.79 (m, 1H), 3.53-3.64 (m, 2H), 3.26 (s, 3H).

### Example 220

### 2-O-(4-Benzyloxy-benzoyl)-methyl-α-D-galactopyranoside

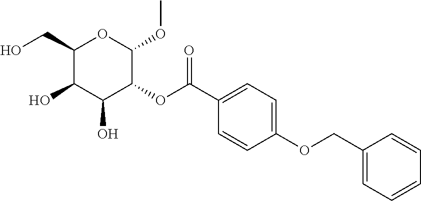 [SMILES: CO[C@H]1O[C@H](CO)[C@H](O)[C@H](O)[C@H]1OC(=O)c1ccc(OCc2ccccc2)cc1]

### Step-1: 6-(t-Butyl-dimethyl-silyloxy)-3,4-O-isopropylidene-methyl-α-D-galactopyranoside

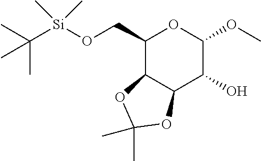 [SMILES: CO[C@H]1O[C@H](CO[Si](C)(C)C(C)(C)C)[C@@H]2OC(C)(C)O[C@@H]2[C@H]1O]

To 3,4-O-isopropylidene-methyl-α-D-galactopyranoside (6.5 g, 27.75 mmol) in CH₂Cl₂ (100 mL), triethyl amine (5.8 mL, 41.62 mmol), t-butyl-dimethyl-silyl-chloride (4.16 g, 27.75 mmol) and dimethylaminopyridine (677 mg, 5.549 mmol) were added at 25° C. and stirred for 18 h. After completion of the reaction, the reaction mixture was concentrated under reduced pressure. The crude compound was purified by silica gel column chromatography eluting with 0-5% MeOH in CH₂Cl₂.

Yield: 7.5 g (77%), white solid.

¹H NMR (400 MHz, DMSO-d₆) δ [ppm]=5.00-5.03 (m, 1H), 4.50 (d, J=3.42 Hz, 1H), 4.14 (dd, J=2.20, 5.62 Hz, 1H), 3.96 (dd, J=5.62, 7.58 Hz, 1H), 3.80-3.85 (m, 1H), 3.71-3.76 (m, 1H), 3.61-3.67 (m, 1H), 3.42 (dt, J=3.67, 7.21 Hz, 1H), 3.25 (s, 3H), 1.36 (s, 3H), 1.21 (s, 3H), 0.84 (s, 9H), 0.03 (s, 6H).

### Step-2: 2-O-(4-Benzyloxy-benzoyl)-6-(t-butyl-dimethyl-silyloxy)-methyl-α-D-galactopyranoside

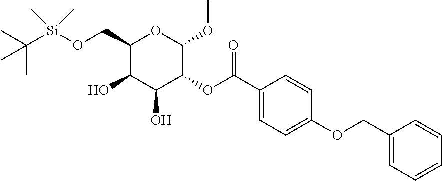 [SMILES: CO[C@H]1O[C@H](CO[Si](C)(C)C(C)(C)C)[C@H](O)[C@H](O)[C@H]1OC(=O)c1ccc(OCc2ccccc2)cc1]

To 4-benzyloxybenzoic acid (2.5 g, 10.96 mmol) in CH₂Cl₂ (30 mL) at 0° C., oxalyl chloride (2.2 mL, 21.92 mmol) was added followed by a catalytic amount of N,N-dimethylformamide (0.5 mL). The reaction mixture was stirred at 25° C. for 2 h. The reaction mixture was concentrated under reduced pressure resulting in the formation of the corresponding acid chloride. To 4-benzyloxy-benzoyl chloride (2.7 g, 10.95 mmol) in CH₂Cl₂ (30 mL) a mixture of triethyl amine (6.1 mL, 43.81 mmol), dimethylaminopyridine (267 mg, 2.188 mmol) and 6-(t-butyl-dimethyl-silyloxy)-3,4-O-isopropylidene-methyl-α-D-galactopyranoside (4.2 g, 12.03 mmol) in CH₂Cl₂ (10 mL) was added at 25° C. and the reaction mixture stirred for 1 h. The reaction mixture was diluted with H₂O and extracted with CH₂Cl₂. The combined organic layers were dried (Na₂SO₄), filtered and concentrated under reduced pressure. The crude compound was purified by column chromatography Silica (MPLC), (0-50% ethyl acetate in n-hexane).

Yield: 4.5 g (72%), off white solid.

LC/MS: m/z=519.20 [M]⁺; calculated: 518.69 t_{R} (λ=220 nm): 2.73 min (LC/MS-method E).

¹H NMR (400 MHz, DMSO-d₆) δ [ppm]=7.92 (d, J=9.29 Hz, 2H), 7.44-7.47 (m, 2H), 7.39 (t, J=7.34 Hz, 2H), 7.30-7.36 (m, 1H), 7.14 (d, J=8.80 Hz, 2H), 5.20 (s, 2H), 4.93 (dd, J=3.67, 8.07 Hz, 1H), 4.85 (d, J=3.42 Hz, 1H), 4.39 (dd, J=5.38, 7.83 Hz, 1H), 3.92-4.35 (m, 3H), 3.71-3.86 (m, 3H), 3.28 (s, 3H), 0.87 (s, 9H), 0.06 (s, 6H).

### Step-3: 2-O-(4-Benzyloxy-benzoyl)-methyl-α-D-galactopyranoside

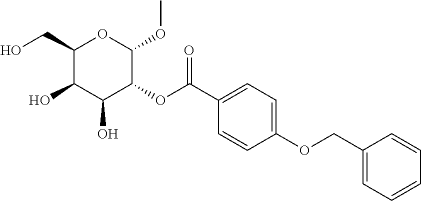 [SMILES: CO[C@H]1O[C@H](CO)[C@H](O)[C@H](O)[C@H]1OC(=O)c1ccc(OCc2ccccc2)cc1]

To 2-O-(4-benzyloxy-benzoyl)-6-(t-butyl-dimethyl-silyloxy)-methyl-α-D-galactopyranoside (4.5 g, 8.675 mmol) in acetonitrile (50 mL), a 2 M HCl solution (20 mL) was added and the mixture stirred at 25° C. for 1 h. After completion of the reaction, the reaction mixture was evaporated under reduced pressure resulting in the crude compound. The crude compound was purified by MPLC (Silica, CH₂Cl₂./3-5% MeOH) Yield: 2.8 g (81%), off white solid.

LC/MS: m/z=427.05 [M+Na]⁺; calculated: 427.42 t_{R} (λ=220 nm): 1.59 min (LC/MS-method E).

¹H NMR (400 MHz, DMSO-d₆) δ [ppm]=7.95 (d, J=8.80 Hz, 2H), 7.45-7.48 (m, 2H), 7.40 (t, J=7.34 Hz, 2H), 7.31-7.37 (m, 1H), 7.14 (d, J=8.80 Hz, 2H), 5.20 (s, 2H), 4.99-5.04 (m, 2H), 4.86 (d, J=3.42 Hz, 1H), 4.77 (d, J=4.40 Hz, 1H), 4.64 (t, J=5.62 Hz, 1H), 3.90 (ddd, J=2.93, 6.60, 10.03 Hz, 1H), 3.83 (d, J=3.91 Hz, 1H), 3.61-3.67 (m, 1H), 3.48-3.60 (m, 2H), 3.25 (s, 3H).

¹H NMR (400 MHz, DMSO-d₆; D₂O exchange) δ [ppm]=7.91 (d, J=8.78 Hz, 2H), 7.38-7.43 (m, 2H), 7.35 (t, J=7.40 Hz, 2H), 7.27-7.33 (m, 1H), 7.08 (d, J=8.78 Hz, 2H), 5.14 (s, 2H), 4.97 (dd, J=3.51, 10.29 Hz, 1H), 4.83 (d, J=3.26 Hz, 1H), 3.89 (dd, J=2.89, 10.42 Hz, 1H), 3.80 (d, J=2.51 Hz, 1H), 3.61-3.66 (m, 1H), 3.50-3.54 (m, 2H), 3.20 (s, 3H).

### Example 221

### 2-Deoxy-2-amino-(3,5-dichloro-4-((3-((3,12-dioxo-2,5,8,14,17-pentaoxa-11-azanonadecan-19-yl)carbamoyl)benzyl)oxy)-2-methylbenzoyl)-1-O-methyl-α-D-glucopyranoside

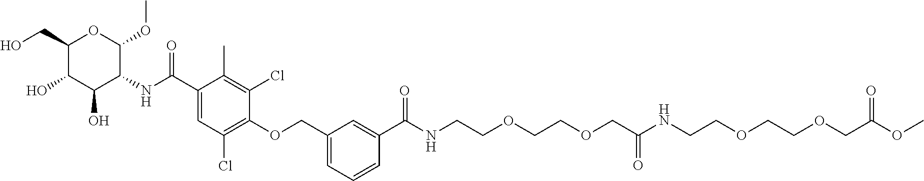 [SMILES: COC(=O)COCCOCCNC(=O)COCCOCCNC(=O)c1cccc(COc2c(Cl)cc(C(=O)N[C@H]3[C@@H](OC)O[C@H](CO)[C@@H](O)[C@@H]3O)c(C)c2Cl)c1]

To Example 209 (12 g, 20.3 mmol) in CH₂Cl₂ (80 mL), 20 mL trifluoroacetic acid (12 eq., 260 mmol) was added. After completion of the reaction (TLC, CH₂Cl₂/MeOH=8/1, R_{f}=0.3), the reaction mixture was concentrated under reduced pressure resulting in the formation of corresponding acid.

The crude acid (210 mg, 0.4 mmol), obtained above, and methyl-17-amino-10-oxo-3,6,12,15-tetraoxa-9-azaheptadecanoate hydrochloride were coupled following the procedure described in synthesis method L. The crude product was purified using MPLC (Silica, SiO₂60, 30 g, eluents: CH₂Cl₂ and MeOH, gradient: 0-5 min: 100% CH₂Cl₂, 5-20 min: 0-10% MeOH, 20-30 min: 10% MeOH, 30-45 min: 10-20% MeOH).

Yield: 100 mg (331 μmol, 30%).

LC/MS (ES-API): m/z=834.2/836.2 [M+H]⁺; calculated: 834.2/836.2; t_{R} (λ=220 nm): 0.67 min (LC/MS-method C).

¹H-NMR (400 MHz, DMSO-d₆): δ [ppm]=8.58 (t, J=5.50, 5.50 Hz, 1H), 8.41 (d, J=8.19 Hz, 1H), 8.02 (s, 1H), 7.87 (d, J=7.82 Hz, 1H), 7.71 (d, J=7.70 Hz, 1H), 7.63 (br t, J=5.69 Hz, 1H), 7.53 (t, J=7.64, 7.64 Hz, 1H), 7.45 (s, 1H), 5.06 (s, 2H), 5.02 (d, J=5.62 Hz, 1H), 4.86 (d, J=5.75 Hz, 1H), 4.71 (d, J=3.55 Hz, 1H), 4.54 (t, J=5.99 Hz, 1H), 4.12 (s, 2H), 3.88 (s, 2H), 3.83 (m, 1H), 3.66 (m, 1H), 3.64 (s, 3H), 3.60-315 (m, 26H), 2.36 (s, 3H).

### Example 222

### 2-Deoxy-2-amino-(3,5-dichloro-4-((3-(1,10-dioxo-5,8,14,17-tetraoxa-2,11-diazanonadecan-19-oicacid)benzyl)oxy)-2-methylbenzoyl)-1-O-methyl-α-D-glucopyranoside

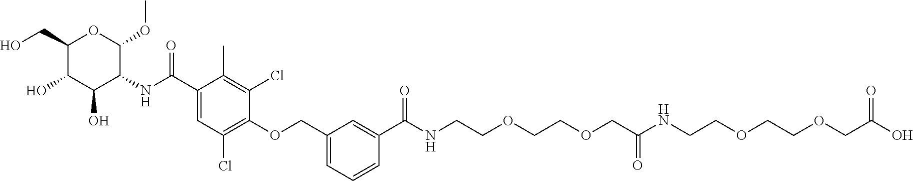 [SMILES: CO[C@H]1O[C@H](CO)[C@@H](O)[C@H](O)[C@H]1NC(=O)c1cc(Cl)c(OCc2cccc(C(=O)NCCOCCOCC(=O)NCCOCCOCC(=O)O)c2)c(Cl)c1C]

To Example 221 (60 mg, 71 μmol) in 4 mL tetrahydrofuran/MeOH (3:1), LiOH (2 eq., in 1 mL H₂O) was added. After completion of the reaction (LC/MS method C), the reaction mixture was acidified with Dowex Marathon H. The Ion exchanger was filtered off and the solvent was removed under reduced pressure.

Yield: 59 mg (63.4 μmol, 88%).

LC/MS (ES-API): m/z=820.2/822.2 [M+H]⁺; calculated: 820.2/822.2; t_{R} (λ=220 nm): 0.63 min (LC/MS-method C).

¹H-NMR (400 MHz, DMSO-d₆): δ [ppm]=8.63 (br. s, 1H), 8.03 (s, 1H), 7.87 (d, J=7.70 Hz, 1H), 7.71 (d, J=7.52 Hz, 1H), 7.63 (m, 1H), 7.52 (t, J=7.61, 7.61 Hz, 1H), 7.44 (s, 1H), 5.05 (m, 3H), 4.73 (d, J=3.48 Hz, 1H), 4.53 (br. s, 1H), 3.87 (m, 4H), 3.79 (m, 1H), 3.67 (br, m, 1H), 3.60-3.15 (m, 28H), 2.35 (s, 3H).

### Method P Coupling to Insulins Using Click Chemistry

General Description:

To a solution of the alkynes (1.2 eq.) and 4-azido-butan-(human Insulin-B29Lys)-amide (described in Example 111 of WO2017207754A1 published on 7 Dec. 2017, 1 eq.) in N,N-dimethylformamide and H₂O, a mixture of the premixed click reagents is added in this order: CuSO₄*5H₂O (0.5 eq.), THPTA (0.8 eq.), and sodium ascorbate (1 eq.). The reaction mixture is agitated at 25° C. for 2 h and freeze dried.

### Example 223

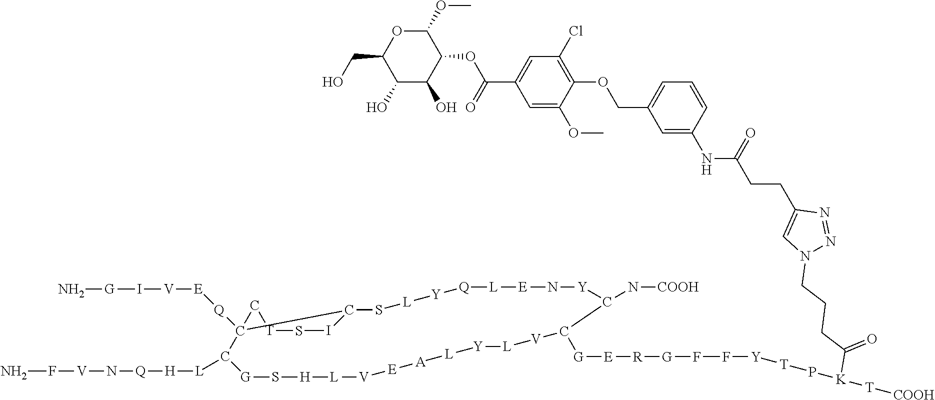

Example 263 was synthesized as described in synthesis method P. The product was purified by HPLC (RP, Kinetex C₁₈, 100 A, 5 μm, 21.1×250 mm, flow 6.2 mL/min, eluents: A: H₂O+0.5% acetic acid, B: 60% acetonitrile+39.5% H₂O+0.5% acetic acid, gradient: 0-15 min 0 to 20% B, 15-189 min 20% to 80% B, 189-190 min 80% to 100% B, 190-220 min 100% B).

Yield: 46 mg (7.1 μmol, 21%), white powder.

LC/MS (ES-API): m/z=1297.1 [M+5H]⁵⁺; calculated: 1296.58; t_{R} (λ=215 nm): 5.65 min (LC/MS-method F).

### Method Q Conjugation with Insulin Using TSTU

General Description:

Insulin (1 eq.) is dissolved in acetonitrile (38 mL) and water (20 mL), and the pH is adjusted to 10.5 with triethylamine. To a separate solution of the carbohydrate derivative dissolved in dimethylformamide and triethylamine (2 eq.). O—(N-Succinimidyl)-N,N,N′,N′-tetramethyluronium tetrafluoroborate (TSTU, 1 eq.) and 4-dimethylaminopyridine (0.05 eq.) are added. The reaction is stirred for 30 minutes at room temperature to yield the N-hydroxysuccinimidyl ester of the acid precursor, which is then added to the insulin solution. The reaction is stirred for 1 h at room temperature before diluting to a final volume of 200 mL with water. The pH is adjusted to 6.5 with 1 M acetic acid and the crude mixture is purified by RP-chromatography (Kinetex, 5 μm. C₁₈, 100 A, 21.1×250 mm). The purified fractions are collected, pooled and freeze-dried (26.5% yield, 90% purity).

### Synthesis of Intermediates

### 2-[3,5-Dichloro-4-hydroxy-2-methyl-benzoyl]-methyl-α-D-glucopyranoside

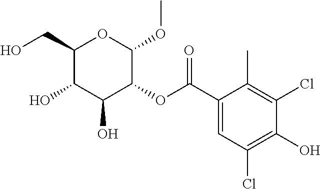 [SMILES: CO[C@H]1O[C@H](CO)[C@@H](O)[C@H](O)[C@H]1OC(=O)c1cc(Cl)c(O)c(Cl)c1C]

To a solution of 2-[4-benzyloxy-3,5-dichloro-3-methyl-benzoyl]-methyl-α-D-glucopyranoside (3.37 g, 5.86 mmol, synthesized following the procedure described in method L) in EtOH (50 mL) and ethyl acetate (50 mL) under argon atmosphere, Pd-C 10% (50% water) (623 mg, 585.6 μmol) was added. Hydrogen was added (0.2 bar) for 2 h. The reaction was monitored by LC/MS. The catalyst was filtered and the reaction mixture was evaporated to yield 2.33 g (5.66 mmol, 97% yield) of the desired product.

### 2-[3-Chloro-4-hydroxy-2-methoxy-benzoyl]-methyl-α-D-glucopyranoside

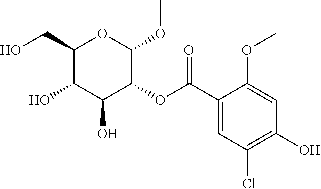 [SMILES: COc1cc(O)c(Cl)cc1C(=O)O[C@H]1[C@@H](OC)O[C@H](CO)[C@@H](O)[C@@H]1O]

2-[3-Chloro-4-hydroxy-2-methoxy-benzoyl]-methyl-α-D-glucopyranoside was synthesized from 2-[4-benzyloxy-3-chloro-3-methoxy-benzoyl]-methyl-α-D-glucopyranoside (1.65 g, 2.96 mmol) following the procedure described above in 70% yield.

### 2-[3-Chloro-4-hydroxy-2-methoxy-benzamidyl]-methyl-α-D-glucosamine

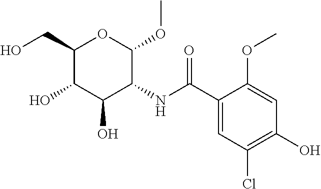 [SMILES: COc1cc(O)c(Cl)cc1C(=O)N[C@H]1[C@@H](OC)O[C@H](CO)[C@@H](O)[C@@H]1O]

To a solution of 2-[5-chloro-4-(benzyloxy)-3-methoxy-benzoyl]-methyl-α-D-glucosamine (5.83 g, 12.46 mmol, synthesized following the procedure described in method L) in CH₂Cl₂ (440 mL) and methanol (440 mL) under argon atmosphere, zinc bromide (140.43 mg, 623.00 μmol) and Pd-C 10% (50% water) (1.33 g, 623.00 μmol) were added. Hydrogen was advected for 30 minutes and the reaction mixture was stirred for 2.5 h. The reaction was monitored by LC/MS. The catalyst was filtered off and washed with methanol and water. The reaction mixture was evaporated to yield the desired product quantitatively.

### 3,5-Dichloro-4-hydroxy-2-methyl-benzamidyl-methyl-α-D-glucopyranoside

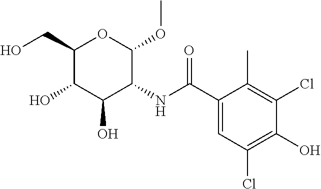 [SMILES: CO[C@H]1O[C@H](CO)[C@@H](O)[C@H](O)[C@H]1NC(=O)c1cc(Cl)c(O)c(Cl)c1C]

3,5-Dichloro-4-hydroxy-2-methyl-benzamidyl-methyl-α-D-glucopyranoside was synthesized from 2-[4-benzyloxy-3,5-dichloro-3-methyl-benzoyl]-methyl-α-D-glucosamine (4.99 g, 10.26 mmol) following the procedure described above in quantitative yield.

Additional synthetic methods are described below:

### Method R Alkylation of Glucopyranoside- or Glucosamine-Phenol Building Blocks

General Description:

2-[3,5-dichloro-4-hydroxy-2-methyl-benzoyl]-methyl-α-D-glucopyranoside (266 mg, 0.67 mmol), N-(5-(bromomethyl)-2-methoxyphenyl)pent-4-ynamide (198 mg, 0.67 mmol) and K₂CO₃ (101.64 mg, 735.40 μmol) were dissolved in DMF (2 mL) and stirred at 25° C. for 2 h. The reaction was monitored by LC/MS. After 16 h, ethyl acetate and water were added to the reaction mixture. The organic phase was extracted twice with ethyl acetate. The organic phase was dried with Na₂SO₄, filtered and evaporated.

### Method S Alkylation of Benzyl Amines

General Description:

To a solution of 4-((3-(aminomethyl)benzyl)oxy)-3,5-dichloro-2-methylbenzoyl-methyl-α-D-glucopyranoside hydrochloride (100 mg, 169.70 μmol, synthesized following the procedure described in method L*¹⁵) in DMF (0.5 mL) K₂CO₃ (70.36 mg, 509.09 μmol) and tert-butyl 4-bromobutanoate (45 μl, 254.55 μmol) were added. The reaction mixture was stirred at room temperature for 2 h. The reaction was monitored by LC/MS. After 16 h at room temperature the reaction mixture was evaporated and purified by preparative HPLC (YMC-Actus Triart Prep C18-S 250×30 S-10 μm, 12 nm, 70 mL/min; 0-2 min 5% acetonitrile in H₂O+0.05% trifluoroacetic acid, 2-10 min 5 to 100% acetonitrile in H₂O+0.05% trifluoroacetic acid, 20-22 min: 100% acetonitrile) to yield 12 mg (18.8 μmol, 11% yield) of the desired product.

### Method T Alkylation of Anilines

General Description:

To a suspension of 4-((3-aminobenzyl)oxy)-3,5-dichloro-2-methylbenzoate-methyl-α-D-glucopyranoside hydrochloride (100 mg, 185.60 μmol, synthesized following the procedure described in method L*¹⁵) and 4-(tert-butoxy)-4-oxobutanoic acid (38.80 mg, 222.72 μmol) in CH₂Cl₂ (1 mL), triethylamine (77.61 μl, 556.79 μmol) was added and the reaction mixture was cooled to 0° C. Isobutyl chloroformate (43.87 μl, 334.08 μmol) in CH₂Cl₂ (1 mL) was added over a period of 10 minutes at 0° C. After warming up to 25° C. the reaction mixture was stirred for 16 h. The reaction mixture was evaporated and purified by preparative HPLC (Merck Hibar PurospherSTAR RP-18e 3 μm 25*75 mm, 0-1 min: 5% acetonitrile in H₂O+0.05% trifluoroacetic acid, 1-20 min from 5 to 100% acetonitrile in H₂O+0.05% trifluoroactic acid, 20-22 min: 100% acetonitrile) to yield 11 mg (16.7 μmol, 9% yield) of the desired product.

The following examples were synthesized using the methods described above:

| | | | | | m/z | t_{R} | LC/MS- |
|---|---|---|---|---|---|---|---|
| Ex. | Structure | Synth Method | yield | m/z | calc. | (λ = 220 nm) | method |
| 2 | 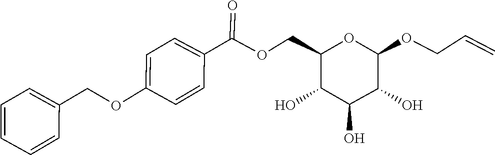 [SMILES: C=CCO[C@@H]1O[C@H](COC(=O)c2ccc(OCc3ccccc3)cc2)[C@@H](O)[C@H](O)[C@H]1O] | A | 43% | 475.30 [M − H + formic acid]⁻ | | 1.70 | A |
| 3 | 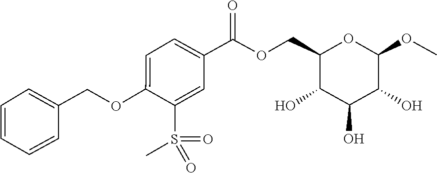 [SMILES: CO[C@@H]1O[C@H](COC(=O)c2ccc(OCc3ccccc3)c(S(C)(=O)=O)c2)[C@@H](O)[C@H](O)[C@H]1O] | A | 33% | 527.20 [M − H + formic acid]⁻ | 527.16 | 1.41 | A |
| 4 | 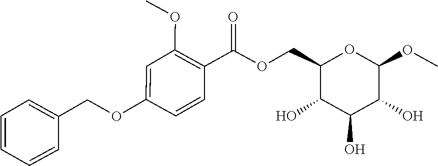 [SMILES: COc1cc(OCc2ccccc2)ccc1C(=O)OC[C@H]1O[C@@H](OC)[C@H](O)[C@@H](O)[C@@H]1O] | A | 31% | 435.27 [M + H]⁺ | 435.16 | 1.53 | A |
| 5 | 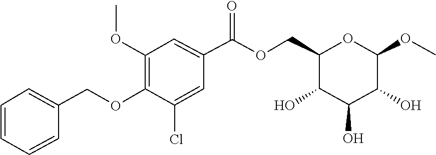 [SMILES: COc1cc(C(=O)OC[C@H]2O[C@@H](OC)[C@H](O)[C@@H](O)[C@@H]2O)cc(Cl)c1OCc1ccccc1] | A | 52% | 513.20 [M − H + formic acid]⁻ | 413.15 | 1.67 | A |
| 6 | 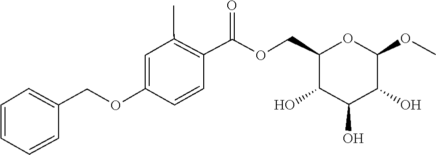 [SMILES: CO[C@@H]1O[C@H](COC(=O)c2ccc(OCc3ccccc3)cc2C)[C@@H](O)[C@H](O)[C@H]1O] | A | 53% | 463.24 [M − H + formic acid]⁻ | 463.19 | 1.63 | A |
| 7 | 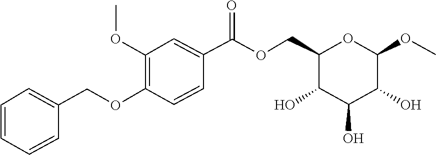 [SMILES: COc1cc(C(=O)OC[C@H]2O[C@@H](OC)[C@H](O)[C@@H](O)[C@@H]2O)ccc1OCc1ccccc1] | A | 53% | 479.24 [M − H + formic acid]⁻ | 479.19 | 1.52 | A |
| 8 | 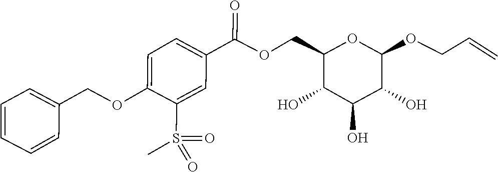 [SMILES: C=CCO[C@@H]1O[C@H](COC(=O)c2ccc(OCc3ccccc3)c(S(C)(=O)=O)c2)[C@@H](O)[C@H](O)[C@H]1O] | A | 33% | 553.21 [M − H + formic acid]⁻ | 553.17 | 1.51 | A |
| 9 | 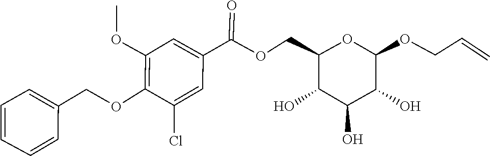 [SMILES: C=CCO[C@@H]1O[C@H](COC(=O)c2cc(Cl)c(OCc3ccccc3)c(OC)c2)[C@@H](O)[C@H](O)[C@H]1O] | A | 40% | 539.21 [M − H + formic acid]⁻ | 539.16 | 1.75 | A |
| 10 | 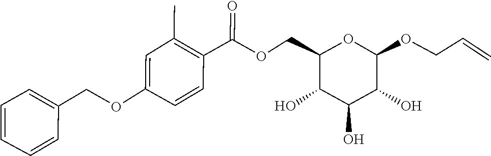 [SMILES: C=CCO[C@@H]1O[C@H](COC(=O)c2ccc(OCc3ccccc3)cc2C)[C@@H](O)[C@H](O)[C@H]1O] | A | 35% | 489.24 [M − H + formic acid]⁻ | 489.21 | 1.72 | A |
| 11 | 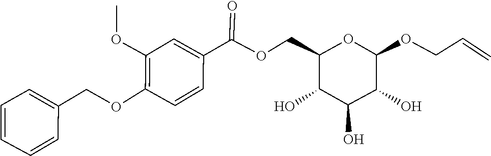 [SMILES: C=CCO[C@@H]1O[C@H](COC(=O)c2ccc(OCc3ccccc3)c(OC)c2)[C@@H](O)[C@H](O)[C@H]1O] | A | 44% | 505.25 [M − H + formic acid]⁻ | 505.20 | 1.61 | A |
| 12 | 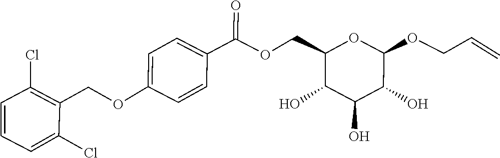 [SMILES: C=CCO[C@@H]1O[C@H](COC(=O)c2ccc(OCc3c(Cl)cccc3Cl)cc2)[C@@H](O)[C@H](O)[C@H]1O] | A | 38% | 543.16 [M − H + formic acid]⁻ | 543.12 | 1.76 | A |
| 13 | 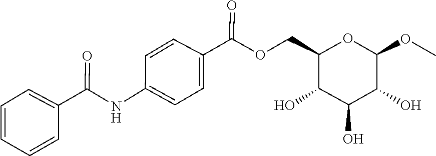 [SMILES: CO[C@@H]1O[C@H](COC(=O)c2ccc(NC(=O)c3ccccc3)cc2)[C@@H](O)[C@H](O)[C@H]1O] | A | 6% | 416.21 [M − H]⁻ | 416.14 | 1.39 | A |
| 14 | 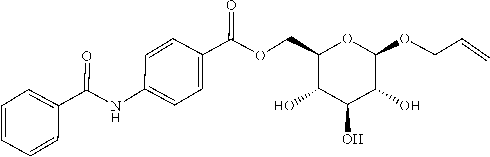 [SMILES: C=CCO[C@@H]1O[C@H](COC(=O)c2ccc(NC(=O)c3ccccc3)cc2)[C@@H](O)[C@H](O)[C@H]1O] | A | 7% | 442.23 [M − H]⁻ | 442.16 | 1.50 | A |
| 15 | 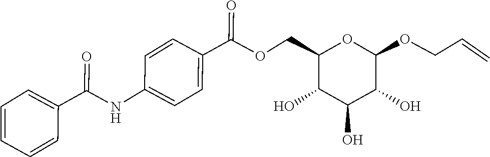 [SMILES: C=CCO[C@@H]1O[C@H](COC(=O)c2ccc(NC(=O)c3ccccc3)cc2)[C@@H](O)[C@H](O)[C@H]1O] | A | 16% | 449.36 [M − H + formic acid]⁻ | 449.18 | 1.62 | A |
| 16 | 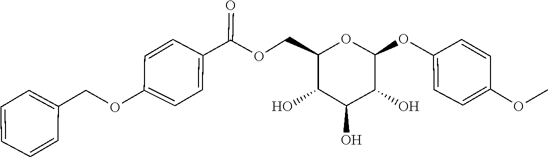 [SMILES: COc1ccc(O[C@@H]2O[C@H](COC(=O)c3ccc(OCc4ccccc4)cc3)[C@@H](O)[C@H](O)[C@H]2O)cc1] | A | 63% | 497.34 [M + H]⁺ | 497.17 | 1.74 | A |
| 17 | 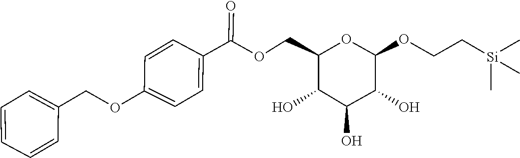 [SMILES: C[Si](C)(C)CCO[C@@H]1O[C@H](COC(=O)c2ccc(OCc3ccccc3)cc2)[C@@H](O)[C@H](O)[C@H]1O] | A | 50% | 491.33 [M + H]⁺ | 491.20 | 1.88 | A |
| 18 | 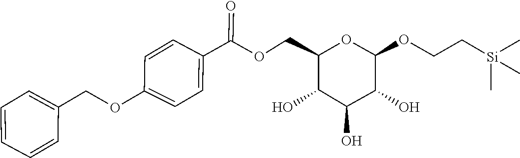 | A | 48% | 431.28 [M + H]⁺ | 431.16 | 1.67 | A |
| 19 | 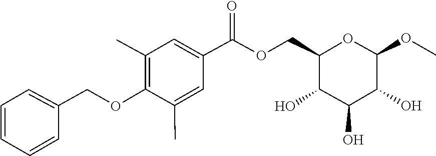 [SMILES: CO[C@@H]1O[C@H](COC(=O)c2cc(C)c(OCc3ccccc3)c(C)c2)[C@@H](O)[C@H](O)[C@H]1O] | A | 54% | 477.20 [M − H + formic acid]⁻ | 477.21 | 1.68 | A |
| 20 | 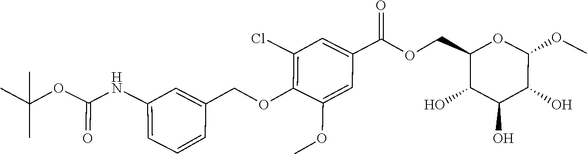 [SMILES: COc1cc(C(=O)OC[C@H]2O[C@H](OC)[C@H](O)[C@@H](O)[C@@H]2O)cc(Cl)c1OCc1cccc(NC(=O)OC(C)(C)C)c1] | A | 68% | 628.2/630.2 [M − H + formic acid]⁻ | 628.21 | 1.76 | A |
| 21 | 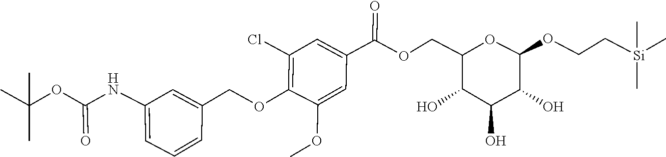 [SMILES: COc1cc(C(=O)OCC2O[C@@H](OCC[Si](C)(C)C)[C@H](O)[C@@H](O)[C@@H]2O)cc(Cl)c1OCc1cccc(NC(=O)OC(C)(C)C)c1] | A | 25% | 714.3/715.3 [M − H + formic acid]⁻ | 714.27 | 1.96 | A |
| 22 | 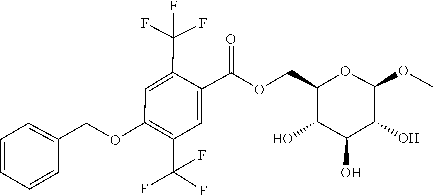 [SMILES: CO[C@@H]1O[C@H](COC(=O)c2cc(C(F)(F)F)c(OCc3ccccc3)cc2C(F)(F)F)[C@@H](O)[C@H](O)[C@H]1O] | A | 48 % | 585.15 [M − H + formic acid]⁻ | 585.15 | 1.78 | A |
| 23 | 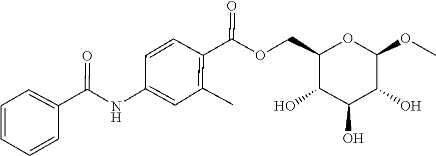 [SMILES: CO[C@@H]1O[C@H](COC(=O)c2ccc(NC(=O)c3ccccc3)cc2C)[C@@H](O)[C@H](O)[C@H]1O] | A | 57% | 430.19 [M − H]⁻ | 430.16 | 1.40 | A |
| 24 | 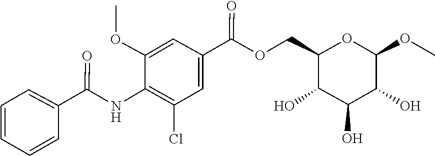 [SMILES: COc1cc(C(=O)OC[C@H]2O[C@@H](OC)[C@H](O)[C@@H](O)[C@@H]2O)cc(Cl)c1NC(=O)c1ccccc1] | A | 41% | 480.12 [M − H]⁻ | 480.11 | 1.38 | A |
| 25 | 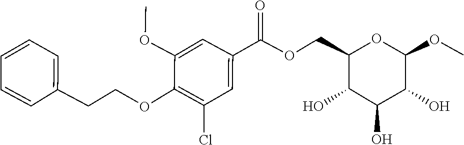 [SMILES: COc1cc(C(=O)OC[C@H]2O[C@@H](OC)[C@H](O)[C@@H](O)[C@@H]2O)cc(Cl)c1OCCc1ccccc1] | A | 26% | 527.17 [M − H + formic acid]⁻ | 527.16 | 1.74 | A |
| 26 | 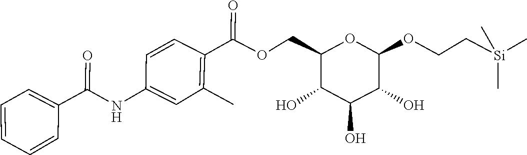 [SMILES: Cc1cc(NC(=O)c2ccccc2)ccc1C(=O)OC[C@H]1O[C@@H](OCC[Si](C)(C)C)[C@H](O)[C@@H](O)[C@@H]1O] | A | 60% | 516.23 [M − H]⁻ | 516.21 | 1.74 | A |
| 27 | 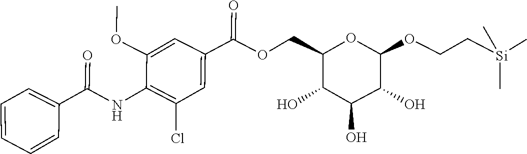 [SMILES: COc1cc(C(=O)OC[C@H]2O[C@@H](OCC[Si](C)(C)C)[C@H](O)[C@@H](O)[C@@H]2O)cc(Cl)c1NC(=O)c1ccccc1] | A | 64% | 566.14 [M − H]⁻ | 566.17 | 1.73 | A |
| 28 | 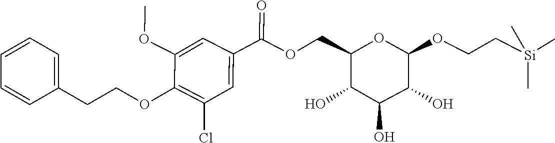 [SMILES: COc1cc(C(=O)OC[C@H]2O[C@@H](OCC[Si](C)(C)C)[C@H](O)[C@@H](O)[C@@H]2O)cc(Cl)c1OCCc1ccccc1] | A | 24% | 613.15 [M − H + formic acid]⁻ | 613.22 | 1.99 | A |
| 29 | 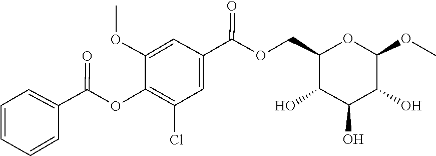 [SMILES: COc1cc(C(=O)OC[C@H]2O[C@@H](OC)[C@H](O)[C@@H](O)[C@@H]2O)cc(Cl)c1OC(=O)c1ccccc1] | A | 40% | 527.09 [M − H + formic acid]⁻ | 527.13 | 1.66 | A |
| 30 | 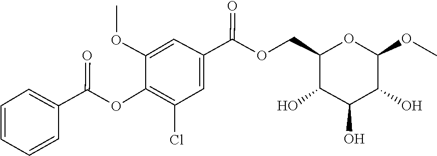 | A | 22% | 433.17 [M + H]⁺ | 433.14 | 1.61 | A |
| 31 | 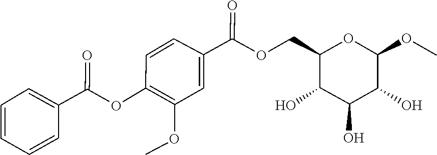 [SMILES: COc1cc(C(=O)OC[C@H]2O[C@@H](OC)[C@H](O)[C@@H](O)[C@@H]2O)ccc1OC(=O)c1ccccc1] | A | 33% | 449.16 [M + H]⁺ | 449.14 | 1.56 | A |
| 32 | 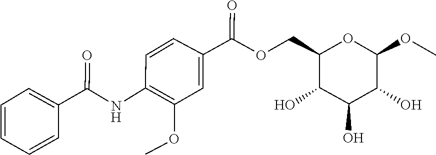 [SMILES: COc1cc(C(=O)OC[C@H]2O[C@@H](OC)[C@H](O)[C@@H](O)[C@@H]2O)ccc1NC(=O)c1ccccc1] | A | 71% | 448.19 [M + H]⁺ | 448.15 | 1.46 | A |
| 33 | 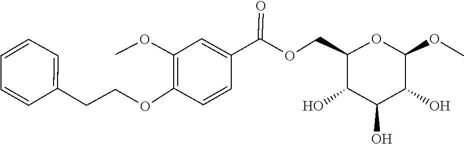 [SMILES: COc1cc(C(=O)OC[C@H]2O[C@@H](OC)[C@H](O)[C@@H](O)[C@@H]2O)ccc1OCCc1ccccc1] | A | 62% | 493.19 [M − H + formic acid]⁻ | 493.20 | 1.58 | A |
| 34 | 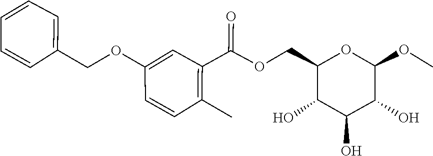 [SMILES: CO[C@@H]1O[C@H](COC(=O)c2cc(OCc3ccccc3)ccc2C)[C@@H](O)[C@H](O)[C@H]1O] | A | 52% | 463.16 [M − H + formic acid]⁻ | 463.19 | 1.63 | A |
| 35 | 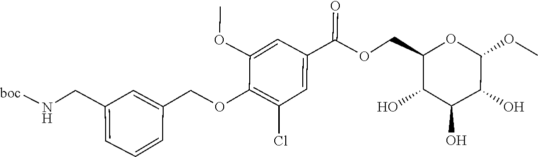 | A | 39% | 642.15 [M − H + formic acid]⁻ | 642.23 | 1.73 | A |
| 36 | 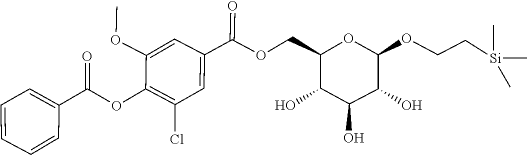 [SMILES: COc1cc(C(=O)OC[C@H]2O[C@@H](OCC[Si](C)(C)C)[C@H](O)[C@@H](O)[C@@H]2O)cc(Cl)c1OC(=O)c1ccccc1] | A | 43% | 613.10 [M − H + formic acid]⁻ | 613.18 | 1.94 | A |
| 37 | 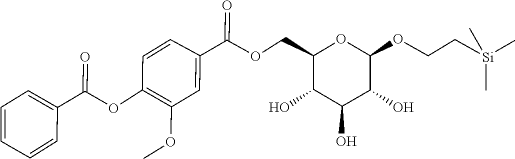 [SMILES: COc1cc(C(=O)OC[C@H]2O[C@@H](OCC[Si](C)(C)C)[C@H](O)[C@@H](O)[C@@H]2O)ccc1OC(=O)c1ccccc1] | A | 48% | 579.14 [M − H + formic acid]⁻ | 579.22 | 1.86 | A |
| 38 | 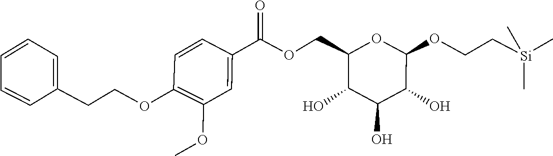 [SMILES: COc1cc(C(=O)OC[C@H]2O[C@@H](OCC[Si](C)(C)C)[C@H](O)[C@@H](O)[C@@H]2O)ccc1OCCc1ccccc1] | A | 67 % | 532.18 [M − H]⁻ | 532.21 | 1.79 | A |
| 39 | 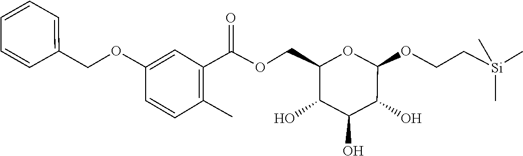 [SMILES: Cc1ccc(OCc2ccccc2)cc1C(=O)OC[C@H]1O[C@@H](OCC[Si](C)(C)C)[C@H](O)[C@@H](O)[C@@H]1O] | A | 67% | 579.19 [M − H + formic acid]⁻ | 579.26 | 1.87 | A |
| 40 | 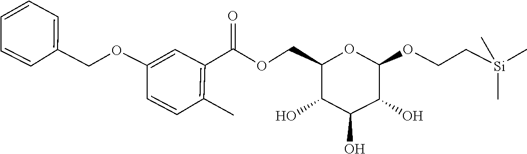 [SMILES: Cc1ccc(OCc2ccccc2)cc1C(=O)OC[C@H]1O[C@@H](OCC[Si](C)(C)C)[C@H](O)[C@@H](O)[C@@H]1O] | A | 63% | 549.20 [M − H + formic acid]⁻ | 549.25 | 1.93 | A |
| 41 | 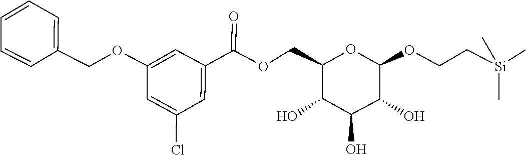 [SMILES: C[Si](C)(C)CCO[C@@H]1O[C@H](COC(=O)c2cc(Cl)cc(OCc3ccccc3)c2)[C@@H](O)[C@H](O)[C@H]1O] | A | 54% | 569.13 [M − H + formic acid]⁻ | 569.19 | 1.97 | A |
| 42 | 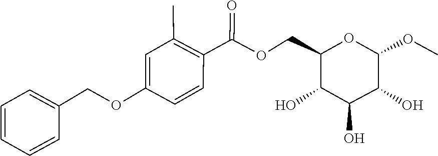 [SMILES: CO[C@H]1O[C@H](COC(=O)c2ccc(OCc3ccccc3)cc2C)[C@@H](O)[C@H](O)[C@H]1O] | A | 54% | 419.16 [M + H]⁺ | 419.17 | 1.95 | A |
| 43 | 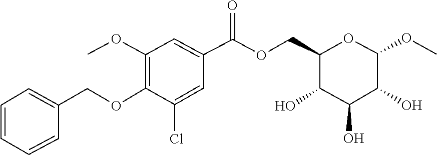 [SMILES: COc1cc(C(=O)OC[C@H]2O[C@H](OC)[C@H](O)[C@@H](O)[C@@H]2O)cc(Cl)c1OCc1ccccc1] | A | 44% | 513.1/515.0 [M − H + formic acid]⁻ | 513.14 | 2.04 | A |
| 44 | 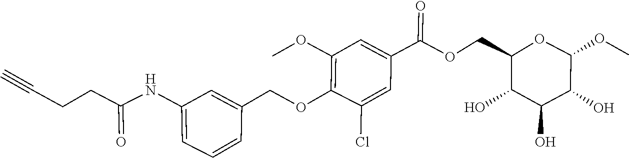 [SMILES: C#CCCC(=O)Nc1cccc(COc2c(Cl)cc(C(=O)OC[C@H]3O[C@H](OC)[C@H](O)[C@@H](O)[C@@H]3O)cc2OC)c1] | A | 38% | 564.1/566.1 [M + H]⁺ | 564.16 | 1.80 | A |
| 45 | 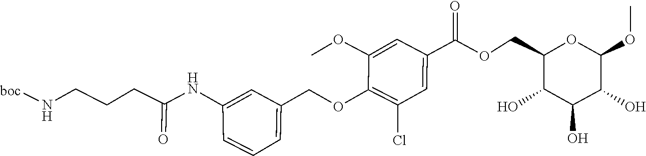 | A | 34% | 669.2/671.1 [M + H]⁺ | 669.24 | 1.98 | A |
| 46 | 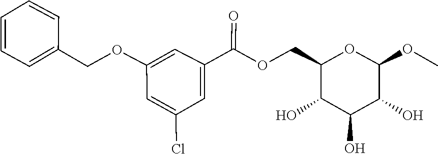 [SMILES: CO[C@@H]1O[C@H](COC(=O)c2cc(Cl)cc(OCc3ccccc3)c2)[C@@H](O)[C@H](O)[C@H]1O] | A | 18% | 483.25 [M − H + formic acid]⁻ | 483.11 | 1.73 | A |
| 48 | 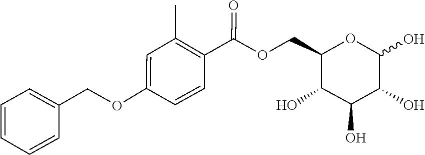 [SMILES: Cc1cc(OCc2ccccc2)ccc1C(=O)OC[C@H]1OC(O)[C@H](O)[C@@H](O)[C@@H]1O] | B | 19% | 403.17 [M − H]⁻ | 403.15 | 1.54/1.52 | A |
| 49 | 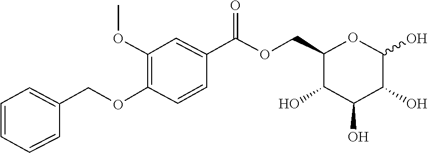 [SMILES: COc1cc(C(=O)OC[C@H]2OC(O)[C@H](O)[C@@H](O)[C@@H]2O)ccc1OCc1ccccc1] | B | 29% | 419.19 [M − H]⁻ | 419.14 | 1.43/1.41 | A |
| 50 | 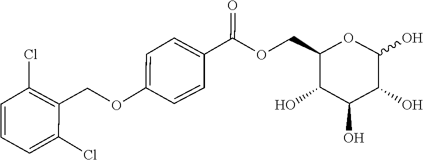 [SMILES: O=C(OC[C@H]1OC(O)[C@H](O)[C@@H](O)[C@@H]1O)c1ccc(OCc2c(Cl)cccc2Cl)cc1] | B | 20% | 459.2/461.2 [M + H]⁺ | 459.05 | 1.59/1.57 | A |
| 51 | 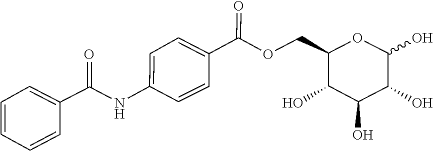 [SMILES: O=C(Nc1ccc(C(=O)OC[C@H]2OC(O)[C@H](O)[C@@H](O)[C@@H]2O)cc1)c1ccccc1] | B | 45% | 402.13 [M − H]⁻ | 402.18 | 1.21/1.23 | A |
| 52 | 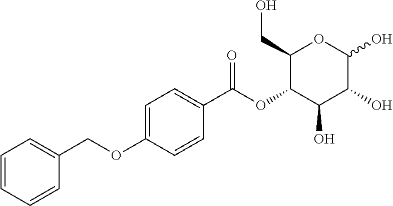 [SMILES: O=C(O[C@H]1[C@H](O)[C@@H](O)C(O)O[C@@H]1CO)c1ccc(OCc2ccccc2)cc1] | B | 43% | 391.15 [M + H]⁺ | 391.13 | 1.45 | A |
| 53 | 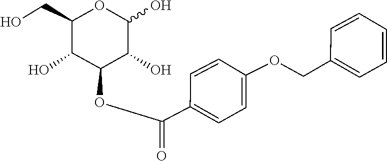 [SMILES: O=C(O[C@H]1[C@H](O)[C@@H](CO)OC(O)[C@@H]1O)c1ccc(OCc2ccccc2)cc1] | B | 18% | 391.18 [M + H]⁺ | 391.14 | 1.42 | A |
| 55 | 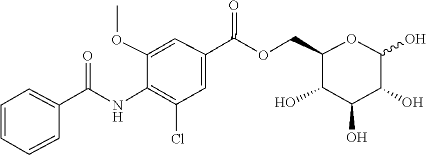 [SMILES: COc1cc(C(=O)OC[C@H]2OC(O)[C@H](O)[C@@H](O)[C@@H]2O)cc(Cl)c1NC(=O)c1ccccc1] | C | 79% | 468.2/470.2 [M + H]⁺ | 468.10 | 1.29/1.27 | A |
| 56 | 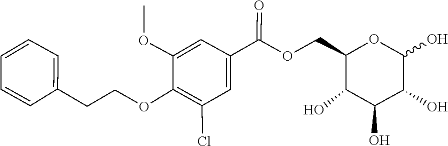 [SMILES: COc1cc(C(=O)OC[C@H]2OC(O)[C@H](O)[C@@H](O)[C@@H]2O)cc(Cl)c1OCCc1ccccc1] | C | 65% | 513.1/515.1 [M − H + formic acid]⁻ | 513.15 | 1.65/1.64 | A |
| 57 | 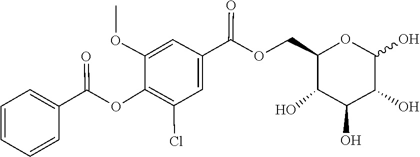 [SMILES: COc1cc(C(=O)OC[C@H]2OC(O)[C@H](O)[C@@H](O)[C@@H]2O)cc(Cl)c1OC(=O)c1ccccc1] | C | 89% | 513.1/515.1 [M − H + formic acid]⁻ | 513.11 | 1.58/1.56 | A |
| 58 | 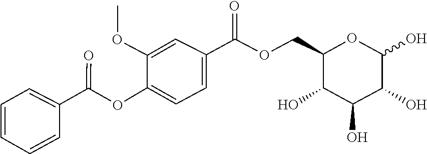 [SMILES: COc1cc(C(=O)OC[C@H]2OC(O)[C@H](O)[C@@H](O)[C@@H]2O)ccc1OC(=O)c1ccccc1] | C | 77% | 433.06 [M − H]⁻ | 433.12 | 1.48/1.46 | A |
| 59 | 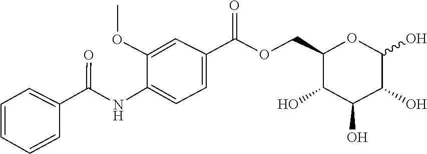 [SMILES: COc1cc(C(=O)OC[C@H]2OC(O)[C@H](O)[C@@H](O)[C@@H]2O)ccc1NC(=O)c1ccccc1] | C | 87% | 432.12 [M − H]⁻ | 432.14 | 1.37/1.34 | A |
| 60 | 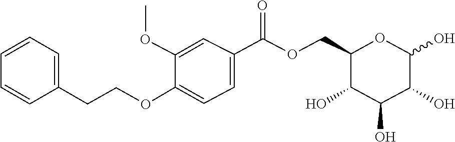 [SMILES: COc1cc(C(=O)OC[C@H]2OC(O)[C@H](O)[C@@H](O)[C@@H]2O)ccc1OCCc1ccccc1] | C | 62% | 433.08 [M − H]⁻ | 433.16 | 1.50/1.49 | A |
| 61 | 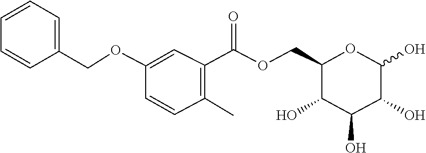 [SMILES: Cc1ccc(OCc2ccccc2)cc1C(=O)OC[C@H]1OC(O)[C@H](O)[C@@H](O)[C@@H]1O] | C | 87% | 403.09 [M − H]⁻ | 403.15 | 1.53/1.51 | A |
| 62 | 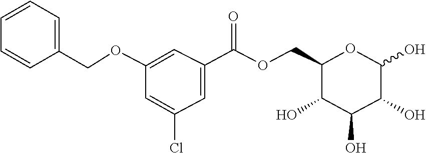 [SMILES: O=C(OC[C@H]1OC(O)[C@H](O)[C@@H](O)[C@@H]1O)c1cc(Cl)cc(OCc2ccccc2)c1] | C | 36% | 469.0/471.0 [M − H + formic acid]⁻ | 469.12 | 1.60/1.58 | A |
| 63 | 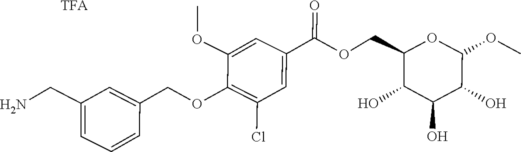 | C | 90% | 498.2/500.1 [M + H]⁺ | 498.15 | 1.13 | A |
| 64 | 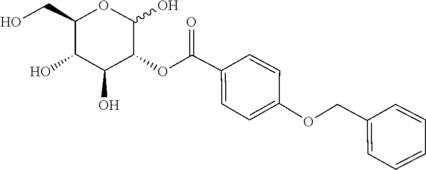 [SMILES: O=C(O[C@H]1C(O)O[C@H](CO)[C@@H](O)[C@@H]1O)c1ccc(OCc2ccccc2)cc1] | C | 44% | 373.1 [M + H − H₂O]⁺ | 373.12 | 1.10 | B |
| 66 | 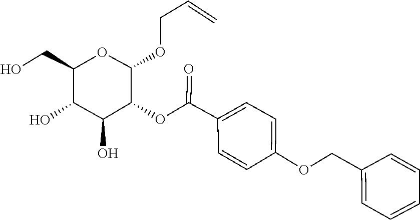 [SMILES: C=CCO[C@H]1O[C@H](CO)[C@@H](O)[C@H](O)[C@H]1OC(=O)c1ccc(OCc2ccccc2)cc1] | D | 13% | 475.28 [M − H + formic acid]⁻ | 475.19 | 1.67 | A |
| 67 | 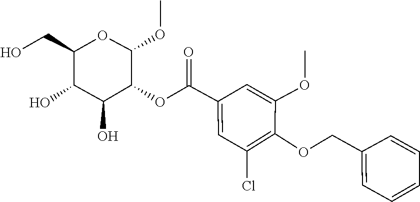 [SMILES: COc1cc(C(=O)O[C@H]2[C@@H](OC)O[C@H](CO)[C@@H](O)[C@@H]2O)cc(Cl)c1OCc1ccccc1] | D | 32% | 513.2/515.1 [M − H + formic acid]⁻ | 513.15 | 1.68 | A |
| 68 | 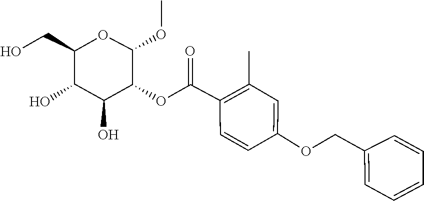 [SMILES: CO[C@H]1O[C@H](CO)[C@@H](O)[C@H](O)[C@H]1OC(=O)c1ccc(OCc2ccccc2)cc1C] | D | 51% | 463.14 [M − H + formic acid]⁻ | 463.19 | 1.64 | A |
| 69 | 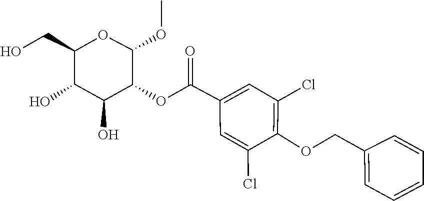 [SMILES: CO[C@H]1O[C@H](CO)[C@@H](O)[C@H](O)[C@H]1OC(=O)c1cc(Cl)c(OCc2ccccc2)c(Cl)c1] | D | 49% | 517.0/519.0 [M − H + formic acid]⁻ | 517.10 | 1.74 | A |
| 70 | 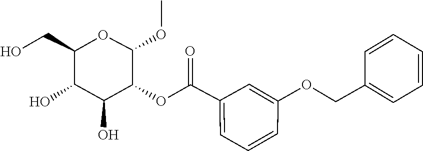 [SMILES: CO[C@H]1O[C@H](CO)[C@@H](O)[C@H](O)[C@H]1OC(=O)c1cccc(OCc2ccccc2)c1] | D | 43% | 405.15 [M + H]⁺ | 405.16 | 1.80 | A |
| 71 | 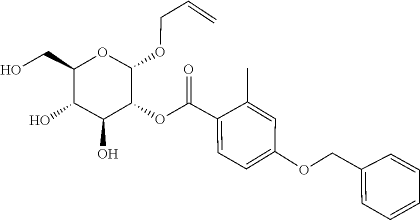 [SMILES: C=CCO[C@H]1O[C@H](CO)[C@@H](O)[C@H](O)[C@H]1OC(=O)c1ccc(OCc2ccccc2)cc1C] | D | 11% | 489.28 [M − H + formic acid]⁻ | 489.20 | 2.13 | A |
| 72 | 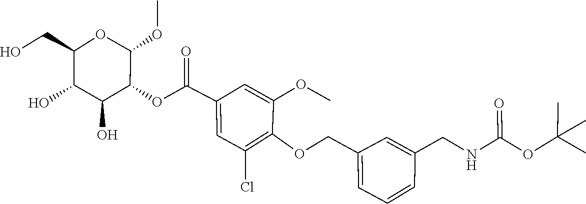 [SMILES: COc1cc(C(=O)O[C@H]2[C@@H](OC)O[C@H](CO)[C@@H](O)[C@@H]2O)cc(Cl)c1OCc1cccc(CNC(=O)OC(C)(C)C)c1] | D | 20% | 642.3/644.3 [M − H + formic acid]⁻ | 642.22 | 2.15 | A |
| 73 | 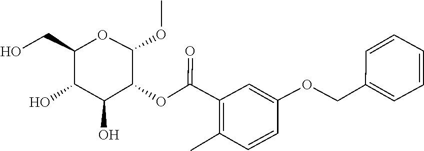 [SMILES: CO[C@H]1O[C@H](CO)[C@@H](O)[C@H](O)[C@H]1OC(=O)c1cc(OCc2ccccc2)ccc1C] | D | 50% | 463.08 [M − H + formic acid]⁻ | 463.18 | 1.93 | A |
| 74 | 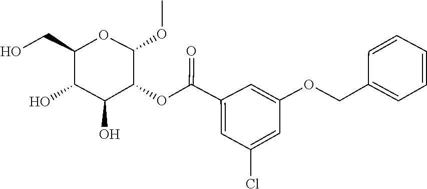 [SMILES: CO[C@H]1O[C@H](CO)[C@@H](O)[C@H](O)[C@H]1OC(=O)c1cc(Cl)cc(OCc2ccccc2)c1] | D | 49% | 483.0/485.1 [M − H + formic acid]⁻ | 483.13 | 2.05 | A |
| 75 | 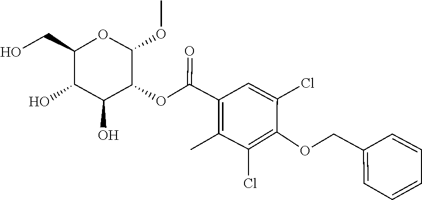 [SMILES: CO[C@H]1O[C@H](CO)[C@@H](O)[C@H](O)[C@H]1OC(=O)c1cc(Cl)c(OCc2ccccc2)c(Cl)c1C] | D | 47% | 531.0/533.0/535.0 [M − H + formic acid]⁻ | 531.10 | 2.26 | A |
| 76 | 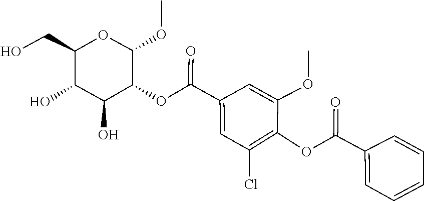 [SMILES: COc1cc(C(=O)O[C@H]2[C@@H](OC)O[C@H](CO)[C@@H](O)[C@@H]2O)cc(Cl)c1OC(=O)c1ccccc1] | D | 50% | 527.0/529.0 [M − H + formic acid]⁻ | 527.12 | 2.00 | A |
| 77 | 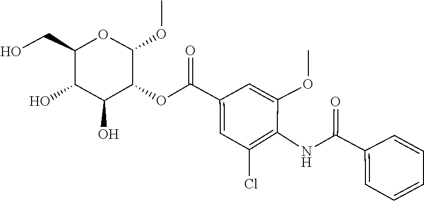 [SMILES: COc1cc(C(=O)O[C@H]2[C@@H](OC)O[C@H](CO)[C@@H](O)[C@@H]2O)cc(Cl)c1NC(=O)c1ccccc1] | D | 34% | 482.1/484.1 [M + H]⁺ | 482.12 | 1.49 | A |
| 78 | 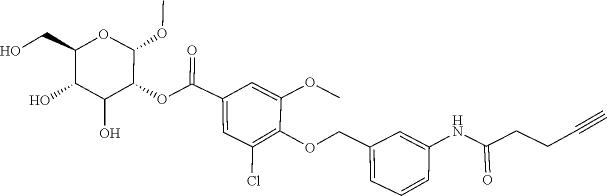 [SMILES: C#CCCC(=O)Nc1cccc(COc2c(Cl)cc(C(=O)O[C@@H]3C(O)[C@H](O)[C@@H](CO)O[C@@H]3OC)cc2OC)c1] | D | 22% | 564.2/565.9 [M + H]⁺ | 564.16 | 1.80 | A |
| 79 | 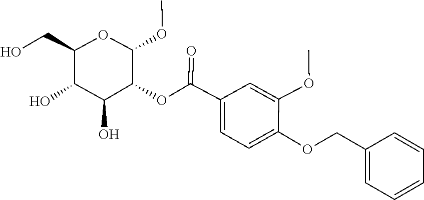 [SMILES: COc1cc(C(=O)O[C@H]2[C@@H](OC)O[C@H](CO)[C@@H](O)[C@@H]2O)ccc1OCc1ccccc1] | D | 43% | 479.11 [M − H + formic acid]⁻ | 479.18 | 1.72 | A |
| 80 | 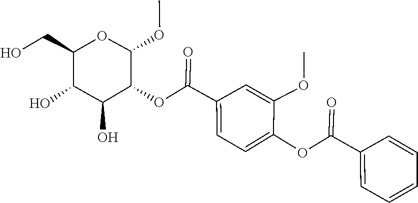 [SMILES: COc1cc(C(=O)O[C@H]2[C@@H](OC)O[C@H](CO)[C@@H](O)[C@@H]2O)ccc1OC(=O)c1ccccc1] | D | 42% | 493.09 [M − H + formic acid]⁻ | 493.15 | 1.80 | A |
| 81 | 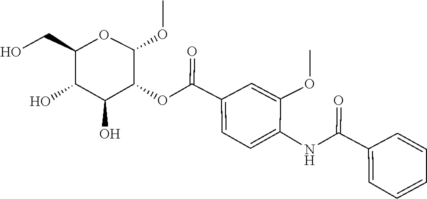 [SMILES: COc1cc(C(=O)O[C@H]2[C@@H](OC)O[C@H](CO)[C@@H](O)[C@@H]2O)ccc1NC(=O)c1ccccc1] | D | 43% | 446.10 [M − H]⁻ | 446.15 | 1.61 | A |
| 82 | 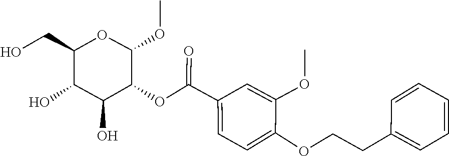 [SMILES: COc1cc(C(=O)O[C@H]2[C@@H](OC)O[C@H](CO)[C@@H](O)[C@@H]2O)ccc1OCCc1ccccc1] | D | 44% | 493.15 [M − H + formic acid]⁻ | 493.19 | 1.84 | A |
| 83 | 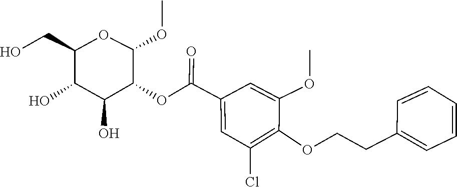 [SMILES: COc1cc(C(=O)O[C@H]2[C@@H](OC)O[C@H](CO)[C@@H](O)[C@@H]2O)cc(Cl)c1OCCc1ccccc1] | D | 36% | 527.1/529.0 [M − H + formic acid]⁻ | 527.16 | 2.13 | A |
| 84 | 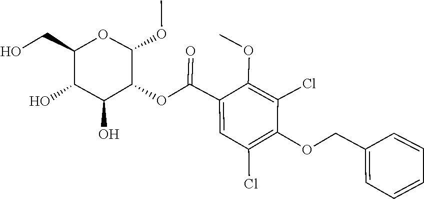 [SMILES: COc1c(C(=O)O[C@H]2[C@@H](OC)O[C@H](CO)[C@@H](O)[C@@H]2O)cc(Cl)c(OCc2ccccc2)c1Cl] | D | 39% | 547.1/549.0/551.1 [M − H + formic acid]⁻ | 547.11 | 2.15 | A |
| 85 | 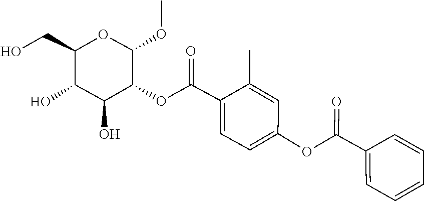 [SMILES: CO[C@H]1O[C@H](CO)[C@@H](O)[C@H](O)[C@H]1OC(=O)c1ccc(OC(=O)c2ccccc2)cc1C] | D | 36% | 477.1 [M − H + formic acid]⁻ | 477.17 | 1.89 | A |
| 86 | 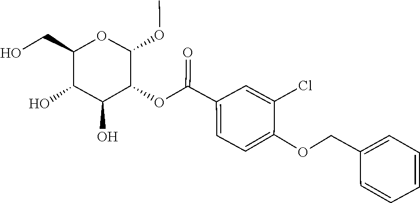 [SMILES: CO[C@H]1O[C@H](CO)[C@@H](O)[C@H](O)[C@H]1OC(=O)c1ccc(OCc2ccccc2)c(Cl)c1] | D | 16% | 483.1/485.1 [M − H + formic acid]⁻ | 483.14 | 1.95 | A |
| 87 | 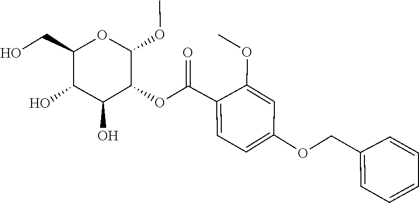 [SMILES: COc1cc(OCc2ccccc2)ccc1C(=O)O[C@H]1[C@@H](OC)O[C@H](CO)[C@@H](O)[C@@H]1O] | D | 6% | 435.21 [M + H]⁺ | 434.16 | 1.76 | A |
| 88 | 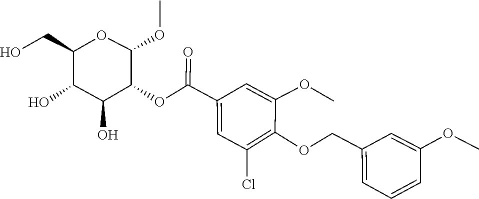 | D | 78% | 467.2/469.1 [M + H − OCH₃]⁺ | 467.09 | 2.01 | A |
| 90 | 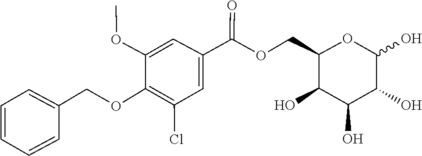 [SMILES: COc1cc(C(=O)OC[C@H]2OC(O)[C@H](O)[C@@H](O)[C@H]2O)cc(Cl)c1OCc1ccccc1] | E | 69% | 499.1/501.1 [M − H + formic acid]⁻ | 499.13 | 1.84/1.86 | A |
| 91 | 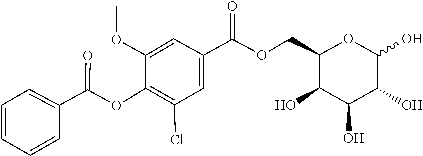 [SMILES: COc1cc(C(=O)OC[C@H]2OC(O)[C@H](O)[C@@H](O)[C@H]2O)cc(Cl)c1OC(=O)c1ccccc1] | E | 72% | 513.0/515.0 [M − H + formic acid]⁻ | 514.11 | 1.83/1.85 | A |
| 92 | 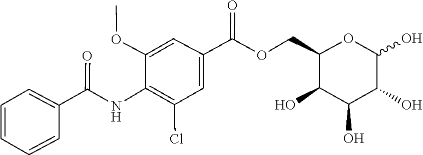 [SMILES: COc1cc(C(=O)OC[C@H]2OC(O)[C@H](O)[C@@H](O)[C@H]2O)cc(Cl)c1NC(=O)c1ccccc1] | E | 99% | 468.1/470.1 [M + H]⁺ | 468.10 | 1.33/1.35 | A |
| 93 | 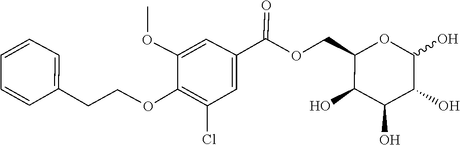 [SMILES: COc1cc(C(=O)OC[C@H]2OC(O)[C@H](O)[C@@H](O)[C@H]2O)cc(Cl)c1OCCc1ccccc1] | E | 85% | 451.0/452.1 [M + H − H₂O]⁺ | 451.10 | 1.97/1.99 | A |
| 94 | 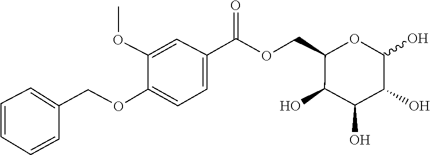 [SMILES: COc1cc(C(=O)OC[C@H]2OC(O)[C@H](O)[C@@H](O)[C@H]2O)ccc1OCc1ccccc1] | E | 44% | 421.13 [M + H]⁺ | 421.14 | 1.57/1.58 | A |
| 95 | 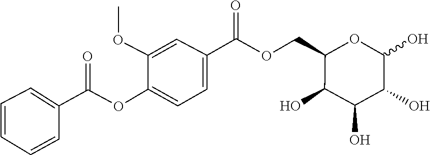 [SMILES: COc1cc(C(=O)OC[C@H]2OC(O)[C@H](O)[C@@H](O)[C@H]2O)ccc1OC(=O)c1ccccc1] | E | 29% | 452.2 [M + NH4]⁺ | 452.16 | 1.64/1.66 | A |
| 96 | 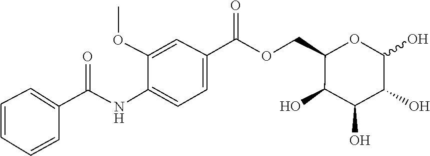 | E | 93% | 417.2 [M + H − H₂O]⁺ | 417.10 | 1.64/1.66 | A |
| 97 | 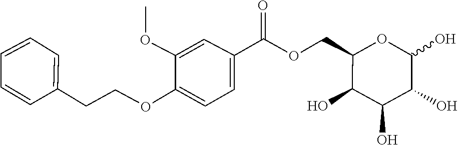 [SMILES: COc1cc(C(=O)OC[C@H]2OC(O)[C@H](O)[C@@H](O)[C@H]2O)ccc1OCCc1ccccc1] | E | 28% | 417.2 [M + H − H₂O]⁺ | 417.14 | 1.69/1.70 | A |
| 98 | 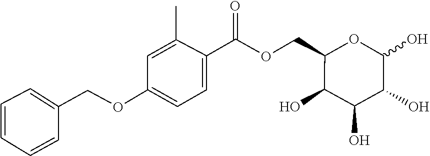 [SMILES: Cc1cc(OCc2ccccc2)ccc1C(=O)OC[C@H]1OC(O)[C@H](O)[C@@H](O)[C@H]1O] | E | 21% | 449.1 [M − H + formic acid]⁻ | 449.18 | 1.76/1.78 | A |
| 99 | 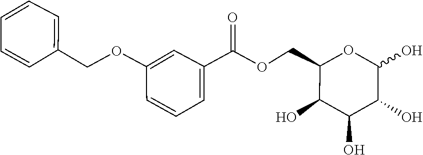 [SMILES: O=C(OC[C@H]1OC(O)[C@H](O)[C@@H](O)[C@H]1O)c1cccc(OCc2ccccc2)c1] | E | 23% | 373.2 [M + H − H₂O]⁺ | 373.11 | 1.64/1.65 | A |
| 100 | 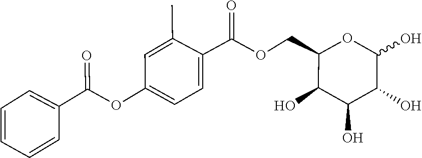 [SMILES: Cc1cc(OC(=O)c2ccccc2)ccc1C(=O)OC[C@H]1OC(O)[C@H](O)[C@@H](O)[C@H]1O] | E | 34% | 401.1 [M + H − H₂O]⁺ | 401.11 | 1.72/1.74 | A |
| 101 | 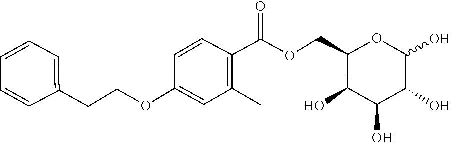 [SMILES: Cc1cc(OCCc2ccccc2)ccc1C(=O)OC[C@H]1OC(O)[C@H](O)[C@@H](O)[C@H]1O] | E | 21% | 463.1 [M − H + formic acid]⁻ | 463.19 | 1.88/1.90 | A |
| 102 | 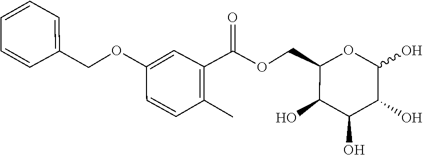 [SMILES: Cc1ccc(OCc2ccccc2)cc1C(=O)OC[C@H]1OC(O)[C@H](O)[C@@H](O)[C@H]1O] | E | 45% | 449.1 [M − H + formic acid]⁻ | 449.18 | 1.74/1.76 | A |
| 103 | 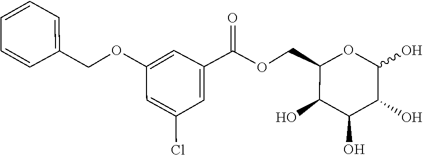 [SMILES: O=C(OC[C@H]1OC(O)[C@H](O)[C@@H](O)[C@H]1O)c1cc(Cl)cc(OCc2ccccc2)c1] | E | 43% | 407.1/409.1 [M + H − H₂O]⁺ | 407.07 | 1.87/1.89 | A |
| 105 | 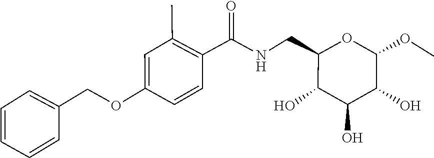 [SMILES: CO[C@H]1O[C@H](CNC(=O)c2ccc(OCc3ccccc3)cc2C)[C@@H](O)[C@H](O)[C@H]1O] | F*¹ | 7% | 418.29 [M + H]⁺ | 417.18 | 1.46 | A |
| 106 | 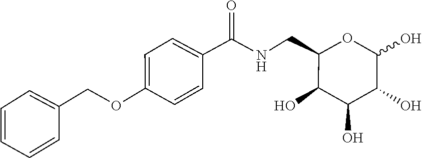 [SMILES: O=C(NC[C@H]1OC(O)[C@H](O)[C@@H](O)[C@H]1O)c1ccc(OCc2ccccc2)cc1] | F | 88% | 390.24 [M + H]⁺ | 390.15 | 1.40 | A |
| 107 | 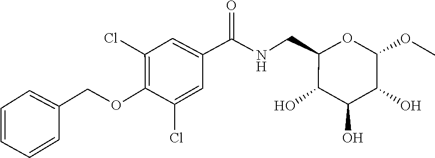 [SMILES: CO[C@H]1O[C@H](CNC(=O)c2cc(Cl)c(OCc3ccccc3)c(Cl)c2)[C@@H](O)[C@H](O)[C@H]1O] | F | 40% | 472.2/474.1 [M + H]⁺ | 472.09 | 1.68 | A |
| 108 | 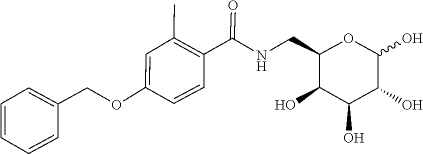 [SMILES: Cc1cc(OCc2ccccc2)ccc1C(=O)NC[C@H]1OC(O)[C@H](O)[C@@H](O)[C@H]1O] | F | 79% | 404.30 [M + H]⁺ | 404.16 | 1.43 | A |
| 109 | 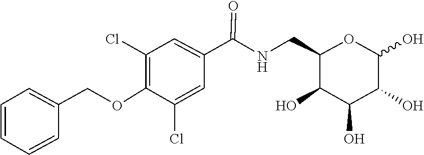 [SMILES: O=C(NC[C@H]1OC(O)[C@H](O)[C@@H](O)[C@H]1O)c1cc(Cl)c(OCc2ccccc2)c(Cl)c1] | F | 72% | 458.2/460.1 [M + H]⁺ | 458.07 | 1.58 | A |
| 110 | 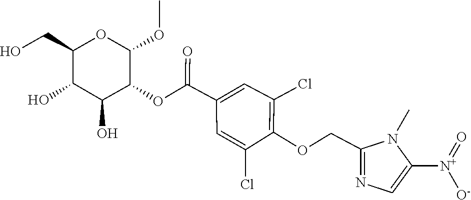 [SMILES: CO[C@H]1O[C@H](CO)[C@@H](O)[C@H](O)[C@H]1OC(=O)c1cc(Cl)c(OCc2ncc([N+](=O)[O-])n2C)c(Cl)c1] | D | 6% | 522.08 [M + H]⁺ | 522.07 | 1.62 | A |
| 111 | 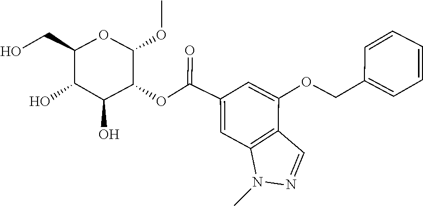 [SMILES: CO[C@H]1O[C@H](CO)[C@@H](O)[C@H](O)[C@H]1OC(=O)c1cc(OCc2ccccc2)c2cnn(C)c2c1] | D | 37% | 459.15 [M + H]⁺ | 459.18 | 1.76 | A |
| 112 | 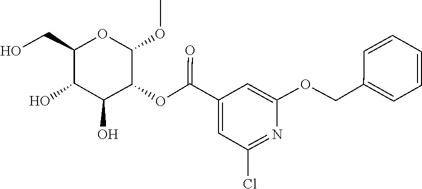 [SMILES: CO[C@H]1O[C@H](CO)[C@@H](O)[C@H](O)[C@H]1OC(=O)c1cc(Cl)nc(OCc2ccccc2)c1] | D | 17% | 484.03 [M − H + formic acid]⁻ | 484.10 | 1.88 | A |
| 113 | 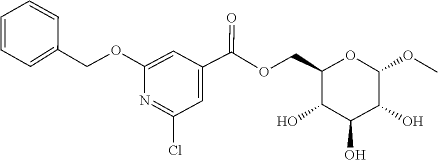 [SMILES: CO[C@H]1O[C@H](COC(=O)c2cc(Cl)nc(OCc3ccccc3)c2)[C@@H](O)[C@H](O)[C@H]1O] | D | 11% | 440.1 [M + H]⁺ | 440.11 | 2.03 | A |
| 114 | 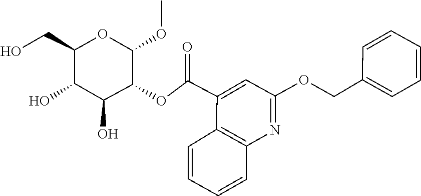 [SMILES: CO[C@H]1O[C@H](CO)[C@@H](O)[C@H](O)[C@H]1OC(=O)c1cc(OCc2ccccc2)nc2ccccc12] | D | 9% | 456.15 [M + H]⁺ | 456.17 | 2.14 | A |
| 115 | 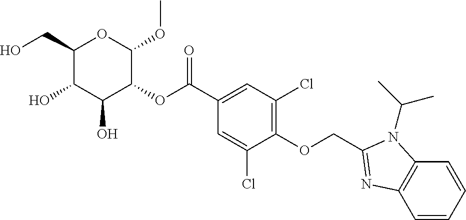 [SMILES: CO[C@H]1O[C@H](CO)[C@@H](O)[C@H](O)[C@H]1OC(=O)c1cc(Cl)c(OCc2nc3ccccc3n2C(C)C)c(Cl)c1] | D | 5% | 555.12 [M + H]⁺ | 555.13 | 1.76 | A |
| 116 | 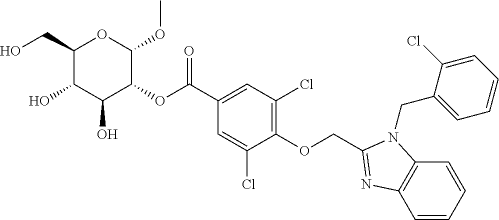 [SMILES: CO[C@H]1O[C@H](CO)[C@@H](O)[C@H](O)[C@H]1OC(=O)c1cc(Cl)c(OCc2nc3ccccc3n2Cc2ccccc2Cl)c(Cl)c1] | D | 17% | 637.06 [M + H]⁺ | 637.09 | 2.25 | A |
| 117 | 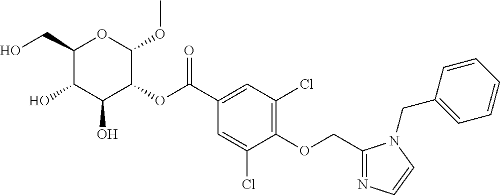 [SMILES: CO[C@H]1O[C@H](CO)[C@@H](O)[C@H](O)[C@H]1OC(=O)c1cc(Cl)c(OCc2nccn2Cc2ccccc2)c(Cl)c1] | D | 15% | 553.11 [M + H]⁺ | 553.11 | 1.39 | A |
| 118 | 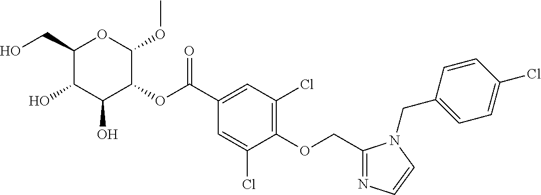 [SMILES: CO[C@H]1O[C@H](CO)[C@@H](O)[C@H](O)[C@H]1OC(=O)c1cc(Cl)c(OCc2nccn2Cc2ccc(Cl)cc2)c(Cl)c1] | D | 13% | 587.05 [M + H]⁺ | 587.08 | 1.55 | A |
| 119 | 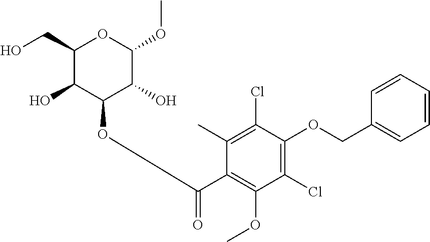 [SMILES: COc1c(Cl)c(OCc2ccccc2)c(Cl)c(C)c1C(=O)O[C@H]1[C@@H](O)[C@@H](CO)O[C@H](OC)[C@@H]1O] | D | 24% | 517.04 [M + H]⁺ | 517.10 | 2.16 | A |
| 120 | 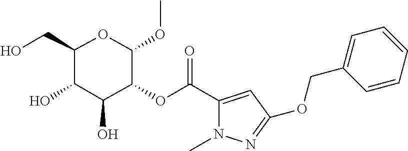 [SMILES: CO[C@H]1O[C@H](CO)[C@@H](O)[C@H](O)[C@H]1OC(=O)c1cc(OCc2ccccc2)nn1C] | D | 64% | 409.17 [M + H]⁺ | 409.16 | 1.59 | A |
| 121 | 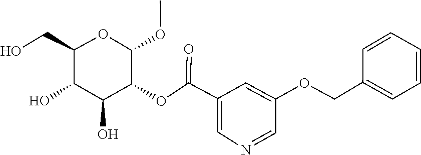 [SMILES: CO[C@H]1O[C@H](CO)[C@@H](O)[C@H](O)[C@H]1OC(=O)c1cncc(OCc2ccccc2)c1] | D | 14% | 406.16 [M + H]⁺ | 406.15 | 1.36 | A |
| 122 | 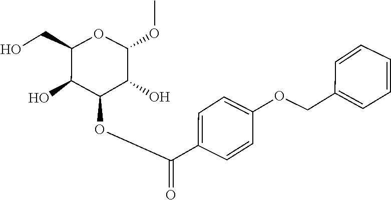 [SMILES: CO[C@H]1O[C@H](CO)[C@H](O)[C@H](OC(=O)c2ccc(OCc3ccccc3)cc2)[C@H]1O] | D | 100% | 405.17 [M + H]⁺ | 405.16 | 1.65 | A |
| 123 | 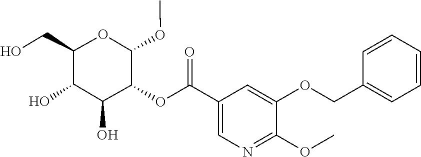 [SMILES: COc1ncc(C(=O)O[C@H]2[C@@H](OC)O[C@H](CO)[C@@H](O)[C@@H]2O)cc1OCc1ccccc1] | D | 8% | 436.15 [M + H]⁺ | 436.16 | 1.66 | A |
| 124 | 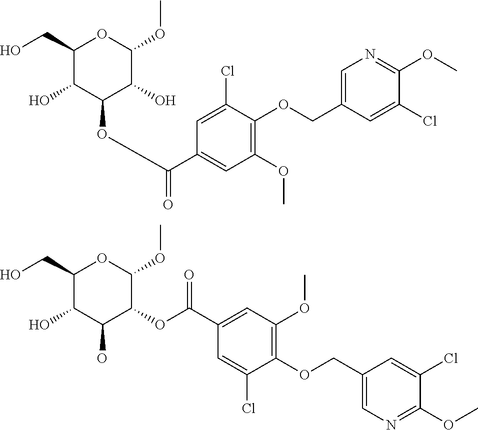 | D | 1% | 534.11 [M + H]⁺ | 534.09 | 1.92 | A |
| 125 | 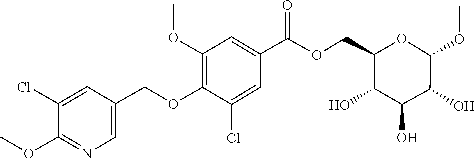 | D | 1% | 534.11 [M + H]⁺ | 534.09 | 2.06 | A |
| 126 | 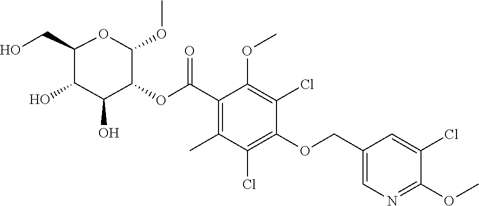 [SMILES: COc1ncc(COc2c(Cl)c(C)c(C(=O)O[C@H]3[C@@H](OC)O[C@H](CO)[C@@H](O)[C@@H]3O)c(OC)c2Cl)cc1Cl] | D | 5% | 582.03 [M + H]⁺ | 582.07 | 2.27 | A |
| 127 | 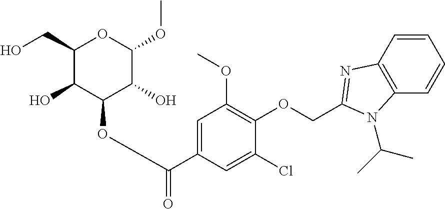 [SMILES: COc1cc(C(=O)O[C@H]2[C@@H](O)[C@@H](CO)O[C@H](OC)[C@@H]2O)cc(Cl)c1OCc1nc2ccccc2n1C(C)C] | D | 67% | 551.17 [M + H]⁺ | 551.18 | 1.49 | A |
| 128 | 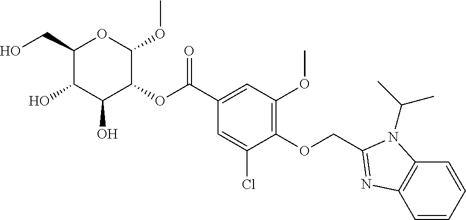 [SMILES: COc1cc(C(=O)O[C@H]2[C@@H](OC)O[C@H](CO)[C@@H](O)[C@@H]2O)cc(Cl)c1OCc1nc2ccccc2n1C(C)C] | D | 6% | 551.17 [M + H]⁺ | 551.18 | 1.47 | A |
| 129 | 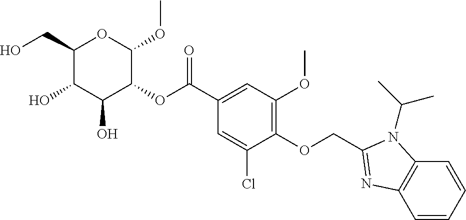 [SMILES: COc1cc(C(=O)O[C@H]2[C@@H](OC)O[C@H](CO)[C@@H](O)[C@@H]2O)cc(Cl)c1OCc1nc2ccccc2n1C(C)C] | D | 2% | 551.17 [M + H]⁺ | 551.18 | 1.53 | A |
| | 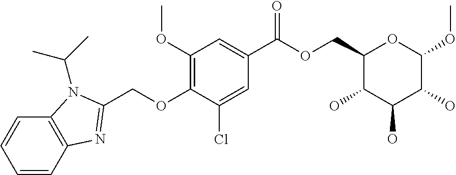 [SMILES: COc1cc(C(=O)OC[C@H]2O[C@H](OC)[C@H](O)[C@@H](O)[C@@H]2O)cc(Cl)c1OCc1nc2ccccc2n1C(C)C] | | | | | | |
| 130 | 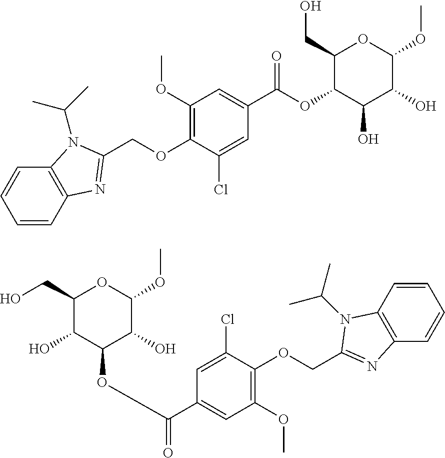 [SMILES: COc1cc(C(=O)O[C@H]2[C@H](O)[C@@H](O)[C@@H](OC)O[C@@H]2CO)cc(Cl)c1OCc1nc2ccccc2n1C(C)C ; COc1cc(C(=O)O[C@@H]2[C@@H](O)[C@@H](OC)O[C@H](CO)[C@H]2O)cc(Cl)c1OCc1nc2ccccc2n1C(C)C] | D | 15% | 551.19 [M + H]⁺ | 551.18 | 1.61 | A |
| 131 | 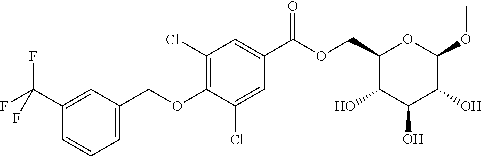 [SMILES: CO[C@@H]1O[C@H](COC(=O)c2cc(Cl)c(OCc3cccc(C(F)(F)F)c3)c(Cl)c2)[C@@H](O)[C@H](O)[C@H]1O] | A | 58% | 585.11 [M − H + formic acid]⁻ | 585.05 | 1.88 | A |
| 132 | 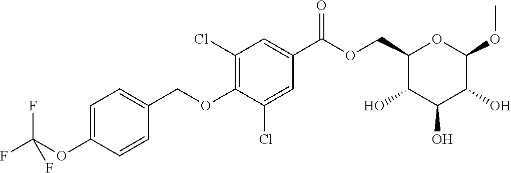 [SMILES: CO[C@@H]1O[C@H](COC(=O)c2cc(Cl)c(OCc3ccc(OC(F)(F)F)cc3)c(Cl)c2)[C@@H](O)[C@H](O)[C@H]1O] | A | 4% | 601.12 [M − H + formic acid]⁻ | 601.05 | 1.91 | A |
| 133 | 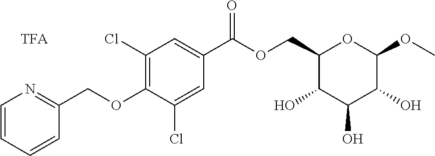 [SMILES: CO[C@@H]1O[C@H](COC(=O)c2cc(Cl)c(OCc3ccccn3)c(Cl)c2)[C@@H](O)[C@H](O)[C@H]1O ; "TFA"] | A | 17% | 474.17 [M + H]⁺ | 474.07 | 1.46 | A |
| 134 | 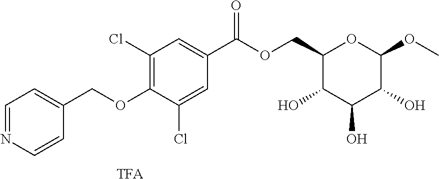 [SMILES: CO[C@@H]1O[C@H](COC(=O)c2cc(Cl)c(OCc3ccncc3)c(Cl)c2)[C@@H](O)[C@H](O)[C@H]1O ; "TFA"] | A | 39% | 474.17 [M + H]⁺ | 474.07 | 1.18 | A |
| 135 | 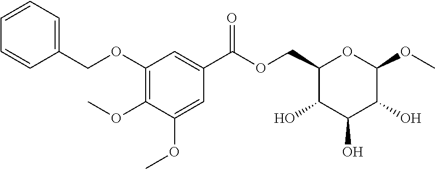 [SMILES: COc1cc(C(=O)OC[C@H]2O[C@@H](OC)[C@H](O)[C@@H](O)[C@@H]2O)cc(OCc2ccccc2)c1OC] | A | 49% | 509.3 [M − H + formic acid]⁻ | 509.17 | 1.57 | A |
| 136 | 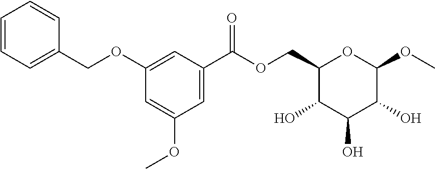 [SMILES: COc1cc(OCc2ccccc2)cc(C(=O)OC[C@H]2O[C@@H](OC)[C@H](O)[C@@H](O)[C@@H]2O)c1] | A | 47% | 479.25 [M − H + formic acid]⁻ | 479.16 | 1.64 | A |
| 137 | 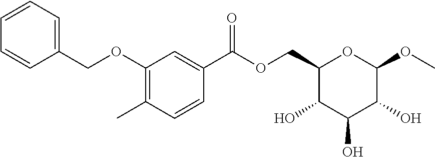 [SMILES: CO[C@@H]1O[C@H](COC(=O)c2ccc(C)c(OCc3ccccc3)c2)[C@@H](O)[C@H](O)[C@H]1O] | A | 56% | 463.24 [M − H + formic acid]⁻ | 463.16 | 1.70 | A |
| 138 | 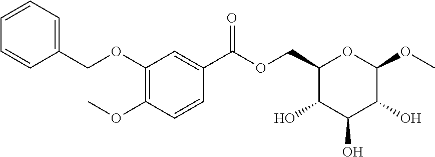 [SMILES: COc1ccc(C(=O)OC[C@H]2O[C@@H](OC)[C@H](O)[C@@H](O)[C@@H]2O)cc1OCc1ccccc1] | A | 37% | 479.28 [M − H + formic acid]⁻ | 479.16 | 1.54 | A |
| 139 | 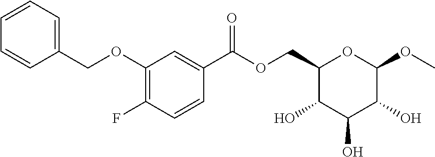 [SMILES: CO[C@@H]1O[C@H](COC(=O)c2ccc(F)c(OCc3ccccc3)c2)[C@@H](O)[C@H](O)[C@H]1O] | A | 38% | 467.26 [M − H + formic acid]⁻ | 467.14 | 1.63 | A |
| 140 | 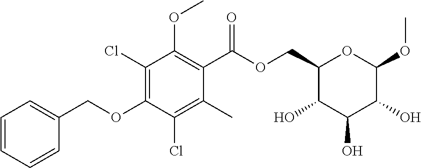 [SMILES: COc1c(Cl)c(OCc2ccccc2)c(Cl)c(C)c1C(=O)OC[C@H]1O[C@@H](OC)[C@H](O)[C@@H](O)[C@@H]1O] | A | 9% | 561.11 [M − H + formic acid]⁻ | 561.09 | 1.79 | A |
| 141 | 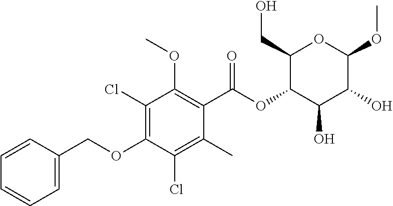 [SMILES: COc1c(Cl)c(OCc2ccccc2)c(Cl)c(C)c1C(=O)O[C@H]1[C@H](O)[C@@H](O)[C@H](OC)O[C@@H]1CO] | A*² | 78% | 561.17 [M − H + formic acid]⁻ | 561.09 | 1.74 | A |
| 142 | 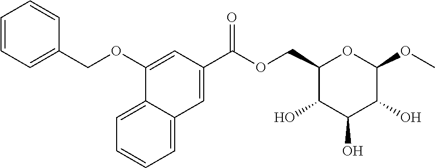 [SMILES: CO[C@@H]1O[C@H](COC(=O)c2cc(OCc3ccccc3)c3ccccc3c2)[C@@H](O)[C@H](O)[C@H]1O] | A | 40% | 499.25 [M − H + formic acid]⁻ | 499.16 | 1.76 | A |
| 143 | 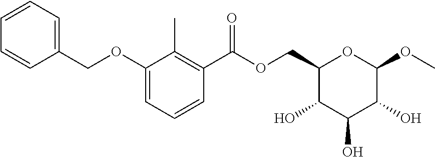 [SMILES: CO[C@@H]1O[C@H](COC(=O)c2cccc(OCc3ccccc3)c2C)[C@@H](O)[C@H](O)[C@H]1O] | A | 48% | 463.22 [M − H + formic acid]⁻ | 463.16 | 1.63 | A |
| 144 | 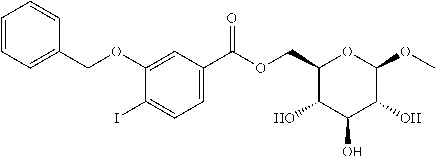 [SMILES: CO[C@@H]1O[C@H](COC(=O)c2ccc(I)c(OCc3ccccc3)c2)[C@@H](O)[C@H](O)[C@H]1O] | A | 36% | 575.1 [M − H + formic acid]⁻ | 575.04 | 1.72 | A |
| 145 | 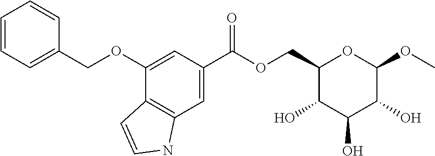 [SMILES: CO[C@@H]1O[C@H](COC(=O)c2cc(OCc3ccccc3)c3cc[nH]c3c2)[C@@H](O)[C@H](O)[C@H]1O] | A | 39% | 442.23 [M − H]⁻ | 442.15 | 1.56 | A |
| 146 | 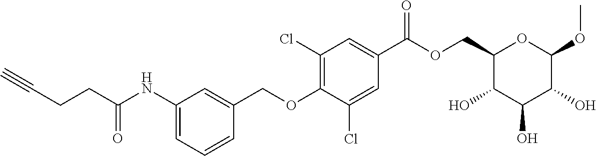 [SMILES: C#CCCC(=O)Nc1cccc(COc2c(Cl)cc(C(=O)OC[C@H]3O[C@@H](OC)[C@H](O)[C@@H](O)[C@@H]3O)cc2Cl)c1] | A | 3% | 568.09 [M + H]⁺ | 568.11 | 1.87 | A |
| 147 | 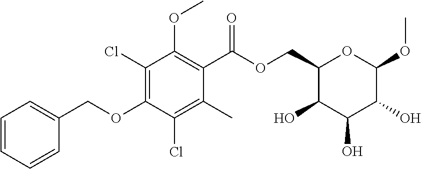 [SMILES: COc1c(Cl)c(OCc2ccccc2)c(Cl)c(C)c1C(=O)OC[C@H]1O[C@@H](OC)[C@H](O)[C@@H](O)[C@H]1O] | A | 24% | 561.06 [M − H + formic acid]⁻ | 561.09 | 2.24 | A |
| 148 | 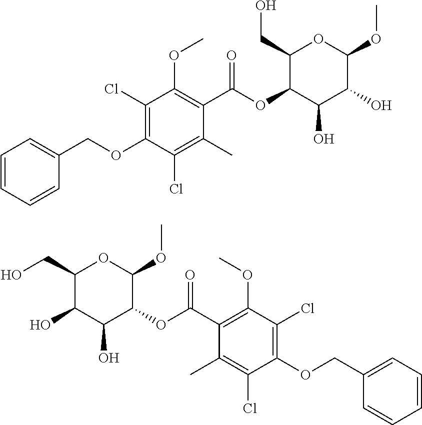 | A | 10% | 561.05 [M − H + formic acid]⁻ | 561.09 | 2.10 | A |
| 149 | 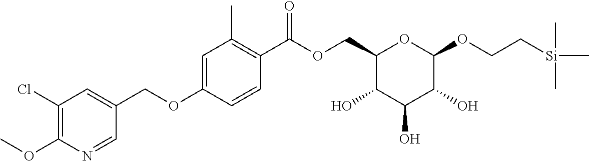 | A | 36% | 570.18 [M + H]⁺ | 570.19 | 2.56 | A |
| 150 | 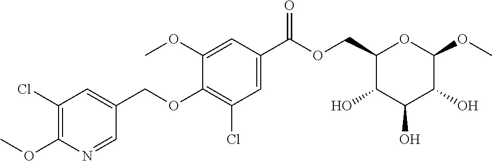 | A | 38% | 534.1 [M + H]⁺ | 534.09 | 2.03 | A |
| 151 | 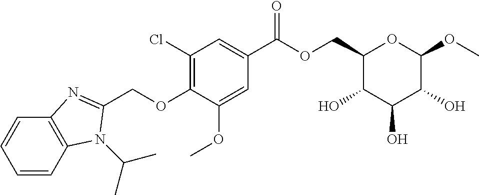 [SMILES: COc1cc(C(=O)OC[C@H]2O[C@@H](OC)[C@H](O)[C@@H](O)[C@@H]2O)cc(Cl)c1OCc1nc2ccccc2n1C(C)C] | A | 35% | 551.19 [M + H]⁺ | 551.18 | 1.59 | A |
| 152 | 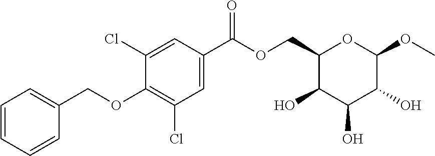 [SMILES: CO[C@@H]1O[C@H](COC(=O)c2cc(Cl)c(OCc3ccccc3)c(Cl)c2)[C@H](O)[C@H](O)[C@H]1O] | E | 68% | 473.05 [M + H]⁺ | 473.08 | 2.15 | A |
| 153b | 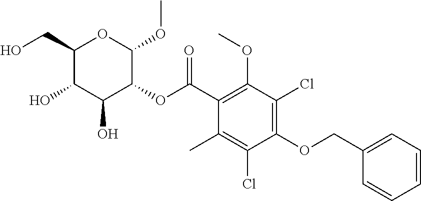 [SMILES: COc1c(Cl)c(OCc2ccccc2)c(Cl)c(C)c1C(=O)O[C@H]1[C@@H](OC)O[C@H](CO)[C@@H](O)[C@@H]1O] | G*³ | 2% | 561.1 [M − H + formic acid]⁻ | 561.09 | 1.79 | A |
| 153c | 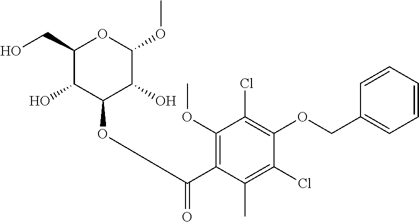 [SMILES: COc1c(Cl)c(OCc2ccccc2)c(Cl)c(C)c1C(=O)O[C@@H]1[C@@H](O)[C@@H](OC)O[C@H](CO)[C@H]1O] | G*³ | 2% | 561.05 [M − H + formic acid]⁻ | 561.11 | 1.75 | A |
| 153d | 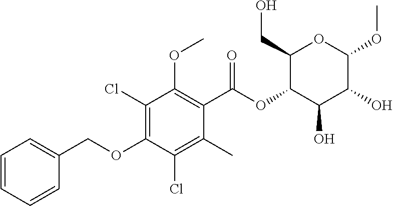 [SMILES: COc1c(Cl)c(OCc2ccccc2)c(Cl)c(C)c1C(=O)O[C@H]1[C@H](O)[C@@H](O)[C@@H](OC)O[C@@H]1CO] | G*³ | 1% | 561.05 [M − H + formic acid]⁻ | 561.09 | 1.76 | A |
| 154a | 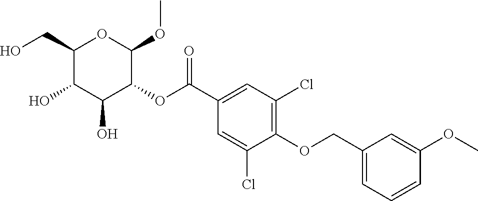 [SMILES: COc1cccc(COc2c(Cl)cc(C(=O)O[C@H]3[C@H](OC)O[C@H](CO)[C@@H](O)[C@@H]3O)cc2Cl)c1] | G*⁴ | 3% | 547.06 [M − H + formic acid]⁻ | 547.08 | 1.65 | A |
| 154b | 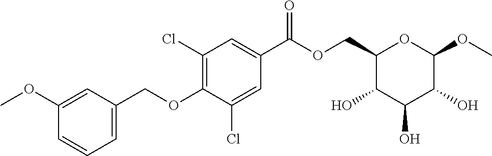 | G*⁴ | 14% | 547.05 [M − H + formic acid]⁻ | 547.08 | 1.72 | A |
| 155 | 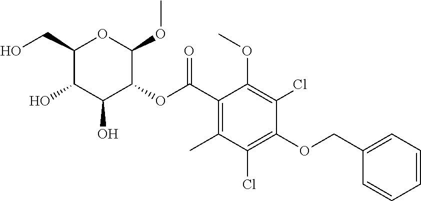 [SMILES: COc1c(Cl)c(OCc2ccccc2)c(Cl)c(C)c1C(=O)O[C@H]1[C@H](OC)O[C@H](CO)[C@@H](O)[C@@H]1O] | G*² | 1% | 561.17 [M − H + formic acid]⁻ | 561.09 | 1.79 | A |
| 156 | 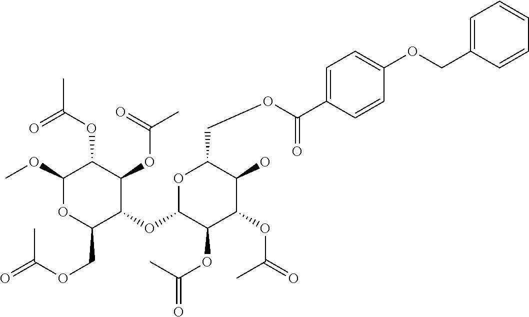 [SMILES: CO[C@@H]1O[C@H](COC(C)=O)[C@@H](O[C@@H]2O[C@H](COC(=O)c3ccc(OCc4ccccc4)cc3)[C@@H](O)[C@H](OC(C)=O)[C@H]2OC(C)=O)[C@H](OC(C)=O)[C@H]1OC(C)=O] | D*⁵ | 25% | 821.34 [M − H + formic acid]⁻ | 821.28 | 1.87 | A |
| 157b | 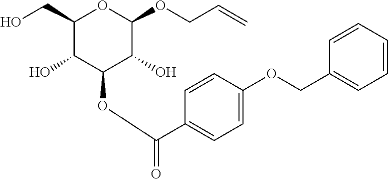 [SMILES: C=CCO[C@@H]1O[C@H](CO)[C@@H](O)[C@H](OC(=O)c2ccc(OCc3ccccc3)cc2)[C@H]1O] | H | 16% | 373.1 [M − OAII]⁺ | 373.09 | 1.24 | B |
| 157c | 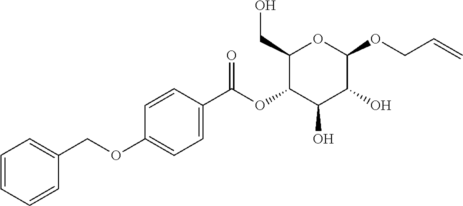 [SMILES: C=CCO[C@@H]1O[C@H](CO)[C@@H](OC(=O)c2ccc(OCc3ccccc3)cc2)[C@H](O)[C@H]1O] | H | 6% | 431.20 [M + H]⁺ | 431.16 | 1.24 | B |
| 158 | 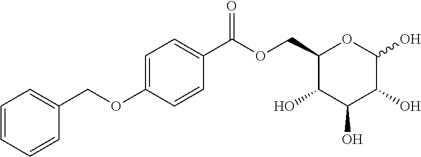 [SMILES: O=C(OC[C@H]1OC(O)[C@H](O)[C@@H](O)[C@@H]1O)c1ccc(OCc2ccccc2)cc1] | H*⁶ | 12% | 389.25 [M − H]⁻ | 389.13 | 1.47/1.45 | A |
| 159 | 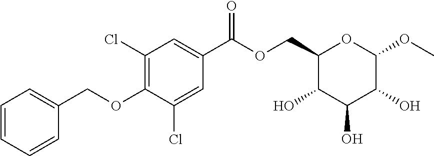 [SMILES: CO[C@H]1O[C@H](COC(=O)c2cc(Cl)c(OCc3ccccc3)c(Cl)c2)[C@@H](O)[C@H](O)[C@H]1O] | G | 52% | 517.05 [M − H + formic acid]⁻ | 517.07 | 1.75 | A |
| 160 | 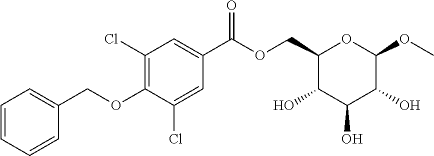 [SMILES: CO[C@@H]1O[C@H](COC(=O)c2cc(Cl)c(OCc3ccccc3)c(Cl)c2)[C@@H](O)[C@H](O)[C@H]1O] | A | 13% | 517.02 [M − H + formic acid]⁻ | 517.07 | 2.14 | A |
| 163a | 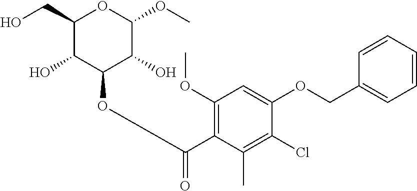 [SMILES: COc1cc(OCc2ccccc2)c(Cl)c(C)c1C(=O)O[C@@H]1[C@@H](O)[C@@H](OC)O[C@H](CO)[C@H]1O] | K*⁷ | 13% | 527.3/529.0 [M − H + formic acid]⁻ | 527.15 | 1.96 | D |
| 163b | 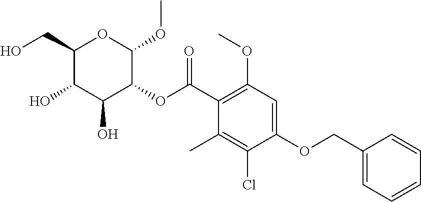 [SMILES: COc1cc(OCc2ccccc2)c(Cl)c(C)c1C(=O)O[C@H]1[C@@H](OC)O[C@H](CO)[C@@H](O)[C@@H]1O] | K*⁷ | 4% | 527.3/529.3 [M − H + formic acid]⁻ | 527.15 | 2.05 | D |
| 164 | 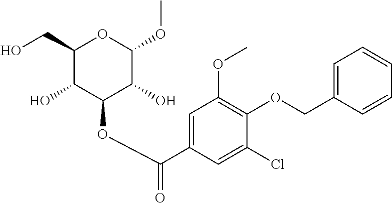 [SMILES: COc1cc(C(=O)O[C@@H]2[C@@H](O)[C@@H](OC)O[C@H](CO)[C@H]2O)cc(Cl)c1OCc1ccccc1] | K*⁷ | 1% | 513.3/515.20 [M − H + formic acid]⁻ | 513.22 | 1.90 | D |
| 165 | 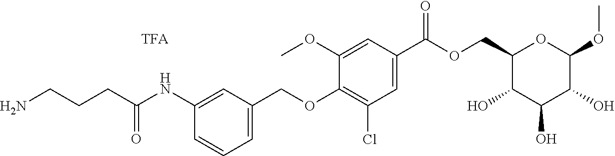 [SMILES: COc1cc(C(=O)OC[C@H]2O[C@@H](OC)[C@H](O)[C@@H](O)[C@@H]2O)cc(Cl)c1OCc1cccc(NC(=O)CCCN)c1 ; "TFA"] | A*⁸ | 25% | 569.2/571.0 [M + H]⁺ | 569.18 | 1.23 | D |
| 166 | 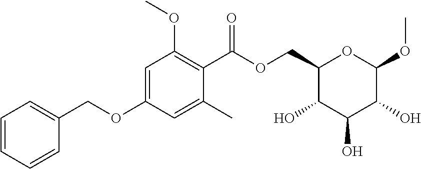 [SMILES: COc1cc(OCc2ccccc2)cc(C)c1C(=O)OC[C@H]1O[C@@H](OC)[C@H](O)[C@@H](O)[C@@H]1O] | A | 61% | 493.25 [M − H + formic acid]⁻ | 493.19 | 1.88 | D |
| 167 | 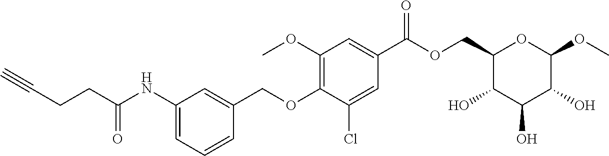 [SMILES: C#CCCC(=O)Nc1cccc(COc2c(Cl)cc(C(=O)OC[C@H]3O[C@@H](OC)[C@H](O)[C@@H](O)[C@@H]3O)cc2OC)c1] | A | 19% | 608.3/610.3 [M − H + formic acid]⁻ | 608.17 | 1.79 | D |
| 168 | 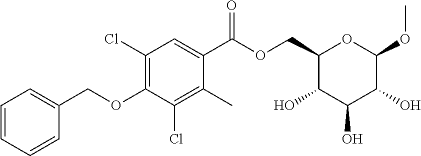 [SMILES: CO[C@@H]1O[C@H](COC(=O)c2cc(Cl)c(OCc3ccccc3)c(Cl)c2C)[C@@H](O)[C@H](O)[C@H]1O] | A | 40% | 531.2/533.2 [M − H + formic acid]⁻ | 531.10 | 2.27 | D |
| 169 | 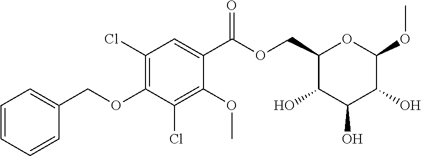 [SMILES: COc1c(C(=O)OC[C@H]2O[C@@H](OC)[C@H](O)[C@@H](O)[C@@H]2O)cc(Cl)c(OCc2ccccc2)c1Cl] | A | 30% | 547.3/549.2/551.2 [M − H + formic acid]⁻ | 547.10 | 2.16 | D |
| 170 | 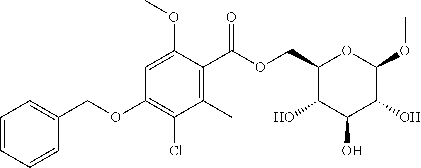 [SMILES: COc1cc(OCc2ccccc2)c(Cl)c(C)c1C(=O)OC[C@H]1O[C@@H](OC)[C@H](O)[C@@H](O)[C@@H]1O] | A | 60% | 527.3/529.22 [M − H + formic acid]⁻ | 527.04 | 2.04 | D |
| 172 | 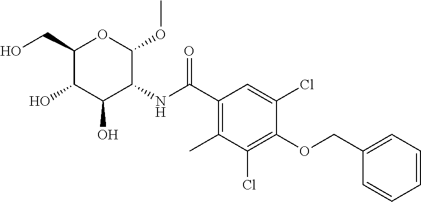 [SMILES: CO[C@H]1O[C@H](CO)[C@@H](O)[C@H](O)[C@H]1NC(=O)c1cc(Cl)c(OCc2ccccc2)c(Cl)c1C] | L | 23% | 484.3/486.227 [M − H]⁻ | 484.09 | 1.98 | D |
| 173 | 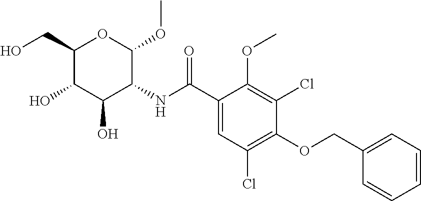 [SMILES: COc1c(C(=O)N[C@H]2[C@@H](OC)O[C@H](CO)[C@@H](O)[C@@H]2O)cc(Cl)c(OCc2ccccc2)c1Cl] | L | 43% | 500.2/502.1 [M − H]⁻ | 500.09 | 2.05 | D |
| 174 | 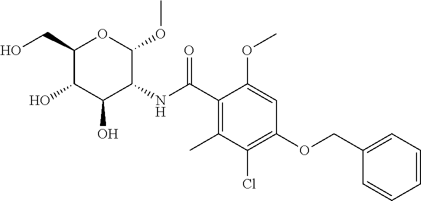 [SMILES: COc1cc(OCc2ccccc2)c(Cl)c(C)c1C(=O)N[C@H]1[C@@H](OC)O[C@H](CO)[C@@H](O)[C@@H]1O] | L | 68% | 480.3/482.3 [M − H]⁻ | 480.14 | 1.81 | D |
| 175 | 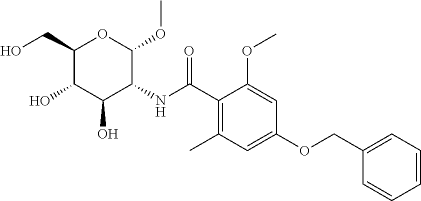 [SMILES: COc1cc(OCc2ccccc2)cc(C)c1C(=O)N[C@H]1[C@@H](OC)O[C@H](CO)[C@@H](O)[C@@H]1O] | L | 72% | 446.33 [M − H]⁻ | 446.18 | 1.67 | D |
| 176 | 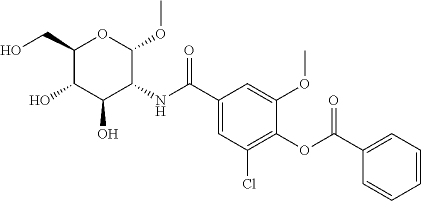 [SMILES: COc1cc(C(=O)N[C@H]2[C@@H](OC)O[C@H](CO)[C@@H](O)[C@@H]2O)cc(Cl)c1OC(=O)c1ccccc1] | L | 59% | 526.3/528.2 [M − H + formic acid]⁻ | 526.15 | 1.84 | D |
| 177 | 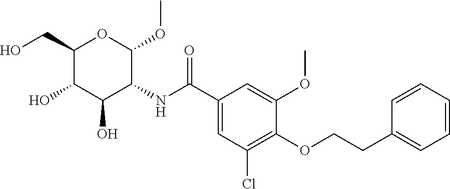 [SMILES: COc1cc(C(=O)N[C@H]2[C@@H](OC)O[C@H](CO)[C@@H](O)[C@@H]2O)cc(Cl)c1OCCc1ccccc1] | L | 65% | 526.3/528.3 [M − H + formic acid]⁻ | 526.17 | 1.97 | D |
| 178 | 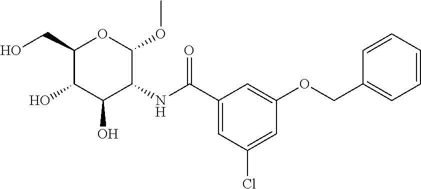 [SMILES: CO[C@H]1O[C@H](CO)[C@@H](O)[C@H](O)[C@H]1NC(=O)c1cc(Cl)cc(OCc2ccccc2)c1] | L | 63% | 482.3/484.2 [M − H + formic acid]⁻ | 482.15 | 1.86 | D |
| 179 | 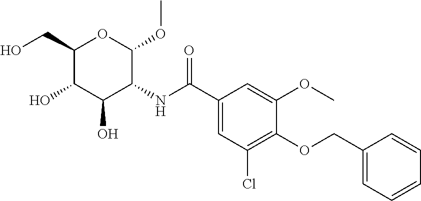 [SMILES: COc1cc(C(=O)N[C@H]2[C@@H](OC)O[C@H](CO)[C@@H](O)[C@@H]2O)cc(Cl)c1OCc1ccccc1] | L | 42% | 512.3/514.2 [M − H + formic acid]⁻ | 512.16 | 1.84 | D |
| 180 | 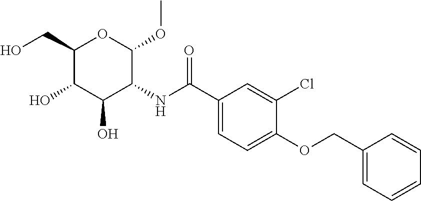 [SMILES: CO[C@H]1O[C@H](CO)[C@@H](O)[C@H](O)[C@H]1NC(=O)c1ccc(OCc2ccccc2)c(Cl)c1] | L | 44% | 482.2/484.2 [M − H + formic acid]⁻ | 482.15 | 1.78 | D |
| 181 | 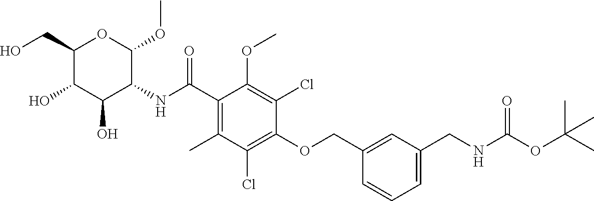 [SMILES: COc1c(Cl)c(OCc2cccc(CNC(=O)OC(C)(C)C)c2)c(Cl)c(C)c1C(=O)N[C@H]1[C@@H](OC)O[C@H](CO)[C@@H](O)[C@@H]1O] | L | 60% | 689.5/691.4 [M − H + formic acid]⁻ | 689.21 | 2.09 | D |
| 182 | 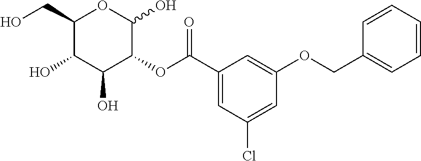 [SMILES: O=C(O[C@H]1C(O)O[C@H](CO)[C@@H](O)[C@@H]1O)c1cc(Cl)cc(OCc2ccccc2)c1] | D | 31% | 469.2/471.1 [M − H + formic acid]⁻ | 469.11 | 1.82 | D |
| 183 | 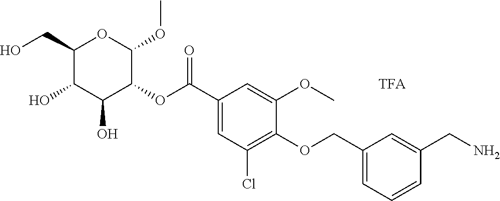 [SMILES: COc1cc(C(=O)O[C@H]2[C@@H](OC)O[C@H](CO)[C@@H](O)[C@@H]2O)cc(Cl)c1OCc1cccc(CN)c1 ; "TFA"] | D*²*⁹ | 15% | 498.14 [M + H]⁺ | 498.15 498.15 | 1.13 | A |
| 184 | 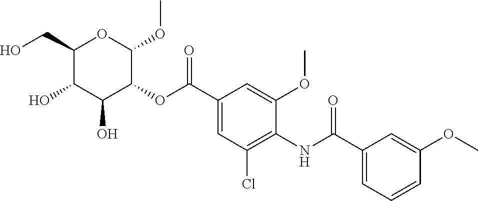 [SMILES: COc1cccc(C(=O)Nc2c(Cl)cc(C(=O)O[C@H]3[C@@H](OC)O[C@H](CO)[C@@H](O)[C@@H]3O)cc2OC)c1] | D | 68% | 512.2/514.1 [M + H]⁺ | 512.13 | 1.57 | D |
| 185 | 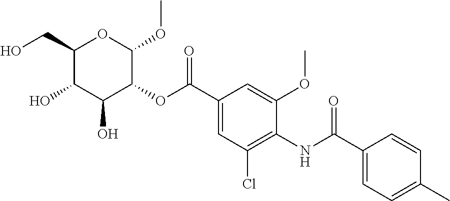 [SMILES: COc1cc(C(=O)O[C@H]2[C@@H](OC)O[C@H](CO)[C@@H](O)[C@@H]2O)cc(Cl)c1NC(=O)c1ccc(C)cc1] | D | 25% | 510.2/512.2 [M − H]⁻ | 510.13 | 1.54 | D |
| 186 | 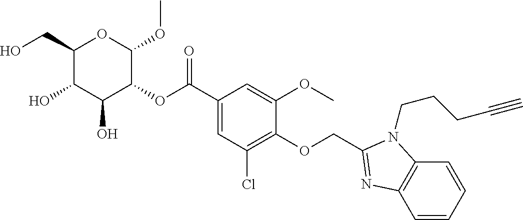 [SMILES: C#CCCCn1c(COc2c(Cl)cc(C(=O)O[C@H]3[C@@H](OC)O[C@H](CO)[C@@H](O)[C@@H]3O)cc2OC)nc2ccccc21] | D | 12% | 575.2/577.2/578.7 [M + H]⁺ | 575.18 | 1.68 | D |
| 187 | 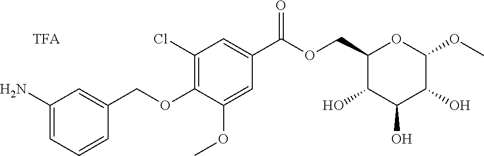 [SMILES: COc1cc(C(=O)OC[C@H]2O[C@H](OC)[C@H](O)[C@@H](O)[C@@H]2O)cc(Cl)c1OCc1cccc(N)c1 ; "TFA"] | A*¹⁰ | 66% | 484.2/486.2 [M + H]⁺ | 483.13 | 1.34 | A |
| 189 | 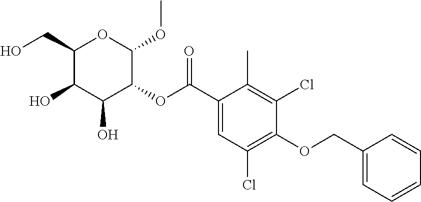 [SMILES: CO[C@H]1O[C@H](CO)[C@H](O)[C@H](O)[C@H]1OC(=O)c1cc(Cl)c(OCc2ccccc2)c(Cl)c1C] | M*¹¹ | 26% | 531.3/533.2/535.4 [M − H + formic acid]⁻ | 531.11 | 2.27 | D |
| 190 | 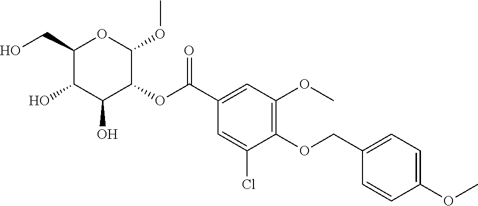 [SMILES: COc1ccc(COc2c(Cl)cc(C(=O)O[C@H]3[C@@H](OC)O[C@H](CO)[C@@H](O)[C@@H]3O)cc2OC)cc1] | L*¹² | 56% | 543.3/545.2 [M − H + formic acid]⁻ | 543.15 | 1.98 | D |
| 191 | 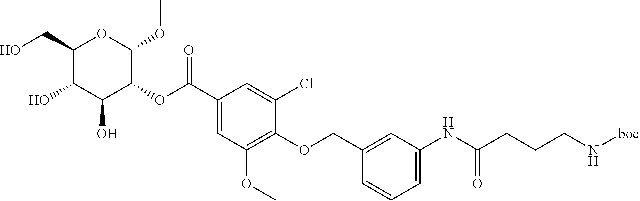 | L*¹² | 25% | 713.5/715.5 [M − H + formic acid]⁻ | 713.26 | 2.00 | D |
| 193 | 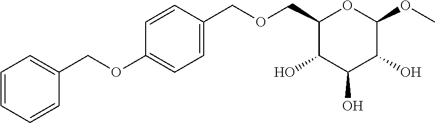 [SMILES: CO[C@@H]1O[C@H](COCc2ccc(OCc3ccccc3)cc2)[C@@H](O)[C@H](O)[C@H]1O] | N | 1% | 435.16 [M − H + formic acid]⁻ | 435.17 | 1.56 | A |
| 194 | 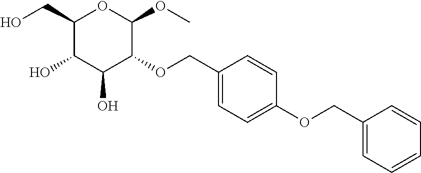 [SMILES: CO[C@@H]1O[C@H](CO)[C@@H](O)[C@H](O)[C@H]1OCc1ccc(OCc2ccccc2)cc1] | N | 19% | 435.15 [M − H + formic acid]⁻ | 435.17 | 1.55 | A |
| 196 | 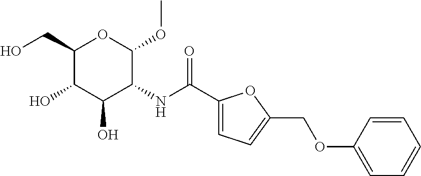 [SMILES: CO[C@H]1O[C@H](CO)[C@@H](O)[C@H](O)[C@H]1NC(=O)c1ccc(COc2ccccc2)o1] | L | 66% | 394.14 [M + H]⁺ | 394.15 | 1.44 | D |
| 197 | 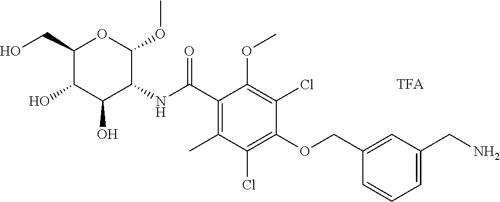 [SMILES: COc1c(Cl)c(OCc2cccc(CN)c2)c(Cl)c(C)c1C(=O)N[C@H]1[C@@H](OC)O[C@H](CO)[C@@H](O)[C@@H]1O ; "TFA"] | L*¹³ | 43% | 589.2/591.3/593.3 [M − H + formic acid]⁻ | 589.16 | 1.04 | D |
| 198 | 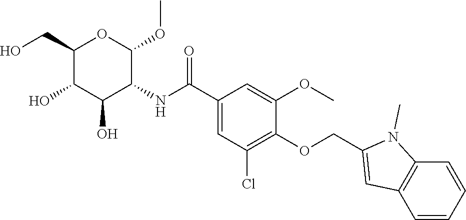 [SMILES: COc1cc(C(=O)N[C@H]2[C@@H](OC)O[C@H](CO)[C@@H](O)[C@@H]2O)cc(Cl)c1OCc1cc2ccccc2n1C] | L | 60% | 565.2/567.2 [M − H + formic acid]⁻ | 565.18 | 2.02 | D |
| 199 | 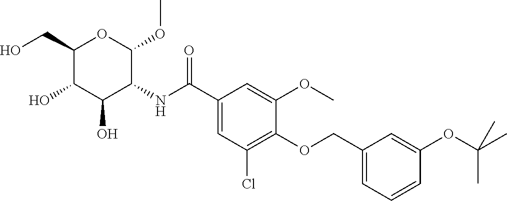 [SMILES: COc1cc(C(=O)N[C@H]2[C@@H](OC)O[C@H](CO)[C@@H](O)[C@@H]2O)cc(Cl)c1OCc1cccc(OC(C)(C)C)c1] | L | 68% | 584.3/586.2 [M − H + formic acid]⁻ | 584.21 | 2.12 | D |
| 200a | 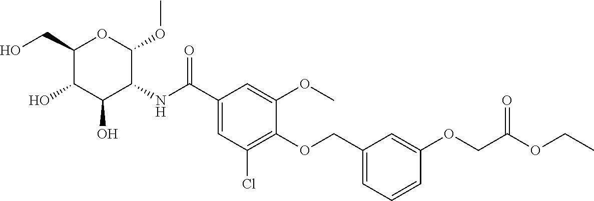 [SMILES: CCOC(=O)COc1cccc(COc2c(Cl)cc(C(=O)N[C@H]3[C@@H](OC)O[C@H](CO)[C@@H](O)[C@@H]3O)cc2OC)c1] | L | 53% | 614.3/616.1 [M − H + formic acid]⁻ | 614.18 | 1.87 | D |
| 200b | 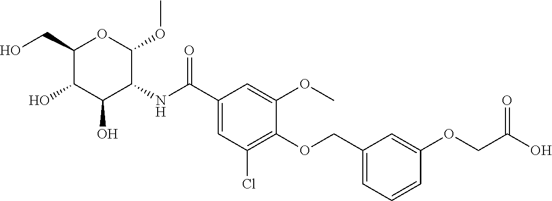 [SMILES: COc1cc(C(=O)N[C@H]2[C@@H](OC)O[C@H](CO)[C@@H](O)[C@@H]2O)cc(Cl)c1OCc1cccc(OCC(=O)O)c1] | L*¹⁴ | 100% | 542.1/543.9 [M + H]⁺ | 540.13 | 1.52 | D |
| 201 | 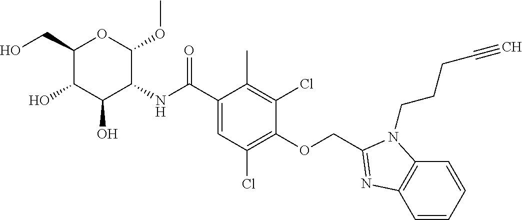 [SMILES: C#CCCCn1c(COc2c(Cl)cc(C(=O)N[C@H]3[C@@H](OC)O[C@H](CO)[C@@H](O)[C@@H]3O)c(C)c2Cl)nc2ccccc21] | L | 35% | 592.1/594.0/596.4 [M + H]⁺ | 592.16 | 1.66 | D |
| 202a | 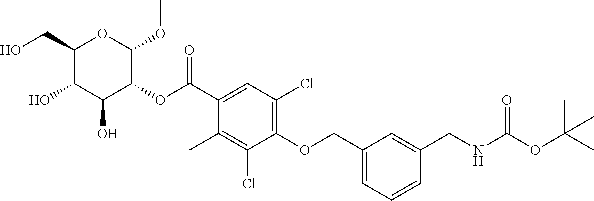 [SMILES: CO[C@H]1O[C@H](CO)[C@@H](O)[C@H](O)[C@H]1OC(=O)c1cc(Cl)c(OCc2cccc(CNC(=O)OC(C)(C)C)c2)c(Cl)c1C] | K*²*¹⁵ | 12% | 660.3/662.2 [M − H + formic acid]⁻ | 660.18 | 2.32 | D |
| 202b | 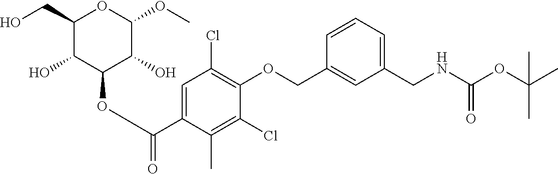 [SMILES: CO[C@H]1O[C@H](CO)[C@@H](O)[C@H](OC(=O)c2cc(Cl)c(OCc3cccc(CNC(=O)OC(C)(C)C)c3)c(Cl)c2C)[C@H]1O] | K*²*¹⁵ | 3% | 660.3/662.2 [M − H + formic acid]⁻ | 660.18 | 2.23 | D |
| 202c | 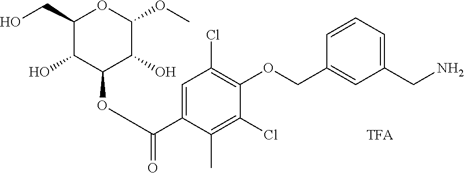 [SMILES: CO[C@H]1O[C@H](CO)[C@@H](O)[C@H](OC(=O)c2cc(Cl)c(OCc3cccc(CN)c3)c(Cl)c2C)[C@H]1O ; "TFA"] | K*²*¹⁶ | 1% | 516.1/518.0/520.2 [M + H]⁺ | 516.12 | 1.12 | D |
| 203 | 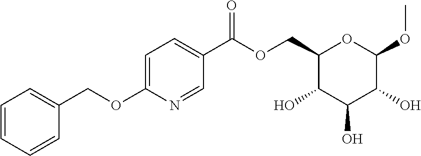 [SMILES: CO[C@@H]1O[C@H](COC(=O)c2ccc(OCc3ccccc3)nc2)[C@@H](O)[C@H](O)[C@H]1O] | G*¹⁷ | 1% | 406.2 [M + H]⁺ | 406.18 | 1.18 | A |
| 204 | 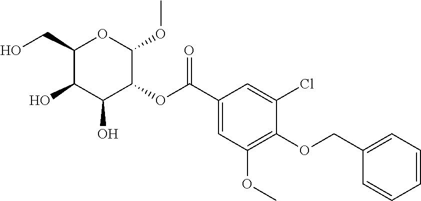 [SMILES: COc1cc(C(=O)O[C@H]2[C@@H](OC)O[C@H](CO)[C@H](O)[C@@H]2O)cc(Cl)c1OCc1ccccc1] | M*¹⁸ | 12% | 513.2/515.1 [M − H + formic acid]⁻ | 513.14 | 2.02 | D |
| 205 | 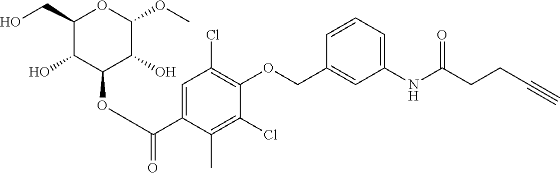 [SMILES: C#CCCC(=O)Nc1cccc(COc2c(Cl)cc(C(=O)O[C@@H]3[C@@H](O)[C@@H](OC)O[C@H](CO)[C@H]3O)c(C)c2Cl)c1] | K*², *¹⁵ | 5% | 582.1/584.0/585.8 [M + H]⁺ | 582.13 | 1.86 | D |
| 206 | 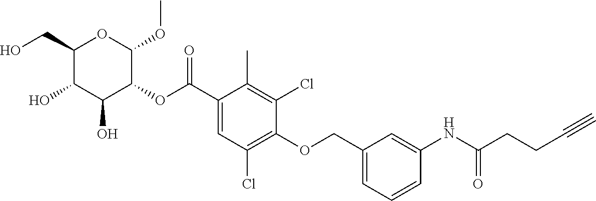 [SMILES: C#CCCC(=O)Nc1cccc(COc2c(Cl)cc(C(=O)O[C@H]3[C@@H](OC)O[C@H](CO)[C@@H](O)[C@@H]3O)c(C)c2Cl)c1] | K*², *¹⁵ | 5% | 582.1/583.9/586.1 [M + H]⁺ | 582.13 | 1.97 | D |
| 207 | 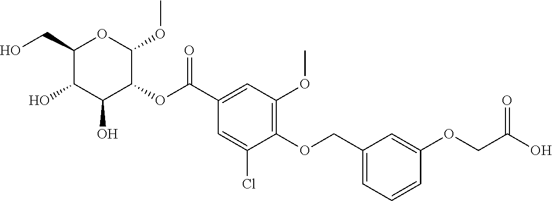 [SMILES: COc1cc(C(=O)O[C@H]2[C@@H](OC)O[C@H](CO)[C@@H](O)[C@@H]2O)cc(Cl)c1OCc1cccc(OCC(=O)O)c1] | L*¹⁹ | 57% | 541.1/543.1 [M − H]⁻ | 541.11 | 1.66 | D |
| 208 | 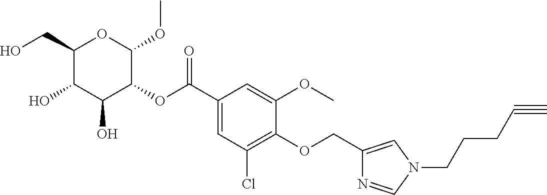 [SMILES: C#CCCCn1cnc(COc2c(Cl)cc(C(=O)O[C@H]3[C@@H](OC)O[C@H](CO)[C@@H](O)[C@@H]3O)cc2OC)c1] | L*²⁰ | 32% | 525.1/527.0 [M + H]⁺ | 525.16 | 1.14 | D |
| 209 | 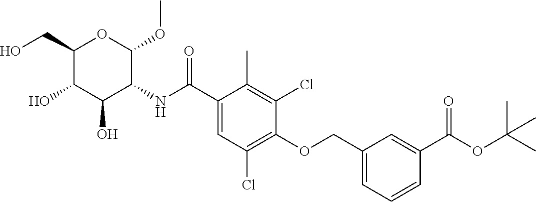 [SMILES: CO[C@H]1O[C@H](CO)[C@@H](O)[C@H](O)[C@H]1NC(=O)c1cc(Cl)c(OCc2cccc(C(=O)OC(C)(C)C)c2)c(Cl)c1C] | L | 30% | 586.1/588.1/589.7 [M + H]⁺ | 586.16 | 2.35 | D |
| 210 | 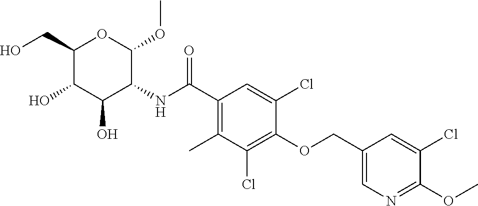 [SMILES: COc1ncc(COc2c(Cl)cc(C(=O)N[C@H]3[C@@H](OC)O[C@H](CO)[C@@H](O)[C@@H]3O)c(C)c2Cl)cc1Cl] | L | 40% | 552.9/554.8 [M + H]⁺ | 551.08 | 2.01 | D |
| 211 | 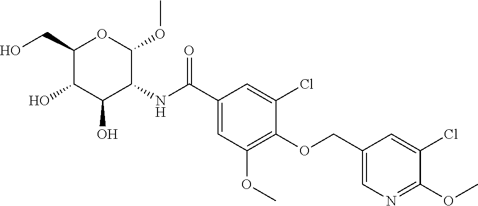 [SMILES: COc1cc(C(=O)N[C@H]2[C@@H](OC)O[C@H](CO)[C@@H](O)[C@@H]2O)cc(Cl)c1OCc1cnc(OC)c(Cl)c1] | L | 48% | 577.2/579.1/581.1 [M − H + formic acid]⁻ | 577.28 | 1.88 | D |
| 212 | 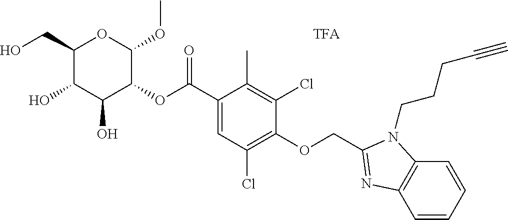 [SMILES: C#CCCCn1c(COc2c(Cl)cc(C(=O)O[C@H]3[C@@H](OC)O[C@H](CO)[C@@H](O)[C@@H]3O)c(C)c2Cl)nc2ccccc21 ; "TFA"] | L*⁷ | 34% | 637.2/639.2 [M − H + formic acid]⁻ | 637.08 | 1.89 | D |
| 213 | 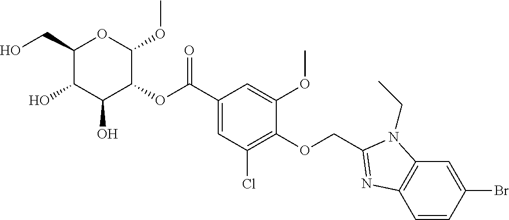 [SMILES: CCn1c(COc2c(Cl)cc(C(=O)O[C@H]3[C@@H](OC)O[C@H](CO)[C@@H](O)[C@@H]3O)cc2OC)nc2ccc(Br)cc21] | D | 1% | 615.1/617.0/619.1 [M + H]⁺ | 615.07 | 2.02 | D |
| 214 | 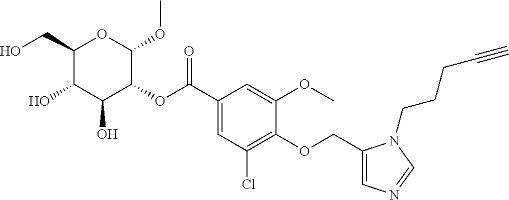 [SMILES: C#CCCCn1cncc1COc1c(Cl)cc(C(=O)O[C@H]2[C@@H](OC)O[C@H](CO)[C@@H](O)[C@@H]2O)cc1OC] | L*⁷ | 5% | 525.1/527.0 [M + H]⁺ | 525.16 | 1.13 | D |
| 215 | 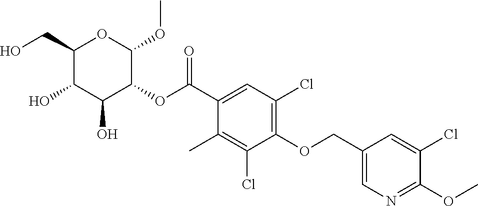 [SMILES: COc1ncc(COc2c(Cl)cc(C(=O)O[C@H]3[C@@H](OC)O[C@H](CO)[C@@H](O)[C@@H]3O)c(C)c2Cl)cc1Cl] | L*⁷ | 19% | 598.1/600.1 [M − H + formic acid]⁻ | 596.00 | 2.32 | D |
| 216 | 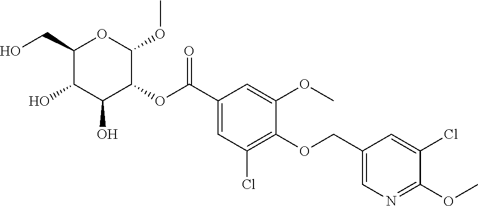 [SMILES: COc1cc(C(=O)O[C@H]2[C@@H](OC)O[C@H](CO)[C@@H](O)[C@@H]2O)cc(Cl)c1OCc1cnc(OC)c(Cl)c1] | L*⁷ | 35% | 578.2/580.1/582.3 [M − H + formic acid]⁻ | 577.11 | 2.07 | D |
| 224 | 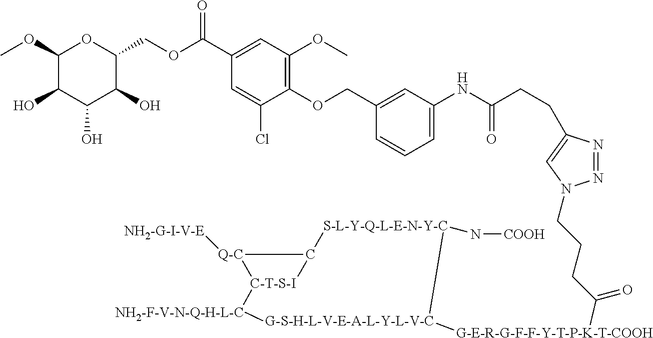 | P | 24% | 1075.7 [M + 6H]⁶⁺ | 1080.72 | λ = 215 nm: 4.76 | F |
| 225 | 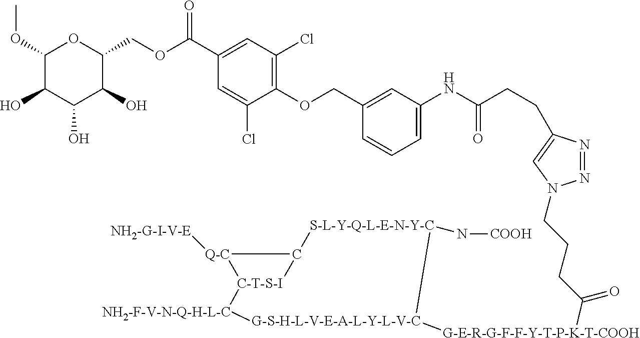 | P | 11% | 1076.5 [M + 6H]⁶⁺ | 1081.38 | λ = 215 nm: 4.76 | F |
| 226 | 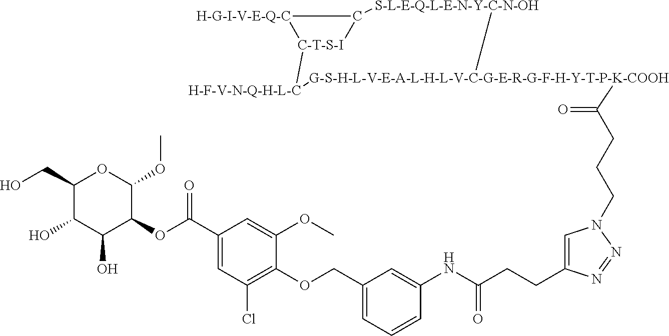 | P | 31% | 1052.6 [M + 6H]⁶⁺ | 1052.13 | λ = 215 nm: 3.62 | F |
| 227 | 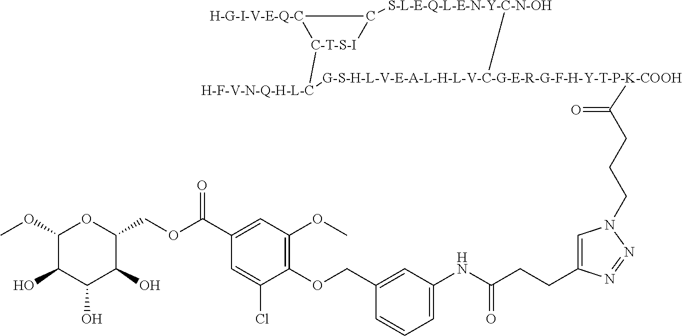 | P | 34% | 1052.5 [M + 6H]⁶⁺ | 1052.13 | λ = 215 nm: 3.64 | F |
| 228 | 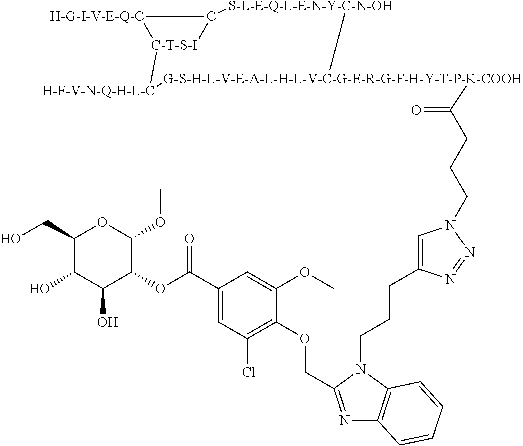 | P | 25% | 1054.3 [M + 6H]⁶⁺ | 1054.04 | λ = 215 nm: 3.46 | F |
| 229 | 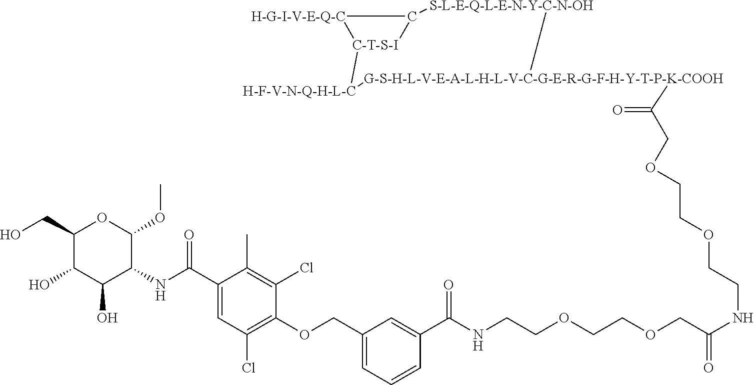 | Q | 27% | 1068.6 [M + 6H]⁶⁺ | 1073.38 | λ = 215 nm: 4.19 | F |
| 230 | 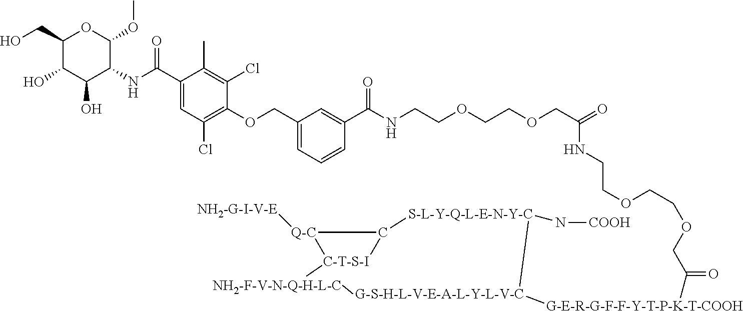 | Q | 16% | 1102.71 [M + 6H]⁶⁺ | 1101.82 | λ = 215 nm: 5.46 | G |
| 231 | 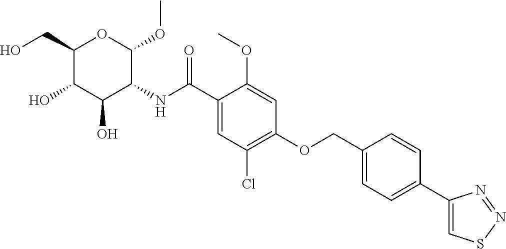 [SMILES: COc1cc(OCc2ccc(-c3csnn3)cc2)c(Cl)cc1C(=O)N[C@H]1[C@@H](OC)O[C@H](CO)[C@@H](O)[C@@H]1O] | R | 29% | 552.21 [M + H]⁺ | 552.12 | 1.87 | D |
| 232 | 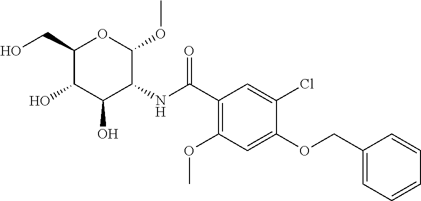 [SMILES: COc1cc(OCc2ccccc2)c(Cl)cc1C(=O)N[C@H]1[C@@H](OC)O[C@H](CO)[C@@H](O)[C@@H]1O] | L | 44% | 468.22 [M + H]⁺ | 468.14 | 1.87 | D |
| 233 | 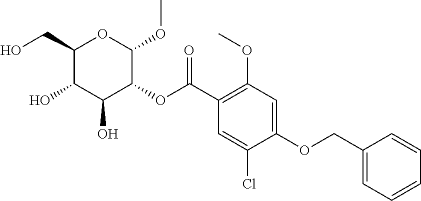 [SMILES: COc1cc(OCc2ccccc2)c(Cl)cc1C(=O)O[C@H]1[C@@H](OC)O[C@H](CO)[C@@H](O)[C@@H]1O] | I*¹⁵ | 34% | 512.99 [M − H + Formic acid]⁻ | 513.21 | 1.90 | D |
| 234 | 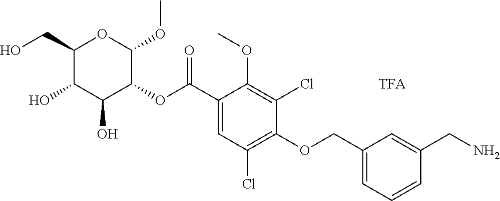 [SMILES: COc1c(C(=O)O[C@H]2[C@@H](OC)O[C@H](CO)[C@@H](O)[C@@H]2O)cc(Cl)c(OCc2cccc(CN)c2)c1Cl ; "TFA"] | I*¹⁶ | 44% | 532.19 [M + H]⁺ | 532.11 | 1.18 | D |
| 235 | 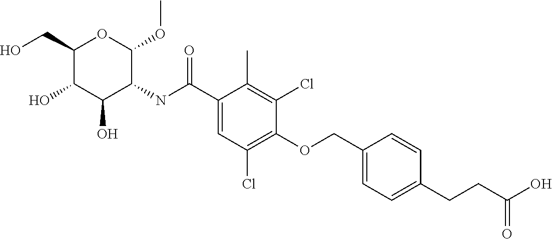 [SMILES: CO[C@H]1O[C@H](CO)[C@@H](O)[C@H](O)[C@H]1NC(=O)c1cc(Cl)c(OCc2ccc(CCC(=O)O)cc2)c(Cl)c1C] | L*¹⁴ | 20% | 557.96 [M + H]⁺ | 558.13 | 1.66 | D |
| 236 | 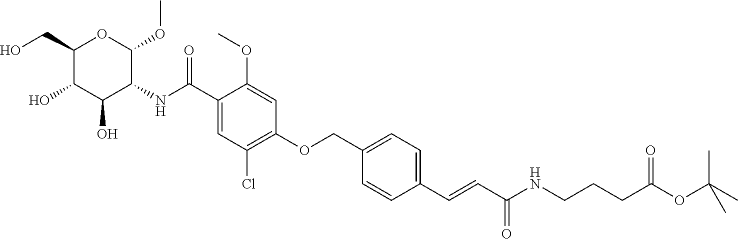 [SMILES: COc1cc(OCc2ccc(/C=C/C(=O)NCCCC(=O)OC(C)(C)C)cc2)c(Cl)cc1C(=O)N[C@H]1[C@@H](OC)O[C@H](CO)[C@@H](O)[C@@H]1O] | L*³⁸ | 30% | 679.39 [M + H]⁺ | 679.26 | 2.00 | D |
| 237 | 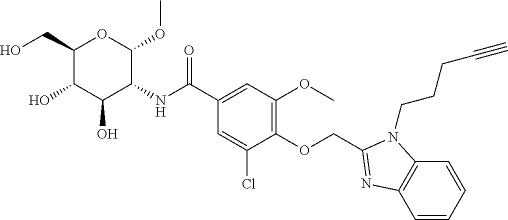 [SMILES: C#CCCCn1c(COc2c(Cl)cc(C(=O)N[C@H]3[C@@H](OC)O[C@H](CO)[C@@H](O)[C@@H]3O)cc2OC)nc2ccccc21] | L | 65% | 574.11 [M + H]⁺ | 574.20 | 1.55 | D |
| 238 | 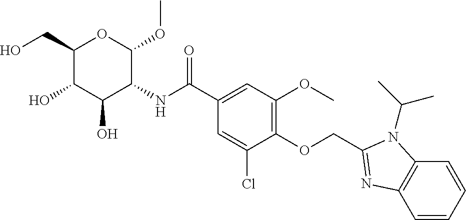 [SMILES: COc1cc(C(=O)N[C@H]2[C@@H](OC)O[C@H](CO)[C@@H](O)[C@@H]2O)cc(Cl)c1OCc1nc2ccccc2n1C(C)C] | L | 53% | 550.10 [M + H]⁺ | 550.20 | 1.46 | D |
| 239 | 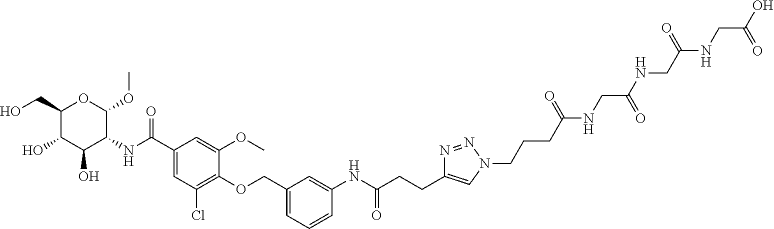 [SMILES: COc1cc(C(=O)N[C@H]2[C@@H](OC)O[C@H](CO)[C@@H](O)[C@@H]2O)cc(Cl)c1OCc1cccc(NC(=O)CCc2cn(CCCC(=O)NCC(=O)NCC(=O)NCC(=O)O)nn2)c1] | P*³⁴ | 42% | 864.3 parent peak | 865.26 | λ= 215 nm: 4.08 | F |
| 240 | 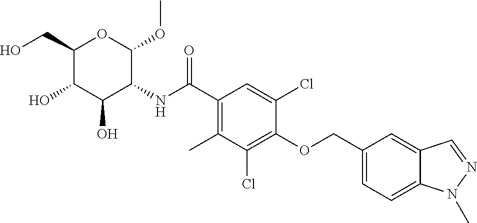 [SMILES: CO[C@H]1O[C@H](CO)[C@@H](O)[C@H](O)[C@H]1NC(=O)c1cc(Cl)c(OCc2ccc3c(cnn3C)c2)c(Cl)c1C] | R | 10% | 540.25 [M + H]⁺ | 540.13 | 1.72 | D |
| 241 | 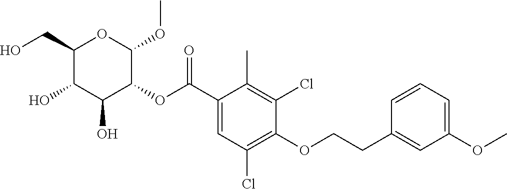 [SMILES: COc1cccc(CCOc2c(Cl)cc(C(=O)O[C@H]3[C@@H](OC)O[C@H](CO)[C@@H](O)[C@@H]3O)c(C)c2Cl)c1] | I | 39% | 575.04 [M − H + Formic acid]⁻ | 575.20 | 2.33 | D |
| 242 | 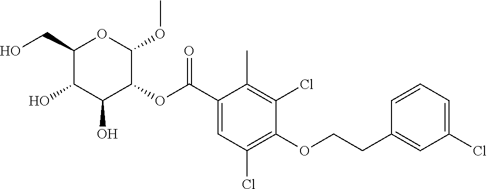 [SMILES: CO[C@H]1O[C@H](CO)[C@@H](O)[C@H](O)[C@H]1OC(=O)c1cc(Cl)c(OCCc2cccc(Cl)c2)c(Cl)c1C] | I*³⁷ | 27% | 579.28 [M − H + Formic acid]⁻ | 579.15 | 2.53 | D |
| 243 | 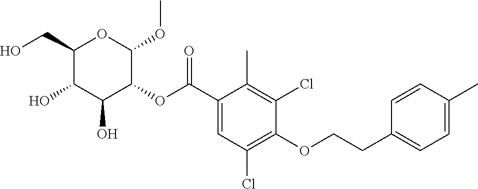 [SMILES: CO[C@H]1O[C@H](CO)[C@@H](O)[C@H](O)[C@H]1OC(=O)c1cc(Cl)c(OCCc2ccc(C)cc2)c(Cl)c1C] | I*³⁷ | 29% | 559.35 [M − H + Formic acid]⁻ | 559.21 | 2.54 | D |
| 244 | 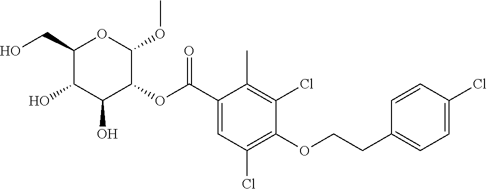 [SMILES: CO[C@H]1O[C@H](CO)[C@@H](O)[C@H](O)[C@H]1OC(=O)c1cc(Cl)c(OCCc2ccc(Cl)cc2)c(Cl)c1C] | I*³⁷ | 42% | 579.01 [M − H + Formic acid]⁻ | 579.15 | 2.55 | D |
| 245 | 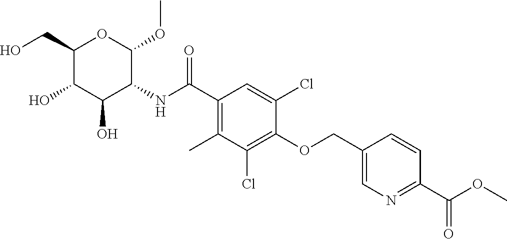 [SMILES: COC(=O)c1ccc(COc2c(Cl)cc(C(=O)N[C@H]3[C@@H](OC)O[C@H](CO)[C@@H](O)[C@@H]3O)c(C)c2Cl)cn1] | R | 29% | 544.97 [M + H]⁺ | 545.11 | 1.50 | D |
| 246 | 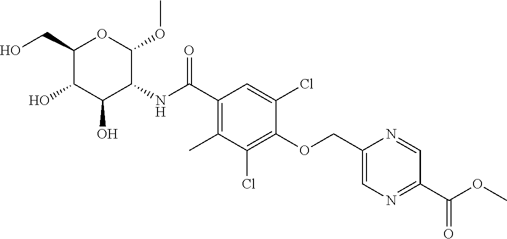 [SMILES: COC(=O)c1cnc(COc2c(Cl)cc(C(=O)N[C@H]3[C@@H](OC)O[C@H](CO)[C@@H](O)[C@@H]3O)c(C)c2Cl)cn1] | R | 54% | 545.96 [M + H]⁺ | 546.11 | 1.45 | D |
| 247 | 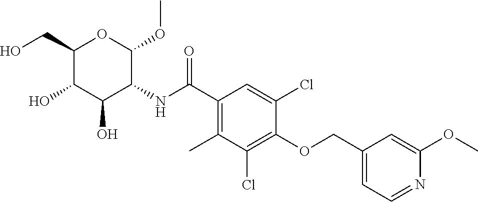 [SMILES: COc1cc(COc2c(Cl)cc(C(=O)N[C@H]3[C@@H](OC)O[C@H](CO)[C@@H](O)[C@@H]3O)c(C)c2Cl)ccn1] | R | 49% | 517.01 [M + H]⁺ | 517.12 | 1.24 | D |
| 248 | 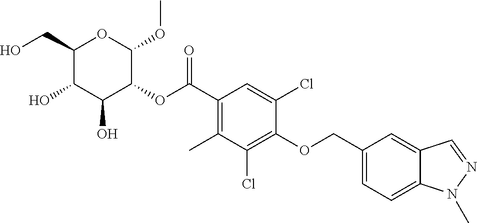 [SMILES: CO[C@H]1O[C@H](CO)[C@@H](O)[C@H](O)[C@H]1OC(=O)c1cc(Cl)c(OCc2ccc3c(cnn3C)c2)c(Cl)c1C] | R | 41% | 540.98 [M + H]⁺ | 541.12 | 1.99 | D |
| 249 | 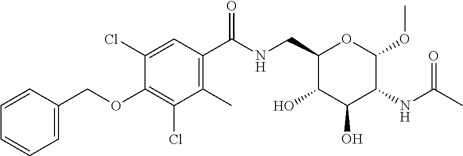 [SMILES: CO[C@H]1O[C@H](CNC(=O)c2cc(Cl)c(OCc3ccccc3)c(Cl)c2C)[C@@H](O)[C@H](O)[C@H]1NC(C)=O] | L | 9% | 527.01 [M + H]⁺ | 527.35 | 2.00 | D |
| 250 | 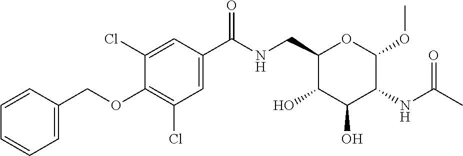 [SMILES: CO[C@H]1O[C@H](CNC(=O)c2cc(Cl)c(OCc3ccccc3)c(Cl)c2)[C@@H](O)[C@H](O)[C@H]1NC(C)=O] | L | 9% | 513.00 [M + H]⁺ | 513.12 | 1.99 | D |
| 251 | 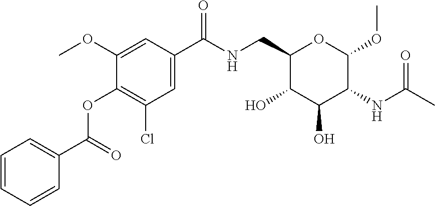 [SMILES: COc1cc(C(=O)NC[C@H]2O[C@H](OC)[C@H](NC(C)=O)[C@@H](O)[C@@H]2O)cc(Cl)c1OC(=O)c1ccccc1] | L | 18% | 523.02 [M + H]⁺ | 523.15 | 1.86 | D |
| 252 | 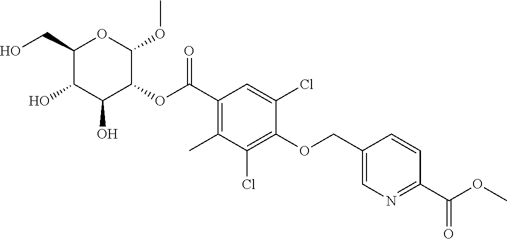 [SMILES: COC(=O)c1ccc(COc2c(Cl)cc(C(=O)O[C@H]3[C@@H](OC)O[C@H](CO)[C@@H](O)[C@@H]3O)c(C)c2Cl)cn1] | R | 17% | 590.06 [M − H + Formic acid]⁻ | 590.08 | 1.63 | D |
| 253 | 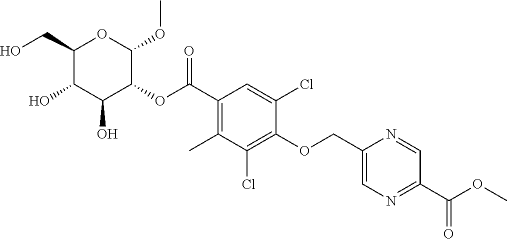 [SMILES: COC(=O)c1cnc(COc2c(Cl)cc(C(=O)O[C@H]3[C@@H](OC)O[C@H](CO)[C@@H](O)[C@@H]3O)c(C)c2Cl)cn1] | R | 17% | 591.04 [M − H + Formic acid]⁻ | 591.08 | 1.59 | D |
| 254 | 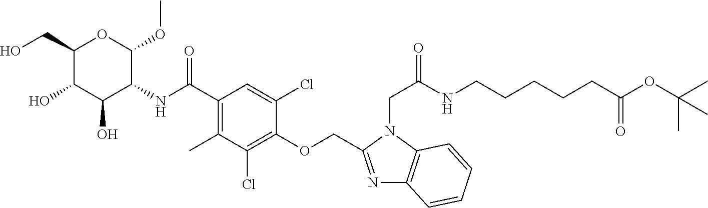 [SMILES: CO[C@H]1O[C@H](CO)[C@@H](O)[C@H](O)[C@H]1NC(=O)c1cc(Cl)c(OCc2nc3ccccc3n2CC(=O)NCCCCCC(=O)OC(C)(C)C)c(Cl)c1C] | L | 31% | 753.16 [M + H]⁺ | 753.27 | 1.96 | D |
| 255 | 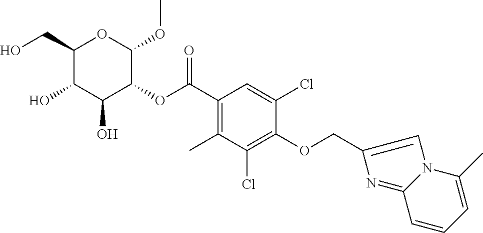 [SMILES: CO[C@H]1O[C@H](CO)[C@@H](O)[C@H](O)[C@H]1OC(=O)c1cc(Cl)c(OCc2cn3c(C)cccc3n2)c(Cl)c1C] | R | 38% | 540.98 [M + H]⁺ | 541.12 | 1.24 | D |
| 256 | 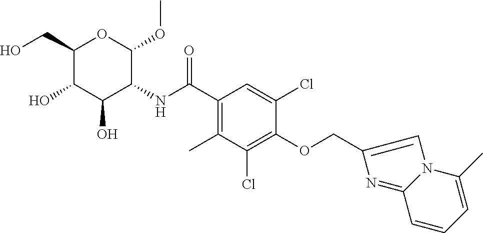 [SMILES: CO[C@H]1O[C@H](CO)[C@@H](O)[C@H](O)[C@H]1NC(=O)c1cc(Cl)c(OCc2cn3c(C)cccc3n2)c(Cl)c1C] | R | 60% | 539.98 [M + H]⁺ | 540.13 | 1.03 | D |
| 257 | 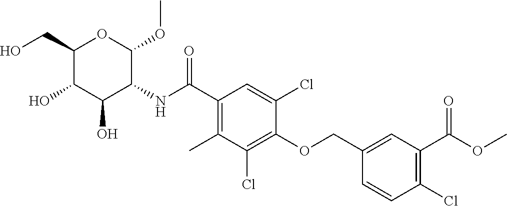 [SMILES: COC(=O)c1cc(COc2c(Cl)cc(C(=O)N[C@H]3[C@@H](OC)O[C@H](CO)[C@@H](O)[C@@H]3O)c(C)c2Cl)ccc1Cl] | R | 48% | 577.91 [M + H]⁺ | 578.08 | 2.04 | D |
| 258 | 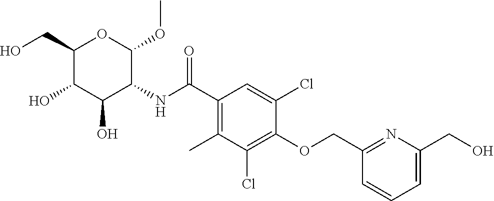 [SMILES: CO[C@H]1O[C@H](CO)[C@@H](O)[C@H](O)[C@H]1NC(=O)c1cc(Cl)c(OCc2cccc(CO)n2)c(Cl)c1C] | R | 54% | 517.01 [M + H]⁺ | 517.12 | 1.27 | D |
| 259 | 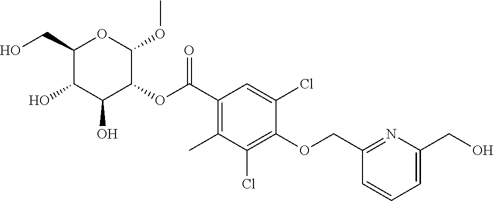 [SMILES: CO[C@H]1O[C@H](CO)[C@@H](O)[C@H](O)[C@H]1OC(=O)c1cc(Cl)c(OCc2cccc(CO)n2)c(Cl)c1C] | R | 14% | 517.97 [M + H]⁺ | 518.10 | 1.54 | D |
| 260 | 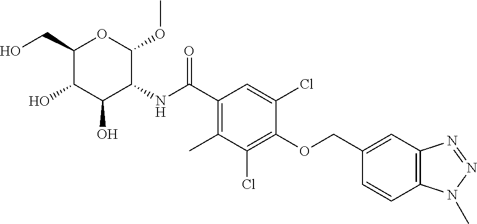 [SMILES: CO[C@H]1O[C@H](CO)[C@@H](O)[C@H](O)[C@H]1NC(=O)c1cc(Cl)c(OCc2ccc3c(c2)nnn3C)c(Cl)c1C] | R | 31% | 540.97 [M + H]⁺ | 541.13 | 1.55 | D |
| 261 | 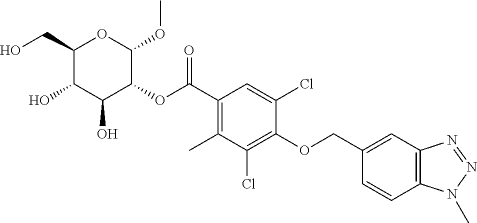 [SMILES: CO[C@H]1O[C@H](CO)[C@@H](O)[C@H](O)[C@H]1OC(=O)c1cc(Cl)c(OCc2ccc3c(c2)nnn3C)c(Cl)c1C] | R | 41% | 586.04 [M − H + Formic acid]⁻ | 586.10 | 1.80 | D |
| 262 | 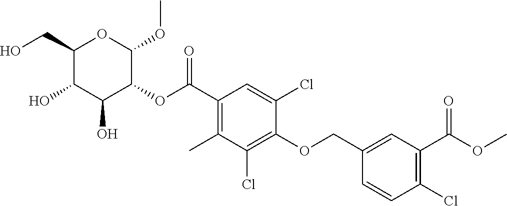 [SMILES: COC(=O)c1cc(COc2c(Cl)cc(C(=O)O[C@H]3[C@@H](OC)O[C@H](CO)[C@@H](O)[C@@H]3O)c(C)c2Cl)ccc1Cl] | R | 48% | 623.06 [M − H + Formic acid]⁻ | 623.05 | 2.29 | D |
| 263 | 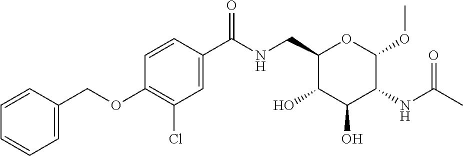 [SMILES: CO[C@H]1O[C@H](CNC(=O)c2ccc(OCc3ccccc3)c(Cl)c2)[C@@H](O)[C@H](O)[C@H]1NC(C)=O] | L | 61% | 479.08 [M + H]⁺ | 479.16 | 1.81 | D |
| 264 | 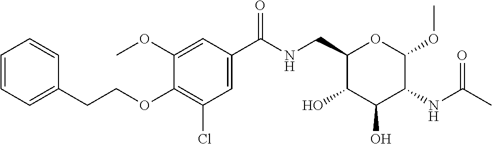 [SMILES: COc1cc(C(=O)NC[C@H]2O[C@H](OC)[C@H](NC(C)=O)[C@@H](O)[C@@H]2O)cc(Cl)c1OCCc1ccccc1] | L | 5% | 523.1 [M + H]⁺ | 523.19 | 1.99 | D |
| 265 | 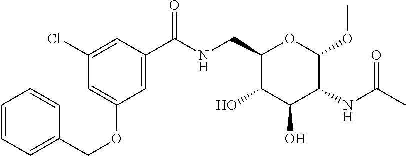 [SMILES: CO[C@H]1O[C@H](CNC(=O)c2cc(Cl)cc(OCc3ccccc3)c2)[C@@H](O)[C@H](O)[C@H]1NC(C)=O] | L | 39% | 479.08 [M + H]⁺ | 479.16 | 1.90 | D |
| 266 | 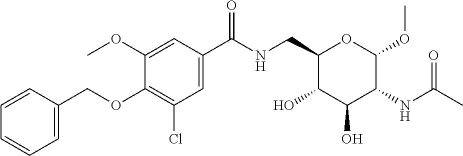 [SMILES: COc1cc(C(=O)NC[C@H]2O[C@H](OC)[C@H](NC(C)=O)[C@@H](O)[C@@H]2O)cc(Cl)c1OCc1ccccc1] | L | 59% | 509.06 [M + H]⁺ | 509.17 | 1.87 | D |
| 267 | 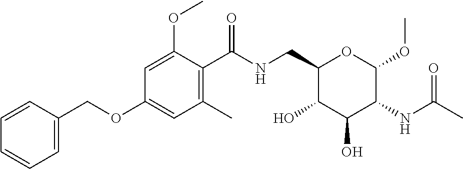 [SMILES: COc1cc(OCc2ccccc2)cc(C)c1C(=O)NC[C@H]1O[C@H](OC)[C@H](NC(C)=O)[C@@H](O)[C@@H]1O] | L | 69% | 489.14 [M + H]⁺ | 489.22 | 1.71 | D |
| 268 | 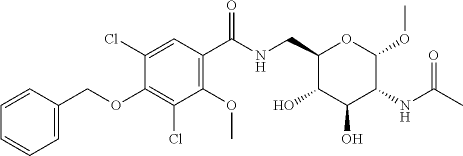 [SMILES: COc1c(C(=O)NC[C@H]2O[C@H](OC)[C@H](NC(C)=O)[C@@H](O)[C@@H]2O)cc(Cl)c(OCc2ccccc2)c1Cl] | L | 63% | 543.01 [M + H]⁺ | 543.13 | 2.07 | D |
| 269 | 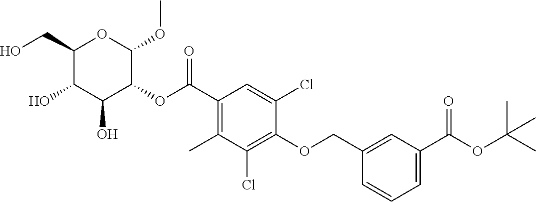 [SMILES: CO[C@H]1O[C@H](CO)[C@@H](O)[C@H](O)[C@H]1OC(=O)c1cc(Cl)c(OCc2cccc(C(=O)OC(C)(C)C)c2)c(Cl)c1C] | R | 34% | 631.07 [M − H + Formic acid]⁻ | 631.13 | 2.60 | D |
| 270 | 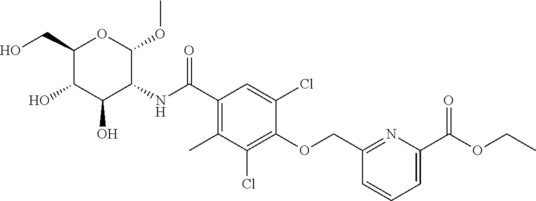 [SMILES: CCOC(=O)c1cccc(COc2c(Cl)cc(C(=O)N[C@H]3[C@@H](OC)O[C@H](CO)[C@@H](O)[C@@H]3O)c(C)c2Cl)n1] | R | 51% | 559.01 [M + H]⁺ | 559.13 | 1.76 | D |
| 271 | 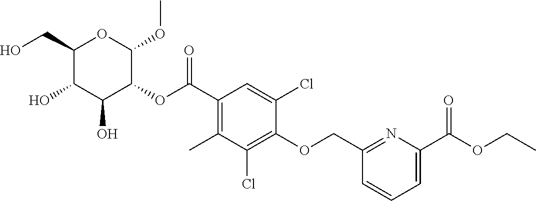 [SMILES: CCOC(=O)c1cccc(COc2c(Cl)cc(C(=O)O[C@H]3[C@@H](OC)O[C@H](CO)[C@@H](O)[C@@H]3O)c(C)c2Cl)n1] | R | 31% | 604.04 [M − H + Formic acid]⁻ | 604.10 | 2.02 | D |
| 272 | 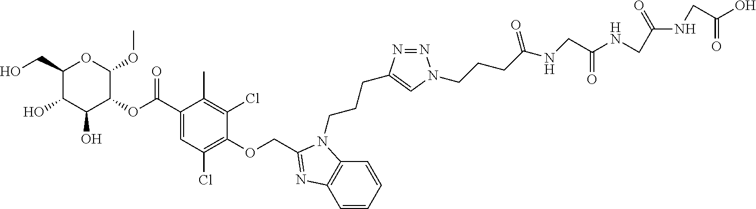 [SMILES: CO[C@H]1O[C@H](CO)[C@@H](O)[C@H](O)[C@H]1OC(=O)c1cc(Cl)c(OCc2nc3ccccc3n2CCCc2cn(CCCC(=O)NCC(=O)NCC(=O)NCC(=O)O)nn2)c(Cl)c1C] | P*³⁴ | 65% | 695.16 [M − H]⁻ | 695.19 | 1.37 | D |
| 273 | 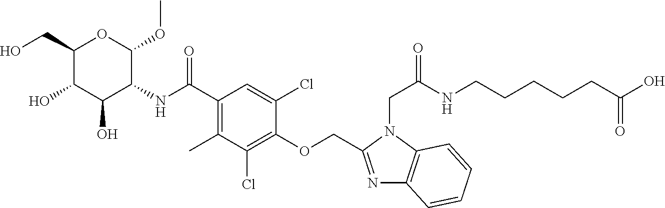 [SMILES: CO[C@H]1O[C@H](CO)[C@@H](O)[C@H](O)[C@H]1NC(=O)c1cc(Cl)c(OCc2nc3ccccc3n2CC(=O)NCCCCCC(=O)O)c(Cl)c1C] | L*⁹ | 15% | 695.16 [M − H]⁻ | 695.19 | 1.37 | D |
| 274 | 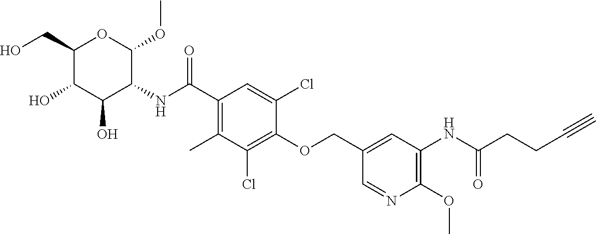 [SMILES: C#CCCC(=O)Nc1cc(COc2c(Cl)cc(C(=O)N[C@H]3[C@@H](OC)O[C@H](CO)[C@@H](O)[C@@H]3O)c(C)c2Cl)cnc1OC] | L | 55% | 611.93 [M + H]⁺ | 612.15 | 1.72 | D |
| 275 | 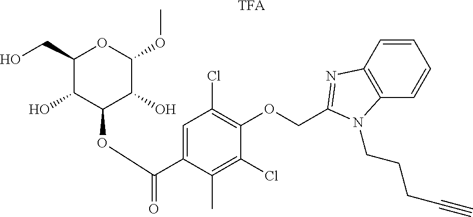 [SMILES: C#CCCCn1c(COc2c(Cl)cc(C(=O)O[C@@H]3[C@@H](O)[C@@H](OC)O[C@H](CO)[C@H]3O)c(C)c2Cl)nc2ccccc21 ; "TFA"] | K*²³*¹⁹ | 2% | 637.09 [M − H + formic acid]⁻ | 637.23 | 1.97 | D |
| 276 | 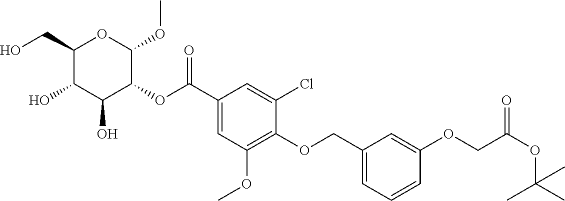 [SMILES: COc1cc(C(=O)O[C@H]2[C@@H](OC)O[C@H](CO)[C@@H](O)[C@@H]2O)cc(Cl)c1OCc1cccc(OCC(=O)OC(C)(C)C)c1] | I*¹⁹ | 16% | 643.13 [M − H + formic acid]⁻ | 643.27 | 2.28 | D |
| 277 | 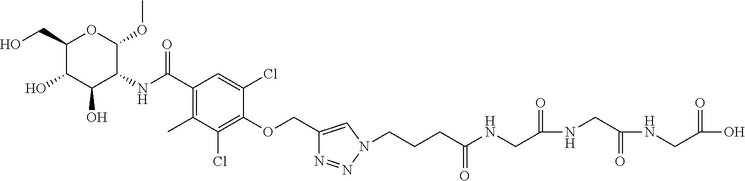 [SMILES: CO[C@H]1O[C@H](CO)[C@@H](O)[C@H](O)[C@H]1NC(=O)c1cc(Cl)c(OCc2cn(CCCC(=O)NCC(=O)NCC(=O)NCC(=O)O)nn2)c(Cl)c1C] | P*³⁴ | 61% | 734.1 parent peak | 735.55 | 2.67 | F |
| 278 | 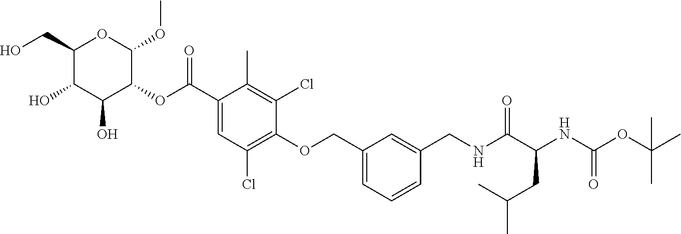 [SMILES: CO[C@H]1O[C@H](CO)[C@@H](O)[C@H](O)[C@H]1OC(=O)c1cc(Cl)c(OCc2cccc(CNC(=O)[C@H](CC(C)C)NC(=O)OC(C)(C)C)c2)c(Cl)c1C] | I*¹⁶ + L | 30% | 729.08 [M + H]⁺ | 729.26 | 2.36 | D |
| 279 | 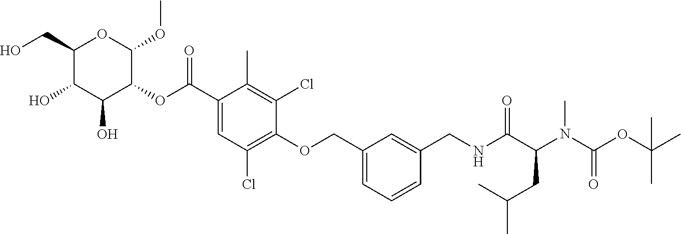 [SMILES: CO[C@H]1O[C@H](CO)[C@@H](O)[C@H](O)[C@H]1OC(=O)c1cc(Cl)c(OCc2cccc(CNC(=O)[C@H](CC(C)C)N(C)C(=O)OC(C)(C)C)c2)c(Cl)c1C] | I*¹⁶ + L | 32% | 743.13 [M + H]⁺ | 743.27 | 2.57 | D |
| 280 | 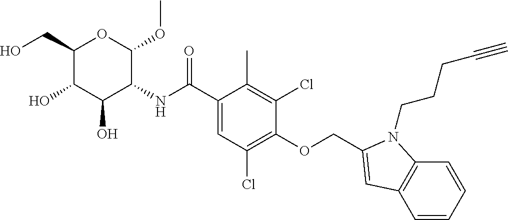 [SMILES: C#CCCCn1c(COc2c(Cl)cc(C(=O)N[C@H]3[C@@H](OC)O[C@H](CO)[C@@H](O)[C@@H]3O)c(C)c2Cl)cc2ccccc21] | L | 19% | 589.07 [M − H]⁻ | 589.15 | 2.31 | D |
| 281 | 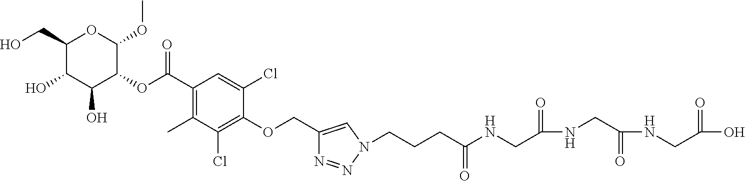 [SMILES: CO[C@H]1O[C@H](CO)[C@@H](O)[C@H](O)[C@H]1OC(=O)c1cc(Cl)c(OCc2cn(CCCC(=O)NCC(=O)NCC(=O)NCC(=O)O)nn2)c(Cl)c1C] | P*³⁴ | 41% | 735.1 parent peak | 735.18 | 2.80 | F |
| 282 | 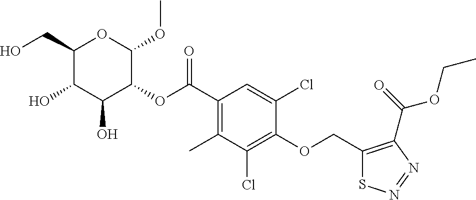 [SMILES: CCOC(=O)c1nnsc1COc1c(Cl)cc(C(=O)O[C@H]2[C@@H](OC)O[C@H](CO)[C@@H](O)[C@@H]2O)c(C)c1Cl] | R | 28% | 610.97 [M − H + Formic acid]⁻ | 611.05 | 2.02 | D |
| 283 | 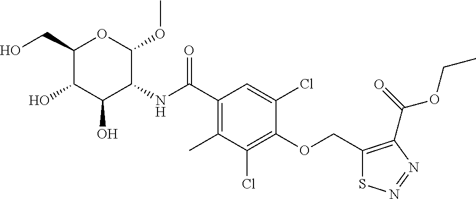 [SMILES: CCOC(=O)c1nnsc1COc1c(Cl)cc(C(=O)N[C@H]2[C@@H](OC)O[C@H](CO)[C@@H](O)[C@@H]2O)c(C)c1Cl] | R | 24% | 564.0 [M − H]⁻ | 564.06 | 1.75 | D |
| 284 | 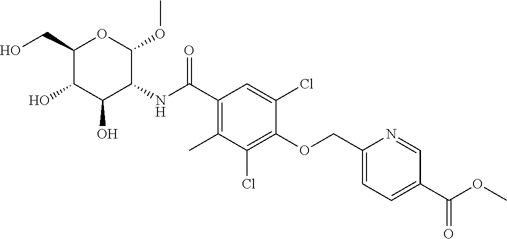 [SMILES: COC(=O)c1ccc(COc2c(Cl)cc(C(=O)N[C@H]3[C@@H](OC)O[C@H](CO)[C@@H](O)[C@@H]3O)c(C)c2Cl)nc1] | R | 55% | 542.95 [M − H]⁻ | 543.09 | 1.65 | D |
| 285 | 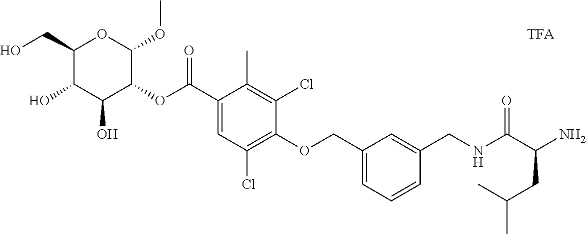 [SMILES: CO[C@H]1O[C@H](CO)[C@@H](O)[C@H](O)[C@H]1OC(=O)c1cc(Cl)c(OCc2cccc(CNC(=O)[C@@H](N)CC(C)C)c2)c(Cl)c1C ; "TFA"] | I*¹⁶ + L*¹⁰ | 18% | 627.02 [M − H]⁻ | 627.19 | 1.52 | D |
| 286 | 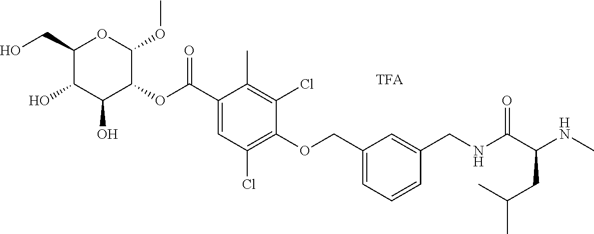 [SMILES: CN[C@@H](CC(C)C)C(=O)NCc1cccc(COc2c(Cl)cc(C(=O)O[C@H]3[C@@H](OC)O[C@H](CO)[C@@H](O)[C@@H]3O)c(C)c2Cl)c1 ; "TFA"] | I*¹⁶ + L*¹⁰ | 22% | 687.06 [M − H + Formic acid]⁻ | 687.30 | 1.55 | D |
| 287 | 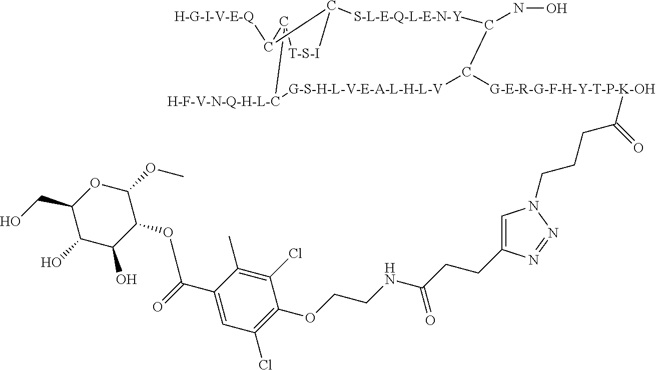 | P | 19% | 1045.3 [M + 6H]⁶⁺ | 1045.65 | λ = 215 nm: 4.04 | F |
| 288 | 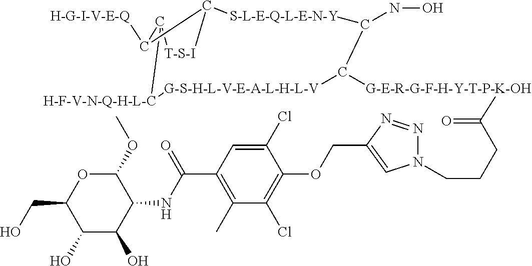 | P | 21% | 1025.6 [M + 6H]⁶⁺ | 1031.30 | λ = 215 nm: 3.90 | F |
| 289 | 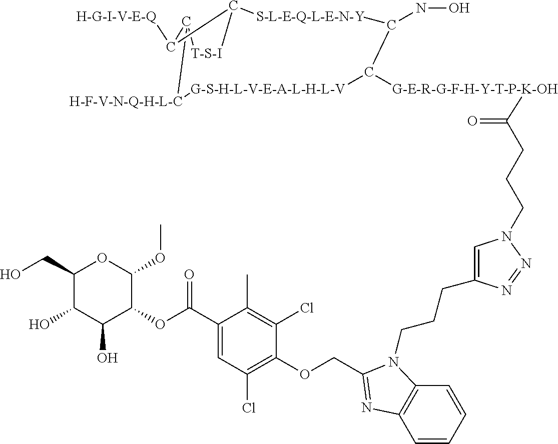 | P | 31% | 1057.5 [M + 6H]⁶⁺ | 1057.83 | λ = 215 nm: 4.37 | F |
| 290 | 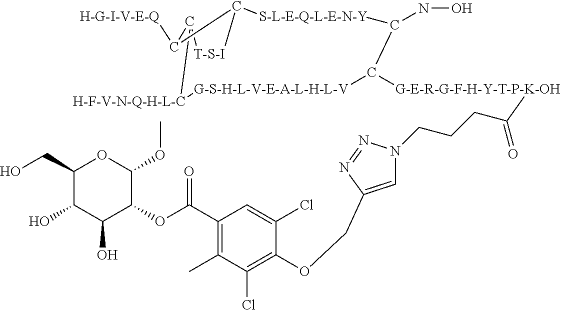 | P | 43% | 1237.2 [M + 5H]⁵⁺ | 1237.56 | λ = 215 nm: 4.14 | F |
| 291 | 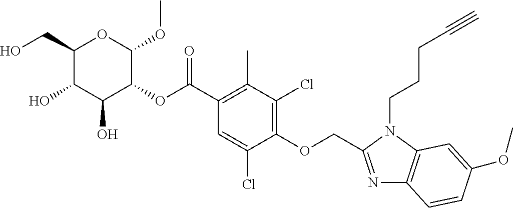 [SMILES: C#CCCCn1c(COc2c(Cl)cc(C(=O)O[C@H]3[C@@H](OC)O[C@H](CO)[C@@H](O)[C@@H]3O)c(C)c2Cl)nc2ccc(OC)cc21] | I*¹⁹ | 63% | 623.00 [M + H]⁺ | 623.15 | 1.88 | D |
| 292 | 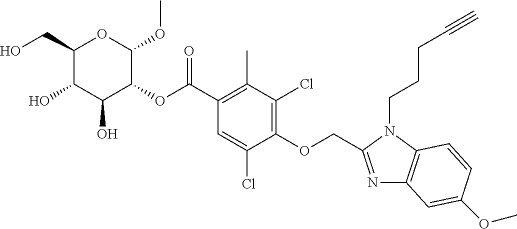 [SMILES: C#CCCCn1c(COc2c(Cl)cc(C(=O)O[C@H]3[C@@H](OC)O[C@H](CO)[C@@H](O)[C@@H]3O)c(C)c2Cl)nc2cc(OC)ccc21] | I*¹⁹ | 81% | 667.10 [M − H + formic acid]⁻ | 667.24 | 1.84 | D |
| 293 | 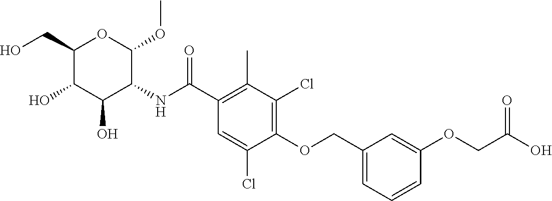 [SMILES: CO[C@H]1O[C@H](CO)[C@@H](O)[C@H](O)[C@H]1NC(=O)c1cc(Cl)c(OCc2cccc(OCC(=O)O)c2)c(Cl)c1C] | R*¹⁹ | 16% | 559.95 [M + H]⁺ | 560.11 | 1.60 | D |
| 294 | 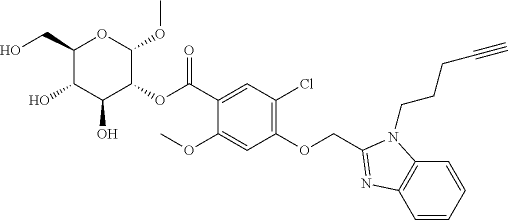 [SMILES: C#CCCCn1c(COc2cc(OC)c(C(=O)O[C@H]3[C@@H](OC)O[C@H](CO)[C@@H](O)[C@@H]3O)cc2Cl)nc2ccccc21] | I*¹⁵ | 69% | 619.17 [M − H + Formic acid]⁻ | 619.26 | 1.74 | D |
| 295 | 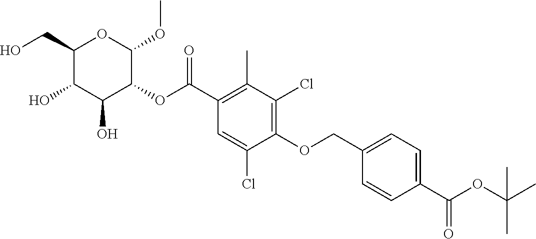 [SMILES: CO[C@H]1O[C@H](CO)[C@@H](O)[C@H](O)[C@H]1OC(=O)c1cc(Cl)c(OCc2ccc(C(=O)OC(C)(C)C)cc2)c(Cl)c1C] | R | 20% | 631.07 [M − H + formic acid]⁻ | 631.23 | 2.64 | D |
| 296 | 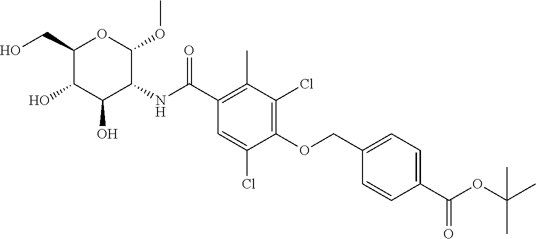 [SMILES: CO[C@H]1O[C@H](CO)[C@@H](O)[C@H](O)[C@H]1NC(=O)c1cc(Cl)c(OCc2ccc(C(=O)OC(C)(C)C)cc2)c(Cl)c1C] | R | 51% | 584.05 [M − H]⁻ | 584.14 | 2.36 | D |
| 297 | 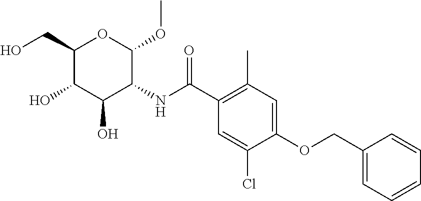 [SMILES: CO[C@H]1O[C@H](CO)[C@@H](O)[C@H](O)[C@H]1NC(=O)c1cc(Cl)c(OCc2ccccc2)cc1C] | L | 59% | 452.02 [M + H]⁺ | 452.14 | 1.80 | D |
| 298 | 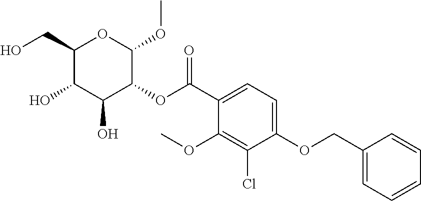 [SMILES: COc1c(C(=O)O[C@H]2[C@@H](OC)O[C@H](CO)[C@@H](O)[C@@H]2O)ccc(OCc2ccccc2)c1Cl] | I*¹⁵ | 90% | 469.00 [M + H]⁺ | 469.13 | 1.97 | D |
| 299 | 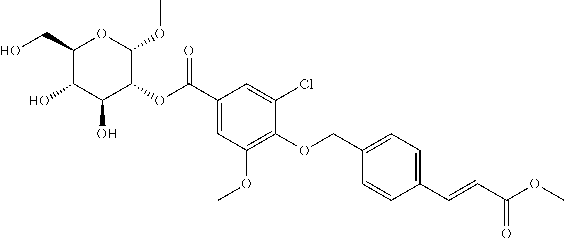 [SMILES: COC(=O)/C=C/c1ccc(COc2c(Cl)cc(C(=O)O[C@H]3[C@@H](OC)O[C@H](CO)[C@@H](O)[C@@H]3O)cc2OC)cc1] | R | 47% | 597.00 [M − H + formic acid]⁻ | 597.23 | 2.10 | D |
| 300 | 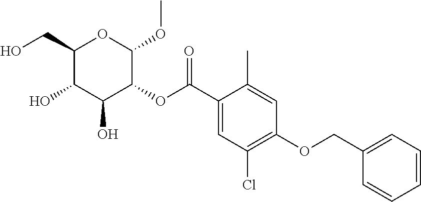 [SMILES: CO[C@H]1O[C@H](CO)[C@@H](O)[C@H](O)[C@H]1OC(=O)c1cc(Cl)c(OCc2ccccc2)cc1C] | I*¹⁵ | 54% | 497.05 [M − H + Formic acid]⁻ | 497.21 | 2.06 | D |
| 301 | 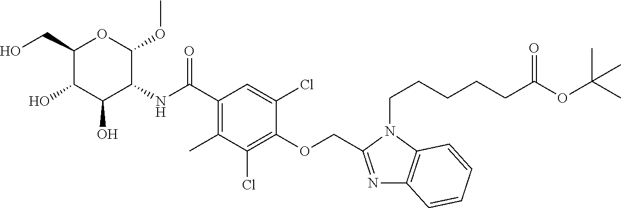 [SMILES: CO[C@H]1O[C@H](CO)[C@@H](O)[C@H](O)[C@H]1NC(=O)c1cc(Cl)c(OCc2nc3ccccc3n2CCCCCC(=O)OC(C)(C)C)c(Cl)c1C] | L | 9% | 696.05 [M + H]⁺ | 696.25 | 2.10 | D |
| 302 | 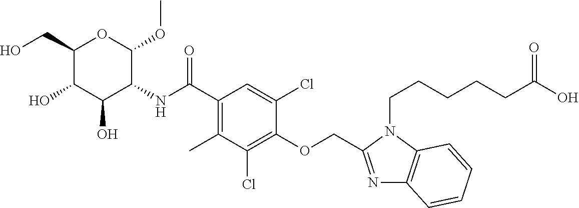 [SMILES: CO[C@H]1O[C@H](CO)[C@@H](O)[C@H](O)[C@H]1NC(=O)c1cc(Cl)c(OCc2nc3ccccc3n2CCCCCC(=O)O)c(Cl)c1C] | L*⁹ | 9% | 640.01 [M + H]⁺ | 640.18 | 1.44 | D |
| 303 | 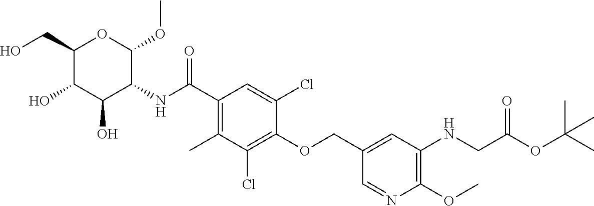 [SMILES: COc1ncc(COc2c(Cl)cc(C(=O)N[C@H]3[C@@H](OC)O[C@H](CO)[C@@H](O)[C@@H]3O)c(C)c2Cl)cc1NCC(=O)OC(C)(C)C] | L | 76% | 646.40 [M + H]⁺ | 646.19 | 2.07 | D |
| 304 | 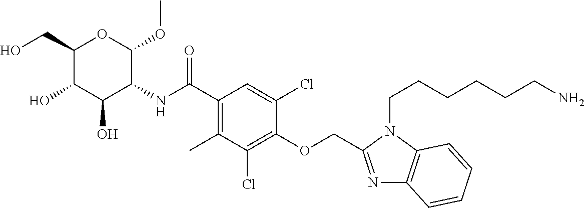 [SMILES: CO[C@H]1O[C@H](CO)[C@@H](O)[C@H](O)[C@H]1NC(=O)c1cc(Cl)c(OCc2nc3ccccc3n2CCCCCCN)c(Cl)c1C] | L*⁹ | 4% | 623.07 [M − H]⁻ | 623.20 | 1.10 | D |
| 305 | 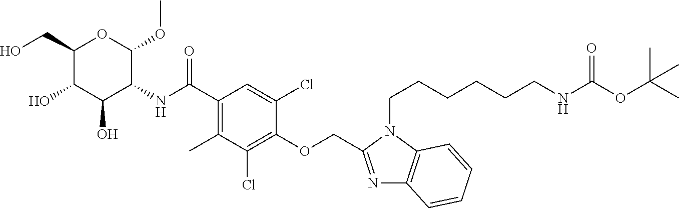 [SMILES: CO[C@H]1O[C@H](CO)[C@@H](O)[C@H](O)[C@H]1NC(=O)c1cc(Cl)c(OCc2nc3ccccc3n2CCCCCCNC(=O)OC(C)(C)C)c(Cl)c1C] | L | 42% | 725.53 [M + H]⁺ | 725.27 | 2.00 | D |
| 306 | 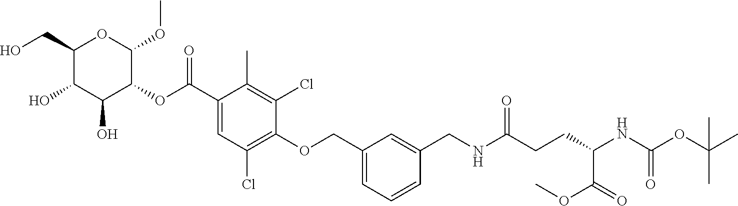 [SMILES: COC(=O)[C@H](CCC(=O)NCc1cccc(COc2c(Cl)cc(C(=O)O[C@H]3[C@@H](OC)O[C@H](CO)[C@@H](O)[C@@H]3O)c(C)c2Cl)c1)NC(=O)OC(C)(C)C] | I*¹⁵ + L | 31% | 803.05 [M − H + Formic acid]⁻ | 803.31 | 2.12 | D |
| 307 | 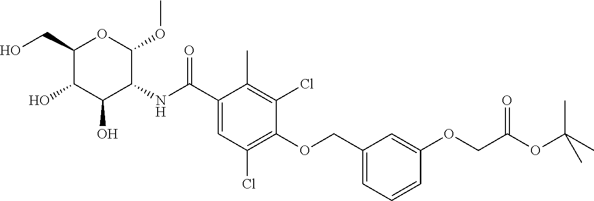 [SMILES: CO[C@H]1O[C@H](CO)[C@@H](O)[C@H](O)[C@H]1NC(=O)c1cc(Cl)c(OCc2cccc(OCC(=O)OC(C)(C)C)c2)c(Cl)c1C] | L | 41% | 659.98 [M − H + formic acid]⁻ | 660.25 | 2.23 | D |
| 308 | 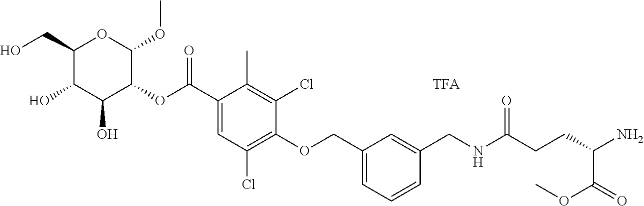 [SMILES: COC(=O)[C@@H](N)CCC(=O)NCc1cccc(COc2c(Cl)cc(C(=O)O[C@H]3[C@@H](OC)O[C@H](CO)[C@@H](O)[C@@H]3O)c(C)c2Cl)c1 ; "TFA"] | I*¹⁶ + L*²⁶ | 17% | 703.12 [M − H + Formic acid]⁻ | 703.26 | 1.41 | D |
| 309 | 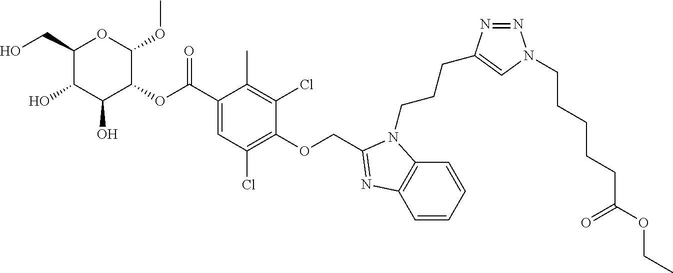 [SMILES: CCOC(=O)CCCCCn1cc(CCCn2c(COc3c(Cl)cc(C(=O)O[C@H]4[C@@H](OC)O[C@H](CO)[C@@H](O)[C@@H]4O)c(C)c3Cl)nc3ccccc32)nn1] | P*³⁰ | 84% | 778.2 parent peak | 779.69 | λ = 215 nm: 6.60 | F |
| 310 | 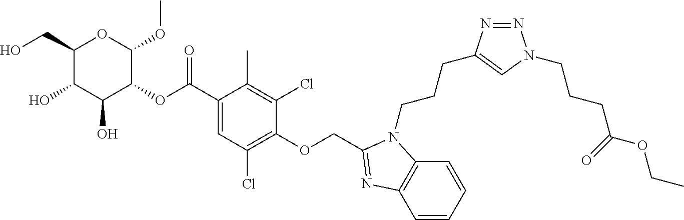 [SMILES: CCOC(=O)CCCn1cc(CCCn2c(COc3c(Cl)cc(C(=O)O[C@H]4[C@@H](OC)O[C@H](CO)[C@@H](O)[C@@H]4O)c(C)c3Cl)nc3ccccc32)nn1] | P*³⁰ | 54% | 752.0 parent peak | 751.63 | λ = 215 nm: 5.70 | F |
| 311 | 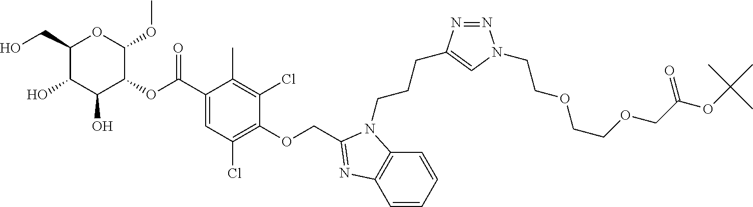 [SMILES: CO[C@H]1O[C@H](CO)[C@@H](O)[C@H](O)[C@H]1OC(=O)c1cc(Cl)c(OCc2nc3ccccc3n2CCCc2cn(CCOCCOCC(=O)OC(C)(C)C)nn2)c(Cl)c1C] | P*³⁰ | 45% | 838.2 parent peak | 839.74 | λ = 215 nm: 6.65 | F |
| 312 | 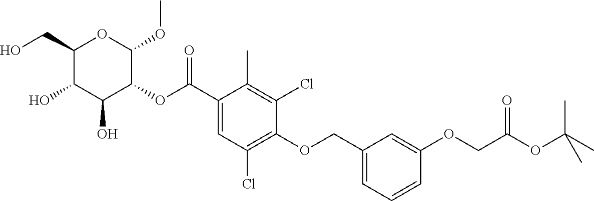 [SMILES: CO[C@H]1O[C@H](CO)[C@@H](O)[C@H](O)[C@H]1OC(=O)c1cc(Cl)c(OCc2cccc(OCC(=O)OC(C)(C)C)c2)c(Cl)c1C] | I*¹⁹ | 43% | 661.05 [M − H + formic acid]⁻ | 661.24 | 2.46 | D |
| 313 | 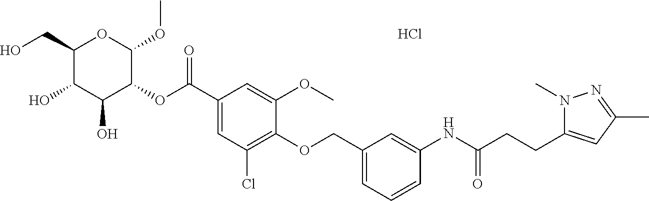 [SMILES: COc1cc(C(=O)O[C@H]2[C@@H](OC)O[C@H](CO)[C@@H](O)[C@@H]2O)cc(Cl)c1OCc1cccc(NC(=O)CCc2cc(C)nn2C)c1 ; Cl] | I*¹⁹ | 82% | 634.01 [M + H]⁺ | 634.22 | 1.72 | D |
| 314 | 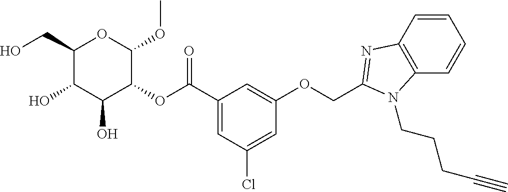 [SMILES: C#CCCCn1c(COc2cc(Cl)cc(C(=O)O[C@H]3[C@@H](OC)O[C@H](CO)[C@@H](O)[C@@H]3O)c2)nc2ccccc21] | I*¹⁵ | 9% | 545.35 [M + H]⁺ | 545.17 | 1.80 | D |
| 315 | 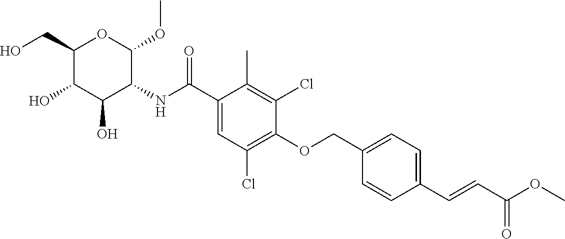 [SMILES: COC(=O)/C=C/c1ccc(COc2c(Cl)cc(C(=O)N[C@H]3[C@@H](OC)O[C@H](CO)[C@@H](O)[C@@H]3O)c(C)c2Cl)cc1] | R | 39% | 570.16 [M + H]⁺ | 570.13 | 2.01 | D |
| 316 | 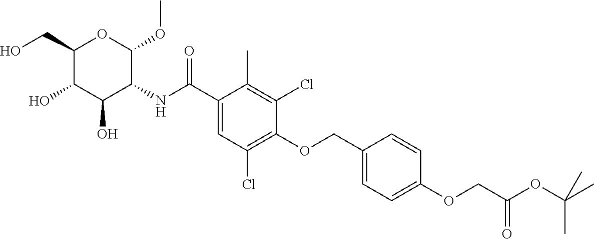 [SMILES: CO[C@H]1O[C@H](CO)[C@@H](O)[C@H](O)[C@H]1NC(=O)c1cc(Cl)c(OCc2ccc(OCC(=O)OC(C)(C)C)cc2)c(Cl)c1C] | L | 59% | 660.36 [M − H + formic acid]⁻ | 660.25 | 2.21 | D |
| 317 | 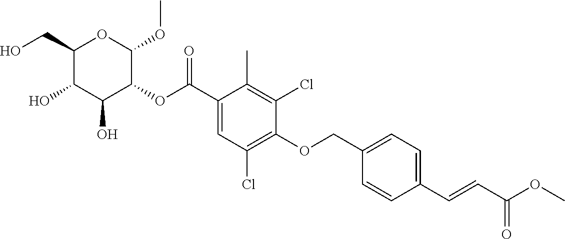 [SMILES: COC(=O)/C=C/c1ccc(COc2c(Cl)cc(C(=O)O[C@H]3[C@@H](OC)O[C@H](CO)[C@@H](O)[C@@H]3O)c(C)c2Cl)cc1] | R | 4% | 571.12 [M + H]⁺ | 571.12 | 2.17 | D |
| 318 | 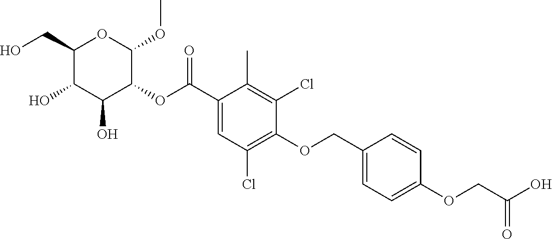 [SMILES: CO[C@H]1O[C@H](CO)[C@@H](O)[C@H](O)[C@H]1OC(=O)c1cc(Cl)c(OCc2ccc(OCC(=O)O)cc2)c(Cl)c1C] | I*¹⁹ | 4% | 578.16 [M + NH4]⁺ | 578.12 | 1.78 | D |
| 319 | 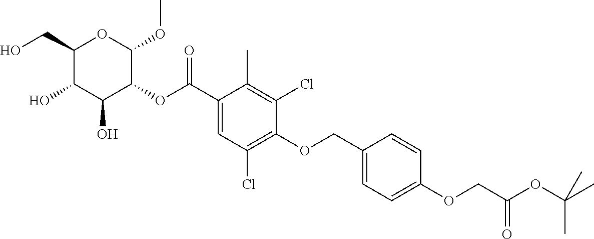 [SMILES: CO[C@H]1O[C@H](CO)[C@@H](O)[C@H](O)[C@H]1OC(=O)c1cc(Cl)c(OCc2ccc(OCC(=O)OC(C)(C)C)cc2)c(Cl)c1C] | I*¹⁹ | 41% | 661.47 [M − H + formic acid]⁻ | 661.24 | 2.45 | D |
| 320 | 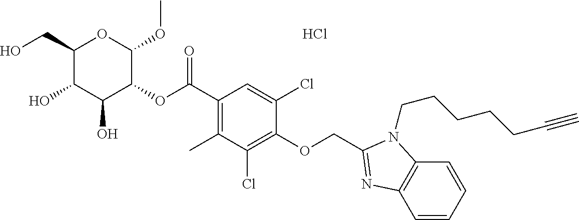 [SMILES: C#CCCCCCn1c(COc2c(Cl)cc(C(=O)O[C@H]3[C@@H](OC)O[C@H](CO)[C@@H](O)[C@@H]3O)c(C)c2Cl)nc2ccccc21 ; Cl] | I*¹⁹ | 57% | 621.31 [M + H]⁺ | 621.18 | 2.08 | D |
| 321 | 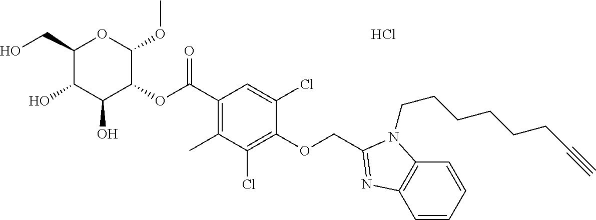 [SMILES: C#CCCCCCCn1c(COc2c(Cl)cc(C(=O)O[C@H]3[C@@H](OC)O[C@H](CO)[C@@H](O)[C@@H]3O)c(C)c2Cl)nc2ccccc21 ; Cl] | I*¹⁹ | 42% | 635.35 [M + H]⁺ | 635.19 | 2.21 | D |
| 322 | 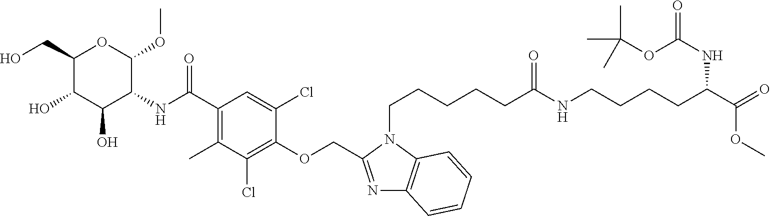 [SMILES: COC(=O)[C@H](CCCCNC(=O)CCCCCn1c(COc2c(Cl)cc(C(=O)N[C@H]3[C@@H](OC)O[C@H](CO)[C@@H](O)[C@@H]3O)c(C)c2Cl)nc2ccccc21)NC(=O)OC(C)(C)C] | L | 20% | 882.56 [M + H]⁺ | 882.35 | 1.82 | D |
| 323 | 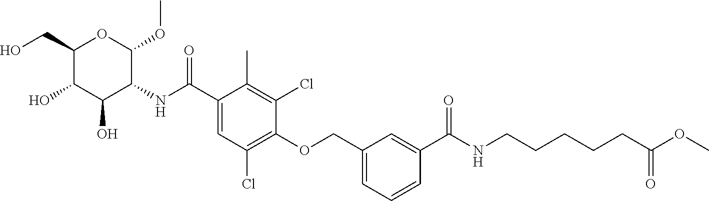 [SMILES: COC(=O)CCCCCNC(=O)c1cccc(COc2c(Cl)cc(C(=O)N[C@H]3[C@@H](OC)O[C@H](CO)[C@@H](O)[C@@H]3O)c(C)c2Cl)c1] | L | 52% | 657.41 [M + H]⁺ | 657.20 | 1.82 | D |
| 324 | 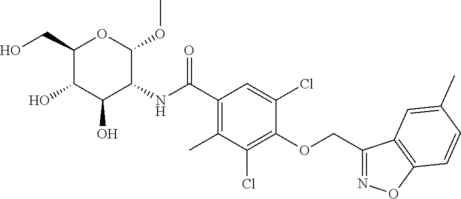 [SMILES: CO[C@H]1O[C@H](CO)[C@@H](O)[C@H](O)[C@H]1NC(=O)c1cc(Cl)c(OCc2noc3ccc(C)cc23)c(Cl)c1C] | R | 37% | 541.23 [M + H]⁺ | 541.12 | 2.03 | D |
| 325 | 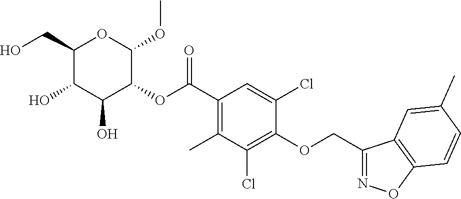 [SMILES: CO[C@H]1O[C@H](CO)[C@@H](O)[C@H](O)[C@H]1OC(=O)c1cc(Cl)c(OCc2noc3ccc(C)cc23)c(Cl)c1C] | R | 31% | 542.2 [M + H]⁺ | 542.10 | 2.28 | D |
| 326 | 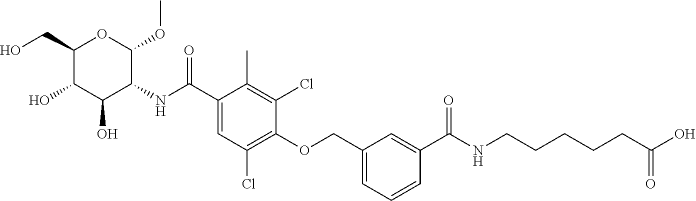 [SMILES: CO[C@H]1O[C@H](CO)[C@@H](O)[C@H](O)[C@H]1NC(=O)c1cc(Cl)c(OCc2cccc(C(=O)NCCCCCC(=O)O)c2)c(Cl)c1C] | L*⁹ | 44% | 643.36 [M + H]⁺ | 643.18 | 1.60 | D |
| 327 | 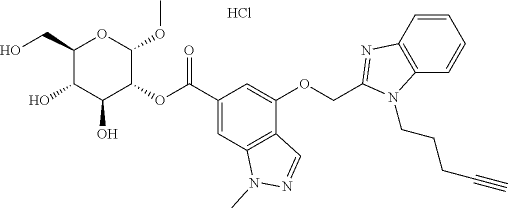 [SMILES: C#CCCCn1c(COc2cc(C(=O)O[C@H]3[C@@H](OC)O[C@H](CO)[C@@H](O)[C@@H]3O)cc3c2cnn3C)nc2ccccc21 ; Cl] | I*¹⁵ | 29% | 609.43 [M − H + Formic acid]⁻ | 609.31 | 1.57 | D |
| 328 | 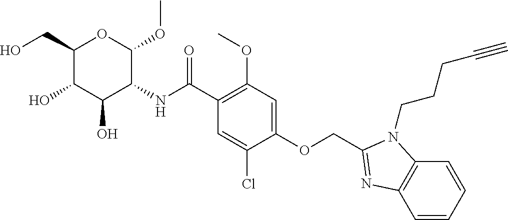 [SMILES: C#CCCCn1c(COc2cc(OC)c(C(=O)N[C@H]3[C@@H](OC)O[C@H](CO)[C@@H](O)[C@@H]3O)cc2Cl)nc2ccccc21] | L | 86% | 574.43 [M + H]⁺ | 574.20 | 1.71 | D |
| 329 | 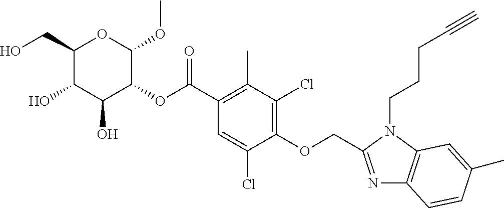 [SMILES: C#CCCCn1c(COc2c(Cl)cc(C(=O)O[C@H]3[C@@H](OC)O[C@H](CO)[C@@H](O)[C@@H]3O)c(C)c2Cl)nc2ccc(C)cc21] | I*¹⁹ | 27% | 607. 26 [M + H]⁺ | 607.16 | 1.97 | D |
| 330 | 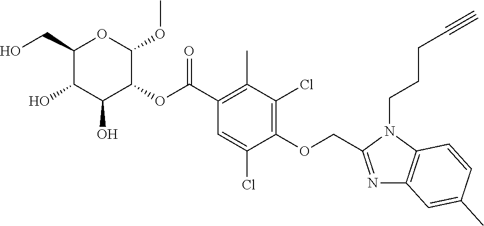 [SMILES: C#CCCCn1c(COc2c(Cl)cc(C(=O)O[C@H]3[C@@H](OC)O[C@H](CO)[C@@H](O)[C@@H]3O)c(C)c2Cl)nc2cc(C)ccc21] | I*¹⁹ | 22% | 607.24 [M + H]⁺ | 607.16 | 1.94 | D |
| 331 | 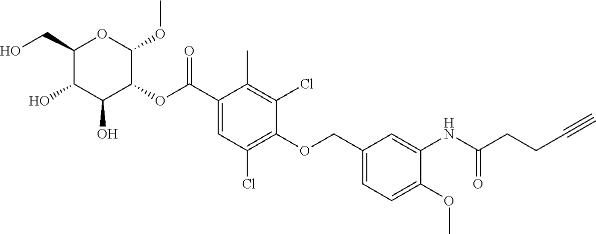 [SMILES: C#CCCC(=O)Nc1cc(COc2c(Cl)cc(C(=O)O[C@H]3[C@@H](OC)O[C@H](CO)[C@@H](O)[C@@H]3O)c(C)c2Cl)ccc1OC] | R | 46% | 612.19 [M + H]⁺ | 612.14 | 2.03 | D |
| 332 | 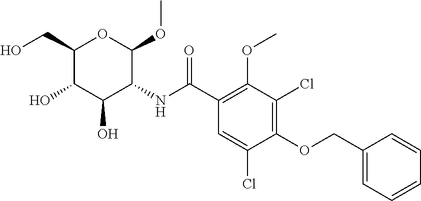 [SMILES: COc1c(C(=O)N[C@H]2[C@H](OC)O[C@H](CO)[C@@H](O)[C@@H]2O)cc(Cl)c(OCc2ccccc2)c1Cl] | L | 29% | 502.1 [M + H]⁺ | 502.10 | 1.23 | B |
| 333 | 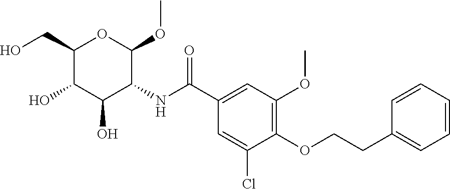 [SMILES: COc1cc(C(=O)N[C@H]2[C@H](OC)O[C@H](CO)[C@@H](O)[C@@H]2O)cc(Cl)c1OCCc1ccccc1] | L | 43% | 482.1 [M + H]⁺ | 482.16 | 1.21 | B |
| 334 | 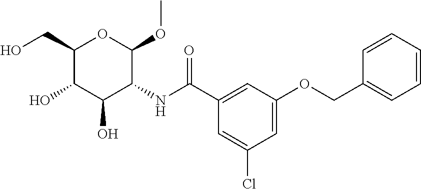 [SMILES: CO[C@@H]1O[C@H](CO)[C@@H](O)[C@H](O)[C@H]1NC(=O)c1cc(Cl)cc(OCc2ccccc2)c1] | L | 47% | 438.1 [M + H]⁺ | 438.13 | 1.16 | B |
| 335 | 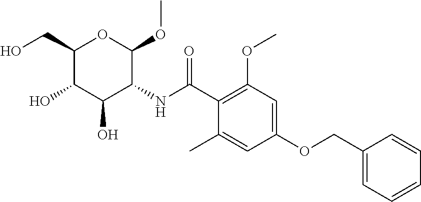 [SMILES: COc1cc(OCc2ccccc2)cc(C)c1C(=O)N[C@H]1[C@H](OC)O[C@H](CO)[C@@H](O)[C@@H]1O] | L | 9% | 448.2 [M + H]⁺ | 448.20 | 1.06 | B |
| 336 | 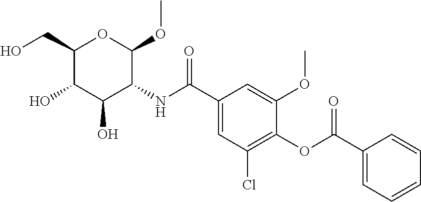 [SMILES: COc1cc(C(=O)N[C@H]2[C@H](OC)O[C@H](CO)[C@@H](O)[C@@H]2O)cc(Cl)c1OC(=O)c1ccccc1] | L | 9% | 482.1 [M + H]⁺ | 482.22 | 1.16 | B |
| 337 | 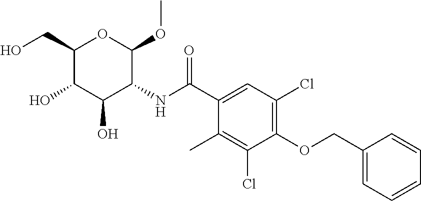 [SMILES: CO[C@@H]1O[C@H](CO)[C@@H](O)[C@H](O)[C@H]1NC(=O)c1cc(Cl)c(OCc2ccccc2)c(Cl)c1C] | L | 9% | 486.1 [M + H]²⁺ | 486.11 | 1.21 | B |
| 338 | 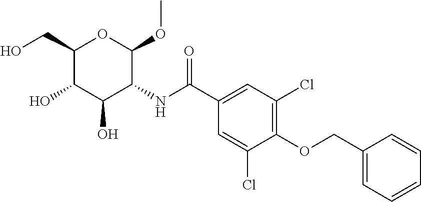 [SMILES: CO[C@@H]1O[C@H](CO)[C@@H](O)[C@H](O)[C@H]1NC(=O)c1cc(Cl)c(OCc2ccccc2)c(Cl)c1] | L | 19% | 472.1 [M + H]⁺ | 472.09 | 1.20 | B |
| 339 | 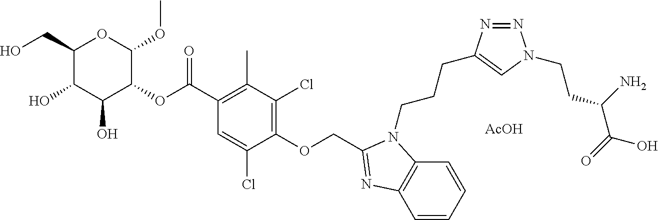 [SMILES: "AcOH" ; CO[C@H]1O[C@H](CO)[C@@H](O)[C@H](O)[C@H]1OC(=O)c1cc(Cl)c(OCc2nc3ccccc3n2CCCc2cn(CC[C@H](N)C(=O)O)nn2)c(Cl)c1C] | P*³⁵ | 33% | 353.2 [M + 2H]⁺ | 399.83 | 1.66 | F |
| 340 | 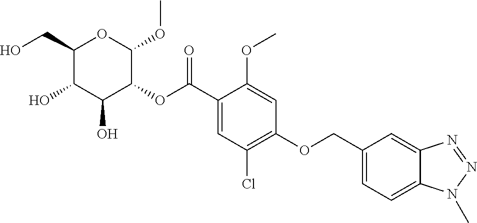 [SMILES: COc1cc(OCc2ccc3c(c2)nnn3C)c(Cl)cc1C(=O)O[C@H]1[C@@H](OC)O[C@H](CO)[C@@H](O)[C@@H]1O] | R | 36% | 524.26 [M + H]⁺ | 524.14 | 1.52 | D |
| 341 | 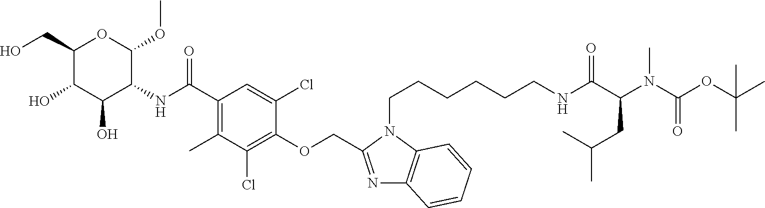 [SMILES: CO[C@H]1O[C@H](CO)[C@@H](O)[C@H](O)[C@H]1NC(=O)c1cc(Cl)c(OCc2nc3ccccc3n2CCCCCCNC(=O)[C@H](CC(C)C)N(C)C(=O)OC(C)(C)C)c(Cl)c1C] | L | 90% | 852.65 [M + H]⁺ | 852.37 | 2.26 | D |
| 342 | 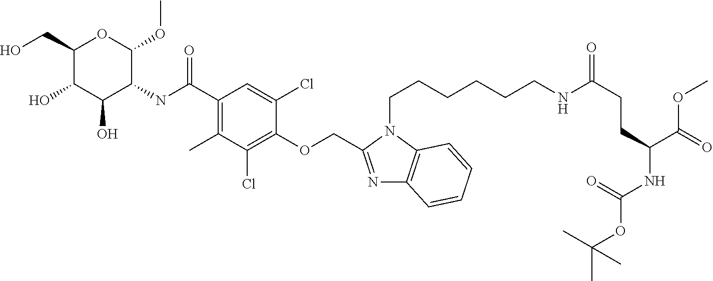 [SMILES: COC(=O)[C@H](CCC(=O)NCCCCCCn1c(COc2c(Cl)cc(C(=O)N[C@H]3[C@@H](OC)O[C@H](CO)[C@@H](O)[C@@H]3O)c(C)c2Cl)nc2ccccc21)NC(=O)OC(C)(C)C] | L | 75% | 868.58 [M + H]⁺ | 868.33 | 1.83 | D |
| 343 | 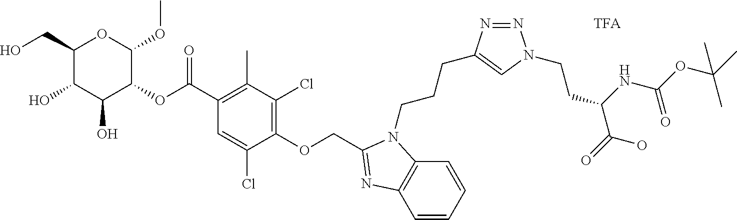 [SMILES: CO[C@H]1O[C@H](CO)[C@@H](O)[C@H](O)[C@H]1OC(=O)c1cc(Cl)c(OCc2nc3ccccc3n2CCCc2cn(CC[C@H](NC(=O)OC(C)(C)C)C(=O)O)nn2)c(Cl)c1C ; "TFA"] | P | 60% | 837.3 parent peak | 878.76 | λ = 215 nm: 5.61 | F |
| 344 | 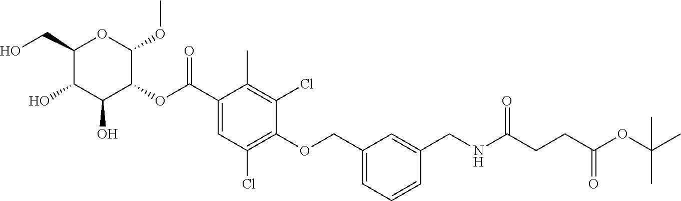 [SMILES: CO[C@H]1O[C@H](CO)[C@@H](O)[C@H](O)[C@H]1OC(=O)c1cc(Cl)c(OCc2cccc(CNC(=O)CCC(=O)OC(C)(C)C)c2)c(Cl)c1C] | I*¹⁵ + L | 33% | 672.43 [M + H]⁺ | 672.20 | 2.15 | D |
| 345 | 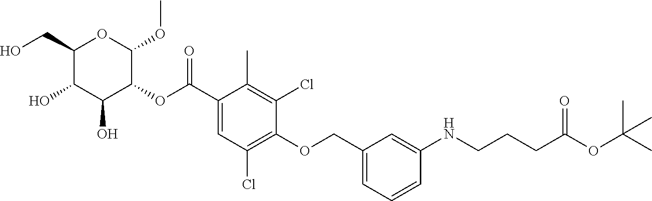 [SMILES: CO[C@H]1O[C@H](CO)[C@@H](O)[C@H](O)[C@H]1OC(=O)c1cc(Cl)c(OCc2cccc(NCCCC(=O)OC(C)(C)C)c2)c(Cl)c1C] | I*¹⁵ + S | 7% | 644.42 [M + H]⁺ | 644.20 | 2.52 | D |
| 346 | 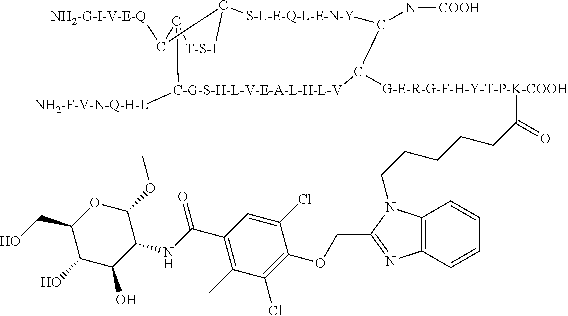 | Q | 28% | 1043.9 [M + 6H]⁶⁺ | 1044.15 | λ = 215 nm: 4.22 | F |
| 347 | 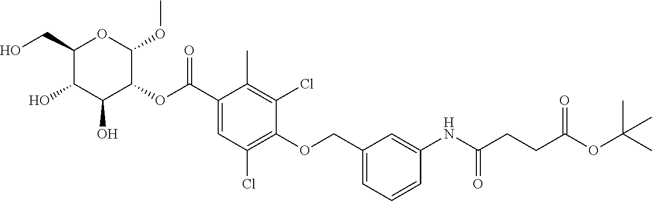 [SMILES: CO[C@H]1O[C@H](CO)[C@@H](O)[C@H](O)[C@H]1OC(=O)c1cc(Cl)c(OCc2cccc(NC(=O)CCC(=O)OC(C)(C)C)c2)c(Cl)c1C] | T | 9% | 702.50 [M − H + Formic acid]⁻ | 702.26 | 2.22 | D |
| 348 | 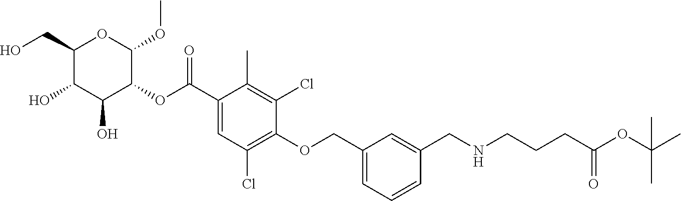 [SMILES: CO[C@H]1O[C@H](CO)[C@@H](O)[C@H](O)[C@H]1OC(=O)c1cc(Cl)c(OCc2cccc(CNCCCC(=O)OC(C)(C)C)c2)c(Cl)c1C] | S | 11% | 658.44 [M + H]⁺ | 658.22 | 1.64 | D |
| 349 | 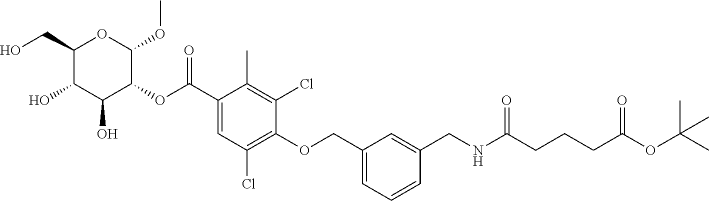 [SMILES: CO[C@H]1O[C@H](CO)[C@@H](O)[C@H](O)[C@H]1OC(=O)c1cc(Cl)c(OCc2cccc(CNC(=O)CCCC(=O)OC(C)(C)C)c2)c(Cl)c1C] | L | 74% | 686.50 [M + H]⁺ | 686.21 | 2.20 | D |
| 350 | 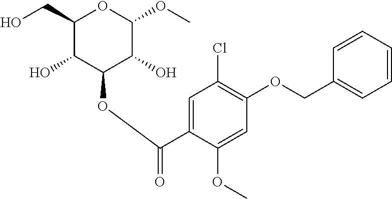 [SMILES: COc1cc(OCc2ccccc2)c(Cl)cc1C(=O)O[C@@H]1[C@@H](O)[C@@H](OC)O[C@H](CO)[C@H]1O] | I*¹⁵ | 1% | 469.23 [M + H]⁺ | 469.13 | 1.80 | D |
| 351 | 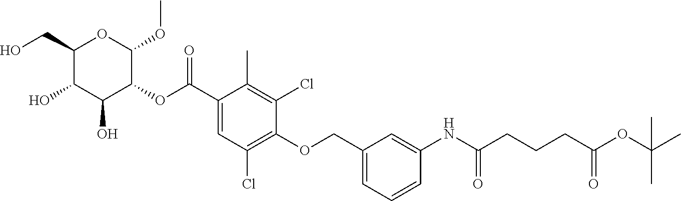 [SMILES: CO[C@H]1O[C@H](CO)[C@@H](O)[C@H](O)[C@H]1OC(=O)c1cc(Cl)c(OCc2cccc(NC(=O)CCCC(=O)OC(C)(C)C)c2)c(Cl)c1C] | T | 48% | 672.45 [M + H]⁺ | 672.20 | 2.29 | D |
| 352 | 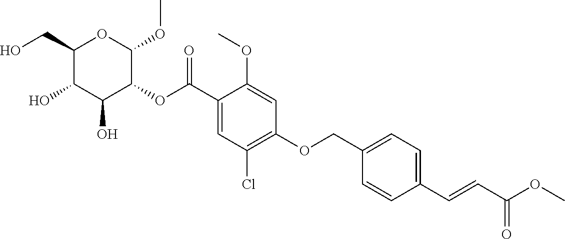 [SMILES: COC(=O)/C=C/c1ccc(COc2cc(OC)c(C(=O)O[C@H]3[C@@H](OC)O[C@H](CO)[C@@H](O)[C@@H]3O)cc2Cl)cc1] | R | 86% | 597.35 [M − H + Formic acid]⁻ | 597.16 | 1.96 | D |
| 353 | 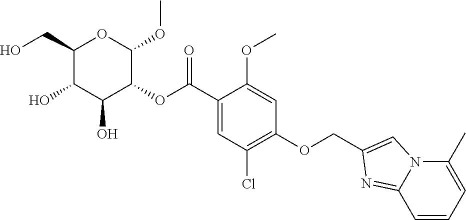 [SMILES: COc1cc(OCc2cn3c(C)cccc3n2)c(Cl)cc1C(=O)O[C@H]1[C@@H](OC)O[C@H](CO)[C@@H](O)[C@@H]1O] | R | 59% | 523.28 [M + H]⁺ | 523.15 | 1.08 | D |
| 354 | 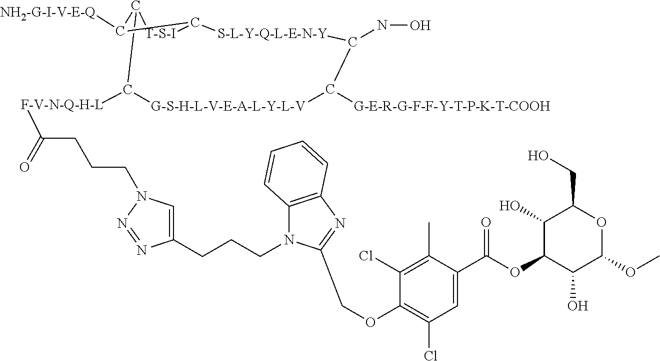 | P*³¹ | 7% | 1086.1 [M + 6H]⁶⁺ | 1086.37 | λ = 215 nm: 5.75 | F |
| 355 | 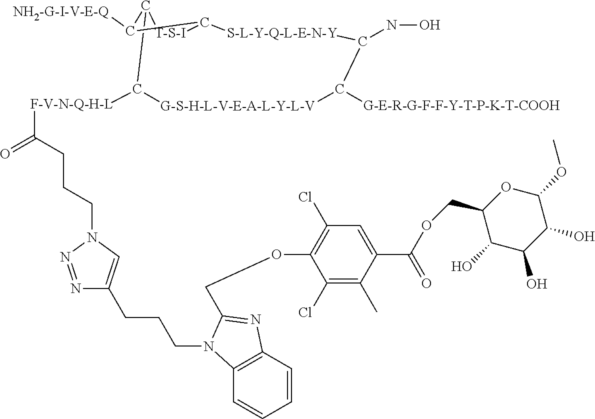 | P*³² | 13% | 1086.1 [M + 6H]⁶⁺ | 1086.37 | λ = 215 nm: 5.86 | F |
| 356 | 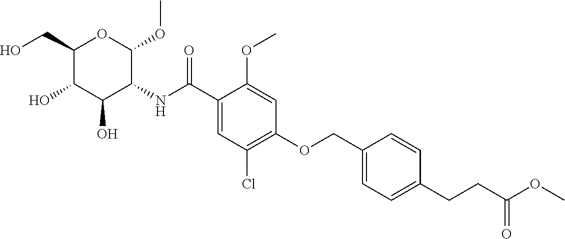 [SMILES: COC(=O)CCc1ccc(COc2cc(OC)c(C(=O)N[C@H]3[C@@H](OC)O[C@H](CO)[C@@H](O)[C@@H]3O)cc2Cl)cc1] | R | 90% | 554.38 [M + H]⁺ | 554.18 | 1.90 | D |
| 357 | 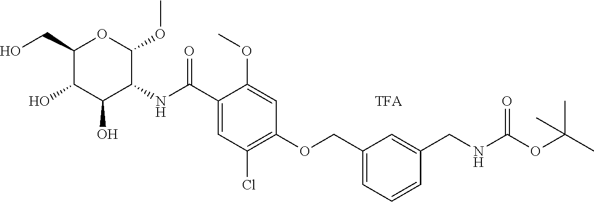 [SMILES: COc1cc(OCc2cccc(CNC(=O)OC(C)(C)C)c2)c(Cl)cc1C(=O)N[C@H]1[C@@H](OC)O[C@H](CO)[C@@H](O)[C@@H]1O ; "TFA"] | L | 85% | 597.44 [M + H]⁺ | 597.22 | 2.00 | D |
| 358 | 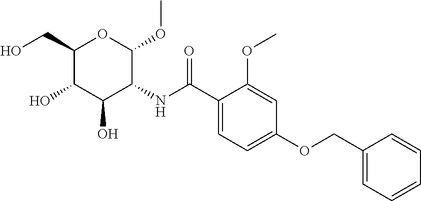 [SMILES: COc1cc(OCc2ccccc2)ccc1C(=O)N[C@H]1[C@@H](OC)O[C@H](CO)[C@@H](O)[C@@H]1O] | R | 74% | 434.39 [M + H]⁺ | 434.18 | 1.73 | D |
| 359 | 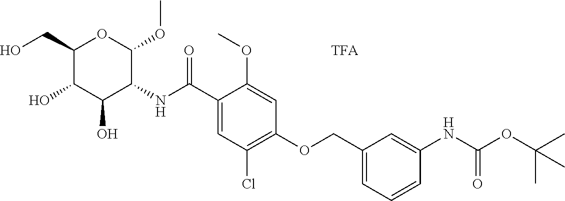 [SMILES: COc1cc(OCc2cccc(NC(=O)OC(C)(C)C)c2)c(Cl)cc1C(=O)N[C@H]1[C@@H](OC)O[C@H](CO)[C@@H](O)[C@@H]1O ; "TFA"] | L | 23% | 583.51 [M + H]⁺ | 583.21 | 2.07 | D |
| 360 | 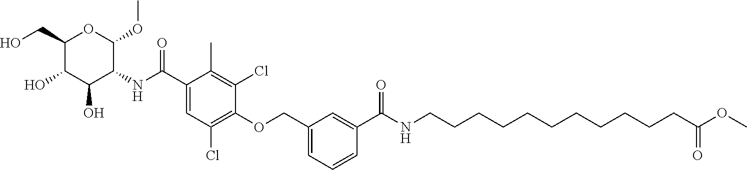 [SMILES: COC(=O)CCCCCCCCCCCNC(=O)c1cccc(COc2c(Cl)cc(C(=O)N[C@H]3[C@@H](OC)O[C@H](CO)[C@@H](O)[C@@H]3O)c(C)c2Cl)c1] | L*⁹ | 29% | 741.75 [M + H]⁺ | 741.29 | 2.51 | D |
| 361 | 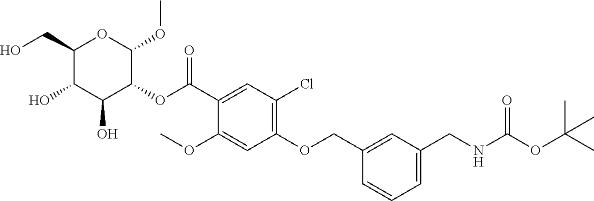 [SMILES: COc1cc(OCc2cccc(CNC(=O)OC(C)(C)C)c2)c(Cl)cc1C(=O)O[C@H]1[C@@H](OC)O[C@H](CO)[C@@H](O)[C@@H]1O] | I | 5% | 642.60 [M − H + Formic acid]⁻ | 642.29 | 2.03 | D |
| 362 | 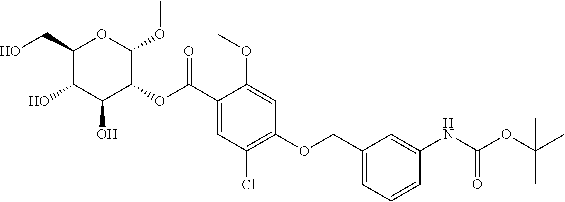 [SMILES: COc1cc(OCc2cccc(NC(=O)OC(C)(C)C)c2)c(Cl)cc1C(=O)O[C@H]1[C@@H](OC)O[C@H](CO)[C@@H](O)[C@@H]1O] | I*²⁷ | 5% | 484.42 [M + H]⁺ | 484.14 | 1.35 | D |
| 363 | 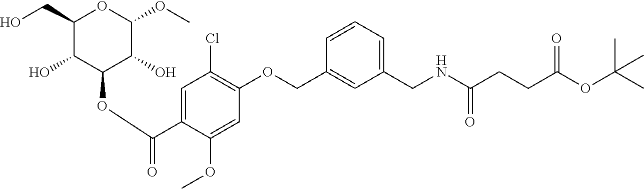 [SMILES: COc1cc(OCc2cccc(CNC(=O)CCC(=O)OC(C)(C)C)c2)c(Cl)cc1C(=O)O[C@@H]1[C@@H](O)[C@@H](OC)O[C@H](CO)[C@H]1O] | I*¹⁶ + L | 1% | 698.72 [M − H + Formic acid]⁻ | 698.31 | 1.84 | D |
| 364 | 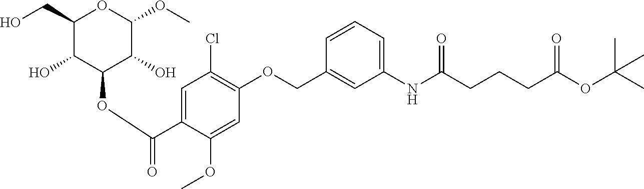 [SMILES: COc1cc(OCc2cccc(NC(=O)CCCC(=O)OC(C)(C)C)c2)c(Cl)cc1C(=O)O[C@@H]1[C@@H](O)[C@@H](OC)O[C@H](CO)[C@H]1O] | I*¹⁶ + L | 2% | 698.70 [M − H + Formic acid]⁻ | 698.31 | 1.99 | D |
| 365 | 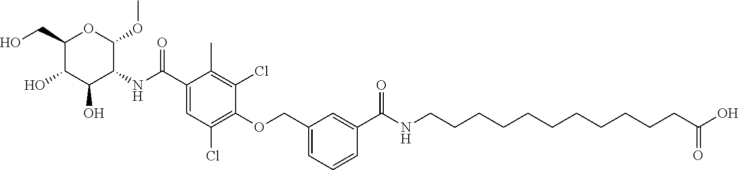 [SMILES: CO[C@H]1O[C@H](CO)[C@@H](O)[C@H](O)[C@H]1NC(=O)c1cc(Cl)c(OCc2cccc(C(=O)NCCCCCCCCCCCC(=O)O)c2)c(Cl)c1C] | L*¹⁴ | 28% | 727.65 [M + H]⁺ | 727.27 | 2.22 | D |
| 366 | 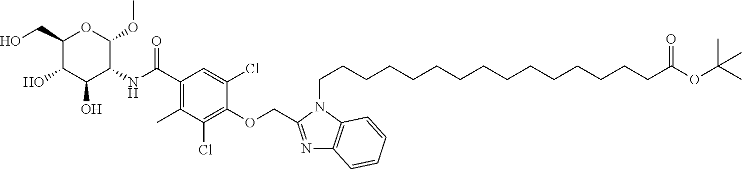 [SMILES: CO[C@H]1O[C@H](CO)[C@@H](O)[C@H](O)[C@H]1NC(=O)c1cc(Cl)c(OCc2nc3ccccc3n2CCCCCCCCCCCCCCCC(=O)OC(C)(C)C)c(Cl)c1C] | L | 76% | 836.61 [M + H]⁺ | 836.40 | 3.32 | D |
| 367 | 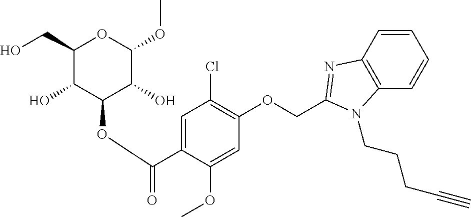 [SMILES: C#CCCCn1c(COc2cc(OC)c(C(=O)O[C@@H]3[C@@H](O)[C@@H](OC)O[C@H](CO)[C@H]3O)cc2Cl)nc2ccccc21] | I*¹⁵ | 1% | 575.51 [M + H]⁺ | 575.18 | 1.66 | D |
| 368 | 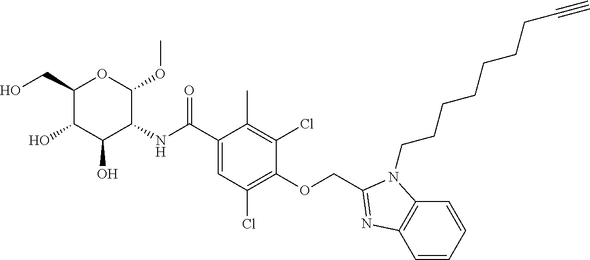 [SMILES: C#CCCCCCCCn1c(COc2c(Cl)cc(C(=O)N[C@H]3[C@@H](OC)O[C@H](CO)[C@@H](O)[C@@H]3O)c(C)c2Cl)nc2ccccc21] | L | 73% | 648.55 [M + H]⁺ | 648.23 | 2.11 | D |
| 369 | 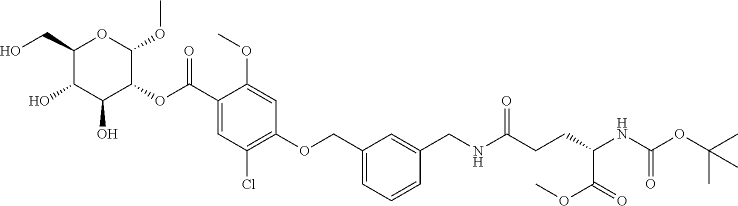 [SMILES: COC(=O)[C@H](CCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L | 31% | 741.66 [M + H]⁺ | 741.26 | 1.89 | D |
| 370 | 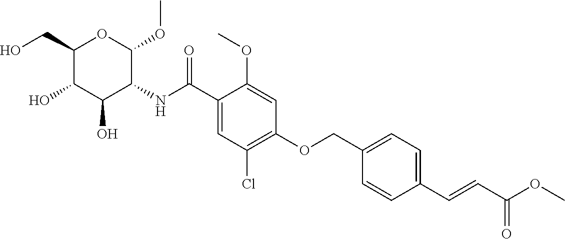 [SMILES: COC(=O)/C=C/c1ccc(COc2cc(OC)c(C(=O)N[C@H]3[C@@H](OC)O[C@H](CO)[C@@H](O)[C@@H]3O)cc2Cl)cc1] | R | 100% | 552.42 [M + H]⁺ | 552.16 | 1.93 | D |
| 371 | 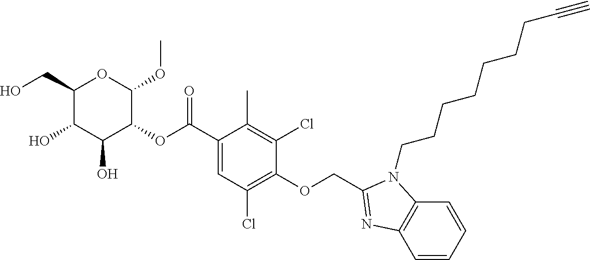 [SMILES: C#CCCCCCCCn1c(COc2c(Cl)cc(C(=O)O[C@H]3[C@@H](OC)O[C@H](CO)[C@@H](O)[C@@H]3O)c(C)c2Cl)nc2ccccc21] | I*¹⁵ | 31% | 693.59 [M − H + Formic acid]⁻ | 693.29 | 2.32 | D |
| 372 | 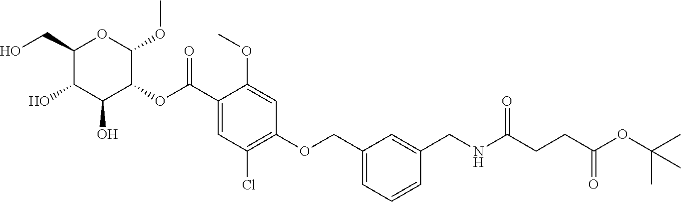 [SMILES: COc1cc(OCc2cccc(CNC(=O)CCC(=O)OC(C)(C)C)c2)c(Cl)cc1C(=O)O[C@H]1[C@@H](OC)O[C@H](CO)[C@@H](O)[C@@H]1O] | I*²⁸ + L | 35% | 698.64 [M − H + Formic acid]⁻ | 698.31 | 1.90 | D |
| 373 | 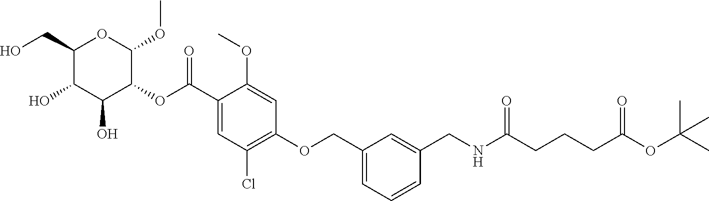 [SMILES: COc1cc(OCc2cccc(CNC(=O)CCCC(=O)OC(C)(C)C)c2)c(Cl)cc1C(=O)O[C@H]1[C@@H](OC)O[C@H](CO)[C@@H](O)[C@@H]1O] | I*²⁸ + L | 36% | 712.67 [M − H + Formic acid]⁻ | 712.33 | 1.96 | D |
| 374 | 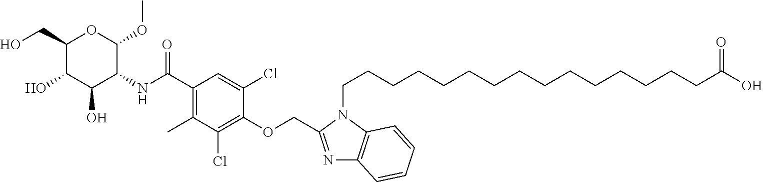 [SMILES: CO[C@H]1O[C@H](CO)[C@@H](O)[C@H](O)[C@H]1NC(=O)c1cc(Cl)c(OCc2nc3ccccc3n2CCCCCCCCCCCCCCCC(=O)O)c(Cl)c1C] | L*⁹ | 36% | 780.48 [M + H]⁺ | 780.34 | 2.66 | D |
| 375 | 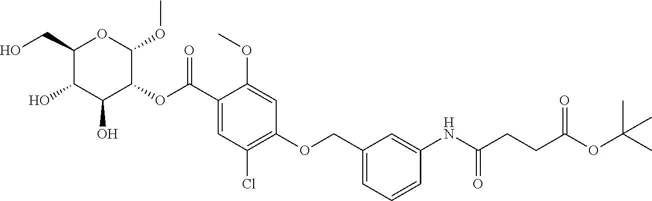 [SMILES: COc1cc(OCc2cccc(NC(=O)CCC(=O)OC(C)(C)C)c2)c(Cl)cc1C(=O)O[C@H]1[C@@H](OC)O[C@H](CO)[C@@H](O)[C@@H]1O] | I*²⁸ + L | 21% | 638.45 [M − H]⁻ | 638.20 | 1.96 | D |
| 376 | 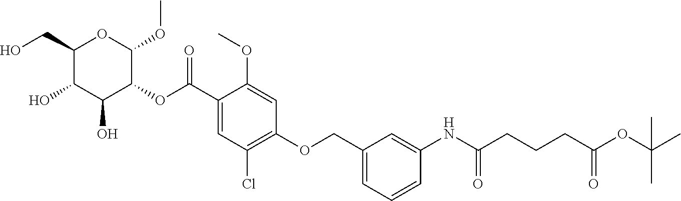 [SMILES: COc1cc(OCc2cccc(NC(=O)CCCC(=O)OC(C)(C)C)c2)c(Cl)cc1C(=O)O[C@H]1[C@@H](OC)O[C@H](CO)[C@@H](O)[C@@H]1O] | I*¹⁵ | 26% | 698.44 [M − H + Formic acid]⁻ | 698.31 | 2.05 | D |
| 377 | 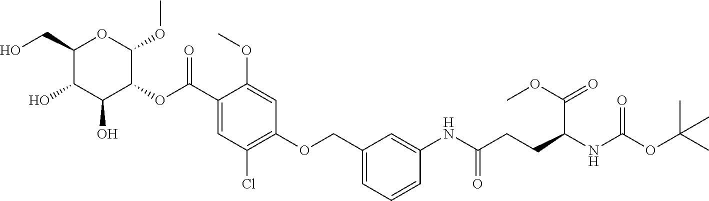 [SMILES: COC(=O)[C@H](CCC(=O)Nc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L | 22% | 727.41 [M + H]⁺ | 727.25 | 1.93 | D |
| 378 | 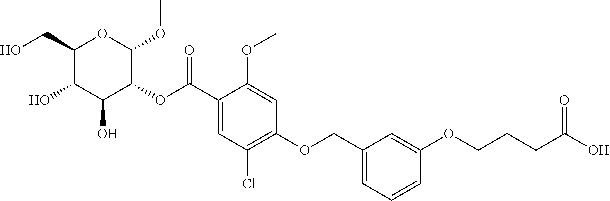 [SMILES: COc1cc(OCc2cccc(OCCCC(=O)O)c2)c(Cl)cc1C(=O)O[C@H]1[C@@H](OC)O[C@H](CO)[C@@H](O)[C@@H]1O] | I*¹⁹ | 18% | 571.40 [M + H]⁺ | 571.16 | 1.75 | D |
| 379 | 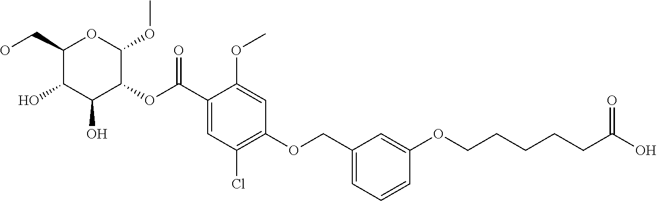 [SMILES: COc1cc(OCc2cccc(OCCCCCC(=O)O)c2)c(Cl)cc1C(=O)O[C@H]1[C@@H](OC)O[C@H](CO)[C@@H](O)[C@@H]1O] | I*¹⁹ | 19% | 599.47 [M + H]⁺ | 599.19 | 1.93 | D |
| 380 | 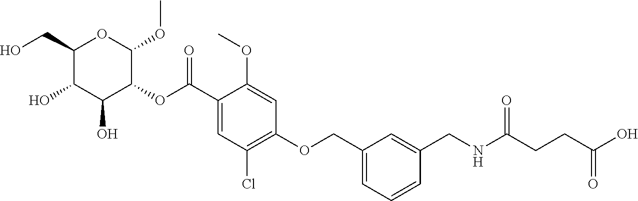 [SMILES: COc1cc(OCc2cccc(CNC(=O)CCC(=O)O)c2)c(Cl)cc1C(=O)O[C@H]1[C@@H](OC)O[C@H](CO)[C@@H](O)[C@@H]1O] | I*²⁸ + L*²⁶ | 35% | 596.36 [M − H]⁻ | 596.15 | 1.46 | D |
| 381 | 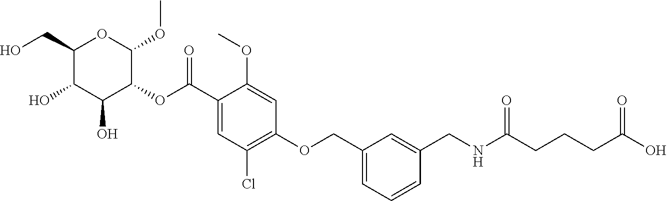 [SMILES: COc1cc(OCc2cccc(CNC(=O)CCCC(=O)O)c2)c(Cl)cc1C(=O)O[C@H]1[C@@H](OC)O[C@H](CO)[C@@H](O)[C@@H]1O] | I*²⁸ + L*²⁶ | 36% | 612.30 [M + H]⁺ | 612.19 | 1.49 | D |
| 382 | 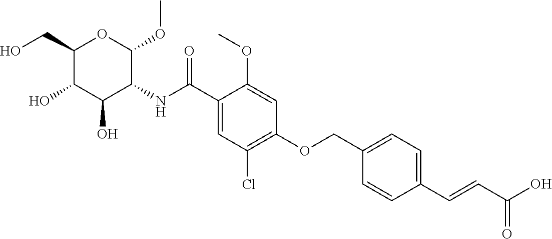 [SMILES: COc1cc(OCc2ccc(/C=C/C(=O)O)cc2)c(Cl)cc1C(=O)N[C@H]1[C@@H](OC)O[C@H](CO)[C@@H](O)[C@@H]1O] | R*¹⁴ | 46% | 538.17 [M + H]⁺ | 538.15 | 1.59 | D |
| 383 | 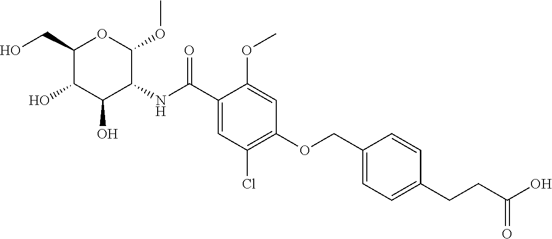 [SMILES: COc1cc(OCc2ccc(CCC(=O)O)cc2)c(Cl)cc1C(=O)N[C@H]1[C@@H](OC)O[C@H](CO)[C@@H](O)[C@@H]1O] | R*¹⁴ | 79% | 540.17 [M + H]⁺ | 540.16 | 1.61 | D |
| 384 | 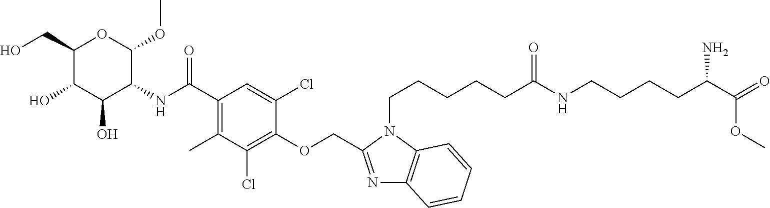 [SMILES: COC(=O)[C@@H](N)CCCCNC(=O)CCCCCn1c(COc2c(Cl)cc(C(=O)N[C@H]3[C@@H](OC)O[C@H](CO)[C@@H](O)[C@@H]3O)c(C)c2Cl)nc2ccccc21] | L*⁹ | 17% | 782.44 [M + H]⁺ | 782.29 | 1.20 | D |
| 385 | 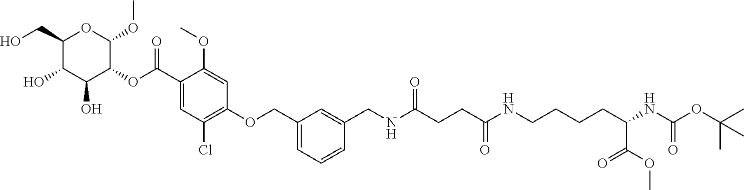 [SMILES: COC(=O)[C@H](CCCCNC(=O)CCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁶ + L | 18% | 840.40 [M + H]⁺ | 840.33 | 1.87 | D |
| 386 | 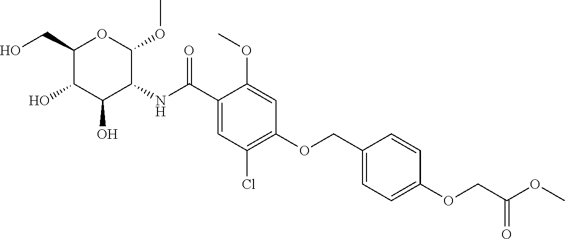 [SMILES: COC(=O)COc1ccc(COc2cc(OC)c(C(=O)N[C@H]3[C@@H](OC)O[C@H](CO)[C@@H](O)[C@@H]3O)cc2Cl)cc1] | R*²¹ | 7% | 556.23 [M + H]⁺ | 556.16 | 1.74 | D |
| 387 | 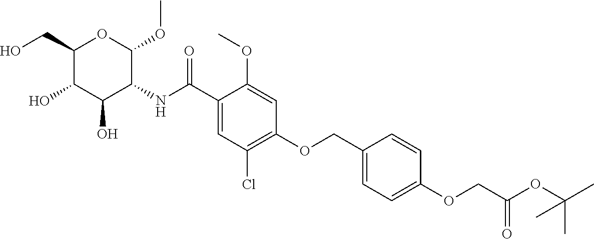 [SMILES: COc1cc(OCc2ccc(OCC(=O)OC(C)(C)C)cc2)c(Cl)cc1C(=O)N[C@H]1[C@@H](OC)O[C@H](CO)[C@@H](O)[C@@H]1O] | R | 43% | 598.27 [M + H]⁺ | 598.21 | 2.12 | D |
| 388 | 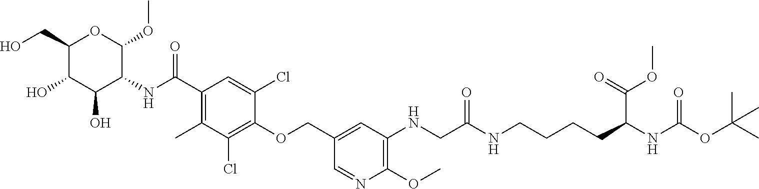 [SMILES: COC(=O)[C@H](CCCCNC(=O)CNc1cc(COc2c(Cl)cc(C(=O)N[C@H]3[C@@H](OC)O[C@H](CO)[C@@H](O)[C@@H]3O)c(C)c2Cl)cnc1OC)NC(=O)OC(C)(C)C] | L*²⁴ + L | 21% | 832.48 [M + H]⁺ | 832.29 | 1.88 | D |
| 389 | 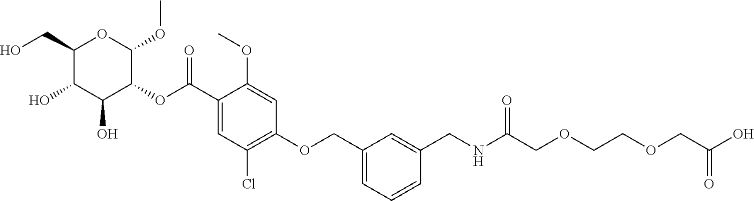 [SMILES: COc1cc(OCc2cccc(CNC(=O)COCCOCC(=O)O)c2)c(Cl)cc1C(=O)O[C@H]1[C@@H](OC)O[C@H](CO)[C@@H](O)[C@@H]1O] | I*²⁸ + L*²⁵ | 18% | 658.34 [M + H]⁺ | 658.19 | 1.47 | D |
| 390 | 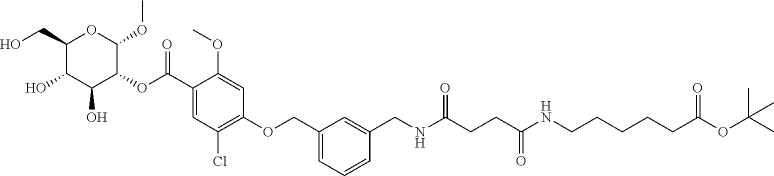 [SMILES: COc1cc(OCc2cccc(CNC(=O)CCC(=O)NCCCCCC(=O)OC(C)(C)C)c2)c(Cl)cc1C(=O)O[C@H]1[C@@H](OC)O[C@H](CO)[C@@H](O)[C@@H]1O] | I*²⁸ + L*²⁵ + L | 17% | 767.49 [M + H]⁺ | 767.33 | 2.00 | D |
| 391 | 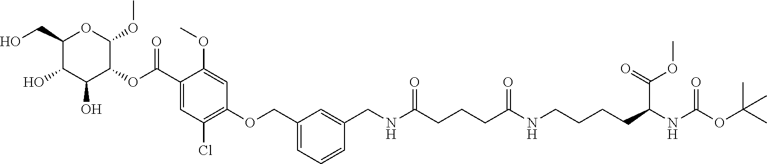 [SMILES: COC(=O)[C@H](CCCCNC(=O)CCCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁶ + L | 22% | 854.55 [M + H]⁺ | 854.35 | 1.88 | D |
| 392 | 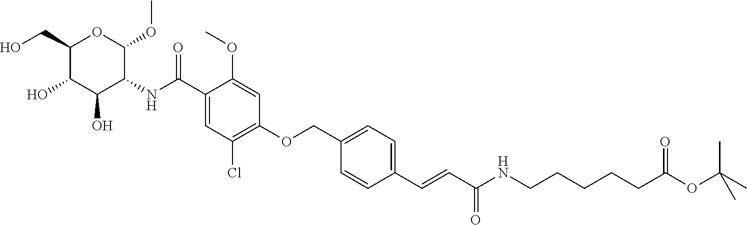 [SMILES: COc1cc(OCc2ccc(/C=C/C(=O)NCCCCCC(=O)OC(C)(C)C)cc2)c(Cl)cc1C(=O)N[C@H]1[C@@H](OC)O[C@H](CO)[C@@H](O)[C@@H]1O] | R*¹⁴ + L | 32% | 707.49 [M + H]⁺ | 707.30 | 2.17 | D |
| 393 | 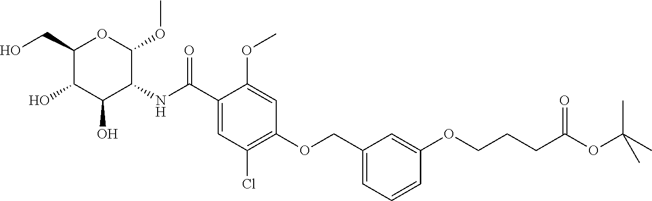 [SMILES: COc1cc(OCc2cccc(OCCCC(=O)OC(C)(C)C)c2)c(Cl)cc1C(=O)N[C@H]1[C@@H](OC)O[C@H](CO)[C@@H](O)[C@@H]1O] | L | 65% | 626.32 [M + H]⁺ | 626.24 | 2.35 | D |
| 394 | 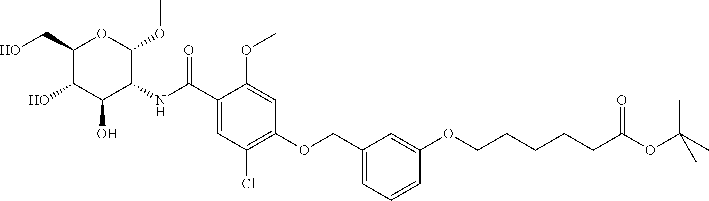 [SMILES: COc1cc(OCc2cccc(OCCCCCC(=O)OC(C)(C)C)c2)c(Cl)cc1C(=O)N[C@H]1[C@@H](OC)O[C@H](CO)[C@@H](O)[C@@H]1O] | L | 76% | 654.38 [M + H]⁺ | 654.27 | 2.54 | D |
| 395 | 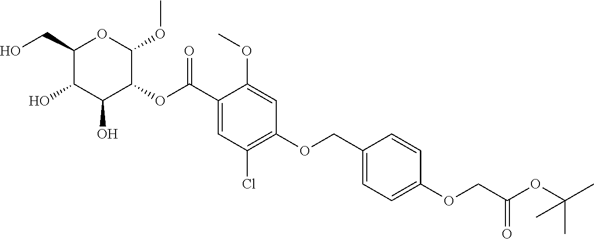 [SMILES: COc1cc(OCc2ccc(OCC(=O)OC(C)(C)C)cc2)c(Cl)cc1C(=O)O[C@H]1[C@@H](OC)O[C@H](CO)[C@@H](O)[C@@H]1O] | R | 40% | 643.29 [M − H + formic acid]⁻ | 643.27 | 2.15 | D |
| 396 | 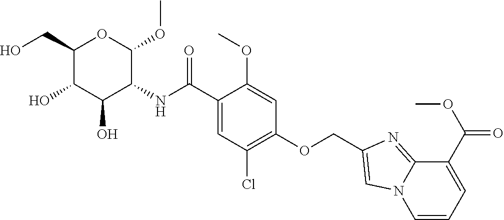 [SMILES: COC(=O)c1cccn2cc(COc3cc(OC)c(C(=O)N[C@H]4[C@@H](OC)O[C@H](CO)[C@@H](O)[C@@H]4O)cc3Cl)nc12] | R | 84% | 566.18 [M + H]⁺ | 566.15 | 1.07 | D |
| 397 | 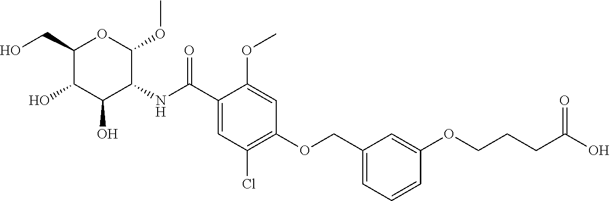 [SMILES: COc1cc(OCc2cccc(OCCCC(=O)O)c2)c(Cl)cc1C(=O)N[C@H]1[C@@H](OC)O[C@H](CO)[C@@H](O)[C@@H]1O] | L*¹⁹ | 42% | 570.26 [M + H]⁺ | 570.17 | 1.71 | D |
| 398 | 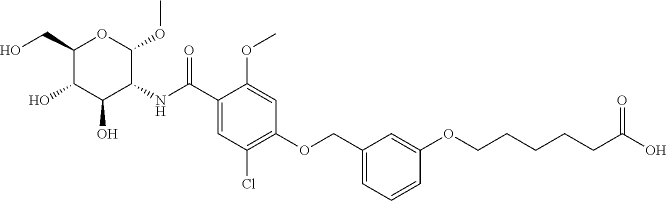 [SMILES: COc1cc(OCc2cccc(OCCCCCC(=O)O)c2)c(Cl)cc1C(=O)N[C@H]1[C@@H](OC)O[C@H](CO)[C@@H](O)[C@@H]1O] | L*¹⁹ | 37% | 598.33 [M + H]⁺ | 598.21 | 1.90 | D |
| 399 | 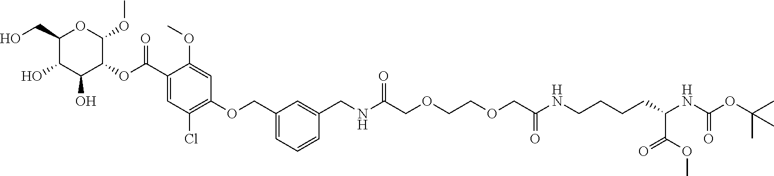 [SMILES: COC(=O)[C@H](CCCCNC(=O)COCCOCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁵ + L | 9% | 900.54 [M + H]⁺ | 900.35 | 1.88 | D |
| 400 | 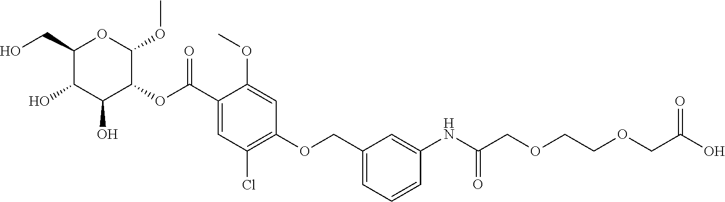 [SMILES: COc1cc(OCc2cccc(NC(=O)COCCOCC(=O)O)c2)c(Cl)cc1C(=O)O[C@H]1[C@@H](OC)O[C@H](CO)[C@@H](O)[C@@H]1O] | I*²⁸ + L*²⁵ | 13% | 642.30 [M − H]⁻ | 642.16 | 1.50 | D |
| 401 | 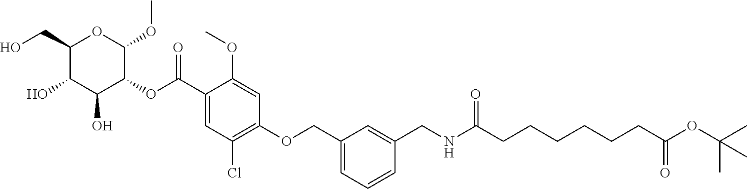 [SMILES: COc1cc(OCc2cccc(CNC(=O)CCCCCCC(=O)OC(C)(C)C)c2)c(Cl)cc1C(=O)O[C@H]1[C@@H](OC)O[C@H](CO)[C@@H](O)[C@@H]1O] | I*²⁸ + L | 17% | 754.50 [M − H + Formic acid]⁻ | 754.38 | 2.21 | D |
| 402 | 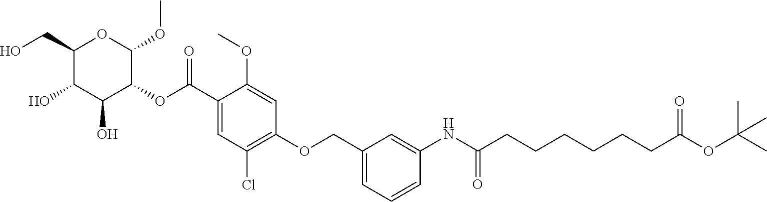 [SMILES: COc1cc(OCc2cccc(NC(=O)CCCCCCC(=O)OC(C)(C)C)c2)c(Cl)cc1C(=O)O[C@H]1[C@@H](OC)O[C@H](CO)[C@@H](O)[C@@H]1O] | I*²⁸ + L | 24% | 740.52 [M − H + Formic acid]⁻ | 740.36 | 2.32 | D |
| 403 | 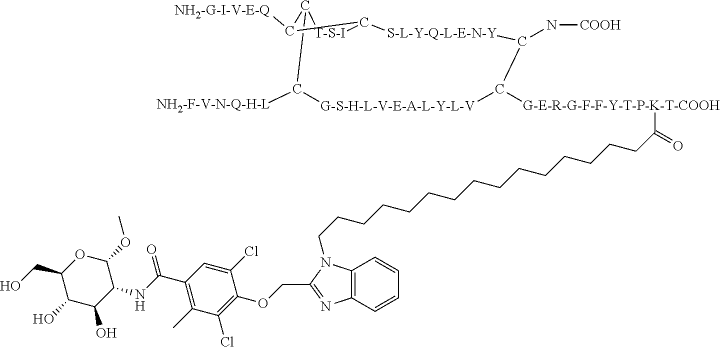 | Q | 32% | 1095.8 [M + 6H]⁶⁺ | 1096.07 | λ = 215 nm: 7.69 | F |
| 404 | 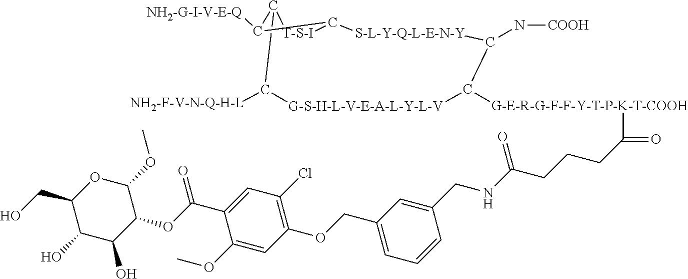 | Q | 24% | 1062.3 [M + 6H]⁶⁺ | 1067.95 | λ = 215 nm: 5.35 | F |
| 405 | 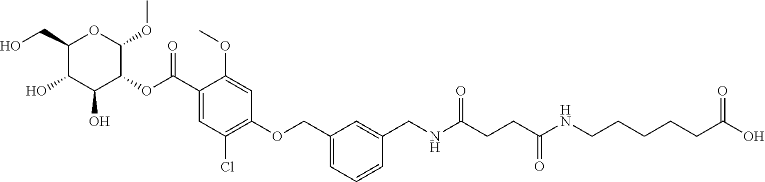 [SMILES: COc1cc(OCc2cccc(CNC(=O)CCC(=O)NCCCCCC(=O)O)c2)c(Cl)cc1C(=O)O[C@H]1[C@@H](OC)O[C@H](CO)[C@@H](O)[C@@H]1O] | I*²⁸ + L*²⁴ + L*²4 | 17% | 711.48 [M + H]⁺ | 711.25 | 1.51 | D |
| 406 | 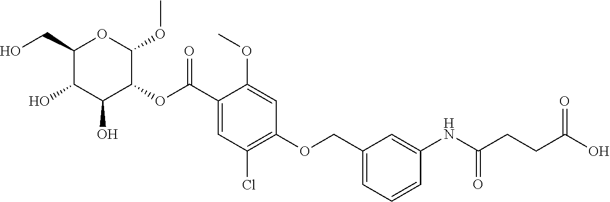 [SMILES: COc1cc(OCc2cccc(NC(=O)CCC(=O)O)c2)c(Cl)cc1C(=O)O[C@H]1[C@@H](OC)O[C@H](CO)[C@@H](O)[C@@H]1O] | I*²⁸ + L*²⁴ | 8% | 582.32 [M − H]⁻ | 582.14 | 1.48 | D |
| 407 | 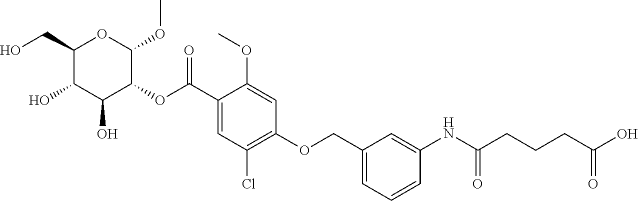 [SMILES: COc1cc(OCc2cccc(NC(=O)CCCC(=O)O)c2)c(Cl)cc1C(=O)O[C@H]1[C@@H](OC)O[C@H](CO)[C@@H](O)[C@@H]1O] | I*²⁸ + L*²⁴ | 22% | 596.33 [M − H]⁻ | 596.15 | 1.52 | D |
| 408 | 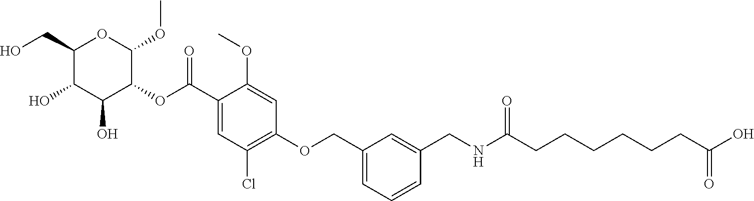 [SMILES: COc1cc(OCc2cccc(CNC(=O)CCCCCCC(=O)O)c2)c(Cl)cc1C(=O)O[C@H]1[C@@H](OC)O[C@H](CO)[C@@H](O)[C@@H]1O] | I*²⁸ + L*²⁴ | 17% | 654.40 [M + H]⁺ | 654.23 | 1.66 | D |
| 409 | 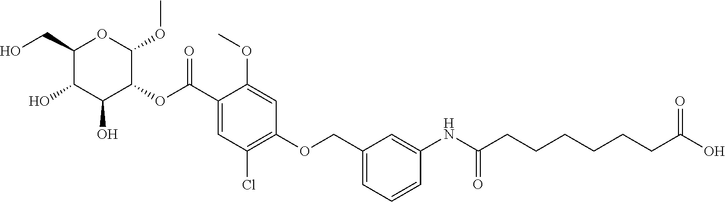 [SMILES: COc1cc(OCc2cccc(NC(=O)CCCCCCC(=O)O)c2)c(Cl)cc1C(=O)O[C@H]1[C@@H](OC)O[C@H](CO)[C@@H](O)[C@@H]1O] | I*²⁸ + L*²⁴ | 22% | 638.43 [M − H]⁻ | 638.20 | 1.73 | D |
| 410 | 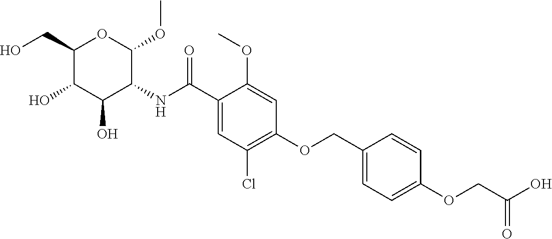 [SMILES: COc1cc(OCc2ccc(OCC(=O)O)cc2)c(Cl)cc1C(=O)N[C@H]1[C@@H](OC)O[C@H](CO)[C@@H](O)[C@@H]1O] | R*¹⁹ | 12% | 542.21 [M + H]⁺ | 542.14 | 1.48 | D |
| 411 | 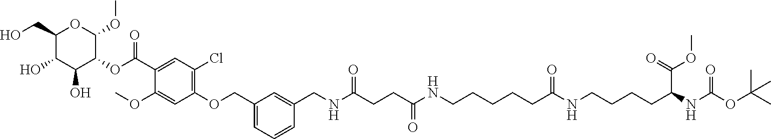 [SMILES: COC(=O)[C@H](CCCCNC(=O)CCCCCNC(=O)CCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁴ + L*²⁴ + L | 4% | 953.70 [M + H]⁺ | 953.42 | 1.86 | D |
| 412 | 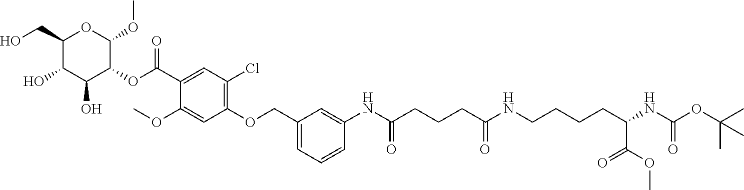 [SMILES: COC(=O)[C@H](CCCCNC(=O)CCCC(=O)Nc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁴ + L | 9% | 840.53 [M + H]⁺ | 840.33 | 1.90 | D |
| 413 | 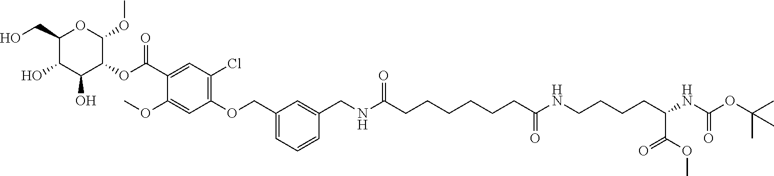 [SMILES: COC(=O)[C@H](CCCCNC(=O)CCCCCCC(=O)NCc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁴ + L | 7% | 896.62 [M + H]⁺ | 896.40 | 1.99 | D |
| 414 | 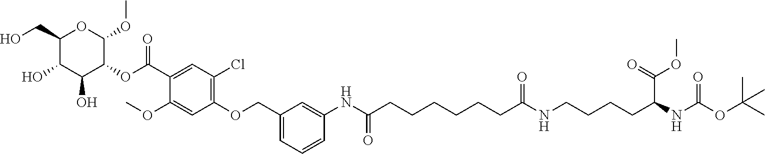 [SMILES: COC(=O)[C@H](CCCCNC(=O)CCCCCCC(=O)Nc1cccc(COc2cc(OC)c(C(=O)O[C@H]3[C@@H](OC)O[C@H](CO)[C@@H](O)[C@@H]3O)cc2Cl)c1)NC(=O)OC(C)(C)C] | I*²⁸ + L*²⁴ + L | 11% | 882.61 [M + H]⁺ | 882.38 | 2.05 | D |
| 415 | 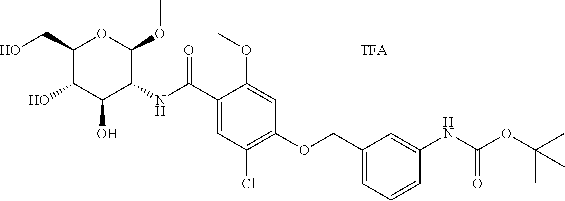 [SMILES: COc1cc(OCc2cccc(NC(=O)OC(C)(C)C)c2)c(Cl)cc1C(=O)N[C@H]1[C@H](OC)O[C@H](CO)[C@@H](O)[C@@H]1O ; "TFA"] | L*²⁹ | 5% | 583.26 [M + H]⁺ | 583.21 | 1.99 | D |
| 416 | 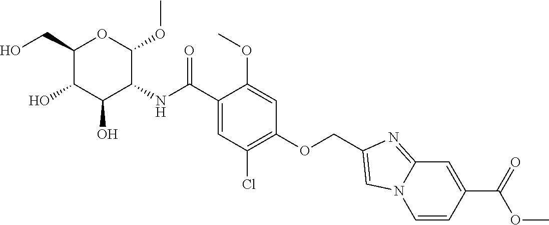 [SMILES: COC(=O)c1ccn2cc(COc3cc(OC)c(C(=O)N[C@H]4[C@@H](OC)O[C@H](CO)[C@@H](O)[C@@H]4O)cc3Cl)nc2c1] | R | 57% | 566.20 [M + H]⁺ | 566.15 | 1.43 | D |
| 417 | 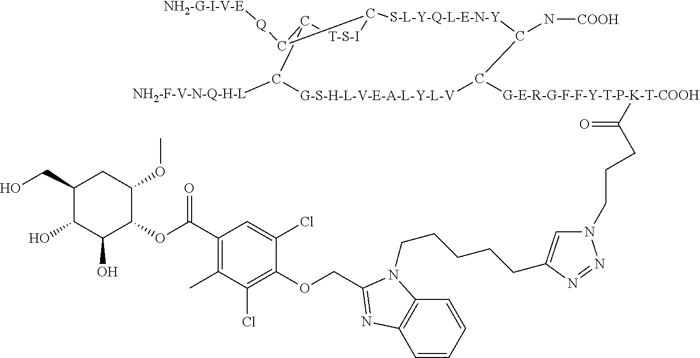 | P | 7% | 1090.7 [M + 6H]⁶⁺ | 1091.04 | λ = 215 nm: 5.80 | F |
| 418 | 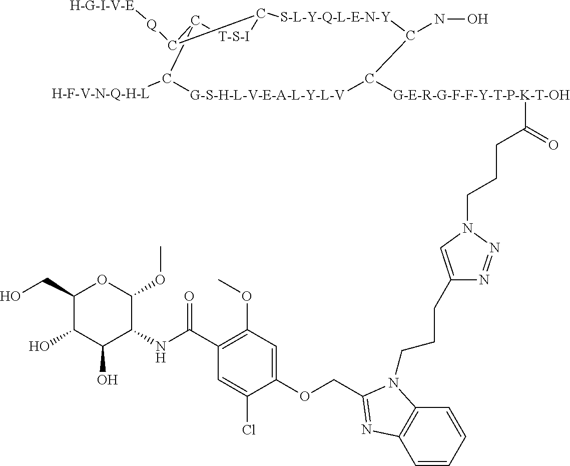 | P | 7% | 1077.49 [M + 6H]⁶⁺ | 1083.13 | λ = 215 nm: 5.33 | F |
| 419 | 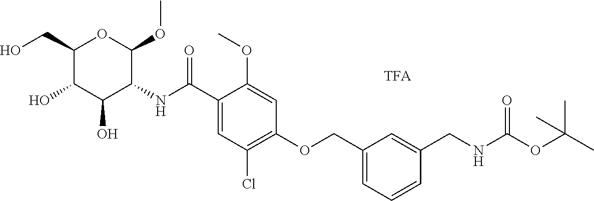 [SMILES: COc1cc(OCc2cccc(CNC(=O)OC(C)(C)C)c2)c(Cl)cc1C(=O)N[C@H]1[C@H](OC)O[C@H](CO)[C@@H](O)[C@@H]1O ; "TFA"] | L*²⁹ | 4% | 597.37 [M + H]⁺ | 597.22 | 1.93 | D |
| 420 | 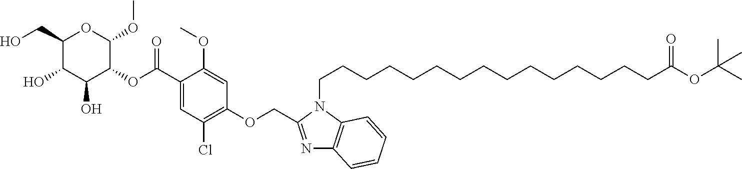 [SMILES: COc1cc(OCc2nc3ccccc3n2CCCCCCCCCCCCCCCC(=O)OC(C)(C)C)c(Cl)cc1C(=O)O[C@H]1[C@@H](OC)O[C@H](CO)[C@@H](O)[C@@H]1O] | L | 61% | 818.63 [M + H]⁺ | 818.44 | 3.31 | D |
| 421 | 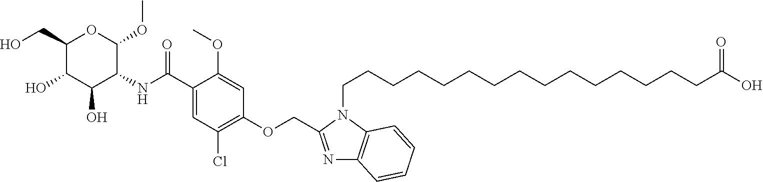 [SMILES: COc1cc(OCc2nc3ccccc3n2CCCCCCCCCCCCCCCC(=O)O)c(Cl)cc1C(=O)N[C@H]1[C@@H](OC)O[C@H](CO)[C@@H](O)[C@@H]1O] | L*⁹ | 16% | 762.59 [M + H]⁺ | 762.37 | 2.67 | D |
| 422 | 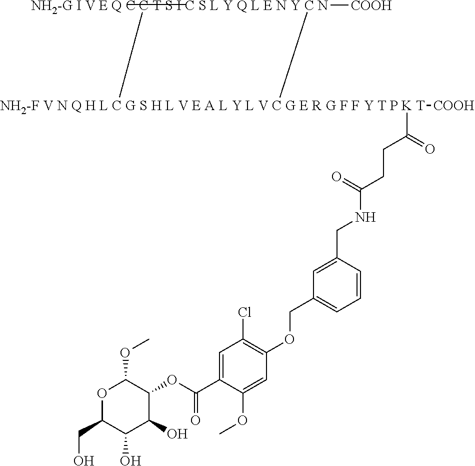 | Q | 13% | 1060.1 [M + 6H]⁶⁺ | 1065.61 | λ = 215 nm: 5.41 | F |
| 423 | 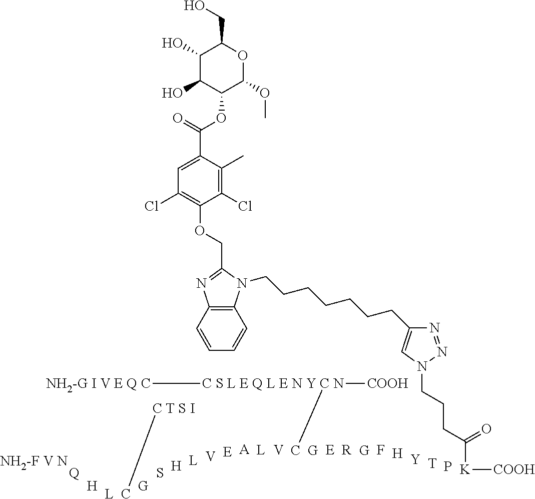 | P | 21% | 1066.8 [M + 6H]⁶⁺ | 1067.18 | λ = 215 nm: 5.08 | F |
| 424 | 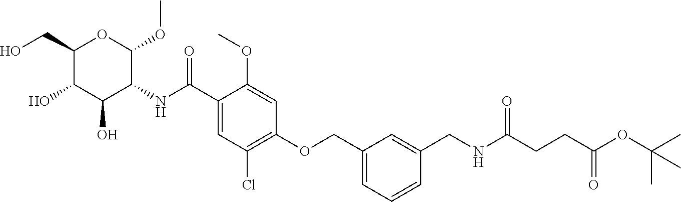 [SMILES: COc1cc(OCc2cccc(CNC(=O)CCC(=O)OC(C)(C)C)c2)c(Cl)cc1C(=O)N[C@H]1[C@@H](OC)O[C@H](CO)[C@@H](O)[C@@H]1O] | L*²⁶ + L | 76% | 653.39 [M + H]⁺ | 653.25 | 1.87 | D |
| 425 | 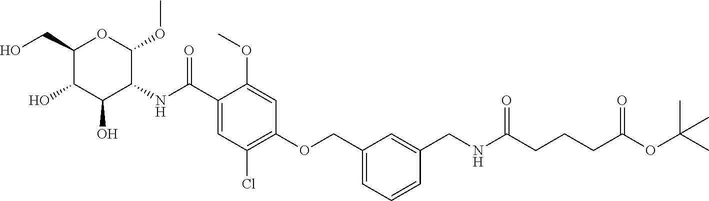 [SMILES: COc1cc(OCc2cccc(CNC(=O)CCCC(=O)OC(C)(C)C)c2)c(Cl)cc1C(=O)N[C@H]1[C@@H](OC)O[C@H](CO)[C@@H](O)[C@@H]1O] | L*²⁶ + L | 60% | 667.44 [M + H]⁺ | 667.26 | 1.93 | D |
| 426 | 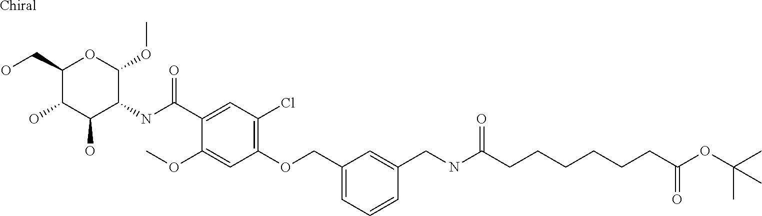 [SMILES: "Chiral" ; COc1cc(OCc2cccc(CNC(=O)CCCCCCC(=O)OC(C)(C)C)c2)c(Cl)cc1C(=O)N[C@H]1[C@@H](OC)O[C@H](CO)[C@@H](O)[C@@H]1O] | L*²⁶ + L | 53% | 709.51 [M + H]⁺ | 709.31 | 2.18 | D |
| 427 | 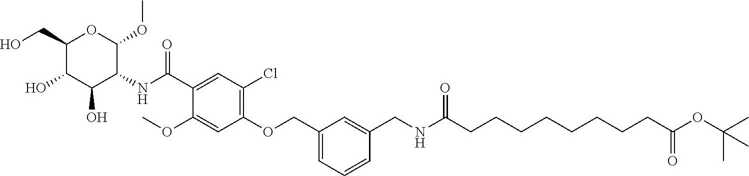 [SMILES: COc1cc(OCc2cccc(CNC(=O)CCCCCCCCC(=O)OC(C)(C)C)c2)c(Cl)cc1C(=O)N[C@H]1[C@@H](OC)O[C@H](CO)[C@@H](O)[C@@H]1O] | L*²⁶ + L | 54% | 737.51 [M + H]⁺ | 737.34 | 2.39 | D |
| 428 | 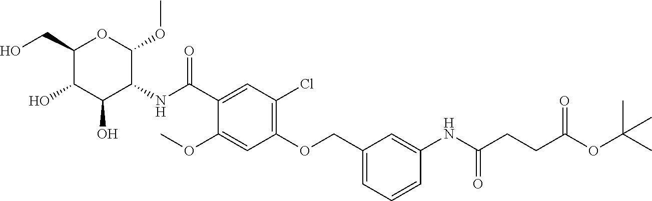 [SMILES: COc1cc(OCc2cccc(NC(=O)CCC(=O)OC(C)(C)C)c2)c(Cl)cc1C(=O)N[C@H]1[C@@H](OC)O[C@H](CO)[C@@H](O)[C@@H]1O] | L*²⁶ + L | 41% | 639.36 [M + H]⁺ | 639.23 | 1.93 | D |
| 429 | 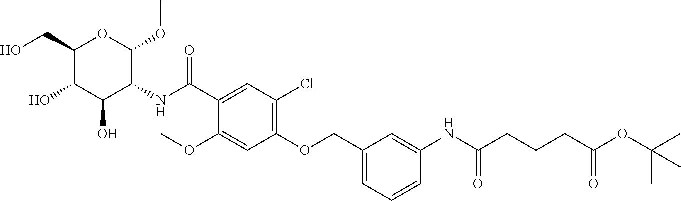 [SMILES: COc1cc(OCc2cccc(NC(=O)CCCC(=O)OC(C)(C)C)c2)c(Cl)cc1C(=O)N[C@H]1[C@@H](OC)O[C@H](CO)[C@@H](O)[C@@H]1O] | L*²⁶ + L | 45% | 653.40 [M + H]⁺ | 653.25 | 2.01 | D |
| 430 | 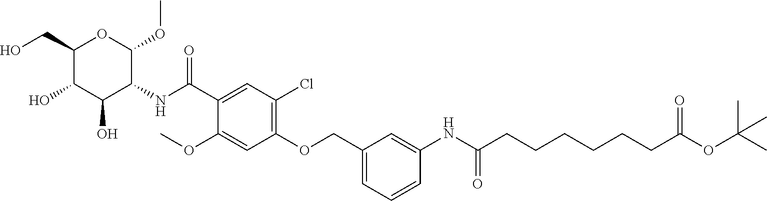 [SMILES: COc1cc(OCc2cccc(NC(=O)CCCCCCC(=O)OC(C)(C)C)c2)c(Cl)cc1C(=O)N[C@H]1[C@@H](OC)O[C@H](CO)[C@@H](O)[C@@H]1O] | L*²⁶ + L | 40% | 695.44 [M + H]⁺ | 695.30 | 2.28 | D |
| 431 | 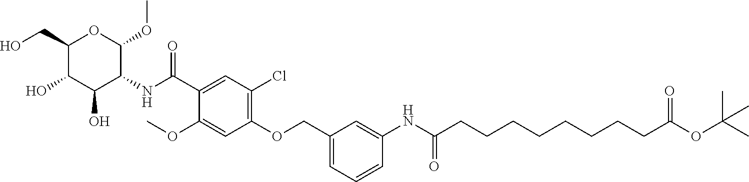 [SMILES: COc1cc(OCc2cccc(NC(=O)CCCCCCCCC(=O)OC(C)(C)C)c2)c(Cl)cc1C(=O)N[C@H]1[C@@H](OC)O[C@H](CO)[C@@H](O)[C@@H]1O] | L*²⁶ + L | 44% | 723.44 [M + H]⁺ | 723.33 | 2.50 | D |
| 432 | 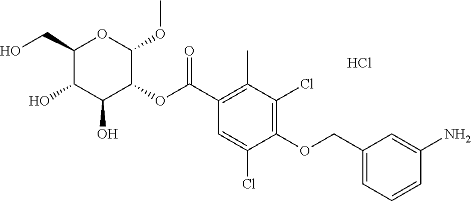 [SMILES: CO[C@H]1O[C@H](CO)[C@@H](O)[C@H](O)[C@H]1OC(=O)c1cc(Cl)c(OCc2cccc(N)c2)c(Cl)c1C ; Cl] | I*¹⁶ | 47% | 502.14 [M + H]⁺ | 502.02 | 1.64 | D |
| 433 | 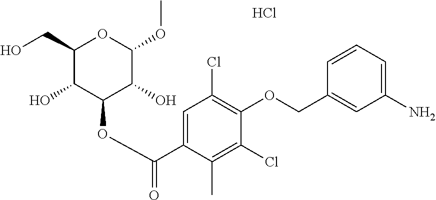 [SMILES: CO[C@H]1O[C@H](CO)[C@@H](O)[C@H](OC(=O)c2cc(Cl)c(OCc3cccc(N)c3)c(Cl)c2C)[C@H]1O ; Cl] | I*¹⁶ | 3% | 502.17 [M + H]⁺ | 502.02 | 1.48 | D |
| 434 | 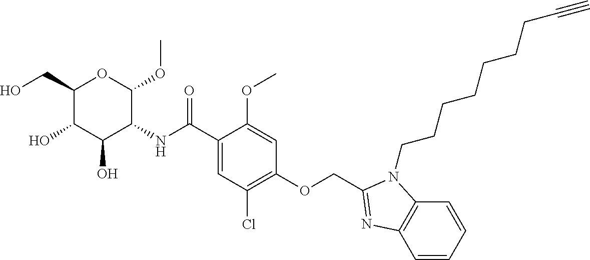 [SMILES: C#CCCCCCCCn1c(COc2cc(OC)c(C(=O)N[C@H]3[C@@H](OC)O[C@H](CO)[C@@H](O)[C@@H]3O)cc2Cl)nc2ccccc21] | L | 53% | 630.41 [M + H]⁺ | 630.26 | 2.15 | D |
| 435 | 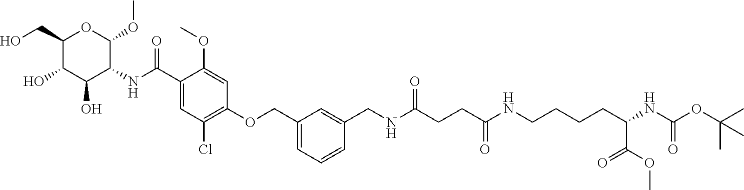 [SMILES: COC(=O)[C@H](CCCCNC(=O)CCC(=O)NCc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 35% | 839.55 [M + H]⁺ | 839.35 | 1.83 | D |
| 436 | 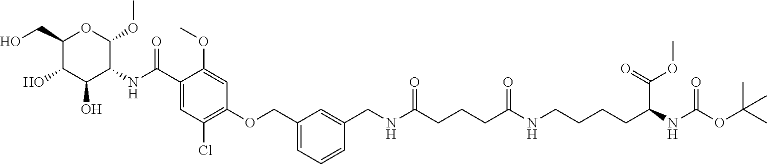 [SMILES: COC(=O)[C@H](CCCCNC(=O)CCCC(=O)NCc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 30% | 853.60 [M + H]⁺ | 853.36 | 1.84 | D |
| 437 | 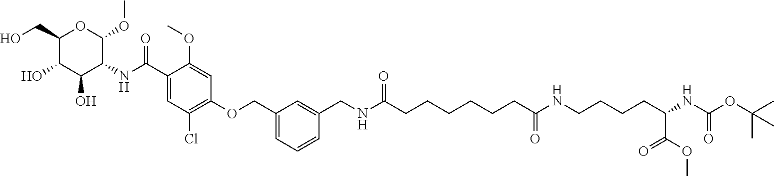 [SMILES: COC(=O)[C@H](CCCCNC(=O)CCCCCCC(=O)NCc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 28% | 895.66 [M + H]⁺ | 895.41 | 1.97 | D |
| 438 | 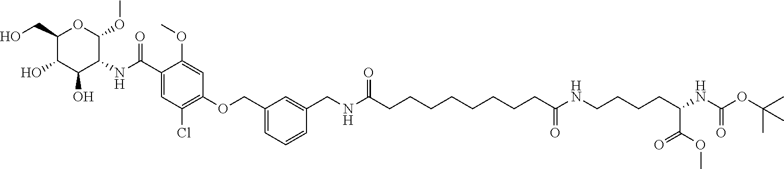 [SMILES: COC(=O)[C@H](CCCCNC(=O)CCCCCCCCC(=O)NCc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 30% | 923.70 [M + H]⁺ | 923.44 | 2.10 | D |
| 439 | 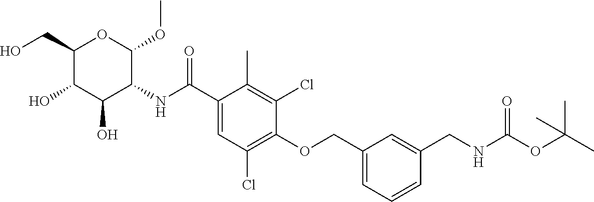 [SMILES: CO[C@H]1O[C@H](CO)[C@@H](O)[C@H](O)[C@H]1NC(=O)c1cc(Cl)c(OCc2cccc(CNC(=O)OC(C)(C)C)c2)c(Cl)c1C] | L | 42% | 615.26 [M + H]⁺ | 615.19 | 2.07 | D |
| 440 | 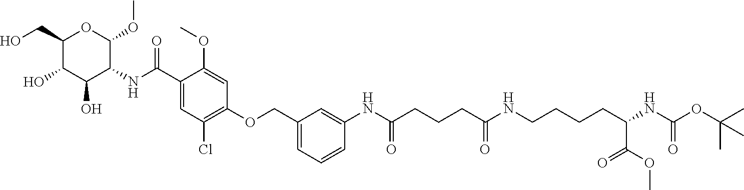 [SMILES: COC(=O)[C@H](CCCCNC(=O)CCCC(=O)Nc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ +L | 19% | 839.57 [M + H]⁺ | 839.35 | 1.88 | D |
| 441 | 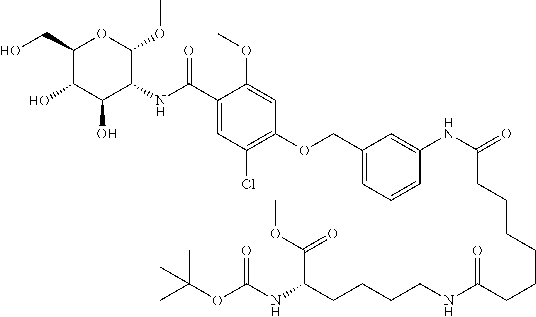 [SMILES: COC(=O)[C@H](CCCCNC(=O)CCCCCCC(=O)Nc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 23% | 881.67 [M + H]⁺ | 881.47 | 2.04 | D |
| 442 | 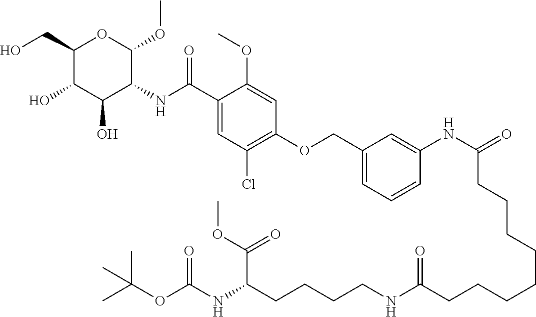 [SMILES: COC(=O)[C@H](CCCCNC(=O)CCCCCCCCC(=O)Nc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L | 22% | 909.72 [M + H]⁺ | 909.43 | 2.18 | D |
| 443 | 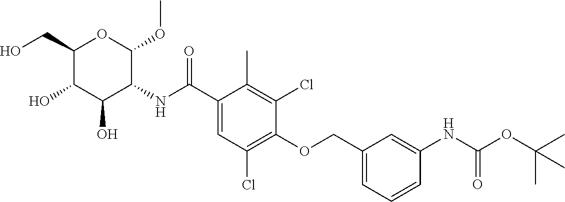 [SMILES: CO[C@H]1O[C@H](CO)[C@@H](O)[C@H](O)[C@H]1NC(=O)c1cc(Cl)c(OCc2cccc(NC(=O)OC(C)(C)C)c2)c(Cl)c1C] | L | 55% | 601.19 [M + H]⁺ | 601.17 | 2.13 | D |
| 444 | 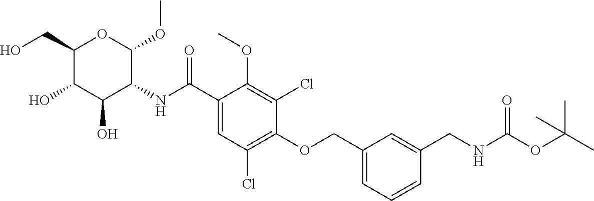 [SMILES: COc1c(C(=O)N[C@H]2[C@@H](OC)O[C@H](CO)[C@@H](O)[C@@H]2O)cc(Cl)c(OCc2cccc(CNC(=O)OC(C)(C)C)c2)c1Cl] | L | 73% | 631.31 [M + H]⁺ | 631.18 | 2.12 | D |
| 445 | 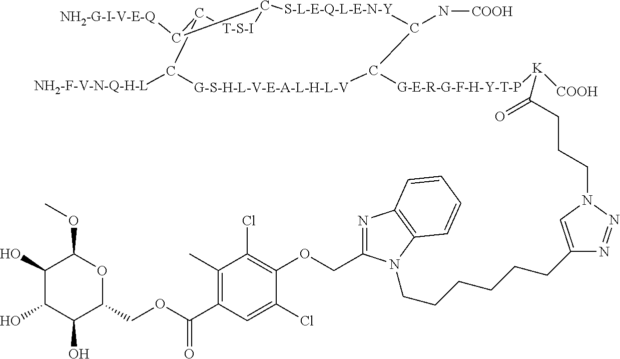 | P*³³ | 2% | 1064.6 [M + 6H]⁶⁺ | 1064.84 | λ = 215 nm: 4.96 | F |
| 446 | 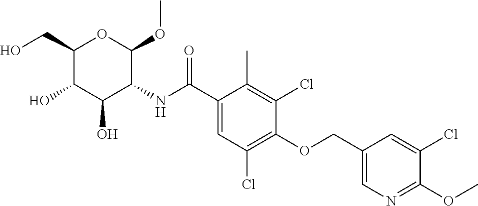 [SMILES: COc1ncc(COc2c(Cl)cc(C(=O)N[C@H]3[C@H](OC)O[C@H](CO)[C@@H](O)[C@@H]3O)c(C)c2Cl)cc1Cl] | L*²⁹ | 3% | 551.09 [M + H]⁺ | 551.07 | 1.93 | D |
| 447 | 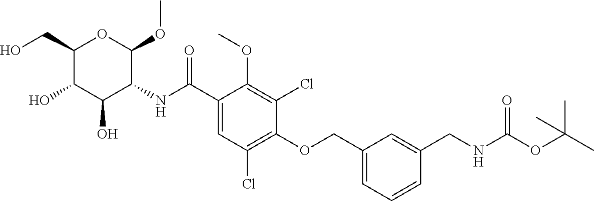 [SMILES: COc1c(C(=O)N[C@H]2[C@H](OC)O[C@H](CO)[C@@H](O)[C@@H]2O)cc(Cl)c(OCc2cccc(CNC(=O)OC(C)(C)C)c2)c1Cl] | L*²⁹ | 3% | 629.34 [M − H]⁻ | 629.17 | 2.04 | D |
| 448 | 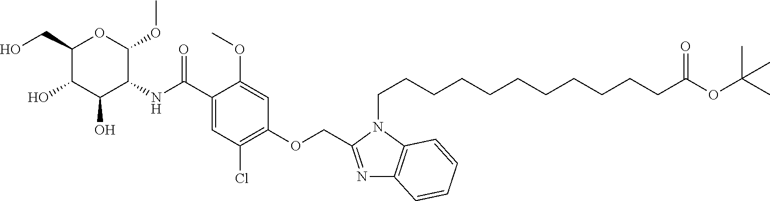 [SMILES: COc1cc(OCc2nc3ccccc3n2CCCCCCCCCCCC(=O)OC(C)(C)C)c(Cl)cc1C(=O)N[C@H]1[C@@H](OC)O[C@H](CO)[C@@H](O)[C@@H]1O] | L | 94% | 762.53 [M + H]⁺ | 762.37 | 2.84 | D |
| 449 | 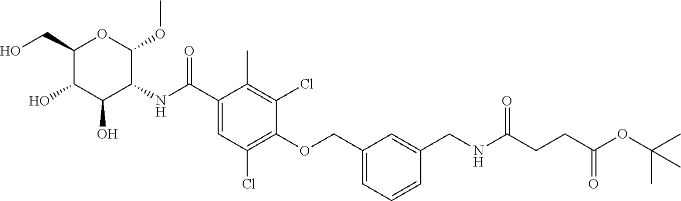 [SMILES: CO[C@H]1O[C@H](CO)[C@@H](O)[C@H](O)[C@H]1NC(=O)c1cc(Cl)c(OCc2cccc(CNC(=O)CCC(=O)OC(C)(C)C)c2)c(Cl)c1C] | L*²⁶ + L | 26% | 671.35 [M + H]⁺ | 671.21 | 1.93 | D |
| 450 | 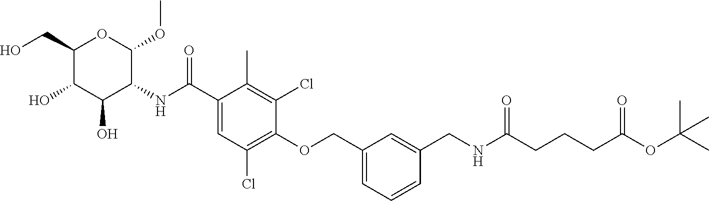 [SMILES: CO[C@H]1O[C@H](CO)[C@@H](O)[C@H](O)[C@H]1NC(=O)c1cc(Cl)c(OCc2cccc(CNC(=O)CCCC(=O)OC(C)(C)C)c2)c(Cl)c1C] | L*²⁶ + L | 23% | 685.38 [M + H]⁺ | 685.23 | 1.99 | D |
| 451 | 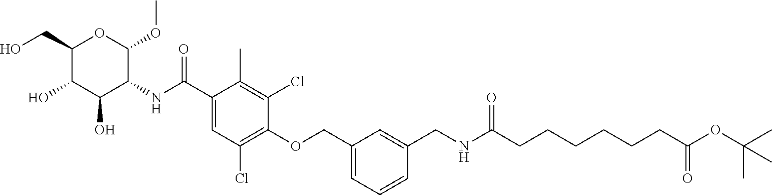 [SMILES: CO[C@H]1O[C@H](CO)[C@@H](O)[C@H](O)[C@H]1NC(=O)c1cc(Cl)c(OCc2cccc(CNC(=O)CCCCCCC(=O)OC(C)(C)C)c2)c(Cl)c1C] | L*²⁶ + L | 25% | 727.45 [M + H]⁺ | 727.28 | 2.23 | D |
| 452 | 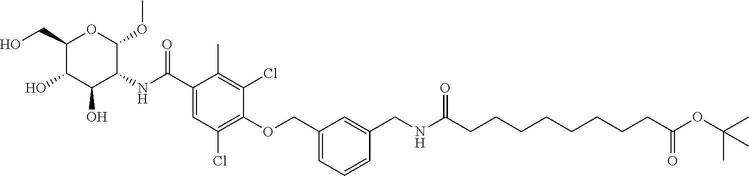 [SMILES: CO[C@H]1O[C@H](CO)[C@@H](O)[C@H](O)[C@H]1NC(=O)c1cc(Cl)c(OCc2cccc(CNC(=O)CCCCCCCCC(=O)OC(C)(C)C)c2)c(Cl)c1C] | L*²⁶ + L | 28% | 755.49 [M + H]⁺ | 755.31 | 2.44 | D |
| 453 | 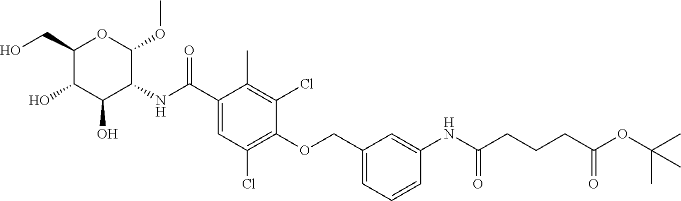 [SMILES: CO[C@H]1O[C@H](CO)[C@@H](O)[C@H](O)[C@H]1NC(=O)c1cc(Cl)c(OCc2cccc(NC(=O)CCCC(=O)OC(C)(C)C)c2)c(Cl)c1C] | L*²⁶ + L | 39% | 671.37 [M + H]⁺ | 671.21 | 2.07 | D |
| 454 | 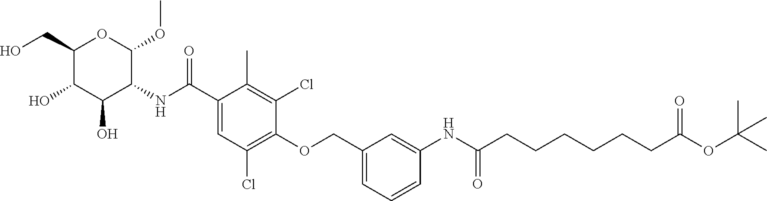 [SMILES: CO[C@H]1O[C@H](CO)[C@@H](O)[C@H](O)[C@H]1NC(=O)c1cc(Cl)c(OCc2cccc(NC(=O)CCCCCCC(=O)OC(C)(C)C)c2)c(Cl)c1C] | L*²⁶ + L | 26% | 713.43 [M + H]⁺ | 713.26 | 2.34 | D |
| 455 | 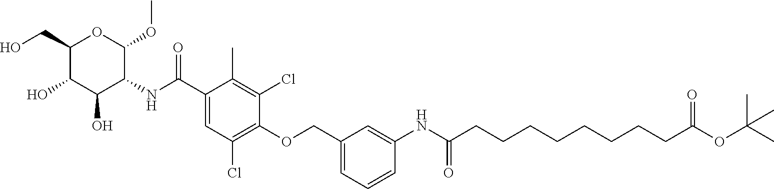 [SMILES: CO[C@H]1O[C@H](CO)[C@@H](O)[C@H](O)[C@H]1NC(=O)c1cc(Cl)c(OCc2cccc(NC(=O)CCCCCCCCC(=O)OC(C)(C)C)c2)c(Cl)c1C] | L*²⁶ + L | 33% | 741.43 [M + H]⁺ | 741.29 | 2.56 | D |
| 456 | 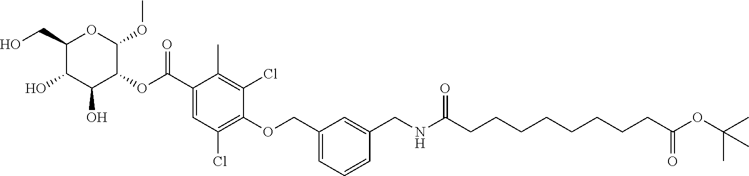 [SMILES: CO[C@H]1O[C@H](CO)[C@@H](O)[C@H](O)[C@H]1OC(=O)c1cc(Cl)c(OCc2cccc(CNC(=O)CCCCCCCCC(=O)OC(C)(C)C)c2)c(Cl)c1C] | I*¹⁶ + L | 31% | 756.46 [M + H]⁺ | 756.29 | 2.61 | D |
| 457 | 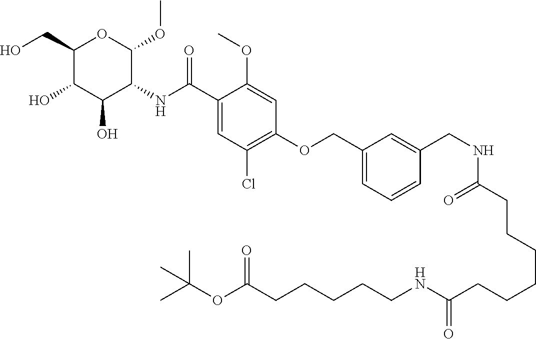 [SMILES: COc1cc(OCc2cccc(CNC(=O)CCCCCCC(=O)NCCCCCC(=O)OC(C)(C)C)c2)c(Cl)cc1C(=O)N[C@H]1[C@@H](OC)O[C@H](CO)[C@@H](O)[C@@H]1O] | L*²⁶ + L*²⁴ + L | 22% | 822.60 [M + H]⁺ | 822.39 | 2.10 | D |
| 458 | 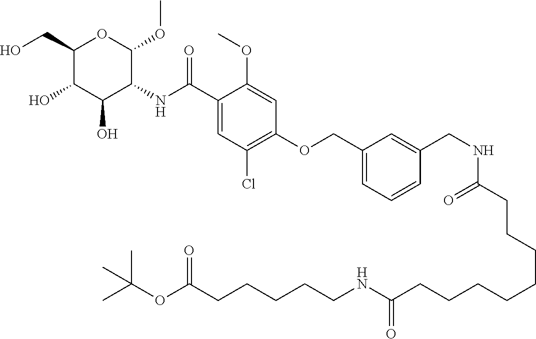 [SMILES: COc1cc(OCc2cccc(CNC(=O)CCCCCCCCC(=O)NCCCCCC(=O)OC(C)(C)C)c2)c(Cl)cc1C(=O)N[C@H]1[C@@H](OC)O[C@H](CO)[C@@H](O)[C@@H]1O] | L*²⁶ + L*²⁴ + L | 24% | 850.62 [M + H]⁺ | 850.43 | 2.24 | D |
| 459 | 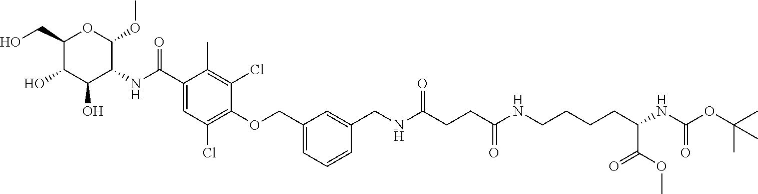 [SMILES: COC(=O)[C@H](CCCCNC(=O)CCC(=O)NCc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 13% | 857.47 [M + H]⁺ | 857.31 | 1.88 | D |
| 460 | 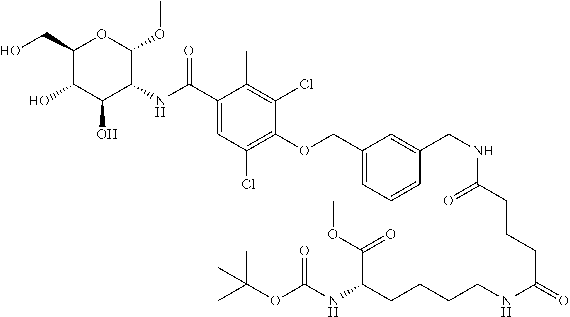 [SMILES: COC(=O)[C@H](CCCCNC(=O)CCCC(=O)NCc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 12% | 871.44 [M + H]⁺ | 871.33 | 1.89 | D |
| 461 | 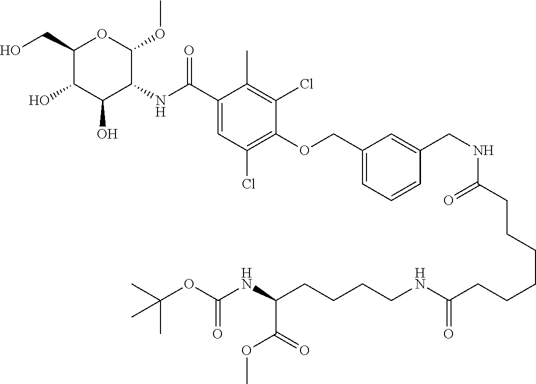 [SMILES: COC(=O)[C@H](CCCCNC(=O)CCCCCCC(=O)NCc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 14% | 913.58 [M + H]⁺ | 913.38 | 2.02 | D |
| 462 | 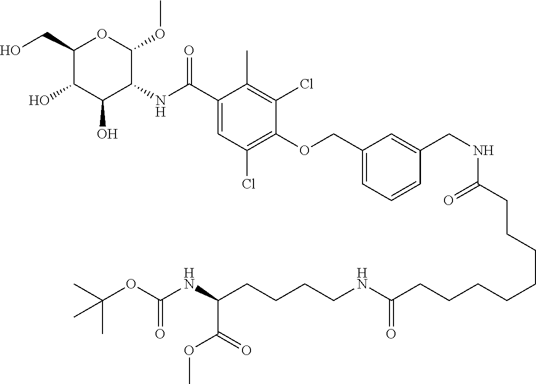 [SMILES: COC(=O)[C@H](CCCCNC(=O)CCCCCCCCC(=O)NCc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 15% | 941.15 [M + H]⁺ | 941.41 | 2.15 | D |
| 463 | 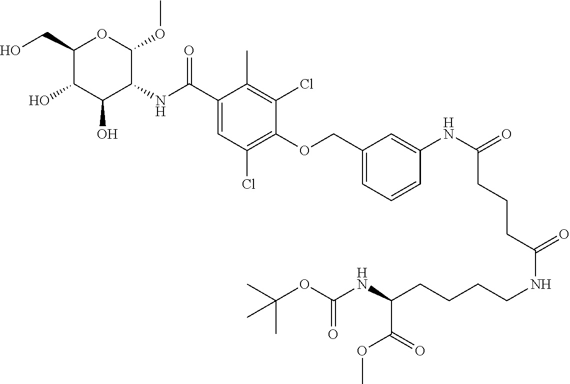 [SMILES: COC(=O)[C@H](CCCCNC(=O)CCCC(=O)Nc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 13% | 857.46 [M + H]⁺ | 857.31 | 1.92 | D |
| 464 | 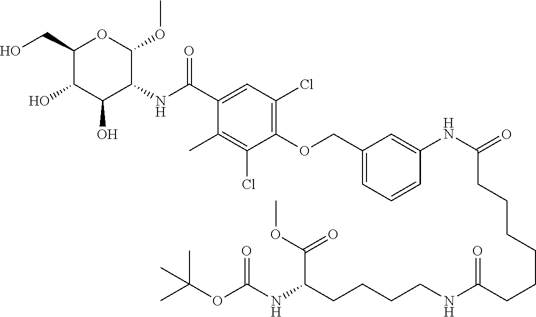 [SMILES: COC(=O)[C@H](CCCCNC(=O)CCCCCCC(=O)Nc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 11% | 899.52 [M + H]⁺ | 899.36 | 2.07 | D |
| 465 | 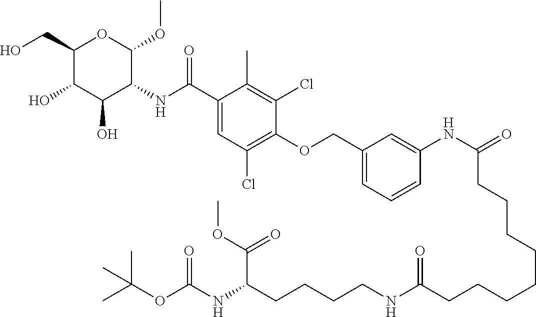 [SMILES: COC(=O)[C@H](CCCCNC(=O)CCCCCCCCC(=O)Nc1cccc(COc2c(Cl)cc(C(=O)N[C@H]3[C@@H](OC)O[C@H](CO)[C@@H](O)[C@@H]3O)c(C)c2Cl)c1)NC(=O)OC(C)(C)C] | L*²⁶ + L*²⁴ + L | 14% | 927.61 [M + H]⁺ | 927.39 | 2.22 | D |
| 466 | 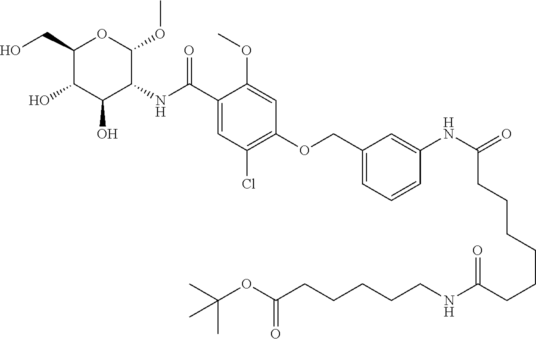 [SMILES: COc1cc(OCc2cccc(NC(=O)CCCCCCC(=O)NCCCCCC(=O)OC(C)(C)C)c2)c(Cl)cc1C(=O)N[C@H]1[C@@H](OC)O[C@H](CO)[C@@H](O)[C@@H]1O] | L*²⁶ + L*²⁴ + L | 18% | 808.41 [M + H]⁺ | 808.38 | 2.16 | D |
| 467 | 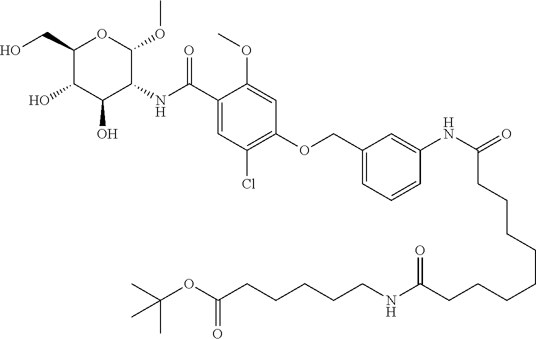 [SMILES: COc1cc(OCc2cccc(NC(=O)CCCCCCCCC(=O)NCCCCCC(=O)OC(C)(C)C)c2)c(Cl)cc1C(=O)N[C@H]1[C@@H](OC)O[C@H](CO)[C@@H](O)[C@@H]1O] | L*²⁶ + L*²⁴ + L | 23% | 836.52 [M + H]⁺ | 836.41 | 2.32 | D |
| 468 | 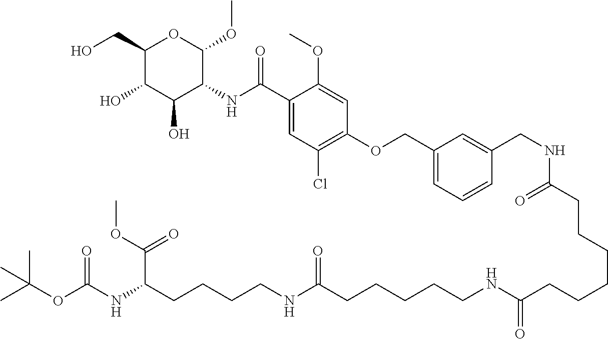 [SMILES: COC(=O)[C@H](CCCCNC(=O)CCCCCNC(=O)CCCCCCC(=O)NCc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L*²⁴ + L | 13% | 1008.67 [M + H]⁺ | 1008.50 | 1.96 | D |
| 469 | 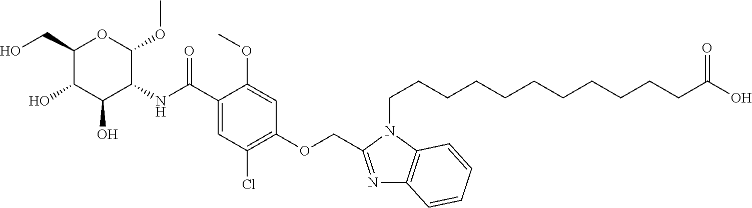 [SMILES: COc1cc(OCc2nc3ccccc3n2CCCCCCCCCCCC(=O)O)c(Cl)cc1C(=O)N[C@H]1[C@@H](OC)O[C@H](CO)[C@@H](O)[C@@H]1O] | L*⁹ | 47% | 706.51 [M + H]⁺ | 706.31 | 2.20 | D |
| 470 | 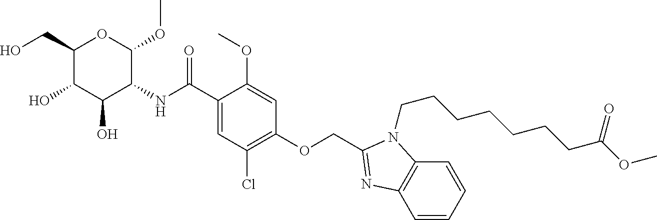 [SMILES: COC(=O)CCCCCCCn1c(COc2cc(OC)c(C(=O)N[C@H]3[C@@H](OC)O[C@H](CO)[C@@H](O)[C@@H]3O)cc2Cl)nc2ccccc21] | R | 20% | 664.42 [M + H]⁺ | 664.26 | 1.99 | D |
| 471 | 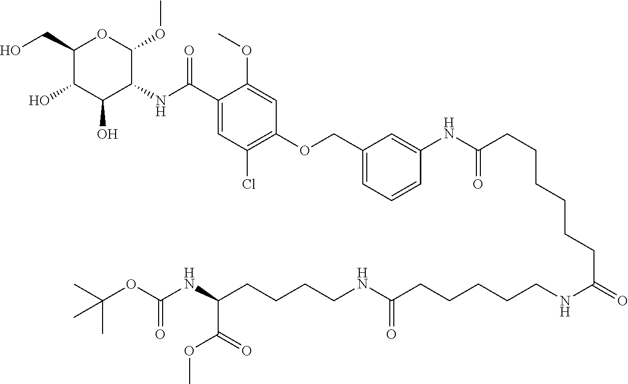 [SMILES: COC(=O)[C@H](CCCCNC(=O)CCCCCNC(=O)CCCCCCC(=O)Nc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L*²⁴ + L | 10% | 994.71 [M + H]⁺ | 994.48 | 2.00 | D |
| 472 | 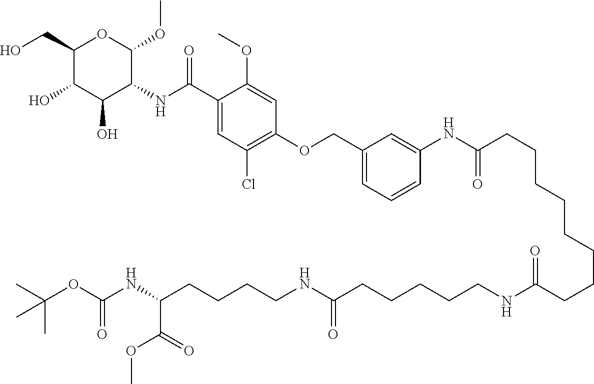 [SMILES: COC(=O)[C@@H](CCCCNC(=O)CCCCCNC(=O)CCCCCCCCC(=O)Nc1cccc(COc2cc(OC)c(C(=O)N[C@H]3[C@@H](OC)O[C@H](CO)[C@@H](O)[C@@H]3O)cc2Cl)c1)NC(=O)OC(C)(C)C] | L*²⁶ + L*²⁴ + L*²⁴ + L | 14% | 1022.77 [M + H]⁺ | 1022.51 | 2.14 | D |
| 473 | 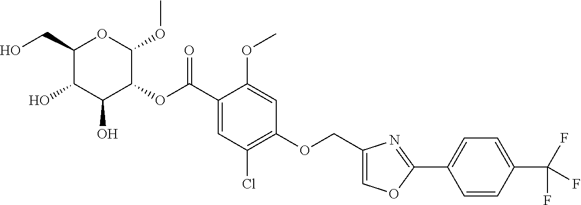 [SMILES: COc1cc(OCc2coc(-c3ccc(C(F)(F)F)cc3)n2)c(Cl)cc1C(=O)O[C@H]1[C@@H](OC)O[C@H](CO)[C@@H](O)[C@@H]1O] | R | 17% | 604.18 [M + H]⁺ | 604.12 | 2.24 | D |
| 474 | 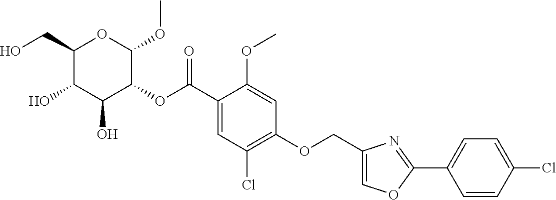 [SMILES: COc1cc(OCc2coc(-c3ccc(Cl)cc3)n2)c(Cl)cc1C(=O)O[C@H]1[C@@H](OC)O[C@H](CO)[C@@H](O)[C@@H]1O] | R | 15% | 570.14 [M + H]⁺ | 570.09 | 2.17 | D |
| 475 | 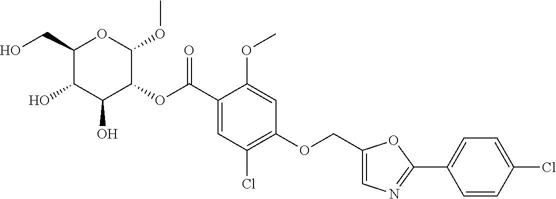 [SMILES: COc1cc(OCc2cnc(-c3ccc(Cl)cc3)o2)c(Cl)cc1C(=O)O[C@H]1[C@@H](OC)O[C@H](CO)[C@@H](O)[C@@H]1O] | R | 21% | 570.16 [M + H]⁺ | 570.09 | 2.17 | D |
| 476 | 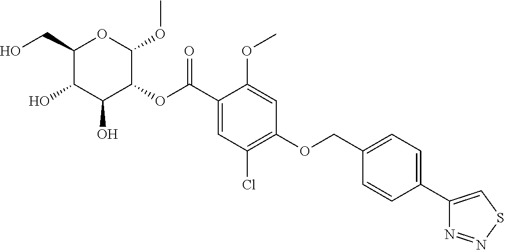 [SMILES: COc1cc(OCc2ccc(-c3csnn3)cc2)c(Cl)cc1C(=O)O[C@H]1[C@@H](OC)O[C@H](CO)[C@@H](O)[C@@H]1O] | R | 18% | 597.25 [M − H + Formic acid]⁻ | 597.09 | 1.86 | D |
| 477 | 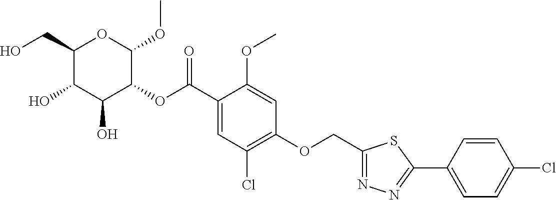 [SMILES: COc1cc(OCc2nnc(-c3ccc(Cl)cc3)s2)c(Cl)cc1C(=O)O[C@H]1[C@@H](OC)O[C@H](CO)[C@@H](O)[C@@H]1O] | R | 12% | 631.24 [M − H + Formic acid]⁻ | 631.06 | 2.08 | D |
| 478 | 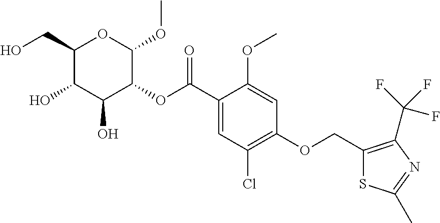 [SMILES: COc1cc(OCc2sc(C)nc2C(F)(F)F)c(Cl)cc1C(=O)O[C@H]1[C@@H](OC)O[C@H](CO)[C@@H](O)[C@@H]1O] | R | 30% | 558.13 [M + H]⁺ | 558.08 | 1.84 | D |
| 479 | 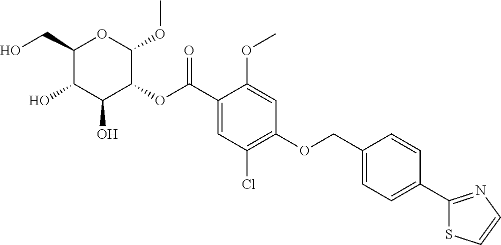 [SMILES: COc1cc(OCc2ccc(-c3nccs3)cc2)c(Cl)cc1C(=O)O[C@H]1[C@@H](OC)O[C@H](CO)[C@@H](O)[C@@H]1O] | R | 43% | 552.17 [M + H]⁺ | 552.11 | 1.93 | D |
| 480 | 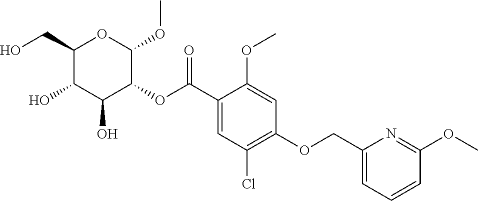 [SMILES: COc1cccc(COc2cc(OC)c(C(=O)O[C@H]3[C@@H](OC)O[C@H](CO)[C@@H](O)[C@@H]3O)cc2Cl)n1] | R | 8% | 500.18 [M + H]⁺ | 500.13 | 1.80 | D |
| 481 | 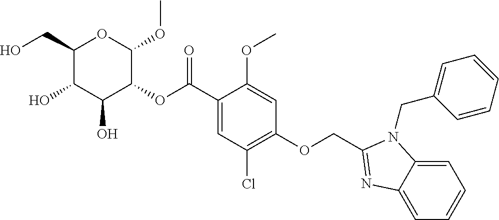 [SMILES: COc1cc(OCc2nc3ccccc3n2Cc2ccccc2)c(Cl)cc1C(=O)O[C@H]1[C@@H](OC)O[C@H](CO)[C@@H](O)[C@@H]1O] | R | 57% | 599.28 [M + H]⁺ | 599.18 | 1.93 | D |
| 482 | 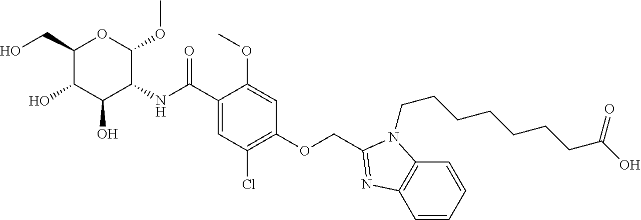 [SMILES: COc1cc(OCc2nc3ccccc3n2CCCCCCCC(=O)O)c(Cl)cc1C(=O)N[C@H]1[C@@H](OC)O[C@H](CO)[C@@H](O)[C@@H]1O] | R*¹⁴ | 18% | 650.35 [M + H]⁺ | 650.25 | 1.73 | D |
| 483 | 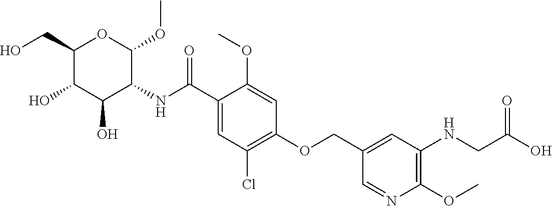 [SMILES: COc1cc(OCc2cnc(OC)c(NCC(=O)O)c2)c(Cl)cc1C(=O)N[C@H]1[C@@H](OC)O[C@H](CO)[C@@H](O)[C@@H]1O] | L | 15% | 572.1 [M + H]⁺ | 572.17 | 0.64 | C |
| 484 | 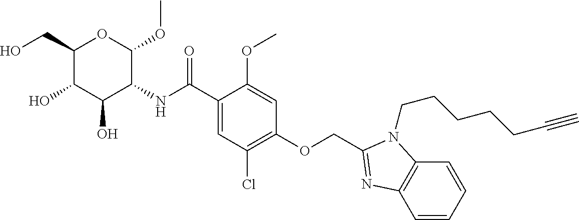 [SMILES: C#CCCCCCn1c(COc2cc(OC)c(C(=O)N[C@H]3[C@@H](OC)O[C@H](CO)[C@@H](O)[C@@H]3O)cc2Cl)nc2ccccc21] | R | 34% | 602.38 [M + H]⁺ | 602.23 | 1.89 | D |
| 485 | 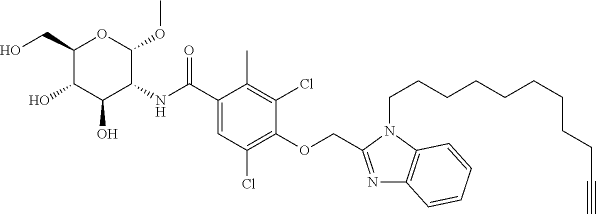 [SMILES: C#CCCCCCCCCCn1c(COc2c(Cl)cc(C(=O)N[C@H]3[C@@H](OC)O[C@H](CO)[C@@H](O)[C@@H]3O)c(C)c2Cl)nc2ccccc21] | R | 11% | 676.40 [M + H]⁺ | 676.26 | 2.37 | D |
| 486 | 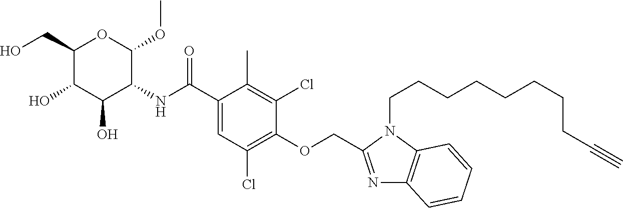 [SMILES: C#CCCCCCCCCn1c(COc2c(Cl)cc(C(=O)N[C@H]3[C@@H](OC)O[C@H](CO)[C@@H](O)[C@@H]3O)c(C)c2Cl)nc2ccccc21] | R | 36% | 662.41 [M + H]⁺ | 662.24 | 2.24 | D |
| 487 | 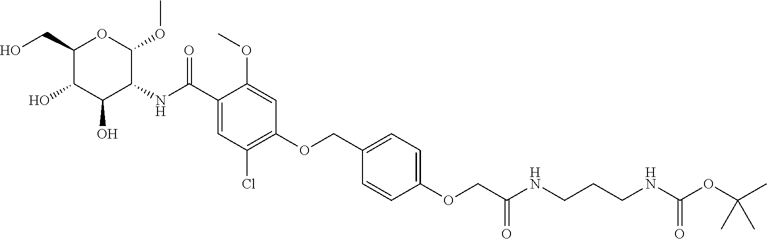 [SMILES: COc1cc(OCc2ccc(OCC(=O)NCCCNC(=O)OC(C)(C)C)cc2)c(Cl)cc1C(=O)N[C@H]1[C@@H](OC)O[C@H](CO)[C@@H](O)[C@@H]1O] | R*²² + L | 4% | 698.41 [M + H]⁺ | 698.27 | 1.84 | D |
| 488 | 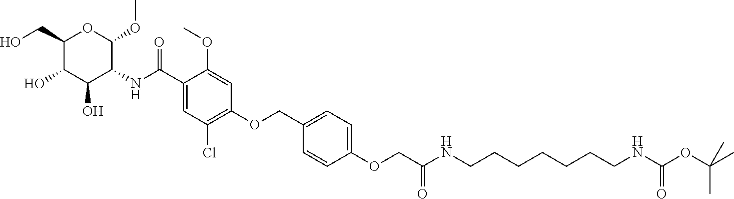 [SMILES: COc1cc(OCc2ccc(OCC(=O)NCCCCCCCNC(=O)OC(C)(C)C)cc2)c(Cl)cc1C(=O)N[C@H]1[C@@H](OC)O[C@H](CO)[C@@H](O)[C@@H]1O] | R*²² + L | 3% | 754.46 [M + H]⁺ | 754.33 | 2.13 | D |
| 489 | 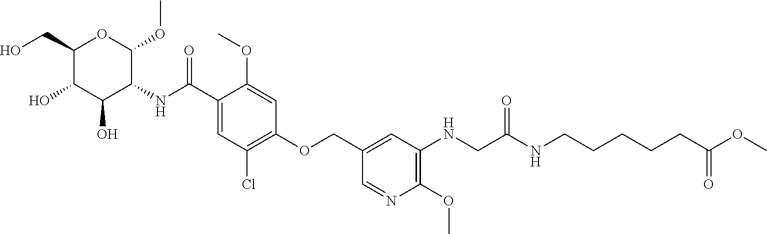 [SMILES: COC(=O)CCCCCNC(=O)CNc1cc(COc2cc(OC)c(C(=O)N[C@H]3[C@@H](OC)O[C@H](CO)[C@@H](O)[C@@H]3O)cc2Cl)cnc1OC] | L | 25% | 699.41 [M + H]⁺ | 699.27 | 1.62 | D |
| 490 | 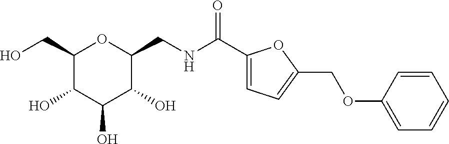 [SMILES: O=C(NC[C@@H]1O[C@H](CO)[C@@H](O)[C@H](O)[C@H]1O)c1ccc(COc2ccccc2)o1] | | | 394.14 [μ + H]⁺ | 394.15 | 1.29 | D |

*¹ = using additionally 0.1 eq. N,N-4-dimethylamino-pyridine.
*² = using 1-chloro-N,N-2-trimethylpropenylamine (Ghosez reagent) for preparation of acid chloride
*³ = examples 153a, 153b, 153c, and 153d were isolated from the same experiment
*⁴ = examples 154a and 154b were isolated from the same experiment
*⁵ = using Me₂SnCl₂ instead of Bu₂SnCl₂, 0.1 eq. 3,5-dimethylpyridine (both for direction to C6 benzoylation), and 1,2,2,6,6-pentamethylpiperidine instead of diisopropylethylamine.
*⁶ = using 1,2,3,4-O-tetra-trimethylsilyl-α-D-glycopyranoside instead of allyl-α-D-glycopyranoside
*⁷ = using HCl for benzylidene cleavage
*⁸ = additional BOC cleavage with trifluoroacetic acid/CH₂Cl₂ 1/100 at 25° C.
*⁹ = additional BOC cleavage with trifluoroacetic acid/CH₂Cl₂ 1/20 at 25° C.
*¹⁰ = additional BOC cleavage with trifluoroacetic acid/CH₂Cl₂ 1/50 at 25° C.
*¹¹ = C6-t-butyl-dimethyl-silylgroup cleavage in first step of method M
*¹² = using tin dichloride for benzylidene cleavage
*¹³ = additional BOC cleavage with trifluoroacetic acid/acetonitrile 1/50 at 25° C.
*¹⁴ = ester cleavage with 2 n NaOH/tetrahydrofuran/MeOH = 1/1/1 at 25° C. from example 200a
*¹⁵ = additional benzylidene cleavage with 2 M HCl/acetonitrile = 1/1 at 25° C.
*¹⁶ = additional benzylidene and BOC cleavage with 2 M HCl/acetonitrile = 1/1 at 25° C.
*¹⁷ = using toluene instead of tetrahydrofuran
*¹⁸ = step 2 of method M: coupling with HOBt like in synthesis method I (13%)
*¹⁹ = additional t-butyl ester and/or benzylidene cleavage with 1 M HCl/acetonitrile = 1/1 at 25° C.
*²⁰ = additional benzylidene cleavage with 1 M HCl/CH₂Cl₂ = 1/4 at 25° C.
*²¹ = isolated as byproduct from example 387
*²² = additional t-butyl ester cleavage with 2 M HCl/acetonitrile = 1/1 at 25° C. and after 2 hours at 40° C. for 2 hours
*²³ = using the respective benzoic acid, DCC, and DMAP (1/1.1/0.2) instead of benzoic acid chloride and triethylamine and additional 75 vol % of DMF as solvent
*²⁴ = additional t-butyl ester cleavage with trifluoroacetic acid/CH₂Cl₂ 1/10 at 25° C.
*²⁵ = 5 eq. bis-carboxylic acid, 5 eq. HATU, 7 eq. DIPEA
*²⁶ = additional BOC cleavage with trifluoroacetic acid/CH₂Cl₂ 1/10 at 25° C.
*²⁷ = additional benzylidene cleavage with triethylsilane/trifluoroacetic acid 10 eq/10 eq in CH₂Cl₂ at 25° C.
*²⁸ = additional benzylidene and BOC cleavage with 6 M HCl/acetonitrile = 1/1 at 25° C.
*²⁹ = isolated as byproduct from slightly impure methyl-α-D-glucosamine
*³⁰ = using the commercially available azid derivative instead of B29-4-azido-butyric acid activated human insulin
*³¹ = starting from A1-BOC, B29-BOC-human insulin, isolated as C3/C4 = 85/15 mixture via benzoyl migration
*³² = starting from A1-BOC, B29-BOC-human insulin, isolated as C2/C6 = 3/7 mixture via benzoyl migration
*³³ = isolated from reaction of C2-benzoyl-glucopyranoside via benzoyl-migration
*³⁴ = using 4-azido-butyric acid activated GGG tripeptide instead of B29-4-azido-butyric acid activated human insulin
*³⁵ = using the commercially available azid derivative instead of B29-4-azido-butyric acid activated human insulin and additional BOC cleavage with trifluoroacetic acid/CH₂Cl₂ 1/2 at 25° C.
*³⁶ = additional benzylidene cleavage with triethylsilane/trifluoroacetic acid 10 eq/10 eq in CH₂Cl₂ at 0° C.
*³⁷ = additional benzylidene cleavage with 2 M HCl/acetonitrile = 1/2 at 25° C.
*³⁸ = ester cleavage with 2 n NaOH/tetrahydrofuran/MeOH = 1/1/1 at 25° C.

Biological Assays

1. Deoxy-Glucose Uptake Assay in A2780 Cells:

Procedure

For measurement of ¹⁴C 2-deoxy-D-glucose transport into A2780 cells, cells were seeded in 96-well plates (Cytostar-T Plates Perkin Elmer, 70,000 cells/180 μl/well) in medium complete (RPMI1640+Glutamax (Life technologies #61870)) and grown for 48 hours. After 48 h, cells were washed once with 180 μL KRB (Krebs Ringer Bicarbonate) buffer, and stimulated in a dose dependent manner by adding 10 μL of test compound dilution 0-1.1 mM (11 times higher concentration than final) or 10 μL of 1.1 mM Cytochalasin B solution as negative control, to 90 μL KRB buffer and incubated for 20 minutes. After compound stimulation, the transport of ¹⁴C 2-deoxy-D-glucose was started by adding of 50 μL 2-¹⁴C[U] 2-deoxy-D-glucose solution (109.1 μM 2-deoxy-D-glucose cold and 33 μM 2-¹⁴C[U] 2-deoxy-D-glucose 0.1 μCi/well) for 20 minutes. Transport was stopped by adding 50 μL/well 96 μM Cytochalasin B solution. Plates were measured in a 96-well Wallac Microbeta device. The cpm (counts per minute) values were used to determine the % inhibition values for the test compounds within each experiment. In a first step, the mean background value generated by Cytochalasin B (a potent glucose transporter inhibitor) treated cells, was subtracted from the mean values of the treated cells. All mean values were obtained from triplicates. For %-Inhibition results, the mean value of the untreated cells (just KRB) was set as 100%. All other mean values were calculated to this relation. IC₅₀ were obtained by regression analysis of dose response curve with 7 or 14 concentrations, respectively, for each compound.

Table of results:

| Example | IC₅₀ [μM] |
|---|---|
| 11 | 67.3 |
| 19 | 62.4 |
| 20 | 28.7 |
| 28 | 82.5 |
| 34 | 40.7 |
| 36 | 47.4 |
| 38 | 57.9 |
| 56 | 43.2 |
| 61 | 29.2 |
| 74 | 52.3 |
| 78 | 20.1 |
| 88 | 22.2 |
| 102 | 13.7 |
| 116 | 2.2 |
| 117 | 10.0 |
| 126 | 5.6 |
| 127 | 32.7 |
| 130 | 4.9 |
| 136 | 76.4 |
| 145 | 45.6 |
| 148 | 25.6 |
| 151 | 18.5 |
| 179 | 38.0 |
| 181 | 17.2 |
| 186 | 6.0 |
| 190 | 10.2 |
| 201 | 4.5 |
| 202a | 1.8 |
| 205 | 27.5 |
| 206 | 8.4 |
| 208 | 12.7 |
| 209 | 2.5 |
| 210 | 0.6 |
| 212 | 0.1 |
| 213 | 1.5 |
| 215 | 1.1 |
| 216 | 2.2 |
| 232 | 7.3 |
| 233 | 1.1 |
| 236 | 0.4 |
| 238 | 21.7 |
| 240 | 6.0 |
| 241 | 24.5 |
| 242 | 19.8 |
| 243 | 27.4 |
| 244 | 22.3 |
| 245 | 63.6 |
| 246 | 51.7 |
| 252 | 6.5 |
| 253 | 7.1 |
| 255 | 0.3 |
| 256 | 1.1 |
| 257 | 7.2 |
| 259 | 12.2 |
| 260 | 75.2 |
| 261 | 0.5 |
| 262 | 1.0 |
| 269 | 13.4 |
| 270 | 27.8 |
| 271 | 4.2 |
| 274 | 17.9 |
| 275 | 0.1 |
| 276 | 14.4 |
| 278 | 1.6 |
| 279 | 3.4 |
| 280 | 5.9 |
| 282 | 0.3 |
| 283 | 6.7 |
| 284 | 14.2 |
| 285 | 23.2 |
| 286 | 4.8 |
| 291 | 0.1 |
| 292 | 0.1 |
| 294 | 0.1 |
| 296 | 7.6 |
| 297 | 2.2 |
| 298 | 27.2 |
| 299 | 45.8 |
| 300 | 6.9 |
| 301 | 0.1 |
| 303 | 0.2 |
| 305 | 0.1 |
| 306 | 3.0 |
| 307 | 24.0 |
| 308 | 35.8 |
| 309 | 0.3 |
| 310 | 0.4 |
| 311 | 0.4 |
| 314 | 9.7 |
| 315 | 12.9 |
| 316 | 1.6 |
| 317 | 14.7 |
| 319 | 7.4 |
| 320 | 0.02 |
| 321 | 0.2 |
| 322 | 20.0 |
| 323 | 28.0 |
| 324 | 2.1 |
| 325 | 0.3 |
| 327 | 9.4 |
| 328 | 1.6 |
| 329 | 0.01 |
| 330 | 0.3 |
| 331 | 1.1 |
| 332 | 31.3 |
| 333 | 29.1 |
| 334 | 30.6 |
| 337 | 36.4 |
| 338 | 32.2 |
| 340 | 0.4 |
| 341 | 1.6 |
| 342 | 34.2 |
| 344 | 1.0 |
| 345 | 12.4 |
| 347 | 2.8 |
| 348 | 31.0 |
| 349 | 2.5 |
| 350 | 18.0 |
| 351 | 0.8 |
| 352 | 0.04 |
| 353 | 0.1 |
| 356 | 2.7 |
| 357 | 0.9 |
| 358 | 91.4 |
| 359 | 0.4 |
| 361 | 0.8 |
| 362 | 0.3 |
| 363 | 27.4 |
| 364 | 4.4 |
| 367 | 3.9 |
| 368 | 1.1 |
| 369 | 11.2 |
| 370 | 0.4 |
| 371 | 0.4 |
| 372 | 2.4 |
| 373 | 4.1 |
| 375 | 1.6 |
| 376 | 0.8 |
| 377 | 2.4 |
| 379 | 8.3 |
| 384 | 44.2 |
| 385 | 22.3 |
| 386 | 5.3 |
| 387 | 2.6 |
| 388 | 52.9 |
| 390 | 7.1 |
| 391 | 34.3 |
| 392 | 1.8 |
| 393 | 0.9 |
| 394 | 4.0 |
| 395 | 1.5 |
| 396 | 17.3 |
| 398 | 85.9 |
| 399 | 20.2 |
| 400 | 47.1 |
| 401 | 0.6 |
| 402 | 0.3 |
| 405 | 60.5 |
| 407 | 8.6 |
| 408 | 45.0 |
| 409 | 4.1 |
| 411 | 25.9 |
| 412 | 2.3 |
| 413 | 5.1 |
| 414 | 0.7 |
| 415 | 0.7 |
| 419 | 1.1 |
| 421 | 15.1 |
| 424 | 3.3 |
| 425 | 2.5 |
| 426 | 2.8 |
| 427 | 60.5 |
| 428 | 4.4 |
| 429 | 2.9 |
| 430 | 1.7 |
| 431 | 6.1 |
| 435 | 45.3 |
| 436 | 34.2 |
| 437 | 17.6 |
| 438 | 13.8 |
| 439 | 3.3 |
| 440 | 7.0 |
| 441 | 2.7 |
| 442 | 1.0 |
| 443 | 6.0 |
| 444 | 1.6 |
| 447 | 30.6 |
| 449 | 18.5 |
| 450 | 25.8 |
| 454 | 25.6 |
| 456 | 25.0 |
| 457 | 31.7 |
| 460 | 84.6 |
| 463 | 25.1 |
| 464 | 44.0 |
| 465 | 49.9 |
| 466 | 3.4 |
| 467 | 27.9 |
| 468 | 27.8 |
| 469 | 1.7 |
| 470 | 61.4 |
| 471 | 0.8 |
| 472 | 1.6 |
| 473 | <0.1 |
| 474 | <0.1 |
| 475 | <0.1 |
| 476 | <0.1 |
| 477 | 3.7 |
| 478 | 0.24 |
| 479 | <0.1 |
| 480 | 4.6 |
| 481 | 0.6 |
| 482 | 8.1 |
| 483 | 36.0 |
| 490 | 20% inh. at 100 μM |

2. Glucose Displacement Assay (ATP Measurement)

| Reagent | Provider | Catalogue n. |
|---|---|---|
| CellTiter-Glo ® Luminescence | Promega | G-7571 |
| Cell Viability Assay | | |
| A2780 Human Carcinoma Cell | ECACC | 93112519 |
| line | | |
| 96-well LIA plate, white | Greiner Bio-one | 655073 |
| RPMI 1640 medium GlutaMAX | Thermo Fisher | 61870 |
| | Scientitic | |
| RPMI 1640 medium (no glucose) | Thermo Fisher | 11879 |
| | Scientific | |
| Fetal Bovine Serum | Pan Biotech | P-30-3305 |
| D-(+)-Glucose solution | Sigma Aldrich | G-8644 |
| PBS buffer | Life Technologies | 14190 |
| KRB buffer | PAN | P05-32500* |
| DMSO | Sigma | D-2650 |
| Rotenone | Sigma | R-8875 |

*Customer Formulation, sterile filtered: 1.7 mM CaCl₂ x2H₂O; 1.2 mM KH₂PO₄; 4.8 mM KCl; 1.2 mM MgSO₄ × 7H₂O; 120 mM NaCl; 26 mM NaHCO₃

30,000 A2780 Human Carcinoma Cells are seeded per well in a Greiner 96-well plate. Cells are expanded and cultured in RPMI 1640 medium+GlutaMAX® with 10% FCS and 11 mM glucose, at 37° C. with 5% CO₂. After 44 h, culture media is changed and washed once with PBS to starvation media consisting of RPMI 1640 medium with 1% FCS without glucose for 2 hours. Cells are then washed with KRB buffer, followed by incubation for 20 min at 37° C. of the treatment mix consisting of 60 μL KRB buffer/well and 10 μL of compound or DMSO 10×. 10 μl of rotenone is added to the mix to a final concentration of 0.5 μM. Cell plates are left for 2 min at room temperature. 20 μL of different glucose concentrations are added to the mix—typically 0.1 to 10 mM range—.

Cells are incubated for another 15 min at 37° C., before measuring ATP with the CellTiter-Glo® Assay, under manufacturer's guidance, but without the equilibration step at room temperature for 30 min. In brief, 100 μl of Cell-Titer-Glo® Reagent is added to the wells containing already 100 μl of the previous reaction mix. Plates are mixed for 2 min at 800 rpm, followed by incubation at room temperature for 10 min to stabilize the luminescent signal. Luminescence is then recorded with the Tekan Ultra Evolution reader.

Table of results:

| | IC₅₀ [μM] at | IC₅₀ [μM] at |
|---|---|---|
| Example | 1 mM glucose | 10 mM glucose |
| 20 | 12.7 | 33.9 |
| 116 | 16.7 | 104.0 |
| 201 | 0.393 | 2.4 |
| 206 | 1.7 | >10 |
| 208 | 21.4 | >100 |
| 212 | <0.12 | 0.288 |
| 232 | 5.60 | >33 |
| 233 | 1.67 | 21.1 |
| 291 | 0.12 | 0.29 |
| 292 | 0.13 | 1.12 |
| 294 | 0.16 | 1.28 |
| 306 | 1.86 | 7.95 |
| 308 | 8.85 | >33 |
| 309 | 0.17 | 0.71 |
| 310 | 0.17 | 0.89 |
| 311 | 0.12 | 0.70 |
| 313 | 3.20 | 18.8 |
| 314 | 2.81 | 28.5 |
| 315 | 2.30 | >33 |
| 316 | 1.52 | >33 |
| 319 | 3.00 | 8.03 |
| 320 | 0.51 | 1.13 |
| 321 | 0.56 | 1.20 |
| 322 | 1.42 | 7.06 |
| 324 | 0.94 | 7.98 |
| 325 | 1.20 | 7.19 |
| 327 | 4.99 | >33 |
| 328 | 0.56 | >33 |
| 329 | 0.20 | 0.94 |
| 330 | 0.92 | 3.52 |
| 331 | 0.36 | 1.79 |
| 340 | 0.35 | 4.55 |
| 341 | 0.72 | 2.61 |
| 342 | 2.18 | >100 |
| 344 | 1.11 | 8.59 |
| 345 | 2.41 | 6.59 |
| 347 | 0.86 | 5.28 |
| 349 | 1.46 | 7.76 |
| 351 | 0.55 | 1.97 |
| 352 | 0.26 | 1.05 |
| 353 | 1.50 | 6.07 |
| 356 | 3.68 | 12.1 |
| 361 | 1.93 | 8.11 |
| 362 | 0.66 | 3.21 |
| 364 | 3.88 | 12.3 |
| 371 | 0.86 | 1.44 |
| 372 | 2.89 | 15.0 |
| 373 | 6.44 | 26.7 |
| 375 | 1.41 | 7.19 |
| 376 | 1.02 | 3.73 |
| 377 | 1.34 | 7.83 |
| 379 | 5.39 | 24.1 |
| 401 | 2.95 | 7.81 |
| 402 | 3.24 | 7.35 |
| 407 | 16.30 | >33 |
| 409 | 7.63 | 14.5 |
| 412 | 6.02 | 13.9 |
| 413 | 10.3 | 30.9 |
| 415 | 3.07 | 9.21 |
| 416 | 1.64 | 22.4 |
| 419 | 4.39 | 11.6 |
| 424 | 9.79 | 25.4 |
| 425 | 7.97 | 14.5 |
| 426 | 5.56 | 19.5 |
| 430 | 3.9 | 7.8 |
| 437 | 19.5 | >33 |
| 438 | 13 | >33 |
| 439 | 2.5 | 11.5 |
| 440 | 18 | >33 |
| 441 | 3.0 | 11.4 |
| 442 | 1.6 | 5.2 |
| 443 | 2.0 | 8.9 |
| 444 | 4.0 | 8.4 |
| 469 | 2.4 | 3.3 |
| 471 | 3.4 | 8.6 |
| 472 | 5.9 | 15.6 |

3. NanoBRET-Based GLUT1-Binding Assays in HEK Cells

Principle:

To measure binding of compounds to the hGLUT1 protein, the newly-developed NanoBRET platform from Promega is used. In this technology binding is measured via Foerster Resonance Energy Transfer. Foerster Resonance Energy Transfer is based on a direct, radiation-free energy transfer from donor to acceptor and can only take place when donor and acceptor are within nm distance. As donor energy the bioluminescence light of nanoluciferase is used, therefore this application is referred to as BRET (Bioluminescence Resonance Energy Transfer). Nanoluciferase is a protein that emits light when the appropriate substrate is available. In contrast to the firefly luciferase, nanoluciferase is not ATP dependent and thus does not compromise the cellular energy metabolism.

Nanoluciferase is attached to GLUT1 via protein complementation using the HiBit protein tagging system (Promega). To enable the protein complementation, the 11 amino acids small HiBit part of nanoluciferase is inserted into the first extracellular loop, as described originally by Kanai et al. (Kanai F., Nishioka Y., Hayashi H., et al. Direct Demonstration of Insulin-induced GLUT4 Translocation to the Surface of Intact Cells by Insertion of a c-myc Epitope into an Exofacial GLUT4 Domain. Journal of Biological Chemistry 1993; 263(19):14523-6) for GLUT4 transporter using the Myc tag. The HiBit peptide tag is used as a landing pad for the so called Large Bit protein that is commercially available from Promega and added to the medium. Large Bit has a high affinity for HiBit tag resulting in protein complementation to a fully-functional nanoluciferase, which is used as energy donor in the NanoBRET assay system.

To obtain a HEK cell line expressing HiBit-tagged GLUT1, cells were transfected with the appropriate construct placed behind the tetracycline-inducible promoter (Flipin T-Rex system from Thermo Fisher, K650001) and a cell line was generated. This cell line was checked for: (1) the correct plasma membrane localization of the tagged-GLUT1, (2) GLUT1 activity, (3) extracellular accessibility of the HiBit tag, (4) a positive and stereoselective BRET interference signal using Glucose (D-Glucose [Sigma G8769] decreases BRET, L-Glucose [Sigma-Aldrich G5500] has no effect on BRET).

As NanoBRET acceptor a GLUT1 inhibitor described by Siebeneicher et al. (Siebeneicher H., Bauser M., Buchmann B., et al. Identification of novel GLUT inhibitors. Bioorganic & Medicinal Chemistry Letters 2016; 26(7):1732-7; compound 53) was reduced to the aminobenzyl derivative and coupled to the fluorophore NanoBRET618 (Promega) and is referred to as “Bayer+NB618”.

Cell Culture:

For the assay 50 μl with 7500 cells (HiBit-tagged GLUT1 HEK cells) are plated into 384 poly-D-lysine-coated black μClear plates (Greiner) in DMEM (Gibco 61966) medium supplemented with 10% tetracycline-free FCS (PAN P30-3602) and 300 ng/ml doxycycline (Sigma 9891) to induce the induction of the HiBit-tagged GLUT1 protein.

Incubation of Compound and Acceptor Molecule

After getting adherent by overnight incubation at 37° C. and 10% CO₂, the medium is replaced by 10 μl imaging medium (1% BSA (Sigma A9576), 5 mM Hepes (Gibco 15630), 0.35 mM Na-bicarbonate (Gibco 11360-039), 1 mM Na-Pyruvate (Gibco 11360) in PBS buffer (Gibco14040)). 5 μl Bayer+NB618 (in imaging medium, final concentration 75 nM) is added and the plates are incubated for 15 minutes statically at room temperature.

A serial dilution of the test compounds is prepared in imaging medium and added in 10 μl to the respective wells. As a positive control for displacement values from wells incubated with 200 mM D-Glucose (Sigma G8769) are used. Plates are incubated for 30 minutes at room temperature without shaking.

Generating Luminescence and Measuring Fluorescence

To generate luminescence, Nano-Glo® HiBiT extracellular reagents (containing the HiBit protein and the Nanoluciferase substrate) are prepared as described by the provider; with the exception that the Nano-Glo® HiBiT extracellular reagent is used at half of the suggested concentration. This detection solution is made in the buffer provided. After addition of the detection reagent, the samples are incubated for 30 minutes without shaking and subsequently the luminescence and fluorescence emissions are measured simultaneously on the PHERAstar FSX (BMG Labtech) using the appropriate dual filter setup. This filter setup is made up of a 450-80 nm band pass filter to measure the donor signal peaking at 460 nm and a long pass filter starting at 610 nm to measure the fluorescence emission of NanoBRET 618, which peaks at 621 nm and continues beyond 700 nm.

Calculations:

From the raw luminescence and fluorescence values, the NanoBRET ratios are calculated, being the ratio of the fluorescence signal divided by the luminescence signal. Mean values are obtained from at least duplicates.

For percentage inhibition results, the mean value of the not-compound-treated cells is set as 0%. The mean BRET value of the cells treated with 200 mM glucose is used for maximal inhibition. All other mean values were calculated to this relation.

The IC₅₀ value results from the inflection point of the dose response curve obtained by measuring 10 concentrations for each compound, starting at 30 μM followed by a two-fold dilution series. Upper asymptotes are logged to 100% when inhibition reaches over 120% or beyond 80%. Lower asymptotes are logged to 0% when the starting values are between −20 and 20% inhibition. For compounds that do not reach saturation the IC₅₀ is stated as being higher than the highest concentration tested.

Measurements were done twice in duplo. Mean IC₅₀ values with their standard deviations are used.

Statistics:

Data were used from plates that have following assay statistics: S/B of 6-8, Z′ values between 0.74 and 0.83 and IC₅₀s for D-glucose of 5.33±0.56 mM (n=6).

Table of results:

| Example | IC₅₀ [μM] | StDev |
|---|---|---|
| 202a | 4.41 *^{A} | 0.51 |
| 212 | 0.24 | 0.01 |
| 305 | 2.83 | 0.27 |
| 306 | 0.19 | 0.04 |
| 320 | 0.22 | 0.03 |
| 328 | 13.6 *^{A} | 1.5 |
| 340 | 9.49 *^{A} | 1.61 |
| 367 | >30 | |
| 370 | 6.98 *^{A} | 0.37 |
| 393 | 17.16 *^{A} | 4.72 |
| 415 | 8.3 *^{A} | 0.51 |
| 416 | 9.77 *^{A} | 2.10 |

*^{A} = No clear saturation of inhibition obtained at the highest compound concentrations used.

1. A conjugate of formula (I)
P-[L₁]ₘ-[A₁]ₒ-[L₂]ₚ-[A₂]-[L₃]-[A₃]-[L₄]-S (I)
wherein P is an insulin or an insulinotropic peptide,
L₁ and L₂ are independently of each other a linker having a chain length of 1-25 atoms,
L₃ is a linker having a chain length of 2 or 3 atoms,
and L₄ is a linker having a chain length of 1, 2 or 3 atoms,
A₁, is a 5 to 6 membered monocyclic ring or a 9 to 12 membered bicyclic ring, wherein each ring is independently a saturated, unsaturated, or aromatic carbocyclic or heterocyclic ring and wherein each ring may carry at least one substituent,
A₂ and A₃ are independently of each other a 5 to 6 membered monocyclic ring or a 9 to 12 membered bicyclic ring, wherein each ring is independently an aromatic carbocyclic or aromatic heterocyclic ring and wherein each ring may carry at least one substituent,
S is a sugar moiety which binds to the insulin independent glucose transporter GLUT1, and
m, o, and p are independently of each other 0 or 1,
or a pharmaceutically acceptable salt or solvate thereof.

2. The conjugate of formula (I) of claim 1,
P-[L₁]ₘ-[A₁]ₒ-[L₂]ₚ-[A₂]-[L₃]-[A₃]-[L₄]-S (I)
wherein P is an insulin or an insulinotropic peptide,
L₁ and L₂ are independently of each other a linker having a chain length of 1-25 atoms,
L₃ is a linker having a chain length of 2 or 3 atoms,
and L₄ is a linker having a chain length of 1, 2 or 3 atoms,
A₁, is a 5 to 6 membered monocyclic ring or a 9 to 12 membered bicyclic ring, wherein each ring is independently a saturated, unsaturated, or aromatic carbocyclic or heterocyclic ring and wherein each ring may carry at least one substituent,
A₂ and A₃ are independently of each other a 5 to 6 membered monocyclic ring or a 9 to 12 membered bicyclic ring, wherein each ring is independently an aromatic carbocyclic or aromatic heterocyclic ring and wherein each ring may carry at least one substituent,
S is a sugar moiety which binds to the insulin independent glucose transporter GLUT1, and comprises a terminal pyranose moiety which is attached via position 2, 3, 4, or 6 to L₄, and
m, o, and p are independently of each other 0 or 1,
or a pharmaceutically acceptable salt or solvate thereof.

3. The conjugate of formula (I) of claim 1 or 2, wherein
L₁ and L₂ are independently of each other (C₁-C₂₅) alkylene, (C₂-C₂₅) alkenylene, or (C₂-C₂₅) alkynylene, wherein one or more C-atoms may be replaced by heteroatoms or heteroatom moieties, selected from O, NH, NH—BOC, N(C₁₋₄) alkyl, S, SO₂, O—SO₂, O-s SO₃, O—PHO₂ or O—PO₃, and/or wherein one or more C-atoms may be substituted with (C₁₋₄) alkyl, (C₁₋₄) alkyloxy, oxo, carboxyl, halogen or a phosphorus-containing group and wherein the carboxyl group may be a free carboxylic acid group or a carboxylic acid C₁-C₄ alkyl ester or a carboxamide or mono(C₁-C₄) alkyl or di(C₁-C₄) alkyl carboxamide group.

4. The conjugate of formula (I) of any one of claims 1-3, wherein
A₂ and A₃ are independently of each other a 5 to 6 membered monocyclic ring, a 9 to 12 membered bicyclic ring, wherein each ring is an aromatic carbocyclic or aromatic heterocyclic ring and wherein each ring is independently of each other unsubstituted or substituted by 1 to 4 substituents selected from halogen, NO₂, CN, CF₃, —OCF₃, (C₁₋₄) alkyl, (C₁₋₄) alkoxy, (C₁₋₄)alkyl-(C₃₋₇)cycloalkyl, (C₃₋₇) cycloalkyl, OH, benzyl, —O-benzyl, carboxyl, (C₁₋₄) alkyl-carboxylester, carboxamide, —SO₂Me, NH₂, NH—BOC or mono (C₁₋₄) alkyl, or di (C₁₋₄) alkyl carboxamide.

5. The conjugate of formula (I) of any one of claims 1-4, wherein
L₃ is selected from —CH₂—CH₂—CH₂—, —CH₂—CH₂—, —CH₂—CH₂—O—, —O—CH₂—CH₂—, —CH₂—O—, —O—CH₂—, —CO—O—, —O—CO—, —CO—NH or —NH—CO—.

6. The conjugate of formula (I) of any one of claims 1-5, wherein
A₂ is an aromatic heterocycle and A₃ is phenyl, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

7. The conjugate of formula (I) of any one of claims 1-5, wherein A₂ is phenyl and A₃ is an aromatic heterocycle, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

8. The conjugate of formula (I) of any one of claims 1-5, wherein
A₂ is phenyl and A₃ is phenyl, wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

9. The conjugate of formula (I) of any one of the preceding claims, wherein
the group -A₂-L₃-A₃-L₄- is selected from 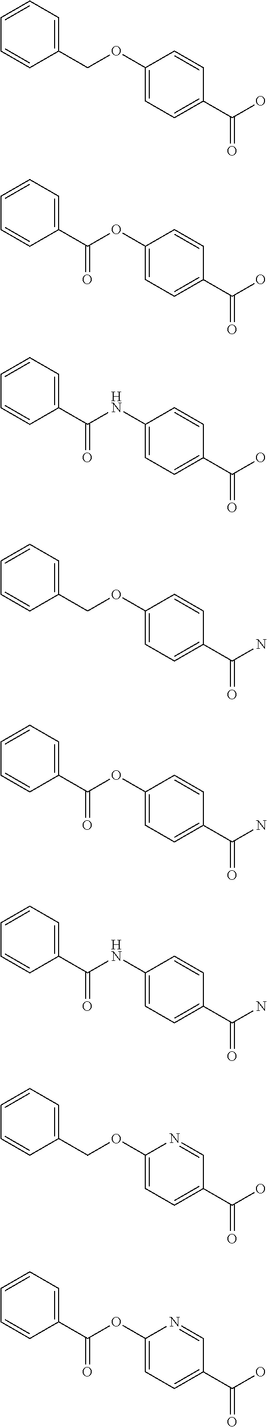 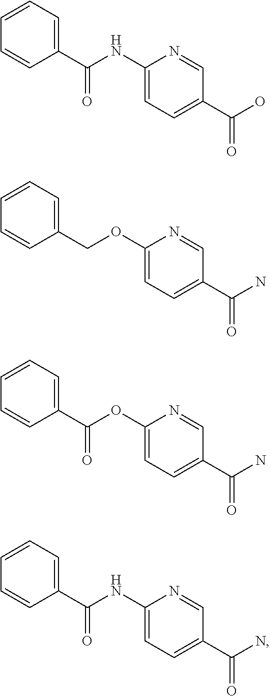
wherein each ring may be unsubstituted or carry one to four substituents selected from halogen, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

10. The conjugate of formula (I) of any one of claims 1-8, 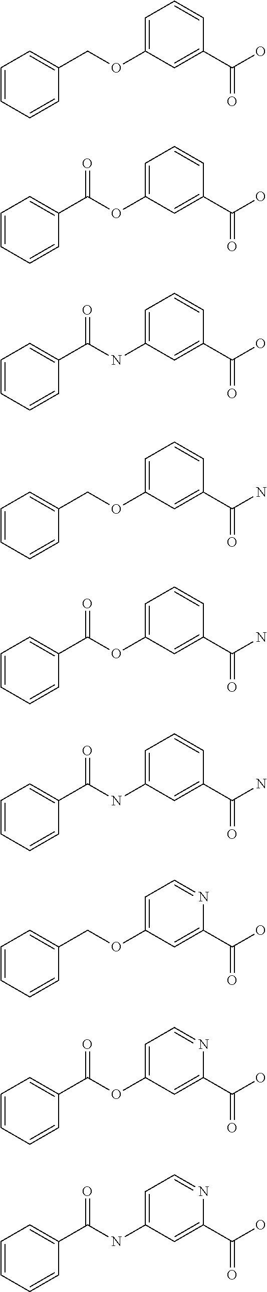 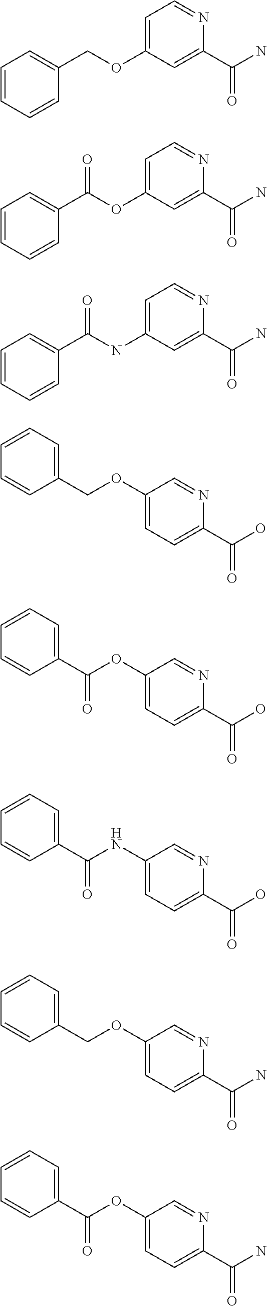 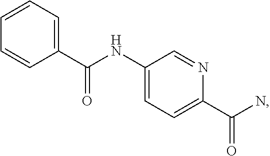
wherein each ring may be unsubstituted or carry one to four substituents selected from halogen, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

11. The conjugate of formula (I) of any one of claims 1-8, wherein
the group -A₂-L₃-A₃-L₄- is selected from 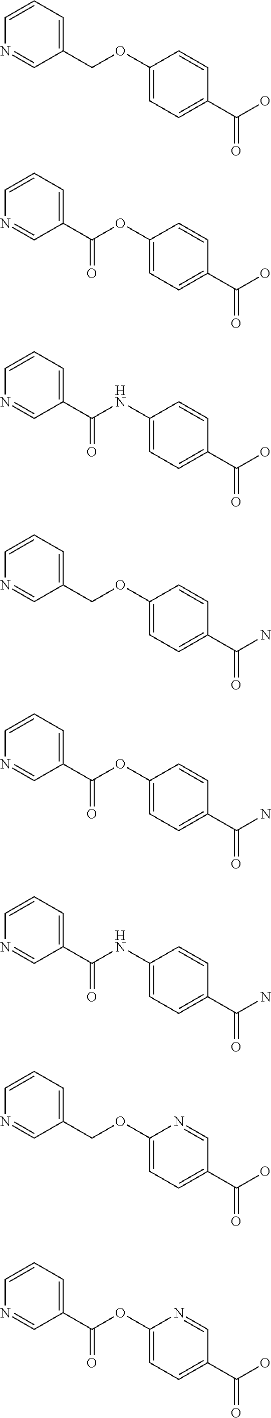 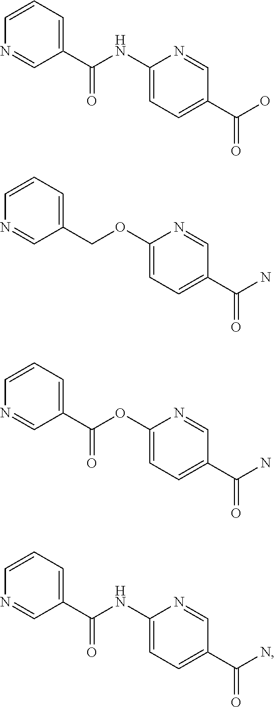
wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

12. The conjugate of formula (I) of any one of claims 1-8, wherein the group -A₂-L₃-A₃-L₄- is selected from 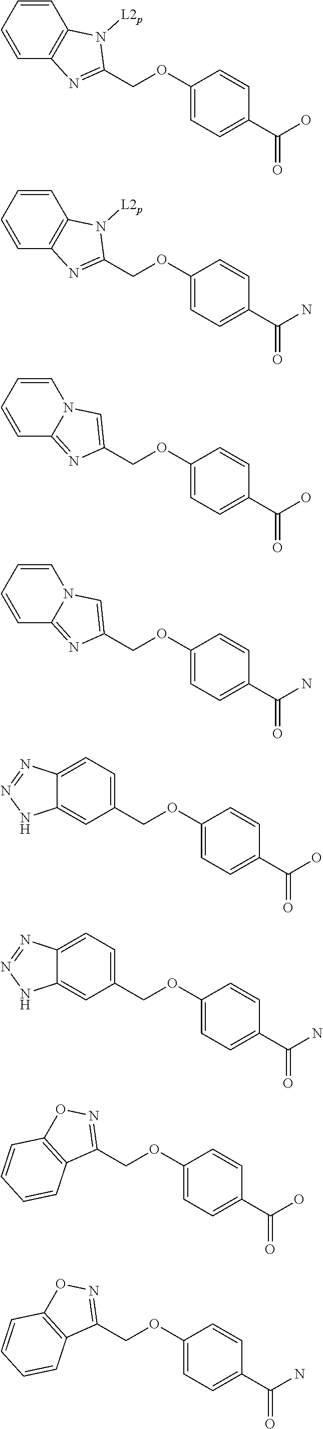
wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

13. The conjugate of formula (I) of any one of claims 1-8, wherein the group -A₂-L₃-A₃-L₄- is selected from 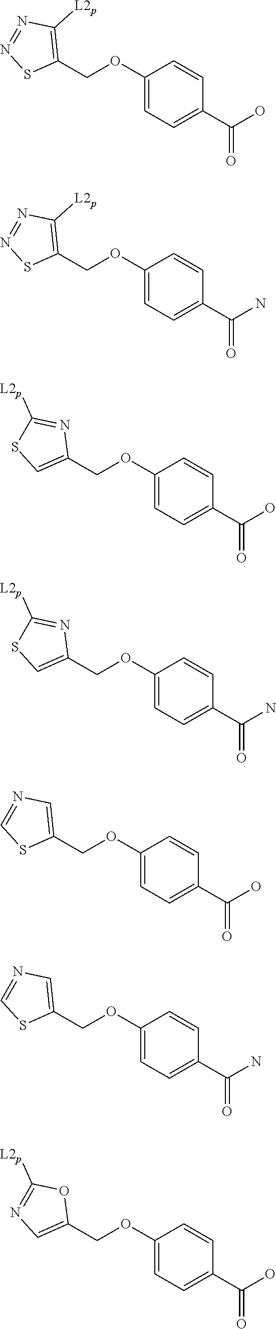 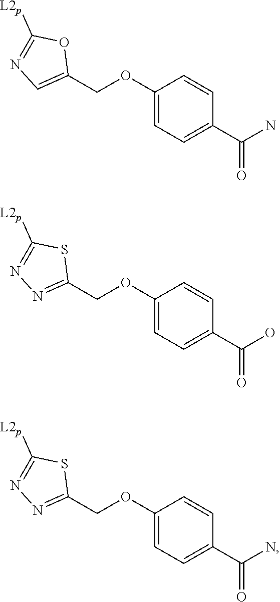
wherein each ring may be unsubstituted or carry one to four substituents selected from halogen, NO₂, NH₂, NH—BOC, CN, (C₁₋₄) alkyl, (C₁₋₄) alkoxy, OH, CF₃, OCF₃, carboxyl, (C₁₋₄) alkyl-carboxylester, carboxamide, or mono (C₁₋₄) alkyl, or di (C₁₋₄) alkyl carboxamide or —SO₂—(C₁₋₄)-alkyl.

14. The conjugate of formula (I) of any one of the preceding claims, wherein
m is 1, o is 1 and p is 1.

15. The conjugate of formula (I) of any one of the preceding claims, wherein m is 1, o is 0 and p is 0.

16. The conjugate of formula (I) of any one of the preceding claims, wherein
S is a terminal pyranose moiety and S is attached via position 3 to L₄.

17. The conjugate of formula (I) of any one of the preceding claims,
S is a terminal pyranose moiety and S is attached via position 4 to L₄.

18. The conjugate of formula (I) of any one of the preceding claims, wherein
S is a terminal pyranose moiety and S is attached via position 6 to L₄.

19. The conjugate of formula (I) of any one of the preceding claims, wherein
S is a terminal pyranose moiety and S is attached via position 2 to L₄.

20. The conjugate of formula (I) of any one of the preceding claims, wherein
S is a terminal pyranose moiety S1 having a backbone structure of Formula (II) 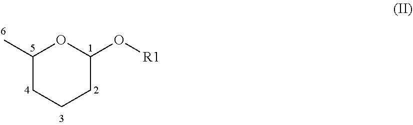
wherein 1, 2, 3, 4, 5, and 6 denote the positions of the C-atoms in the pyranose moiety,
R1 is H or a protecting group,
and wherein S1 is attached via position 2, 3, 4, or 6 to L₄.

21. The conjugate of formula (I) of claim 20, wherein
S1 is of the Formula (III): 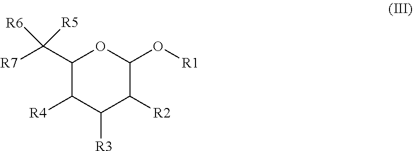
wherein R1 is H or a protecting group such as methyl or acetyl,
R2 and R7 are OR8, or NHR8 or an attachment site to L₄ wherein R8 is H or a protecting group such as acetyl or benzyl,
R3 and R4 are OR8 or an attachment site to L₄, wherein R8 is H or a protecting group such as acetyl or benzyl,
or R1 and R2 and/or R3 and R4 form together with the pyranose ring atoms to which they are bound a cyclic group, e.g. an acetal,
R5 and R6 are H or together form together with the carbon atom to which they are bound a carbonyl group, and
wherein one of R2, R3, R4, and R7 is the attachment site to L₄.

22. The conjugate of formula (I) of any one of claims 20-21, wherein
S1 is of formula (IVa) or (IVb): 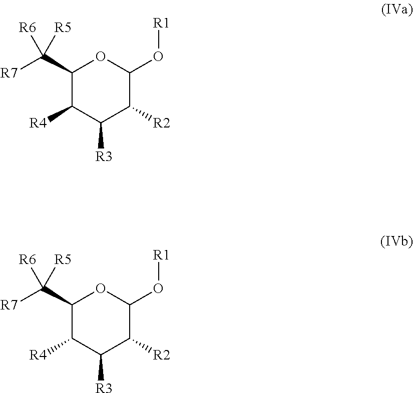
wherein R1, R2, R3, R4, R5, R6, and R7 are defined as in claim 18 or 19.

23. The conjugate of formula (I) of any one of the preceding claims, wherein
S has a structure of formula (V):
—[S2]_{S}-S1 (V)
wherein
S2 is a mono- or disaccharide moiety, particularly comprising at least one hexose or pentose moiety,
S1 is a terminal pyranose moiety as defined in claims 20 to 22, and
s is 0 or 1.

24. The conjugate of formula (I) of claim 23, wherein
S2 is of formula (VIa) or (VIb): 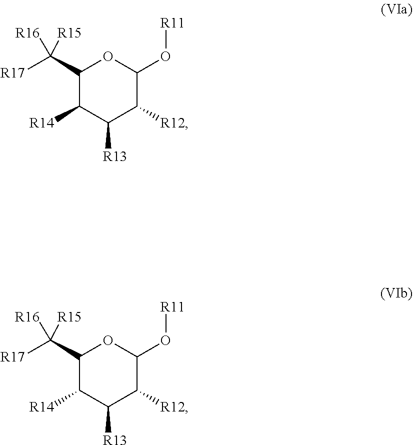
wherein R11 is a bond to S1,
R12 and R17 are OR8 or NHR8 or an attachment site to L₄, wherein R8 is H or a protecting group such as acetyl or benzyl,
R13 and R14 are OR8 or an attachment site to L₄, wherein R8 is H or a protecting group such as acetyl,
R15 and R16 are H or together form with the carbon atom to which they are bound a carbonyl group,
or R11 and R12 and/or R13 and R14 form together with the ring atoms to which they are bound a cyclic group such as an acetal,
and wherein one of R12, R13, R14, and R17 is an attachment site to L₄.

25. The conjugate of formula (I) of any one of claims 20 to 24, wherein the terminal pyranose moiety S1 is selected from glucose and galactose derivatives,
wherein the terminal pyranose moiety S1 is attached via position 2, 3, 4, or 6 to L₄.

26. The conjugate of formula (I) of any one of claims 23-25, the saccharide moiety S2 is a pyranose moiety, selected from glucose and galactose.

27. The conjugate of formula (I) of any one of the preceding claims, which has an affinity of 10-500 nM to the insulin independent glucose transporter GLUT1.

28. The conjugate of formula (I) of any one of the preceding claims which reversibly binds to the insulin independent glucose transporter GLUT1 dependent from the glucose concentration in the surrounding medium.

29. The conjugate of formula (I) of any one of the preceding claims, wherein the sugar moiety S comprises a single terminal saccharide moiety.

30. The conjugate of formula (I) of any one of the preceding claims for use in medicine, particularly in human medicine.

31. The conjugate of formula (I) of any one of claims 1-29 for use in the prevention and/or treatment of disorders associated with, caused by and/or accompanied by a dysregulated glucose metabolism.

32. The conjugate of formula (I) of any one of claims 1-29 for use in the prevention and/or treatment of diabetes, particularly of diabetes type 2 or of diabetes type 1.

33. A pharmaceutical composition comprising a conjugate of formula (I) of any one of claims 1-29 as an active agent and pharmaceutically acceptable carrier.

34. A method of preventing and/or treating a disorder associated with, caused by and/or accompanied by a dysregulated glucose metabolism, comprising administering a conjugate of formula (I) of any one of claims 1-29 or a composition of claim 33 to a subject in need thereof.

35. A compound of formula (Ia)
R—(O═C)-[L₁]ₘ-[A₁]ₒ-[L₂]₂-[A₂]-[L₃]-[A₃]-[L₄]-S (Ia)
wherein L₁, L₂, L₃, L₄, A₁, A₂, A₃, S, m, o and p are defined as in any one of claims 1-28,
R is H, halogen, OH, O-alkyl-, an anhydride forming group or another active ester forming group, like 4-nitrophenylester, succinate or N-hydroxy benzotriazol,
or a pharmaceutically acceptable salt or solvate thereof.

36. A compound of formula (Ib)
[L₁]ₘ-[A₁]ₒ-[L₂]ₚ-[A₂]-[L₃]-[A₃]-[L₄]-S (Ib)
wherein L₁, L₂, L₃, L₄, A₁, A₂, A₃, S, m, o and p are defined as in any one of claims 1-29, or a pharmaceutically acceptable salt or solvate thereof.

37. The compound of formula (Ib) according to claim 36, wherein the sugar moiety S comprises a terminal pyranose moiety which is attached via position 2, 3, 4, or 6 to L₄.
